(19) United States
(12) Patent Application Publication
(10) Pub. No.: US 2026/0276661 A1
(43) Pub. Date: Sep. 17, 2026

(54) METHODS OF ASSESSING METABOLIC HEALTH

(71) Applicant: Baker Heart and Diabetes Institute, Melbourne (AU)

(72) Inventors: Peter John MEIKLE, Lower Plenty (AU); Kevin HUYNH, Sunshine North (AU); Corey GILES, Patterson Lakes (AU); Thomas Geoffrey MEIKLE, South Yarra (AU); Jingqin WU, Carlton (AU); Tingting WANG, Eltham (AU); Habtamu BEYENE, Caulfield North (AU); Aleksandar DAKIC, Elwood (AU)

(21) Appl. No.: 19/167,032

(22) PCT Filed: Mar. 20, 2024
(86) PCT No.: PCT/AU2024/050249

§ 371 Date: Sep. 19, 2025

(30) Foreign Application Priority Data
Mar. 20, 2023 (AU) 2023900769

(51) Int. Cl. G01N 33/92; A61B 5/00; G01N 30/72; G16H 50/30

(52) U.S. Cl. CPC G01N 33/92; A61B 5/7275; G01N 30/7233; G16H 50/30; G01N 2405/04; G01N 2570/00; G01N 2800/042; G01N 2800/32; G01N 2800/50

(57) ABSTRACT

The present invention provides a method for assessing metabolic health in an individual.

### FIELD

The present disclosure generally relates to lipidomics and methods of assessing metabolic health based on a plurality of lipid biomarkers.

### BACKGROUND

Bibliographic details of references referred to by author name in the subject specification are listed at the end of the specification.

Reference to any prior art in this specification is not, and should not be taken as, acknowledgement or any form of suggestion that this prior art forms part of the common general knowledge in any country.

Despite lipid profiling still lagging behind the advances made in the study and application of other types of biomarkers such as nucleic acid and protein biomarkers, lipid biomarkers are increasingly studied for their potential to act as important biomarkers. In particular, levels of certain plasma lipid species have been associated with metabolic health, and with cardiovascular, neurodegenerative, and inflammatory diseases.

There is a need for alternative methods for assessing metabolic health in a subject based on lipidomics.

### SUMMARY

In an aspect of the invention, there is provided a method of determining metabolic health of a subject, the method comprising:
(i) detecting in a biological sample from the subject a level of at least two lipid species selected from Table 4 and/or 6; and
(ii) comparing the levels of the lipid species detected in the biological sample to reference lipid species levels, wherein the comparison is determinative of the metabolic health of the subject.

In one embodiment, the lipid species is selected from one or more lipid classes or sub-classes comprising or consisting of any one or more of LPC, PC, AC, SM, Cer, LPC(O), DG, PE(P), LPE, TG, HexCer, CE, TG(O), S1P, PC(O), and Hex2Cer. In another embodiment, the one or more lipid classes or sub-classes comprise or consist of any one or more of LPC, PC, AC, SM, Cer, LPC(O), DG, PE(P), and LPE.

In one embodiment, the lipid class or subclass LPC comprises or consists of any one or more lipid species of 18:2, 20:0, 18:0, and 22:6.

In one embodiment, the lipid class or subclass PC comprises or consists of any one or more lipid species of 15:0, 40:8, 34:5, 16:0, 36:2, 33:2, and 33:1.

In one embodiment, the lipid class or subclass AC comprises or consists of any one or more lipid species of 14:2, 14:1, 16:1, 13:0, 18:1, and 14:0.

In one embodiment, the lipid class or subclass SM comprises or consists of any one or more lipid species of d18:1/14:0, d16:1/16:0, d17:1/16:0, d17:1/14:0, d18:0/18:1, and d16:1/20:0.

In one embodiment, the lipid class or subclass Cer comprises or consists of any one or more lipid species of d18:4/24:0 and m18:0/22:0.

In one embodiment, the lipid class or subclass LPC(O) comprises or consists of any one or more lipid species of 24:2.

In one embodiment, the lipid class or subclass DG comprises or consists of any one or more lipid species of 18:1/18:1, 18:2/18:2, and 16:0/16:0.

In one embodiment, the lipid class or subclass PE(P) comprises or consists of any one or more lipid species of 18:0/22:6 and 16:0/22:4.

In one embodiment, the lipid class or subclass LPE comprises or consists of any one or more lipid species of 16:0, 22:6, and 18:1.

In one embodiment, the lipid class or subclass TG comprises or consists of any one or more lipid species of 48:3, 54:6, and 52:2.

In one embodiment, the lipid class or subclass HexCer comprises or consists of any one or more lipid species of d18:1/24:1.

In one embodiment, the lipid class or subclass CE comprises or consists of any one or more lipid species of 20:2 and 15:0.

In one embodiment, the lipid class or subclass TG(O) comprises or consists of any one or more lipid species of 50:1.

In one embodiment, the lipid class or subclass S1P comprises or consists of any one or more lipid species of d18:1.

In one embodiment, the lipid class or subclass PC(O) comprises or consists of any one or more lipid species of 36:5.

In one embodiment, the lipid class or subclass Hex2Cer comprises or consists of any one or more lipid species of d16:1/16:0.

In an embodiment, the method further comprises a step of assessing metabolic health in the subject based on the comparison of the lipid species in the biological sample to reference lipid species levels.

In an embodiment, metabolic health of the subject is at least one of:
(i) metabolic age;
(ii) metabolic body mass index (mBMI);
(iii) 2 hr post load glucose level;
(iv) plasmalogen level or plasmalogen relative to the phospholipid level;
(v) risk of cardiovascular disease; and
(vi) risk of diabetes.

In an embodiment, the metabolic health of a subject is determined based on a cardiovascular disease risk score. In one embodiment, the metabolic health of a subject is determined based on a diabetes risk score. In one embodiment, the metabolic health of a subject is determined based on a glucose intolerance risk score.

In one embodiment, the risk scores are used to calculate a subject's metabolic health are adjusted for age. In one embodiment, the risk scores are used to calculate a subject's metabolic health are adjusted for gender. In one embodiment, the risk scores are used to calculate a subject's metabolic health are adjusted for age and gender.

In one embodiment, the metabolic health of a subject is determined based on their metabolic BMI. In one embodiment, the metabolic health of a subject is determined based on their metabolic age.

In one embodiment, the metabolic health of a subject is determined based on their level of plasmalogen, either plasmalogen levels of the ratio of plasmalogens to phospholipids or phospholipids to plasmalogen.

In one embodiment, the metabolic health of a subject is determined based on any combination of one or more or all of metabolic age (mAge); metabolic body mass index (mBMI); plasmalogen score (PS); risk of developing cardiovascular disease (CVD); risk of developing glucose intolerance; and/or risk of developing diabetes (e.g. type 2 diabetes, T2D).

In one embodiment, the metabolic health of a subject is determined based on any combination of one or more of metabolic age (mAge); metabolic body mass index (mBMI); plasmalogen score (PS); risk of developing cardiovascular disease (CVD); risk of developing glucose intolerance; and/or risk of developing diabetes (e.g. type 2 diabetes, T2D) and adjusted for age, gender, or age and gender or any combination of disease risk factors, including but not limited to age, sex, BMI, waste circumference, diabetes status, cholesterol, glucose tolerance, HDL-cholesterol, triglycerides, blood pressure, blood pressure medication, fasting glucose, HbA1C, ethnicity, family history of disease, smoking status, exercise levels, diet, place of birth.

In another aspect, there is provided method of determining the metabolic body mass index (mBMI) of a subject, the method comprising:
(i) detecting in a biological sample from the subject a level of at least two lipid species selected from Table 4 and/or 6; and
(ii) comparing the levels of the lipid species detected in the biological sample to reference lipid species levels;
   wherein the comparison is determinative of the mBMI of the subject.

In an embodiment, the method further comprises a step of assessing mBMI in the subject based on the comparison of the lipid species in the biological sample to reference lipid species levels.

In another aspect, there is provided method of calculating a cardiovascular disease (CVD) risk score of a subject, the method comprising:
(i) detecting in a biological sample from the subject a level of at least two lipid species selected from Table 4 and/or 6; and;
(ii) comparing the levels of the lipid species detected in the biological sample to reference lipid species levels;
   wherein the comparison is determinative of the risk of developing cardiovascular disease in the subject.

In another embodiment, the method further comprises:
(i) standardising each continuous variable;
(ii) log transforming each lipid concentration; and
(iii) further standardising the variables prior to their use as the predictors of a CVD event.

In an embodiment, the method further comprises a step of confirming that the subject has, or is likely to develop cardiovascular disease based on the comparison of the lipid species in the biological sample to reference lipid species levels.

In another aspect, there is provided a method of treating a subject at risk of cardiovascular disease, wherein the method comprises:
(i) detecting in a biological sample from the subject a level of at least two lipid species selected from Table 4 and/or 6; and
(ii) comparing the levels of the lipid species detected in the biological sample to reference lipid species levels of the lipid species; and
(iii) treating the subject if the risk score confirms that the subject has or is likely to develop cardiovascular disease.

In an embodiment, the treatment comprises blood-thinning medicine, statins, beta blockers, nitrates, angiotensin-converting enzyme (ACE) inhibitors, angiotensin-2 receptor blockers, calcium channel blockers and diuretics.

In an embodiment, the method comprises further assessment such as blood tests, electrocardiogram (ECG), exercise stress test, echocardiogram (ultrasound), nuclear cardiac stress test, coronary angiogram, magnetic resonance imaging (MRI), coronary computed tomography angiogram (CCTA) and CT scans.

In an embodiment, the treatment comprises lifestyle changes including changes to diet, exercise regime, aerobic activity, quitting or reducing smoking, and weight loss, weight loss surgery.

In another aspect, there is provided a method of generating a lipid profile in a sample from a subject, the method comprising:
(i) detecting in a biological sample from the subject a level of at least two lipid species selected from Table 4 and/or 6,
   wherein the lipid species detected in the biological sample can be compared to reference levels of the lipid species such that the comparison is determinative of the metabolic health of the subject.

In another aspect, there is provided a method of calculating a metabolic health score of a subject, the method comprising:
(i) refining a discriminatory power of one or more lipid species by statistically weighting one or several of the numeric values associated therewith according to a predetermined equation; and
(ii) normalising the resulting metabolic health score of the subject to a reference population,
   wherein lipid profile data can be obtained from a biological sample taken from the subject; and
   wherein the numeric values of the lipid profile data can be normalised against a reference sample.

In an embodiment, the method optionally comprises use of an algorithm as defined herein, preferably in any one of Tables 8-11, 13 or 14. In another embodiment, the algorithms can be prepared from any subset of the lipids defined in Table 4 and/or 6.

In another aspect, there is provided a method of determining metabolic health of a subject, the method comprising:
(i) comparing levels of at least two lipid species detected in a biological sample of the subject to reference levels of the lipid species, wherein the comparison is determinative of the metabolic health of the subject,
   wherein the levels of at least two lipid species selected from Table 4 and/or 6 can be detected in the biological sample from the subject.

In another aspect, there is provided a method of determining the risk of developing diabetes or impaired glucose tolerance in a subject, the method comprising:
(i) detecting in a biological sample from the subject a level of at least two lipid species selected from Table 4 and/or 6; and
(ii) comparing the levels of the lipid species detected in the biological sample to reference lipid species levels;
   wherein the comparison is determinative of the risk of developing diabetes or impaired glucose tolerance in the subject.

In an embodiment, the method further comprises a step of confirming that the subject has, or is likely to develop diabetes or impaired glucose tolerance based on the comparison of the lipid species in the biological sample to reference lipid species levels.

In another aspect, there is provided a method of treating a subject at risk of diabetes or impaired glucose tolerance, wherein the method comprises:
(i) detecting in a biological sample from the subject a level of at least two lipid species selected from Table 4 and/or 6; and;
(ii) comparing the levels of the lipid species detected in the biological sample to lipid species reference levels;
(iii) treating the subject if the risk score confirms that the subject is likely to develop diabetes or impaired glucose tolerance.

In an embodiment, the treatment comprises insulin pumps, islet cell transplant, tablets and medication, weight loss including weight loss surgery, diet and exercise, insulin treatment, and emotional and mental health support.

In an embodiment, the diabetes is type II diabetes.

In another aspect, there is provided a method of determining metabolic age of a subject, the method comprising:
(i) detecting in a biological sample from the subject a level of at least two lipid species selected from Table 4 and/or 6; and
(ii) comparing the levels of the lipid species detected in the biological sample to reference lipid species levels,
   wherein the comparison is determinative of the metabolic age in the subject.

In an embodiment, the method further comprises a step of confirming metabolic age in the subject based on the comparison of the lipid species in the biological sample to reference lipid species levels.

In an embodiment, the method further comprises detecting in a biological sample from the subject a level of at least 10 lipid species, at least 20 lipid species, at least 50 lipid species or at least 100 lipid species selected from Table 4 and/or 6.

In an embodiment, the method further comprises detecting in the biological sample from the subject a level of at least one additional lipid species not defined in Table 4 and/or 6.

In another aspect, there is provided a method of calculating a metabolic health score of a subject comprising:
(i) obtaining lipid profile data from a biological sample taken from the subject;
(ii) normalising the numeric values of the lipid profile data against a reference sample; and
(iii) refining the discriminatory power of one or more lipid species by statistically weighting one or several of the numeric values associated therewith according to a predetermined equation; and
(iv) normalising the resulting metabolic health score of the subject to a reference population.

In another aspect, there is provided a method of calculating a metabolic health score of a subject comprising:
(i) obtaining lipid profile data from a biological sample taken from the subject;
(ii) normalising the numeric values of the lipid profile data against a reference sample; and
(iii) refining the discriminatory power of one or more lipid species by statistically weighting one or several of the numeric values associated therewith according to a predetermined equation;
(iv) optionally adding one or more additional, statistically weighted, risk factors into the model, taken from the list of, age, sex, BMI, waste circumference, diabetes status, cholesterol, glucose tolerance, HDL-cholesterol, triglycerides, blood pressure, blood pressure medication, fasting glucose, HbA1C, ethnicity, family history of disease, smoking status, exercise levels, diet, place of birth; and
(iv) normalising the resulting metabolic health score of the subject to a reference population

In an embodiment, the lipid profile data comprises two or more lipid species selected from the group set forth in one or more of Tables 3, 4, 5, and 6.

In an embodiment, the method further comprises calculating the sum of the weighted lipids.

In one aspect, provided herein is a method of calculating a metabolic health score of a subject, the method comprising:
(i) refining a discriminatory power of one or more lipid species by statistically weighting one or several of the numeric values associated therewith according to a predetermined equation; and
(ii) normalising the resulting metabolic health score of the subject to a reference population,
   wherein lipid profile data can be obtained from a biological sample taken from the subject; and wherein the numeric values of the lipid profile data can be normalised against a reference sample.

In an embodiment, the metabolic health score is at least one of:
(i) metabolic age;
(ii) metabolic body mass index (mBMI);
(iii) 2 hr post load glucose level;
(iv) plasmalogen level or plasmalogen relative to the phospholipid level;
(v) risk of cardiovascular disease; and
(vi) risk of diabetes.

In an embodiment, the biological sample is selected from the group consisting of blood, plasma, serum, dried blood spots and dried plasma spots.

In another aspect, there is provided a method of generating a lipid profile in a sample from a subject, the method comprising:
(i) detecting in a biological sample from the subject a level of at least two lipid species selected from Table 4 and/or 6,
   wherein the lipid species detected in the biological sample can be compared to reference lipid species levels such that the comparison is determinative of the mBMI of the subject.

In another aspect, there is provided a method of determining metabolic body mass index (mBMI) of a subject, the method comprising:
(i) comparing levels of at least two lipid species detected in a biological sample of the subject to reference lipid species levels, wherein the comparison is determinative of the mBMI of the subject,
   wherein the levels of at least two lipid species selected from Table 4 and/or 6 can be detected in the biological sample from the subject.

In another aspect, there is provided a method of generating a lipid profile in a sample from a subject, the method comprising:
(i) detecting in a biological sample from the subject a level of at least two lipid species selected from Table 4 and/or 6, wherein the lipid species detected in the biological sample can be compared to reference lipid species levels such that the comparison is determinative of a cardiovascular disease (CVD) risk score of the subject.

In another aspect, there is provided a method of calculating a cardiovascular disease (CVD) risk score of a subject, the method comprising:
(i) comparing levels of at least two lipid species detected in a biological sample of the subject to reference lipid species levels, wherein the comparison is determinative of the cardiovascular disease (CVD) risk score of the subject, wherein the levels of at least two lipid species selected from Table 4 and/or 6 can be detected in the biological sample from the subject.

In another aspect, there is provided a method of generating a lipid profile in a sample from a subject, the method comprising:
(i) detecting in a biological sample from the subject a level of at least two lipid species selected from Table 4 and/or 6, wherein the lipid species detected in the biological sample can be compared to reference lipid species levels such that the comparison is determinative of the presence or risk of developing diabetes or impaired glucose tolerance of the subject.

In another aspect, there is provided a method of determining the presence or risk of developing diabetes or impaired glucose tolerance in a subject, the method comprising:
(i) comparing levels of at least two lipid species detected in a biological sample of the subject to reference lipid species levels, wherein the comparison is determinative of the presence or risk of developing diabetes or impaired glucose tolerance in the subject,
   wherein the levels of at least two lipid species selected from Table 4 and/or 6 can be detected in the biological sample from the subject.

In another aspect, there is provided a method of generating a lipid profile in a sample from a subject, the method comprising:
(i) detecting in a biological sample from the subject a level of at least two lipid species selected from Table 4 and/or 6,
   wherein the lipid species detected in the biological sample can be compared to reference lipid species levels such that the comparison is determinative of metabolic age of the subject.

In another aspect, there is provided a method of determining metabolic age of a subject, the method comprising:
(i) comparing levels of at least two lipid species detected in a biological sample of the subject to reference lipid species levels, wherein the comparison is determinative of the metabolic age of the subject,
   wherein the levels of at least two lipid species selected from Table 4 and/or 6 can be detected in the biological sample from the subject.

In one embodiment, the detection of a plurality or population of lipid species in the subject according to any method described herein is performed using a high throughput lipidomics assay.

In one embodiment, the detection of a plurality or population of lipid species in the subject according to any method described herein is performed using a high throughput lipidomics assay wherein the total run time is less than 15 minutes.

In one embodiment, the detection of a plurality or population of lipid species in the subject according to any method described herein is performed using a high throughput lipidomics assay wherein the total run time is not more than 5 minutes.

In one embodiment, the detection of a plurality or population of lipid species in the subject according to any method described herein is performed using a high throughput lipidomics assay wherein the total run time is less than 5 minutes.

In one embodiment, the detection of a plurality or population of lipid species in the subject according to any method described herein is performed using a high throughput lipidomics assay, wherein the assay is liquid chromatography with tandem mass spectrometry and the total run time is less than 15 minutes.

In one embodiment, the detection of a plurality or population of lipid species in the subject according to any method described herein is performed using a high throughput lipidomics assay, wherein the assay is liquid chromatography with tandem mass spectrometry and the total run time is not more than 5 minutes.

In one embodiment, the detection of a plurality or population of lipid species in the subject according to any method described herein is performed using a high throughput lipidomics assay, wherein the assay is liquid chromatography with tandem mass spectrometry and the total run time is less than 5 minutes.

In one embodiment, the lipids analysed to generate a metabolic health score, a cardiovascular risk score, a diabetes risk score, a glucose intolerance risk score, and a plasmalogen score are selected based on their suitability for high throughput analysis, including of 5 minutes or less, including by liquid chromatography with tandem mass spectrometry.

In one embodiment, the lipids analysed to determine metabolic health, including by calculating a metabolic BMI, a metabolic age, a cardiovascular risk score, a diabetes risk score, a glucose intolerance risk score, and a plasmalogen score are selected based on their suitability for high throughput analysis, including of 5 minutes or less, including by liquid chromatography with tandem mass spectrometry.

In another aspect, there is provided a lipidomics assay for determining metabolic health of a subject according to a method described herein, the method comprising: (i) separating in a sample obtained from a subject the sample analytes by liquid chromatographic separation; (ii) analysing the analytes using a mass spectrometer; wherein the total run time is less than 15 minutes; and wherein the resulting data can accurately assign individual analytes to a single lipid species.

In one embodiment, the run time of the lipidomics assay is not more than 5 minutes.

In one embodiment, the biological sample of the lipidomics assay is at least one of plasma, serum, dried blood spots, and dried plasma spots.

Panels or kits or compositions for use in the methods described herein are also provided. In an embodiment, the kit comprises one or more or all of:
a set of stable labelled isotopes or non-physiological lipid standards for quantification of the lipid species;
reference plasma samples for standardisation of the resulting lipid measures.

It will be appreciated that other aspects, embodiments, or examples, of the methods and/or lipid biomarkers may be provided according to any aspects, embodiments, or examples thereof as described below and herein.

Any embodiment herein shall be taken to apply mutatis mutandis to any other embodiment unless specifically stated otherwise.

The present invention is not to be limited in scope by the specific embodiments described herein, which are intended for the purpose of exemplification only.

Functionally-equivalent products, compositions and methods are clearly within the scope of the invention, as described herein.

The invention is hereinafter described by way of the following non-limiting Examples and with reference to the accompanying FIGURES.

### BRIEF DESCRIPTION OF ACCOMPANYING DRAWINGS

FIG. 1. Stepped, linear solvent gradient for Research Protocol LC-MS/MS. Flow rates and percentage solvent B is detailed in the table and visually depicted in the graph. Briefly, a flow rate of 0.4 ml/minute at 10% solvent B increased to 45% solvent B over 2.7 minutes, then to 53% over 0.1 minutes, to 65% over 6.2 minutes, to 89% over 0.1 minute, to 92% over 1.9 minutes and finally to 100% over 0.1 minute. The solvent was then held at 100% B for 0.8 minutes. Equilibration was as follows, solvent was decreased from 100% B to 10% B over 0.1.

FIG. 2. Stepped, linear solvent gradient for Clinical Protocol LC-MS/MS. Flow rates and percentage solvent B is detailed in the table and visually depicted in the graph. Briefly, flow rate was initiated at 1 ml/minute, 0% B, maintained until 0.3 min, at which point % B was increased to 40% over 0.1 min. % B was further increased linearly to 48% B at 2.5 min, then stepped to 58% B at 2.51 min. % B was increased linearly to 60% at 3.1 min, then stepped to 76% B at 3.11, before increasing linearly to 80% B at 4.4 min. At 4.41 min the % B was increased to 100%, maintained until 5 min, then immediately dropped to 0% B at 5.01 for equilibration.

FIG. 3. Overview of the study design for the development of metabolic body mass index (mBMI) scores using CLP and the subsequent downstream analyses. Either the Research Lipid List (717 species, Table 2) or CLP1s (Table 4) or CLP2s (Table 6) lists were used for the generation of the metabolic BMI (mBMI) score using data from the Australian Diabetes, Obesity and Lifestyle Study (AusDiab) cohort (n=10,339) with a ridge regression model. The downstream analyses, consisting of association of resulting mBMIΔ with cardio-metabolic traits and/or diseases were subsequently performed. Abbreviations: T2DM, type 2 diabetes mellitus; CVD, cardiovascular disease; CVE, cardiovascular event; IHD, ischemic heart disease; LC-MS/MS, liquid chromatography tandem mass spectrometry.

FIG. 4: The performance of ridge and LASSO models. (A) The number of features incorporated in the ridge (red line) and LASSO (blue line) models for different lambda values. (B) The correlation (R2) of BMI and pBMI (dashed lines) or BMI and mBMI (solid lines) in ridge (red line) and LASSO models (blue line) for different lambda values. (C) MSE of the difference between the observed and predicted values for ridge (red line) and LASSO models (blue line). The vertical dashed red and blue lines represent the minimum MSE, for ridge and LASSO models respectively (i.e. the optimum lambda used to make the models). (D) A plot of beta coefficients from the optimum ridge model. (E) A plot of beta coefficients from the optimum LASSO model.

FIG. 5: The correlation among pBMI or mBMI scores derived using the full lipidome and the CLP. (A, B) The correlation of pBMI (full lipidome) with pBMI (CLP1s) and pBMI (CLP2s). (C) The correlation of pBMI (CLP2s) with pBMI (CLP1s). (D, E) The correlation between mBMI (full lipidome) and mBMI (CLP1s) or mBMI (CLP2s) respectively. (F) The correlation between mBMI (CLP1s) and mBMI (CLP2s).

FIG. 6: Modelling of the metabolic BMI score using the CLP or the full lipidome and comparison of the captured lipid biology with BMI. (A-C) Associations of mBMIΔ (full lipidome), mBMIΔ (CLP1s) and mBMIΔ (CLP2s) with plasma lipid species and (D) Association of BMI with plasma lipid species using linear regression analysis adjusting for age and sex. Grey open circles show species (p>0.05), grey and dark closed circles show species with p<0.05 after correction for multiple comparisons using the method of Benjamini and Hochberg. Dark circles, diamonds, triangles and squares represent the top 15 most significant lipids associated with mBMIΔ (full lipidome) and mBMIΔ (CLP1s), mBMIΔ (CLP2s) and BMI respectively. The whiskers represent 95% confidence intervals.

FIG. 7: Similarity of the biological signal captured by mBMIΔ (CLP), mBMIΔ (full lipidome) and BMI. (A-B) The correlation of effect sizes of each lipid associated with mBMIΔ (full lipidome) and mBMIΔ (CLP1s) and mBMIΔ (CLP2s) respectively. (C) The correlation between effect sizes of each lipid associated with mBMIΔ (CLP1s) and mBMIΔ (CLP2s). (D-F) The correlation between effect sizes of each lipid associated with BMI and with mBMIΔ (Research Lipoid List), mBMIΔ (CLP1s) and mBMIΔ (CLP2s) respectively.

FIG. 8: The relationship of mBMIΔ derived from the CLP1s and CLP2s lists and the Research Lipid List with cardiometabolic traits. Pearson's correlation coefficients between mBMIΔ derived from CLP1s, CLP2s and the Research Lipid List (horizontal axis) and each cardio-metabolic trait (vertical axis). BMI, body mass index, HDL-C, high density cholesterol, HOMA-IR, homeostatic model assessment of insulin resistance, FBG, fasting blood glucose, 2 h-PLG, 2-hour post load glucose, SBP, systolic blood pressure, DBP, diastolic blood pressure, HbA1C, haemoglobin Alc.

FIG. 9: Association between the quintiles of mBMIΔ and cardio-metabolic risk factors. Linear regression analyses of mBMIΔ quintile (predictor) against cardio-metabolic traits; HDL-C(A), triglycerides (B) and HOMA-IR (C) were performed adjusting for age, sex and BMI. -coefficients and the CIs, from the linear regression analyses are presented. HDL-C, high density cholesterol; HOMA-IR, homeostatic model assessment of insulin resistance; CI, confidence interval; CLP, clinical lipidomic platform.

FIG. 10: The relationship between mBMIΔ and cardio-metabolic diseases. The odds ratio (x-axis) for the newly diagnosed prevalent T2DM (A) (N=395/7733 NGT) and (B) 5-year incident T2DM (N=218 incident cases/5354 controls) across the quintiles of mBMIΔ (y-axis). The odds ratios were computed from a multiple logistic regression between a newly diagnosed prevalent T2DM, against NGT subjects or between 5-year incident T2DM against controls and the quintiles of the mBMIΔ (Q1 as a reference) adjusted for age, sex, and BMI. The odds ratio/hazard ratios (x-axis) for the prevalent CVD (C) or 10-year incident CVE (D) across the quintiles of mBMIΔ (y-axis). The odds ratios were computed from a multiple logistic regression between a prevalent CVD, n=577 versus 9,690 controls at baseline or COX regression between incident CVE, n=414 events versus 7,936 non-events and the quintiles of the mBMIΔ (Q1 as a reference) adjusted for age, sex, BMI, smoking status and diabetes. CI, confidence interval; CLP, clinical lipidomic platform.

FIG. 11: An overview of the study design for the development of metabolic age scores using CLP and the subsequent downstream analyses. The full lipidomic data or CLP lipids were used for the generation of the metabolic Age score in the AusDiab cohort (n=10,339) using ridge model, and the downstream analyses (association of the mAgeΔ with cardio-metabolic traits/diseases) were performed. AusDiab, Australian Diabetes, Obesity and Lifestyle Study; mAge, metabolic age; mAgeΔ, metabolic age delta; T2DM, type 2 diabetes mellitus; CVD, cardiovascular disease; CVE, cardiovascular event; LC-MS/MS, liquid chromatography tandem mass spectrometry.

FIG. 12: The correlation among pAge or mAge scores derived using the Research Lipid List and the CLP.

FIG. 13: Association between the quintiles of mAgeΔ and cardio-metabolic risk factors. Linear regression analyses of mAgeΔ quintile (predictor) against cardio-metabolic traits; triglycerides, cholesterol and SBP were performed adjusting for age, sex and BMI. β-coefficients and the CIs, from the linear regression analyses are presented. SBP, systolic blood pressure; CI, confidence interval; CLP, clinical lipidomic platform.

FIG. 14: The relationship between mAgeΔ and cardiometabolic diseases. The odds ratio (x-axis) for the newly diagnosed prevalent T2DM (N=395/7733 NGT) and 5-year incident T2DM (N=218 incident cases/5354 controls) across the quintiles of mAgeΔ (y-axis). The odds ratios were computed from a multiple logistic regression between a newly diagnosed prevalent T2DM, against NGT subjects or between 5-year incident T2DM against controls and the quintiles of the mAgeΔ (Q1 as a reference) adjusted for age, sex, and BMI. The odds ratio/hazard ratios (x-axis) for the prevalent CVD or 10-year incident CVE across the quintiles of mAgeΔ (y-axis). The odds ratios were computed from a multiple logistic regression between a prevalent CVD, n=577 versus 9,690 controls at baseline or COX regression between incident CVD, n=414 events versus 7,936 non-events and the quintiles of the mAgeΔ (Q1 as a reference) adjusted for age, sex, BMI, smoking status and diabetes. CI, confidence interval; CLP, clinical lipidomic platform.

FIG. 15: The relationship between mAgeΔ and all-cause mortality. The hazard ratios (x-axis) for all-cause mortality risk across the quintiles of mAgeΔ (y-axis). The hazard ratios were computed from a Cox regression between time to death and 17-year all-cause mortality, n=1706 and the quintiles of the mAgeΔ (Q1 as a reference) adjusted for age, sex, BMI, smoking status and diabetes. CI, confidence interval; CLP, clinical lipidomic platform.

FIG. 16: An overview of Plasmalogen Score development. (a) Principal components were generated using a compositional data of all PE and PE(P) species (n=29 lipids) in the AusDiab cohort. (b) A loadings plot showing the separation of PE(P) and PE lipid species. (c) The correlations of Plasmalogen Score with PE and PE(P) species used to derive the principal components.

FIG. 17: The correlation of the plasmalogen scores derived using the full lipid list and the CLP1s lipid list.

FIG. 18: The relationship of plasmalogen scores derived from the CLPs and the full lipidome with cardiometabolic traits. (a) Pearson's correlation coefficients between risk factors and plasmalogen scores derived from the lipidome; plasmalogen scores derived from CLP1 (b) and a PS derived using the CLP2 (c). BMI, body mass index, HDL-C, high density cholesterol, HOMA-IR, homeostatic model assessment of insulin resistance, FBG, fasting blood glucose, 2 h-PLG, 2-hour post load glucose, SBP, systolic blood pressure, DBP, diastolic blood pressure, HbA1C, haemoglobin Alc.

FIG. 19: Association between the quintiles of Plasmalogen Score and cardio-metabolic risk factors. Linear regression analyses of PS quintile (predictor) against cardio-metabolic risk factors (outcome); age, BMI, triglycerides and HDL-C were performed adjusting for age, sex and BMI (excluding the outcome). β-coefficients and the CIs, from the linear regression analyses are presented. HDL-C, high density cholesterol; CT, confidence interval.

FIG. 20: The relationship between quintiles of plasmalogen score (PC1) and cardio-metabolic diseases. The odds/hazard ratio (x-axis) for the newly diagnosed prevalent T2DM (N=395/7733 NGT), 5-year incident T2DM (N=218 incident cases/5354 controls), prevalent CVD (N=577/9630), and all-cause mortality over 17 years (n=1706/8625) across the quintiles of mBMIΔ (y-axis). The odds ratios were computed from a multiple logistic regression between a newly diagnosed prevalent T2DM, against NGT subjects or between 5-year incident T2DM against controls and the quintiles of the PC1 (Q1 as a reference) adjusted for age, sex, and BMI. The odds ratio/hazard ratios (x-axis) for the prevalent CVD (C) or all-cause mortality across the quintiles of PC1 (y-axis) were adjusted for age, sex, BMI, smoking status and diabetes. CI, confidence interval; CLP, clinical lipidomic platform.

FIG. 21: AUC plots of the intermediate risk group in Ausdiab for the full lipids panel and clinical platform 1-5 showing all the clinical platforms achieved the same performance level as the full lipids panel. CLP0 refers to the full lipids panel and CLP1-5 refers to the clinical platform1-5, respectively. FRS AUC refers to the AUC of the Framingham score and LRS AUC refers to the AUC of the LARS score.

FIG. 22: AUC plots of the whole Ausdiab population for the full lipids panel and clinical platform 1-5 showing all the clinical platforms achieved the same performance level as the full lipids panel. CLP0 refers to the full lipids panel and CLP1-5 refers to the clinical platform1-5, respectively. FRS AUC refers to the AUC of the Framingham score and LRS AUC refers to the AUC of the LARS score.

FIG. 23: The NRI metric for the intermediate-risk group and the whole population in Ausdiab. The x axis refers to the clinical lipid platform index, 0 for whole lipidome and 1-5 refers to clinical platform 1-5. The y axis is for the net reclassification improvement (NRI). The NRI values are represented by the dots and the confidence intervals are shown by error bars. In the intermediate risk group, the NRI of LARS as compared to FRS was significantly improved across all of the clinical platforms.

FIG. 24: Current screening approach for prediabetes and T2DM in Australia. Adapted from the current position statement in Australia (Position Statement for Management of Prediabetes 30 Apr. 2020).

FIG. 25: Performance of multivariate models to predict BMI. Models were developed using a Ridge model of the ~250 CLP2 lipids (A) and a 50-lipid subset of the CLP2 lipids (B) in the AusDiab cohort. Predicted BMI was plotted against measured BMI for each model.

FIG. 26: Comparison of predicted BMI and mBMIΔ between models containing ~250 CLP2 lipids and a subset of ~50 CLP2 lipids. The pBMI determined using a Ridge model of the CLP2 lipids, ~250 (x-axis) was plotted against the predicted BMI determined using a LASSO model of ~50 lipid subset of the CLP2 (y-axis) in the AusDiab cohort (A). The mBMIΔ determined using a Ridge model of the CLP2 lipids, ~250 (x-axis) was plotted against the mBMIΔ determined using a LASSO model of ~50 lipid subset of the CLP2 (y-axis) in the AusDiab cohort (B).

FIG. 27: Performance of multivariate models to predict Age. Models were developed using a Ridge model of the ~250 CLP2 lipids (A) and a 50-lipid subset of the CLP2 lipids (B) in the AusDiab cohort. Predicted Age was plotted against chronological age for each model.

FIG. 28: Comparison of predicted Age (pAge) and mAgeΔ between models containing ~250 CLP2 lipids and a subset of ~50 CLP2 lipids. The pAge determined using a Ridge model of the CLP2 lipids, ~250 (x-axis) was plotted against the predicted Age determined using a LASSO model of ~50 lipid subset of the CLP2 (y-axis) in the AusDiab cohort (A). The mAgeΔ determined using a Ridge model of the CLP2 lipids, ~250 (x-axis) was plotted against the mAgeΔ determined using a LASSO model of ~50 lipid subset of the CLP2 (y-axis) in the AusDiab cohort (B).

FIG. 29: Performance of the LRS50 and the FRS to predict incident cardiovascular events. AUC plots of the whole Ausdiab population (left panel) and intermediate-risk group (right panel) for the LRS model using 50 lipids (LRS50, dashed line)), which had improved performance compared to the FRS (solid line). FRS: Framingham risk score. LRS: LRS50 lipidomic risk score.

FIG. 30: Reclassification of the intermediate risk group from the AusDiab cohort using the LRS (full lipidome) or the LRS50. Bar plots showing the raw counts of the reclassification by the LRS derived from full lipids panel (left plot) and LRS50 (right plot) for the intermediate-risk defined by the FRS in the Ausdiab cohort. The LRS model using 50 lipids (LRS50) showed a similar reclassification pattern to the LRS model derived from full lipids panel, with both reclassifying 34% (n=36) of cases from intermediate-risk to high-risk. Black and grey bars represent CVD events and non-events, respectively. Low, intermediate and high, represent individuals reclassified into low-risk, intermediate-risk, and high-risk groups, respectively. The number above each bar represents the raw counts of the individuals after the reclassification.

FIG. 31: Relationship between predicted and originally measured 2 h-PLG A. The relationship between the original 2 h-PLG measurement in AusDiab and the 50-CLP predicted 2 h-PLG. B—Comparison of 2 h-PLG predicted by the 50-lipid CLP and the 2h-PLG predicted by the full CLP set of lipids.

FIG. 32: AUC plots contrasting the prediction of impaired glucose tolerance. Base model comprises of AUSDRISK, HbA1c and fasting glucose, while the base+lipid model includes the 50-CLP predicted 2 h-PLG (left) or the ~250-CLP predicted 2 h-PLG (right). Inflection point for the curve was derived using Youden's index.

### BRIEF DESCRIPTION OF TABLES

Table 1 lists internal standards and volumes used.

Table 2 lists the Research Lipid List.

Table 3 lists the internal standards used with Research Lipid List analyses.

Table 4 lists the clinical lipid platform (CLP1) lipid list.

Table 5 lists the internal standards used with the CLP1 and CLP2 list analyses.

Table 6 lists the clinical platform subset (CLP2) lipid list.

Table 7 details the predictive performances of ridge and LASSO models for the prediction of BMI.

Table 8 lists predictive models of BMI developed using Ridge regression incorporating age and sex together with the CLP1s lipids.

Table 9 lists predictive models of BMI developed using LASSO regression incorporating age and sex together with the CLP1s lipids.

Table 10: lists predictive models of BMI developed using Ridge regression incorporating age and sex together with the CLP2s lipids.

Table 11: lists predictive models of BMI developed using LASSO regression incorporating age and sex together with the CLP2s lipids.

Table 12 details the predictive performances of ridge model for the prediction of age.

Table 13: lists predictive models of age developed using Ridge regression incorporating BMI and sex together with the CLP1s lipids.

Table 14: lists predictive models of age developed using LASSO regression incorporating BMI and sex together with the CLP1s lipids.

Table 15: lists predictive models of age developed using Ridge regression incorporating BMI and sex together with the CLP2s lipids.

Table 16: lists predictive models of age developed using LASSO regression incorporating BMI and sex together with the CLP2s lipids.

Table 17: lists weights for the PE and PE(P) species in the Plasmalogen Scores derived from CLP1s and CLP2s lipid lists.

Table 18 details the performance metrics in Ausdiab for LARS models derived from whole lipids panel and clinical platforms 1 to 5.

Table 19: lists predictive models of cardiovascular disease events developed using Ridge regression with the CLP1s lipids.

Table 20: lists predictive models of cardiovascular disease events developed using LASSO regression with the CLP1s lipids.

Table 21: lists predictive models of cardiovascular disease events developed using Ridge or LASSO regression with the CLP2s lipids.

Table 22: lists predictive models of cardiovascular disease events developed using Ridge or LASSO regression with the CLP2s lipids.

Table 23: lists predictive models of 2 h-PLG developed using Ridge regression with the CLP1s lipids.

Table 24: lists predictive models of 2 h-PLG developed using LASSO regression with the CLP1s lipids.

Table 25: lists predictive models of 2 h-PLG developed using Ridge regression with the CLP2s lipids.

Table 26: lists predictive models of 2 h-PLG developed using LASSO regression with the CLP2s lipids.

Table 27 details IGT classification performance of base and lipid models.

Table 28 details 5-year-IGT classification performance of base and lipid models Table 29 details 5-year-T2DM classification performance of base and lipid models.

Table 30 List of lipid species and their transitions monitored via MRM in the CLP2 lipid list and in CLP2s, CLP3, CLP4 and CLP5.

Table 31 [M+2+H] MRM transitions used as an alternative means to monitor three high abundance PC species and an internal standard.

Table 32 Predictive models of BMI developed using LASSO regression with an approximately 50-lipid subset the CLP2s lipids.

Table 33 Predictive model of Age developed using LASSO regression with an approximately 50-lipid subset the CLP2 lipids.

Table 34 Model performance and net reclassification of the LRS models.

Table 35 Predictive models of LRS developed using LASSO regression with an approximately 50-lipid subset the CLP2 lipids.

Table 36 Predictive models of 2 h-PLG developed using LASSO regression with an approximately 50-lipid subset the CLP2 lipids.

Table 37 Sensitivity and Specificity in classifying individuals with impaired glucose tolerance using the 50-CLP predicted 2 h-PLG.

Table 38 Sensitivity and Specificity in classifying individuals with impaired glucose tolerance using the full CLP predicted 2 h-PLG.

LIST OF ABBREVIATIONS

| Abbreviation | Full description |
|---|---|
| AC | Acylcarnitines |
| AC-OH | Hydroxylated Acylcarnitine |
| BA | Bile acid |
| Cer | Ceramide |
| Cer(d) | Ceramide(d) |
| Cer(m) | Ceramide(m) |
| CE | Cholesteryl ester |
| C1P | Ceramide-1-phosphate |
| COH | Free cholesterol |
| DE | Dehydrocholesterol ester |
| DG | Diacylglycerol |
| deDE | Dehydrodesmosteryl ester |
| dhCer | Dihydroceramide |
| FFA | Free fatty acid |
| dimethyl-CE | Dimethyl cholesteryl ester |
| GM1 | G_{M1} Ganglioside |
| GM3 | G_{M3} Ganglioside |
| HexCer | Monohexosylceramide |
| Hex2Cer | Dihexosylceramide |
| Hex3Cer | Trihexosylcermide |
| ISTD | Internal Standard |
| LPC(P) | Lysoalkenylphosphatidylcholine |
| LPC | Lysophosphatidylcholine |
| LPC(O) | Lysoalkylphosphatidylcholine |
| LPC(P) | Lysoalkenylphosphatidylcholine |
| LPE | Lysophosphatidylethanolamine |
| LPE(P) | Lysoalkenylphosphatidylethanolamine |
| LPI | Lysophosphatidylinositol |
| MHDA | 15-methylhexadecanoic acid or 14- |
| | methylhexadecanoic acid |
| methyl-CE | Methylcholesteryl ester |
| methyl-DE | Methyl deoxycholesterol ester |
| OxSpecies | Oxidised lipid species |
| PA | Phosphatidic acid |
| PC | Phosphatidylcholine |
| PC(P) | Alkenylphosphatidylcholine |
| PC(O) | Alkyl phosphatidylcholine |
| PE | Phosphatidylethanolamine |
| PE(P) | Alkenylphosphatidylethanolamine |
| PE(O) | Alkyl phosphatidylethanolamine |
| PG | Phosphatidylglycerol |
| PI | Phosphatidylinositol |
| PIP1 | Phosphatidylinositol-1-phosphate |
| PS | Phosphatidylserine |
| SM | Sphingomyelin |
| SHexCer | Sulfatide |
| S1P | Sphingosine-1-phosphate |
| Sph | Sphingosine |
| SM | Sphingomyelin |
| TG | Triacylglycerol |
| TG(O) | Alkyldiacylglycerol |
| Ubiquinone | Ubiquinone |

Lipid classes and subclasses

| Lipid category | Lipid class | Lipid class/subclass | Abbreviation |
|---|---|---|---|
| Sphingolipids | Ceramide | Dihydroceramide | dhCer |
| Sphingolipids | Ceramide | Ceramide | Cer |
| Sphingolipids | Ceramide | Ceramide | Cer(d) |
| Sphingolipids | Ceramide | Deoxyceramide | Cer(m) |
| Sphingolipids | Ceramide | Ceramide-1-phosphate | C1P |
| Sphingolipids | Neutral glycosphingolipids | Monohexosylceramide | HexCer |
| Sphingolipids | Neutral glycosphingolipids | Dihexosylceramide | Hex2Cer |
| Sphingolipids | Neutral glycosphingolipids | Trihexosylcermide | Hex3Cer |
| Sphingolipids | Acid glycosphingolipids | G_{M3} Ganglioside | GM3 |
| Sphingolipids | Acid glycosphingolipids | G_{M1} Ganglioside | GM1 |
| Sphingolipids | Acid glycosphingolipids | Sulfatide | Sulfatide |
| Sphingolipids | Phosphosphingolipids | Sphingomyelin | SM |
| Glycerophospholipids | Glycerophosphocholines | Phosphatidylcholine | PC |
| Glycerophospholipids | Glycerophosphocholines | Alkylphosphatidylcholine | PC(O) |
| Glycerophospholipids | Glycerophosphocholines | Alkenylphosphatidylcholine | PC(P) |
| Glycerophospholipids | Glycerophosphocholines | Lysophosphatidylcholine | LPC |
| Glycerophospholipids | Glycerophosphocholines | Lysoalkylphosphatidylcholine | LPC(O) |
| Glycerophospholipids | Glycerophosphocholines | Lysoalkenylphosphatidylcholine | LPC(P) |
| Glycerophospholipids | Glycerophosphoethanol- | Phosphatidylethanolamine | PE |
| | amines | | |
| Glycerophospholipids | Glycerophosphoethano- | Alkylphosphatidylethanolamine | PE(O) |
| | lamines | | |
| Glycerophospholipids | Glycerophosphoethanol- | Alkenylphosphatidylethanol- | PE(P) |
| | amines | amine | |
| Glycerophospholipids | Glycerophosphoethanol- | Lysophosphatidylethanolamine | LPE |
| | amines | | |
| Glycerophospholipids | Glycerophosphoethanol- | Lysoalkenylphosphatidylethanol- | LPE(P) |
| | amines | amine | |
| Glycerophospholipids | Glycerophosphoinositols | Phosphatidylinositol | PI |
| Glycerophospholipids | Glycerophosphoinositols | Lysophosphatidylinositol | LPI |
| Glycerophospholipids | Glycerophosphoserines | Phosphatidylserine | PS |
| Glycerophospholipids | Glycerophosphoglycerols | Phosphatidylglycerol | PG |
| Sterol lipids | Sterols | Free cholesterol | COH |
| Sterol lipids | Sterols | Cholesteryl ester | CE |
| Sterol lipids | Sterols | Dimethyl cholesteryl ester | dimethyl-CE |
| Sterol lipids | Sterols | Methylcholesteryl ester | methyl-CE |
| Sterol lipids | Sterols | Dehydrocholesteryl ester | DE |
| Sterol lipids | Sterols | Methyl deoxycholesterol ester | methyl-DE |
| Sterol lipids | Sterols | Dehydrodesmosteryl ester | deDE |
| Fatty acyls | Fatty esters | Acylcarnitines | AC |
| Fatty acyls | Fatty esters | Hydroxylated acylcarnitine | AC-OH |
| Glycerolipids | Diradylglycerols | Diacylglycerol | DG |
| Glycerolipids | Triradylglycerols | Triacylglycerol | TG |
| Glycerolipids | Triradylglycerols | Alkyldiacylglycerol | TG(O) |
| Prenol Lipids | Quinones and | Ubiquinone | Ubiquinone |
| | hydroquinones | | |

### DETAILED DESCRIPTION

The present disclosure describes the following various non-limiting embodiments, which relate to research undertaken into identifying and developing lipidomics based methods for assessing metabolic health in a subject.

### Terms

Unless defined otherwise, all technical and scientific terms used herein have the same meaning as commonly understood by those of ordinary skill in the art to which the disclosure belongs.

As used herein the singular forms “a”, “an” and “the” include plural aspects unless the context clearly dictates otherwise. Thus, for example, reference to “a lipid species” includes a single lipid species, as well as two or more lipid species, reference to “the disclosure” includes single and multiple aspects of the disclosure and so forth.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, or +/−5%, of the designated value.

The naming convention for lipids used herein follows the guidelines established by the Lipid Maps Consortium and the shorthand notation of Liebisch et al. (see for example Fahy et al. 2005. J. Lipid Res. 46: 839-861; Fahy et al 2009. J. Lipid Res. 50 (Suppl.): S9-S14; Liebisch et al. 2013. J. Lipid Res. 54: 1523-1530; Liebisch et al. 2020. J. Lipid Res. 2020 December; 61(12): 1539-1555). Lipids can be divided into primary categories including fatty acyls (FA), glycerolipids (GL), glycerophospholipids (GP), sphingolipids (SP), sterol lipids (ST), prenol lipids (PR), saccharolipids (SL), and polyketides (PK), which can be divided into molecule classes, subclasses and species representing chemical structures.

Fatty acyls (FA) are a diverse group of molecules synthesized by chain elongation of an acetyl-CoA primer with malonyl-CoA (or methylmalonyl-CoA) groups that may contain a cyclic functionality and/or are substituted with heteroatoms. Structures with a glycerol group are represented by two distinct categories: the glycerolipids (GL), which include acylglycerols but also encompass alkyl and 1Z-alkenyl variants, and the glycerophospholipids (GP), which are defined by the presence of a phosphate (or phosphonate) group esterified to one of the glycerol hydroxyl groups. Sterol lipids (ST) and prenol lipids (PR) share a common biosynthetic pathway via the polymerization of dimethylallyl pyrophosphate/isopentenyl pyrophosphate but otherwise differ in structure and function. Sphingolipids (SP) contain a long-chain base as their core structure. Saccharolipids” (SL) contain fatty acyl groups linked directly to a sugar backbone.

Glycerophospholipids (GP) typically contain two fatty acid chains and in the absence of detailed characterisation are expressed as the sum composition of carbon atoms and double bonds (i.e. PC(38:6)). However, where an acyl chain composition has been determined the naming convention indicates this (i.e. PC(38:6) is changed to PC(16:0_22:6)). This is also extended into other lipid classes or subclasses.

The present disclosure refers to lipid molecules using the numbering system X:Y. The number X represents the number of carbon atoms present in the chain.

In the context of alkylglycerols, alkyacylglycerols or alkyldiacylglycerols, the number Y represents the number of double bonds present in the chain. For example, an alkylglycerol numbered as 16:0 contains a hydrocarbon group having a 16 carbon chain with no double bonds. As a further example, an alkylglycerol numbered as 18:1 contains a hydrocarbon group having an 18 carbon chain with 1 double bond.

In the context of plasmalogens/plasmenyl phospholipids, the number Y in the first listed alkenyl chain (i.e. PE(P—X:Y/X:Y) represents the number of double bonds present in the alkenyl chain in addition to the vinyl ether group. For example, a plasmalogen numbered as PE(P-16:0/20:4) the 16:0 alkenyl group contains a hydrocarbon group having a 16 carbon chain with no double bonds other than the vinyl ether group (i.e. there is a double bond between the first 2 carbons and the remaining 14 carbons are saturated). As another example, a plasmalogen numbered as PE(P-18:1/20:4) the 18:1 alkenyl group contains a hydrocarbon group having an 18 carbon chain with 1 double bond in addition to the vinyl ether group (i.e. there is a double bond between the first 2 carbon atoms, and there is one other double bond between 2 carbons out of the remaining 16 carbons).

Where ether lipids contain one or more double bonds, the double bonds may be located at various positions in the hydrocarbon chains. For example, an alkylglycerol numbered as 18:1 may contain a mixture of species, e.g. with cis-n7 and cis-n9 double bonds. As another example, a plasmalogen (e.g. PE(P)) numbered as 18:1 may contain a mixture of species, e.g. with cis-n7 and cis-n9 double bonds.

As used herein, the term “plasmanyl” refers to phospholipids having an ether bond in the sn-1 position to an alkyl group.

As used herein, the term “plasmenyl” refers to phospholipids having an ether bond in the sn-1 position to an alkenyl group. The plasmenyl phospholipids are also referred to as “plasmalogens”.

A plasmalogen having a “16:0” alkenyl group is typically a molecule having an ether bond in the sn-1 position to an 16 carbon chain which contains a double bond between carbons 1 and 2 (i.e. typically a cis-vinyl ether group), and no other double bonds in the chain.

A plasmalogen having an “18:0” alkenyl group is typically a molecule having an ether bond in the sn-1 position to an 18 carbon chain which contains a double bond between carbons 1 and 2 (i.e. typically a cis-vinyl ether group), and no other double bonds in the chain.

A plasmalogen having an “18:1” alkenyl group is typically a molecule having an ether bond in the sn-1 position to an 18 carbon chain which contains a double bond between carbons 1 and 2 (i.e. typically a cis-vinyl ether group), and having one additional double bond, typically between carbons 7 and 8 (e.g. n7), between carbons 9 and 10 (e.g. n9), or between carbons 11 and 12 (e.g. n11), and typically a cis-double bond.

A plasmalogen having an “18:2” alkenyl group is typically a molecule having an ether bond in the sn-1 position to an 18 carbon chain which contains a double bond between carbons 1 and 2 (i.e. typically a cis-vinyl ether group), and having two additional double bonds, typically between carbons 9 and 10, and between carbons 11 and 12, and typically cis-double bonds.

A plasmalogen having an “18:2” acyl alkenyl group is typically a molecule having an ester bond in the sn-2 position to an 18 carbon chain which has two double bonds, typically between carbons 9 and 10, and between carbons 11 and 12, and typically cis-double bonds.

A plasmalogen having a “20:4” acyl alkenyl group is typically a molecule having a ester bond in the sn-2 position to a 20 carbon chain which has four double bonds, typically between carbons 5 and 6, carbons 8 and 9, carbons 11 and 12, and carbons 14 and 15, and typically cis-double bonds.

As used herein, “acyl” refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C₁-C₂₄ acyl-group include acetyl, benzoyl-, nicotinoyl-, propionyl-, isobutyryl-, oxalyl-, and the like. Lower-acyl refers to acyl groups containing one to four carbons. An acyl group can be unsubstituted or substituted, for example with one or more groups selected from halogen, —OH, —NH₂, —CN, —OC₁₋₄ alkyl and —CO₂H. Additional examples or generally applicable substituents are illustrated by the specific compounds described herein.

The term “aliphatic” as used herein, includes saturated, unsaturated, straight chain (i.e., unbranched), or branched, aliphatic hydrocarbons, which are optionally substituted with one or more functional groups. In some embodiments, the aliphatic may contain one or more functional groups such as double bond, triple bond, or a combination thereof. As will be appreciated by one of ordinary skill in the art, “aliphatic” is intended herein to include, but is not limited to, alkyl, alkenyl, alkynyl, or acyl moieties. Thus, as used herein, the term “alkyl” includes straight and branched saturated groups. An analogous convention applies to other generic terms such as “alkenyl”, “alkynyl”, “acyl” and the like. Furthermore, as used herein, the terms “alkyl”, “alkenyl”, “alkynyl”, “acyl” and the like encompass both substituted and unsubstituted groups.

As used herein, “alkenyl” refers to a straight or branched chain hydrocarbon containing, for example, from 2 to 30 carbons and containing at least one carbon-carbon double bond. In some embodiments, the alkenyl group contains 10 to 25, 14 to 22, or 16 to 20 carbon atoms. In some embodiments, the alkenyl group contains 15, 16, 17, 18, 19 or 20 carbon atoms. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, 3-undecenyl, 4-dodecenyl, 4-tridecenyl, 9-tetradecenyl, 8-pentadecenyl, 5-hexadecenyl, 8-heptadecenyl, 9-octadecenyl, 9-nonadecenyl and the like. Additional examples or generally applicable substituents are illustrated by the specific compounds described herein.

As used herein, “alkyl” refers to a straight or branched chain hydrocarbon containing, for example, from 1 to 30 carbon atoms. In some embodiments, the alkyl group contains 10 to 25, 14 to 22, or 16 to 20 carbon atoms. In some embodiments, the alkyl group contains 15, 16, 17, 18, 19 or 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, noctyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and the like. Additional examples or generally applicable substituents are illustrated by the specific compounds described herein.

As used herein, “acyl alkenyl” refers to a straight or branched chain hydrocarbon containing, for example, from 2 to 30 carbons and containing at least one carbon-carbon double bond, which is covalently bonded to an acyl group. The use of nomenclature 22:6 or 18:2 and the like in the context of an acyl alkenyl group refers to an acyl alkenyl group having 22 carbons or 18 carbons respectively, and having 6 or 2 double bonds respectively. An example of an acyl alkenyl group is:

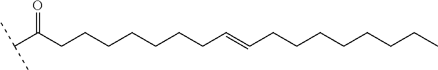

Acyl alkenyl groups may be present in species such as alkylacylglycerols or alkyldiacylglycerols (as an acyl group), or as an acyl group in plasmanyl- or plasmenyl-phospholipids. Typically, when present in those species, there is no double bond between the carbons which are α- and β- to the acyl group.

As used herein, “acyl alkyl” refers to a straight or branched chain hydrocarbon containing, for example, from 1 to 30 carbons, which is covalently bonded to an acyl group. The use of nomenclature 22:0 or 18:0 and the like in the context of an acyl alkyl group refers to an acyl alkyl group having 22 carbons or 18 carbons respectively. An example of an acyl alkyl group is:

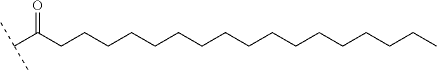

It will also be recognised that the compounds described herein may possess asymmetric centres and are therefore capable of existing in more than one stereoisomeric form. The disclosure thus also relates to compounds in substantially pure isomeric form at one or more asymmetric centres e.g., greater than 90% ee, such as 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

The present disclosure relates to derivatives of glycerol. Whilst glycerol is achiral, derivatives are typically chiral. Typically the glycerol utilised will have a stereochemical configuration corresponding to that found in nature. In some embodiments, the glycerol derivatives utilised have the following stereochemical configuration:

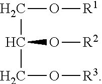

As referred to herein, the term “alkylglycerol” means a compound in which the R¹ group is a hydrocarbon chain, the R² and R³ groups are each hydrogen. Although the term “alkyl” glycerol is used, it will be understood by those of skill in the art that the term encompasses species with hydrocarbon groups at the R¹ position which include unsaturation in the hydrocarbon chain. However, an alkylglycerol does not contain a double bond between carbons 1 and 2 of the hydrocarbon chain, e.g. proximal to the ether linkage.

An alkylglycerol having a “16:0” group is typically a molecule having an ether bond in the sn-1 position to a 16 carbon saturated hydrocarbon chain, and no double bonds in the chain.

An alkylglycerol having an “18:0” group is typically a molecule having an ether bond in the sn-1 position to an 18 carbon saturated hydrocarbon chain, and no double bonds in the chain.

An alkylglycerol having an “18:1” group is typically a molecule having an ether bond in the sn-1 position to an 18 carbon hydrocarbon chain, which contains one double bond, typically between carbons 9 and 10 and typically a cis-double bond.

As referred to herein, the term “alkylacylglycerol” means a compound of Formula 1 in which the R¹ group is a hydrocarbon chain, one of the R² and R³ groups is hydrogen, and the other of the R² and R³ groups is an acyl group, either an acyl alkyl group or an acyl alkenyl group. Although the term “alkyl” acylglycerol is used, it will be understood by those of skill in the art that the term encompasses species with hydrocarbon groups at the R¹ position which include unsaturation in the hydrocarbon chain. However, an alkylacylglycerol does not contain a double bond between carbons 1 and 2 of the R¹ hydrocarbon chain, e.g. proximal to the ether linkage.

As referred to herein, the term “alkyldiacylglycerol” means a compound of Formula 1 in which the R¹ group is a hydrocarbon chain, and the R² and R³ groups are acyl groups, either acyl alkyl or acyl alkenyl. Although the term “alkyl” diacylglycerol is used, it will be understood by those of skill in the art that the term encompasses species with hydrocarbon groups at the R¹ position which include unsaturation in the hydrocarbon chain. However, an alkyldiacylglycerol does not contain a double bond between carbons 1 and 2 of the R¹ hydrocarbon chain, e.g. proximal to the ether linkage.

The skilled person will be aware that specific lipids can be readily substituted for alternate, highly correlated lipids. Highly correlated lipid alternatives to those specified herein are contemplated and encompassed by the present application and form part of the inventive concept of the present application.

The term “BMI” refers to body mass index, and is calculated by dividing the weight of an individual in kg by their height in metres squared.

Reference to “two or more”, incudes 2, 3, 4, 5, 6, 7, 8, 9 or 10 or more lipids.

### Lipidomic Assessment of Metabolic Health

The present disclosure provides methods for assessing metabolic health in a subject based on a level of a plurality or population of lipid species in the subject.

In some examples, the metabolic health that is determined is one or more of: metabolic age (mAge); metabolic body mass index (mBMI); plasmalogen score (PS); risk of developing cardiovascular disease (CVD); and/or risk of developing diabetes (e.g. type 2 diabetes, T2D).

In one example, the present disclosure provides a method of determining metabolic health of a subject, the method comprising:
detecting in a biological sample from the subject a level of a plurality of lipid species comprising at least two lipid species selected from the group consisting of any one or more Tables, lipid classes, and/or lipid species according to any embodiments or examples thereof as described herein;
comparing the levels or ratios of levels of the lipid species detected in the biological sample to reference levels or ratios of the lipid species; and
determining the metabolic health of the subject on the basis of the comparison.

It will be appreciated that the method can comprise detecting a level of a plurality or population of lipid species comprising at least two lipid species selected from any lipid species of the group consisting of any one or more of the Tables, lipid classes, and/or lipid species according to any embodiments or examples thereof as described herein. In some examples, the plurality or population of lipid species detected in the sample can comprise at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300 lipid species. In some examples, the plurality or population of lipid species detected in the sample can comprise less than about 800, 700, 600, 500, 400, 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10 lipid species. In some examples the plurality or population of lipid species detected in the sample can comprise a range selected from any two upper and/or lower amounts as previously described, such as 2 to 20, 2 to 100, 2 to 200, 2 to 235, 2 to 250, 2 to 269, 2 to 298, 2 to 300, 2 to 339, 2 to 400, 2 to 500, 2 to 600, 2 to 700, 2 to 791, 10 to 50, 10 to 100, 10 to 250, 10 to 500, 20 to 100, 100 to 200, 200 to 300, 300 to 400, 400 to 500, 500 to 600, 600 to 700, 25 to 300, or 50 to 250.

The lipid species may be selected from one or more lipid classes comprising or consisting of any one or more of sphingolipids, glycerophospholipids, sterol lipids, fatty acyls, glycerolipids, and prenol lipids. In one example, the lipid species is selected from the group consisting of sphingolipids, glycerophospholipids, and fatty acyls.

In one example, the lipid species is selected from one or more lipid classes or sub-classes comprising or consisting of any one or more of ceramide, neutral glycosphingolipids, acid glycosphingolipids, phosphosphingolipids, glycerophosphocholines, glycerophosphoethanolamines, glycophosphoinositols, glycerophosphoserines, glycerophosphoglycerols, sterols, fatty esters, diradylglycerols, triradylglycerols, quinones, and hydroquinones. In another example, the one or more lipid classes or sub-classes comprise or consist of any one or more of ceramide, neutral glycosphingolipids, acid glycosphingolipids, phosphosphingolipids, glycerophosphocholines, glycerophosphoethanolamines, glycophosphoinositols, and fatty esters.

In one example, the plurality of lipid species detected comprises at least two lipid species of Table 2. In some examples, the lipid species detected from Table 2 comprise at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300 lipid species. In some examples, the lipid species detected from Table 2 comprise less than about 800, 700, 600, 500, 400, 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10 lipid species. In some examples, the lipid species detected from Table 2 comprise a range selected from any two previously described upper and/or lower amounts, such as 2 to 700, 10 to 500, 25 to 300, or 50 to 250 lipid species.

In one example, the plurality of lipid species comprises the lipids set out at Table 4. In some examples, the lipid species detected from Table 4 comprise at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from Table 4 comprise less than about 350, 340, 330, 320, 310 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from Table 4 comprise a range selected from any two previously described upper and/or lower amounts, such as 2 to 300, 10 to 290, 25 to 270, or 50 to 250.

In one example, the plurality of lipid species comprises the lipids set out at Table 6. In some examples, the lipid species detected from Table 6 comprise at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, or 250. In some examples, the lipid species detected from Table 6 comprise less than about 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from Table 6 comprise a range selected from any two previously described upper and/or lower amounts, such as 2 to 250, 10 to 240, 25 to 200, or 50 to 150.

In some examples, the lipid species analysed may form a subset of the total lipid species detected.

As defined herein, normalising numeric values involves adjusting the numeric values obtained for a sample to notionally common scale determined, for example, by running a reference sample of known values simultaneously to a patient sample, thereby enabling comparison across samples taken and/or analysed at different time points and/or from different sources.

As defined herein, a reference sample or reference level is a sample or level obtained from population of individuals with predetermined characteristics. For example, populations of healthy individuals; for example, populations of subjects not known to have one or more of cardiovascular disease, diabetes, impaired glucose tolerance or low plasmalogen levels; for example, populations of male subjects; for example populations of female subjects; for example, populations of subjects of a predetermined chronological age, for example, populations of subjects of known actual BMI. A skilled person will understand a suitable reference sample from which to derive reference values.

In some examples, the model used to generate a metabolic health score comprises calculating the sum of the lipid species, wherein each lipid species is weighted. The sum of the weighted lipid species is then assessed by reference to a reference sample or level in a reference population.

Lipidomics Assessment of Metabolic Body Mass Index (mBMI)

In one example, the present disclosure provides a method of determining the metabolic body mass index (mBMI) of a subject, the method comprising:
detecting in a biological sample from the subject a level of a plurality of lipid species comprising at least two lipid species selected from any lipid species of the group consisting of any one or more Tables, lipid classes, and/or lipid species according to any embodiments or examples thereof as described herein;
comparing the levels or ratios of levels of the lipid species detected in the biological sample to reference levels or ratios of the lipid species; and
determining the mBMI of the subject on the basis of the comparison.

In one example, the mBMI is compared to the actual body mass index of the subject.

In another example, the lipid species is selected from one or more lipid classes or sub-classes comprising or consisting of any one or more of Cer, Hex1Cer, Hex2Cer, Hex3Cer, GM3, GM1, SM, PC, PC(O), PC(P), LPC, LPC(O), LPC(P), PE, PE(O), PE(P), LPE, LPE(P), PI, LPI, PS, PG, COH, CE, DE, AC, AC-OH, DG, TG, and TG(O). In another example, the one or more lipid classes or sub-classes comprise or consist of any one or more of Cer, Hex1Cer, Hex2Cer, Hex3Cer, GM3, GM1, SM, PC, PC(O), PC(P), LPC, LPC(O), LPC(P), PE, PE(O), PE(P), AC, and AC-OH. In another example, the one or more lipid classes or sub-classes comprise or consist of any one or more of SM, PC, PC(P), LPC, LPC(O), and AC.

In one example, the lipid class or subclass SM comprises or consists of any one or more lipid species of 34:2, 34:3, 34:1, 32:2, 33:1, 35:1, 38:1, 44:3, 38:2, 41:1, 32:1, 40:1, 36:3, 35:2, and 36:1. In another example, the lipid class or subclass SM comprises or consists of any one or more lipid species of 34:2, 34:3, 34:1, and 32:2.

In one example, the lipid class or subclass PC(P) comprises or consists of any one or more lipid species of 30:0, 34:1, and 36:5.

In one example, the lipid class or subclass AC comprises or consists of any one or more lipid species of 16:1, 12:0, 14:0, and 18:1.

In one example, the lipid class or subclass PC comprises or consists of any one or more lipid species of 37:6, 40:8, 38:4, 38:6, 32:1, 36:1, 38:6, 34:2, and 40:6.

In one example, the lipid class or subclass LPC comprises or consists of any one or more lipid species of 19:1, 18:0, 17:0, 26:0, 18:3, and 24:0.

In one example, the lipid class or subclass LPC(O) comprises or consists of the lipid species 20:0.

In one example, the lipid class or subclass PI comprises or consists of any one or more lipid species of 32:1, 38:6, 40:6, 36:4, and 36:1.

In one example, the lipid class or subclass Cer(m) comprises or consists of any one or more lipid species of 18:0/22:0 and 18:1/22:0.

In one example, the lipid class or subclass Cer(d) comprises or consists of any one or more lipid species of 19:1/22:0, 16:1/22:0, 17:1/24:0, 18:1/22:0, and 18:2/24:0.

In one example, the lipid class or subclass GM3 comprises or consists of any one or more lipid species of 18:1/24:1, 18:1/22:0, and 18:1/18:0.

In one example, the lipid class or subclass Hex2Cer comprises or consists of any one or more lipid species of 18:1/24:0, 18:1/24:1, and 16:1/16:0.

In one example, the lipid class or subclass Cer comprises or consists of the lipid species of 18:1/16:0.

In one example, the lipid class or subclass PE comprises or consists of any one or more lipid species of 36:1, 36:4, and 38:6.

In one example, the lipid class or subclass PE(P) comprises or consists of any one or more lipid species of 18:0/22:4, 20:0/18:2, 16:0/18:2, 18:0/18:2, 18:0/20:5, 18:0/18:1, and 16:0/18:1.

In one example, the lipid class or subclass CE comprises or consists of any one or more lipid species of 14:0, 16:1, and 18:0.

In one example, the lipid class or subclass TG comprises or consists of any one or more lipid species of 56:6, 56:7, 56:8, 48:3, 52:2, 52:1, 58:10, 54:2, 54:3, 54:4, 56:7, and 51:2.

In one example, the lipid class or subclass Hex1Cer comprises or consists of any one or more lipid species of 18:1/16:0, 18:2/24:0, and 18:1/22:0.

In one example, the lipid class or subclass DG comprises or consists of any one or more lipid species of 38:4 and 34:2.

In one example, the lipid class or subclass PC(O) comprises or consists of the lipid species 32:0.

In other examples, the lipid species may comprise or consist of any one or more combinations of the above lipid species or any classes or subclasses thereof.

In one example, the plurality of lipid species comprises the lipids set out at Tables 3, 4, 5, 6, 8, 9, 10, 11, and 30.

In one example, the plurality of lipid species comprises the lipids set out at Tables 8, 9, 10, 11, and 30. In some examples, the lipid species detected from any one or more of Tables 3, 4, 5, 6, 8, 9, 10, 11, and 30 comprise at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 3, 4, 5, 6, 8, 9, 10, 11, and 30 comprise less than about 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 3, 4, 5, 6, 8, 9, 10, 11, and 30 comprise a range selected from any two previously described upper and/or lower amounts, such as 2 to 300, 10 to 290, 25 to 270, or 50 to 250.

In some examples, the lipid species detected from any one or more of Tables 3, 4, 5, 6, 8, 9, 10, 11, and 30, in order of priority numbering in Tables 8, 9, 10 or 11, comprise at least the first 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 3, 4, 5, 6, 8, 9, 10, 11, and 30, in order of priority numbering in Tables 8, 9, 10 or 11, comprise less than about the first 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 3, 4, 5, 6, 8, 9, 10, 11, and 30, in order of priority numbering in Tables 8, 9, 10 or 11, comprise a range selected from any two previously described upper and/or lower amounts, such as 2 to 300, 10 to 290, 25 to 270, or 50 to 250.

In some examples, the plurality of lipid species comprises at least 20, at least 30, at least 40, or all of the lipid species described in Table 32.

mBMI Calculation

The present disclosure also provides a method of a calculating a metabolic body mass index (mBMI) score. The method may comprise obtaining lipid profile data from a biological sample taken from the subject, which may comprise lipid species according to any aspects, embodiments, or examples thereof as described herein. The method may also comprise standardising or normalising or scaling the numeric values of the lipid profile data against a reference data set. The method may also comprise refining the discriminatory power of one or more lipid species by statistically weighting some of the numeric values associated therewith. The method may also comprise summating or similar the numeric values obtained from steps (above) to provide a composite metabolic body mass score.

In one example, there is provided a method of a calculating a metabolic body mass score comprising:
obtaining lipid profile data from a biological sample taken from the subject;
standardising or normalising or scaling the numeric values of the lipid profile data against a reference data set;
refining the discriminatory power of one or more lipid species by statistically weighting some of the numeric values associated therewith;
Determining a composite metabolic body mass score.

In some examples, the metabolic body mass score can be used to determine the metabolic health of the subject.

In one example, the predicted BMI is standardised to the population and the mBMI is derived follows:

mBMI=BMI+(pBMI−pBMI value on the line of best fit between pBMI and BMI).

In one example, the mBMIΔ is defined as the difference between BMI and mBMI.

In some examples, the metabolic body mass score can be used to determine the risk of the subject developing disease.

In some examples, the metabolic body mass score is standardised for age and/or gender of the subject.

In some examples, the metabolic body mass score is used to determine suitable treatment for the subject.

In some examples, the subject is a mammal.

In some examples, the subject is a human.

In some examples, the lipid profile data is generated form a liquid chromatography-mass spectrometry (LC-MS) protocol.

In some examples, the LC-MS protocol has a total run time of less than about (in minutes) 15, 14, 13, 12, 11, 10, 9, 8, 7, 6, 5, 3, 2, or 1. In one example the total run or elution time is less than about 5 minutes.

Lipidomics Assessment of Metabolic Age (mAge)

In one example, the present disclosure provides a method of determining the metabolic age (mAge) of a subject, the method comprising:
detecting in a biological sample from the subject a level of a plurality of lipid species comprising at least two lipid species selected from any lipid species of the group consisting of any one or more Tables, lipid classes, and/or lipid species according to any embodiments or examples thereof as described herein;
comparing the levels or ratios of levels of the lipid species detected in the biological sample to reference levels or ratios of the lipid species; and
determining the mAge of the subject on the basis of the comparison.
   In one example, the metabolic age is compared to the actual age of the subject.

In one example, the lipid species is selected from one or more lipid classes or sub-classes comprising or consisting of any one or more of PC(P), AC, Hex3Cer, SM, PC, Cer, Hex2Cer, PE, PE(P), LPC(O), LPC, TG, CE, PI, LPC(P), PC(O), TG(O), LPE, S1P, Hex1Cer, sulfatide, DG, and PE(O). In another example, the one or more lipid classes or sub-classes comprise or consist of any one or more of PC(P), AC, Hex3Cer, SM, PC, Cer, Hex2Cer, PE, PE(P), LPC(O), LPC, TG, and CE. In another example, the one or more lipid classes or sub-classes comprise or consist of any one or more of PC(P), AC, Hex3Cer, SM, PC, Cer, Hex2Cer, PE, PE(P).

In one example, the lipid class or subclass PC(P) comprises or consists of any one or more lipid species of 34:2, 34:1, 36:1, and 36:5.

In one example, the lipid class or subclass AC comprises or consists of any one or more lipid species of 14:1, 16:1, 16:0, 18:1, 14:2, 12:0, 14:0, 13:0, and 18:2.

In one example, the lipid class or subclass Hex3Cer comprises or consists of 18:1/16:0.

In one example, the lipid class or subclass SM comprises or consists of any one or more lipid species of 33:1, 34:2, 44:3, 32:1, 34:3, 36:3, 38:1, 34:1, 41:1, 40:!, and 36:1. In another example, the lipid class or subclass SM comprises or consists of any one or more lipid species of 33:1, 34:2, 44:3, 32:1, 34:3.

In one example, the lipid class or subclass PC comprises or consists of any one or more lipid species of 40:6, 35:5, 36:1, 36:5, 37:6, 32:2, 34:0, 38:6, and 32:0.

In one example, the lipid class or subclass Cer(m) comprises or consists of any one or more lipid species of 18:1/24:0, 18:1/24:0, 18:0/22:0, and 18:1/20:0.

In one example, the lipid class or subclass Cer(d) comprises or consists of any one or more lipid species of 18:1/23:0, 18:2/24:0, 18:1/22:0, and 18:2/23:0.

In one example, the lipid class or subclass Hex2Cer comprises or consists of any one or more lipid species of 18:1/16:0, 18:!/24:0, and 18:2/16:0.

In one example, the lipid class or subclass PE comprises or consists of any one or more lipid species of 34:2, 36:1, 32:1, and 40:6.

In one example, the lipid class or subclass PE(P) comprises or consists of any one or more lipid species of 16:0/18:1, 16:0/22:4, 18:0/20:4, 18:0/18:2, 18:0/20:5, 18:0/22:6, 18:0/22:4, 17:0/20:4, and 16:0/20:4.

In one example, the lipid class or subclass LPC(O) comprises or consists of the lipid species 20:0 and/or 24:2.

In one example, the lipid class or subclass LPC comprises or consists of any one or more lipid species of 18:2, 19:!, 20:4, 14:0, 17:0, and 18:1.

In one example, the lipid class or subclass TG comprises or consists of any one or more lipid species of 54:6, 50:3, 48:2, 53:2, 50:3, 56:8, 48:3, 58:9, 50:2, 50:4, 54:5, 56:7, 58:10, and 56:6.

In one example, the lipid class or subclass CE comprises or consists of any one or more lipid species of 17:0, 18:1, 20:2, 15:0, 20:1, 16:0, and 18:0.

In one example, the lipid class or subclass PI comprises or consists of any one or more lipid species of 38:6, 36:1, and 40:6.

In one example, the lipid class or subclass LCP(P) comprises or consists of 16:0.

In one example, the lipid class or subclass PC(O) comprises or consists of any one or more lipid species of 34:2, 32:0, 36:0, and 36:5.

In one example, the lipid class or subclass TG(O) comprises or consists of any one or more lipid species of 50:1 and 54:5.

In one example, the lipid class or subclass LPE comprises or consists of any one or more lipid species of 18:0 and 18:2.

In one example, the lipid class or subclass S1P comprises or consists of any one or more lipid species of 18:1.

In one example, the lipid class or subclass Hex1Cer comprises or consists of any one or more lipid species of 18:2/24:0, 18:1/24:1, and 18:1/16:0.

In one example, the lipid class or subclass sulfatide comprises or consists of any one or more lipid species of 18:1/16:0.

In one example, the lipid class or subclass DG comprises or consists of any one or more lipid species of 34:1, 36:2, and 36:3

In one example, the lipid class or subclass PE(O) comprises or consists of any one or more lipid species of 36:4.

In other examples, the lipid species may comprise or consist of any one or more combinations of the above lipid species or any classes or subclasses thereof.

In one example, the plurality of lipid species comprises the lipids set out at Tables 3, 4, 13, 14, 15, 16, and 30.

In one example, the plurality of lipid species comprises the lipids set out at any one or more of Tables 2, 3, 4, 5, 6, 13, 14, 15, 16, and 30. In some examples, the lipid species detected from any one or more of Tables 2, 3, 4, 5, 6, 13, 14, 15, 16, and 30 comprise at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 2, 3, 4, 5, 6, 13, 14, 15, 16, and 30 comprise less than about 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 2, 3, 4, 5, 6, 13, 14, 15, 16, and 30 comprise a range selected from any two previously described upper and/or lower amounts, such as 2 to 300, 10 to 290, 25 to 270, or 50 to 250.

In some examples, the lipid species detected from any one or more of Tables 2, 3, 4, 5, 6, 13, 14, 15, 16, and 30, in order of priority numbering in any one of Tables 13, 14, 15 and 16, comprise at least the first 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 2, 3, 4, 5, 6, 13, 14, 15, 16, and 30, in order of priority numbering in any one of Tables 13, 14, 15 and 16, comprise less than about the first 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 2, 3, 4, 5, 6, 13, 14, 15, 16, and 30, in order of priority numbering in any one of Tables 13, 14, 15, 16, and 30, comprise a range selected from any two previously described upper and/or lower amounts, such as 2 to 300, 10 to 290, 25 to 270, or 50 to 250.

In some examples, the plurality of lipid species comprises at least 20, at least 30, at least 40, or all of the lipid species described in Table 33.

As defined herein, all-cause mortality provides a measure of the excess mortality attributable to metabolic health factors such as metabolic age and metabolic BMI, especially when metabolic health factors are increase compared to actual factors in a subject, for example, where a subject's metabolic age is greater than their chronological age, or a subject's metabolic BMI is greater than their actual BMI. All-cause mortality integrates mortality across all of these causes, as well as capturing mortality that may come from still unidentified associations of metabolic health factors with disease and through indirect pathways, such as diminished immune function and inflammatory response. All-cause mortality measures general health status, assessing the ongoing impact of metabolic health factors on the health and risk of developing disease of a subject.

In one example, the hazard ratios for all-cause mortality risk across the quintiles of mAgeΔ were computed from a Cox regression between time to death and 17-year all-cause mortality, n=1706 and the quintiles of the mAgeΔ (Q1 as a reference) adjusted for age, sex, BMI, smoking status and diabetes.

mAge Calculation

The present disclosure also provides a method of a calculating a metabolic age score. The method may comprise obtaining lipid profile data from a biological sample taken from the subject, which may comprise lipid species according to any aspects, embodiments, or examples thereof as described herein. The method may also comprise standardising or normalising or scaling the numeric values of the lipid profile data against a reference data set. The method may also comprise refining the discriminatory power of one or more lipid species by statistically weighting some of the numeric values associated therewith. The method may also comprise summating or similar the numeric values obtained from steps (above) to provide a composite metabolic body mass score.

In one example, there is provided a method of a calculating a metabolic age score comprising:
obtaining lipid profile data from a biological sample taken from the subject;
standardising or normalising or scaling the numeric values of the lipid profile data against a reference data set;
refining the discriminatory power of one or more lipid species by statistically weighting some of the numeric values associated therewith;
calculating a composite metabolic age score.

In some examples, the mAge score can be used to determine the metabolic health of the subject.

In some examples, the mAge score can be used to determine the risk of the subject developing disease, preferably cardiovascular disease.

In some examples, the mAge score can be used to determine a treatment plan or method of treating a subject to improve metabolic health of the subject.

In some examples, the mAge score is standardised for age and/or gender of the subject.

In some examples, the subject is a mammal.

In some examples, the subject is a human.

In some examples, the lipid profile data is generated form a liquid chromatography-mass spectrometry (LC-MS) protocol.

In some examples, the LC-MS protocol has a total run time of less than about (in minutes) 15, 14, 13, 12, 11, 10, 9, 8, 7, 6, 5, 3, 2, or 1. In one example the total run or elution time is less than about 5 minutes.

In some examples, the mAge of a subject is compared to the chronological age of a subject, the difference in mAge and chronological age of a subject defined herein as mAgeΔ.

In one example, the predicted age (pAge) is standardised to the population, the mAge then derived from the pAge scores as follows:

mAge=age+(pAge−(pAge value on the line of best fit between pAge and actual age))

In one example, the mAgeΔ is defined as the difference between age and mAge.

### Lipidomics Assessment of Cardiovascular Disease (CVD) Risk

In one example, the present disclosure provides a method of determining risk of a cardiovascular disease (CVD) event in a subject, the method comprising:
detecting in a biological sample from the subject a level of a plurality of lipid species comprising at least two lipid species selected from any lipid species of the group consisting of any one or more Tables, lipid classes, and/or lipid species according to any embodiments or examples thereof as described herein;
comparing the levels or ratios of levels of the lipid species detected in the biological sample to reference levels or ratios of the lipid species; and
determining risk of a cardiovascular disease (CVD) event in the subject on the basis of the comparison.

In one example, the lipid species is selected from one or more lipid classes or sub-classes comprising or consisting of any one or more of SM, PC, AC, Cer(m), PE, PC, LPC(O), DE, LPC(P), LPC, PE(P), PC(P), CE, TG(O), DG, TG, Cer(d), LPE, PE(O), PI, Hex3Cer, Sph, GM3, and PC(O). In another example, the lipid species is selected from one or more lipid classes or sub-classes comprising or consisting of any one or more of SM, PC, AC, Cer(m), PE, PC, LPC(O), DE, LPC(P), LPC, PE(P), PC(P), CE, TG(O). In another example, the lipid species is selected from one or more lipid classes or sub-classes comprising or consisting of any one or more of SM, PC, AC, Cer(m), PE, PC, LPC(O).

In one example, the lipid class or subclass SM comprises or consists of any one or more lipid species of 31:1, 34:2, 34:1, 36:3, 38:3, 32:2, and 44:2.

In one example, the lipid class or subclass PC comprises or consists of any one or more lipid species of 40:8.

In one example, the lipid class or subclass AC comprises or consists of any one or more lipid species of 12:0 and 14:0.

In one example, the lipid class or subclass Cer(m) comprises or consists of any one or more lipid species of 18:1/24:0 and 18:1/20:0.

In one example, the lipid class or subclass PE comprises or consists of any one or more lipid species of 40:6, 34:1, 32:1, and 36:4.

In one example, the lipid class or subclass PC comprises or consists of any one or more lipid species of 40:6, 32:0, 38:4, 36:5, 36:1, 34:2, 32:2, and 38:6.

In one example, the lipid class or subclass LPC(O) comprises or consists of any one or more lipid species of 24:0, 24:3, 20:0.

In one example, the lipid class or subclass DE comprises or consists of any one or more lipid species of 18:2 and 18:1.

In one example, the lipid class or subclass LPC(P) comprises or consists of any one or more lipid species of 17:0.

In one example, the lipid class or subclass LPC comprises or consists of any one or more lipid species of 14:0, 22:5, 19:1, 18:1, and 18:2.

In one example, the lipid class or subclass PE(P) comprises or consists of any one or more lipid species of 18:0/18:2, 16:0/18:1, 18:0/22:6, 18:0/18:1, and 18:0/22:4.

In one example, the lipid class or subclass PC(P) comprises or consists of any one or more lipid species of 34:2, 30:0, 34:1, and 32:0.

In one example, the lipid class or subclass CE comprises or consists of any one or more lipid species of 18:0, 16:0, 20:4, 17:0, 16:2, 22:6, 22:1, 20:1, and 15:0.

In one example, the lipid class or subclass TG(O) comprises or consists of any one or more lipid species of 52:2.

In one example, the lipid class or subclass DG comprises or consists of any one or more lipid species of 34:1, 36:2, and 36:3.

In one example, the lipid class or subclass TG comprises or consists of any one or more lipid species of 54:4, 48:2, 50:4, 58:10, 58:9, 52:5, 56:8, 54:6, 54:7, 56:7, 54:2, and 50:3.

In one example, the lipid class or subclass Cer(d) comprises or consists of any one or more lipid species of 19:1/22:0, 18:1/24:1, 18:1/24:0, 19:1/24:1, 16:1/24:1, 18:2/24:0, and 18:2/23:0.

In one example, the lipid class or subclass LPE comprises or consists of any one or more lipid species of 18:1 and 20:4.

In one example, the lipid class or subclass PE(O) comprises or consists of any one or more lipid species of 34:1, 34:2, and 36:4.

In one example, the lipid class or subclass PI comprises or consists of any one or more lipid species of 36:1, 36:2, 32:1, and 38:2.

In one example, the lipid class or subclass Hex3Cer comprises or consists of any one or more lipid species of 18:1/16:0.

In one example, the lipid class or subclass Sph comprises or consists of any one or more lipid species of 17:1.

In one example, the lipid class or subclass GM3 comprises or consists of any one or more lipid species of 18:1/16:0.

In one example, the lipid class or subclass PC(O) comprises or consists of any one or more lipid species of 36:0.

In other examples, the lipid species may comprise or consist of any one or more combinations of the above lipid species or any classes or subclasses thereof.

In one example, the plurality of lipid species comprises the lipids set out at Tables 19, 20, 21, 22, and 30.

In one example, the plurality of lipid species comprises the lipids set out at Tables 2, 3, 4, 5, 6, 19, 20, 22, and 30. In some examples, the lipid species detected from any one or more of Tables 2, 3, 4, 5, 6, 19, 20, 22, and 30 comprise at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 2, 3, 4, 5, 6, 19, 20, 22, and 30 comprise less than about 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 2, 3, 4, 5, 6, 19, 20, 22, and 30 comprise a range selected from any two previously described upper and/or lower amounts, such as 2 to 300, 10 to 290, 25 to 270, or 50 to 250.

In some examples, the lipid species detected from any one or more of Tables 2, 3, 4, 5, 6, 19, 20, 22, and 30, in order of priority numbering in any one of Tables 19, 20, 22, and 30, comprise at least the first 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 2, 3, 4, 5, 6, 19, 20, 22, and 30, in order of priority numbering in any one of Tables 19, 20, 21 and 22, comprise less than about the first 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 2, 3, 4, 5, 6, 19, 20, 22, and 30, in order of priority numbering in any one of Tables 19, 20, 21 and 22, comprise a range selected from any two previously described upper and/or lower amounts, such as 2 to 300, 10 to 290, 25 to 270, or 50 to 250.

In some examples, the plurality of lipid species comprises at least 20, at least 30, at least 40, or all of the lipid species described in Table 35.

### CVD Risk Calculation

The present disclosure also provides a method of a calculating a cardiovascular disease risk (CVD) score. The method may comprise obtaining lipid profile data from a biological sample taken from the subject, which may comprise lipid species according to any aspects, embodiments, or examples thereof as described herein. The method may also comprise standardising or normalising or scaling the numeric values of the lipid profile data against a reference data set. The method may also comprise refining the discriminatory power of one or more lipid species by statistically weighting some of the numeric values associated therewith. The method may also comprise summating or similar the numeric values obtained from steps (above) to provide a composite metabolic body mass score.

In one example, there is provided a method of a calculating a cardiovascular disease risk score comprising:
obtaining lipid profile data from a biological sample taken from the subject;
standardising or normalising or scaling the numeric values of the lipid profile data against a reference data set;
refining the discriminatory power of one or more lipid species by statistically weighting some of the numeric values associated therewith;
calculating a CVD score.

In some examples, the method of calculating a CVD risk score comprises:
(i) internal validation of a reference population sample;
(ii) standardising each continuous variable;
(iii) log transforming each lipid concentration; and
(iv) further standardising the variables prior to their use as the predictors of a CVD event.

In one example, the CVD risk score is developed by machine learning.

In one example, the model used to develop the CVD risk score comprises:
(i) performing ridge regression based on the following formula:

$$FRS ∼ μ + {\sum }_{i = 1}^{n} {\hat{β}}_{i} \times {lipid}_{i} + {\hat{β}}_{age} \times age \qquad ( 1 )$$

where alpha is set to 0 and 1, respectively and n referred to the total number of the lipids;
(ii) a term (FRSΔ) calculated as the residuals of pFRS and the best fit of line between pFRS and FRS is introduced according to the following formula:

$$pFRS ∼ μ + \hat{β} \times FRS \qquad ( 2 )$$

(iii) calculating the residual FRSΔ by subtracting the predicted value of (2) from the predicted value of (1).
(iv) residue adjusting FRS (raFRS) by adding the FRSΔ to FRS.
(v) deriving the LRS for the prediction of the incident CVD from the derived and an age of a subject.

In some examples, the CVD risk score can be used to determine the metabolic health of the subject.

In some examples, the CVD risk score can be used to determine the risk of the subject developing cardiovascular disease.

In some examples, the CVD risk score can be used to determine a treatment plan or method of treating a subject at risk of cardiovascular disease.

In some examples, the CVD risk score is standardised for age and/or gender of the subject.

In some examples, the subject is a mammal.

In some examples, the subject is a human.

In some examples, the lipid profile data is generated form a liquid chromatography-mass spectrometry (LC-MS) protocol.

In some examples, the LC-MS protocol has a total run time of less than about (in minutes) 15, 14, 13, 12, 11, 10, 9, 8, 7, 6, 5, 3, 2, or 1. In one example the total run or elution time is less than about 5 minutes.

### Lipidomics Assessment of Diabetes or Impaired Glucose Tolerance Risk

In one example, the present disclosure provides a method of determining the presence or risk of developing diabetes or impaired glucose tolerance in a subject, the method comprising:
detecting in a biological sample from the subject a level of a plurality of lipid species comprising at least two lipid species selected from any lipid species of the group consisting of any one or more Tables, lipid classes, and/or lipid species according to any embodiments or examples thereof as described herein;
comparing the levels or ratios of levels of the lipid species detected in the biological sample to reference levels or ratios of the lipid species; and
determining the presence or risk of developing diabetes or impaired glucose tolerance in the subject on the basis of the comparison.

In other examples, the lipid species may comprise or consist of any one or more combinations of the above lipid species or any classes or subclasses thereof.

In one example, the plurality of lipid species comprises the lipids set out at Tables 3, 4, 5, 6, 23, 24, 25, 26, and 30.

In one example, the plurality of lipid species comprises the lipids set out at Tables 3, 4, 5, 6, 23, 24, 25, 26, and 30. In some examples, the lipid species detected from any one or more of Tables 3, 4, 5, 6, 23, 24, 25, 26, and 30 comprise at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 3, 4, 5, 6, 23, 24, 25, 26, and 30 comprise less than about 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 3, 4, 5, 6, 23, 24, 25, 26, and 30 comprise a range selected from any two previously described upper and/or lower amounts, such as 2 to 300, 10 to 290, 25 to 270, or 50 to 250.

In some examples, the lipid species detected from any one or more of Tables 3, 4, 5, 6, 23, 24, 25, 26, and 30, in order of priority numbering in any one of Tables 23, 24, 25 and 26, comprise at least the first 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 150, 160, 170, 180, 190, 200, 210, 220, 230, 240, 250, 260, 270, 280, 290, or 300. In some examples, the lipid species detected from any one or more of Tables 3, 4, 5, 6, 23, 24, 25, 26, and 30, in order of priority numbering in any one of Tables 23, 24, 25 and 26, comprise less than about the first 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15, or 10. In some examples, the lipid species detected from any one or more of Tables 3, 4, 5, 6, 23, 24, 25, 26, and 30, in order of priority numbering in any one of Tables 23, 24, 25 and 26, comprise a range selected from any two previously described upper and/or lower amounts, such as 2 to 300, 10 to 290, 25 to 270, or 50 to 250.

In some examples, the plurality of lipid species comprises at least 20, at least 30, at least 40, or all of the lipid species described in Table 36.

### Diabetes Risk Calculation

The present disclosure also provides a method of a calculating a Diabetes risk score. The method may comprise obtaining lipid profile data from a biological sample taken from the subject, which may comprise lipid species according to any aspects, embodiments, or examples thereof as described herein. The method may also comprise standardising or normalising or scaling the numeric values of the lipid profile data against a reference data set. The method may also comprise refining the discriminatory power of one or more lipid species by statistically weighting some of the numeric values associated therewith. The method may also comprise summating or similar the numeric values obtained from steps (above) to provide a composite Diabetes risk score.

In one example, there is provided a method of a calculating a Diabetes risk score comprising:
obtaining lipid profile data from a biological sample taken from the subject;
standardising or normalising or scaling the numeric values of the lipid profile data against a reference data set;
refining the discriminatory power of one or more lipid species by statistically weighting some of the numeric values associated therewith;
determining a composite Diabetes risk score.

In some examples, the Diabetes risk score can be used to determine the metabolic health of the subject.

In some examples, the Diabetes risk score can be used to determine the risk of the subject developing diabetes.

In some examples, the Diabetes risk score can be used to determine a treatment plan or method of treating a subject at risk of developing diabetes.

In some examples, the Diabetes risk score can be used to develop a treatment plan for improving the subject's metabolic health.

In some examples, the Diabetes risk score is standardised for age and/or gender of the subject.

In some examples, the subject is a mammal.

In some examples, the subject is a human.

In some examples, the lipid profile data is generated form a liquid chromatography-mass spectrometry (LC-MS) protocol.

In some examples, the LC-MS protocol has a total run time of less than about (in minutes) 15, 14, 13, 12, 11, 10, 9, 8, 7, 6, 5, 3, 2, or 1. In one example the total run or elution time is less than about 5 minutes.

### Plasmalogen Score (PS)

In one example, the present disclosure provides a method of determining a phospholipid score (PhS) in a subject, the method comprising:
detecting in a biological sample from the subject a level of a plurality of lipid species comprising at least two lipid species selected from an ether phospholipid and an acyl phospholipid;
comparing the levels or ratios of levels of the lipid species detected in the biological sample to reference levels or ratios of the lipid species; and
determining the presence or risk of developing diabetes in the subject on the basis of the comparison.

In one example, the phospholipid score is a plasmalogen score (PS). In one example, the acyl phospholipid is a phosphatidylethanolamine (PE). In one example the ether phospholipid is a plasmalogen PE(P).

In one example, the present disclosure provides a method of determining a plasmalogen score (PS) in a subject, the method comprising:
detecting in a biological sample from the subject a level of a plurality of lipid species comprising at least two lipid species selected from a PE and a PE(P) lipid species;
comparing the levels or ratios of levels of the lipid species detected in the biological sample to reference levels or ratios of the lipid species; and
determining the presence or risk of developing diabetes or impaired glucose tolerance in the subject on the basis of the comparison.

In some examples, the lipid species comprises at least one PE and/or at least one PE(P) lipid species. In some examples, the lipid species comprises at least two PE and/or at least two PE(P) lipid species. In some examples, the lipid species comprises at least three PE and/or at least three PE(P) lipid species. In some examples, the lipid species comprises at least four PE and/or at least four PE(P) lipid species. In some examples, the lipid species comprises at least five PE and/or at least five PE(P) lipid species. In some examples, the lipid species comprises at least six PE and/or at least six PE(P) lipid species.

In one example, the PE lipid species is selected from the group consisting of PE(16:0/18:1), PE(16:0/20:4, PE(16:0/18:2), PE(18:0/20:4), PE(16:0/16:1), PE(18:0/18:1), PE(16:0/22:6), PE(16:1/18:2), PE(18:0/22:6), and PE(18:1/18:1).

In one example, the PE(P) lipid species is selected from the group consisting of PE(P-18:0/20:4), PE(P-16:0/20:4), PE(P-18:0/22:6), PE(P-18:1/20:4), PE(P-18:0/18:2), PE(P-18:0/18:1), PE(P-18:0/20:5), PE(P-17:0/22:6), PE(P-17:0/20:4), PE(P-16:0/18:2), PE(P-16:0/20:5), PE(P-18:1/20:5), PE(P-16:0/18:1), PE(P-20:0/20:4), PE(P-16:0/22:6), PE(P-20:0/18:2), PE(P-18:0/22:4), and PE(P-16:0/22:4).

In one example, the plurality of lipid species comprises the lipids set out at Table 17.

In one example, the plurality of lipid species comprises the lipids set out at Tables 3, 4, 5, 6 and 17. In some examples, the lipid species detected from any one or more of Tables 3, 4, 5, 6 and 17 comprise at least 2, 3, 4, 5, 6, 7, 8, 9, 10 or 15. In some examples, the lipid species detected from any one or more of Tables 3, 4, 5, 6 and 17 comprise less than about 25, 20, 15, 10, 9, 8, 7, 6, or 5. In some examples, the lipid species detected from any one or more of Tables 3, 4, 5, 6 and 17 comprise a range selected from any two previously described upper and/or lower amounts, such as 2 to 35, 3 to 30, or 5 to 25.

In some examples, the lipid species detected from any one or more of Tables 3, 4, 5, 6 and 17, in order of priority numbering in Table 17, comprise at least the first 2, 3, 4, 5, 6, 7, 8, 9, 10, 15, 20, 25, 30, or 35. In some examples, the lipid species detected from any one or more of Tables 3, 4, 5, 6 and 17, in order of priority numbering in Table 17, comprise less than about the first 25, 20, 15, 10, 9, 8, 7, 6 or 5. In some examples, the lipid species detected from any one or more of Tables 3, 4, 5, 6 and 17, in order of priority numbering in Table 17, comprise a range selected from any two previously described upper and/or lower amounts, such as 2 to 25, 3 to 20, or 5 to 15.

In one embodiment, the plasmalogen level is used to calculate the risk of the subject developing one or more diseases, including respiratory diseases, neurodegenerative diseases, lipid signalling diseases and inflammatory diseases. For example, cardiovascular disease, diabetes, impaired glucose tolerance metabolic syndrome and chronic inflammation.

In one embodiment, the plasmalogen level is used to calculate the risk of the subject developing cardiovascular disease and/or diabetes and/or impaired glucose tolerance.

### PS Calculation

The present disclosure also provides a method of a calculating a phospholipid score (PhS) or a Plasmalogen score (PS). The method may comprise obtaining lipid profile data from a biological sample taken from the subject, which may comprise lipid species according to any aspects, embodiments, or examples thereof as described herein, and which includes phosphatidylethanolamine plasmalogens (alkenylphosphatidylethanolamine) (PE(P)) species and phosphatidylethanolamine (PE) species. The method may also comprise standardising or normalising or scaling the numeric values of the lipid profile data against a reference data set. The method may also comprise refining the discriminatory power of one or more lipid species by statistically weighting some of the numeric values associated therewith. The method may also comprise summating or similar the numeric values obtained from steps (above) to provide a composite Plasmalogen score. The method may comprise determining the relative levels of both PE(P) and PE species.

In one example, there is provided a method of a calculating a Plasmalogen score comprising:
obtaining lipid profile data from a biological sample taken from the subject;
standardising or normalising or scaling the numeric values of the lipid profile data against a reference data set;
refining the discriminatory power of one or more lipid species by statistically weighting some of the numeric values associated therewith;
determining a Plasmalogen score.

In one example, there is provided a method of calculating a Plasmalogen score comprising:
obtaining lipid profile data from a biological sample taken from the subject;
calculating the relative levels of phosphatidylethanolamine plasmalogens (PE(P) species and phosphatidylethanolamine (PE) species;
generating a ratio of PE(P):PE;
using the ratio to determine a plasmalogen score.

In some examples, the Plasmalogen score can be used to determine the metabolic health of the subject.

In some examples, the Plasmalogen score can be used to determine the risk of the subject developing disease.

In some examples, the Plasmalogen score can be used to determine a treatment plan or method of treating a subject at risk of developing disease.

In some examples, the Plasmalogen score can be used to develop a treatment plan for improving the subject's metabolic health.

In some examples, the Plasmalogen score is standardised for age and/or gender of the subject.

In some examples, the subject is a mammal.

In some examples, the subject is a human.

In some examples, the lipid profile data is generated form a liquid chromatography-mass spectrometry (LC-MS) protocol.

In some examples, the LC-MS protocol has a total run time of less than about (in minutes) 15, 14, 13, 12, 11, 10, 9, 8, 7, 6, 5, 3, 2, or 1. In one example the total run or elution time is less than about 5 minutes.

In some examples, the plasmalogen score is used as a risk marker for disease.

In some example, the Plasmalogen score is used to assess a subject's plasmalogen levels and particularly to identify low phosphatidylethanolamine plasmalogen levels relative to phosphatidylethanolamine levels.

In one example, the plasmalogen score is used to assess risk of the subject developing a disease

### High Throughput Lipidomics Assay

Illustrative methods capable of analysing lipid species include classical lipid extraction methods, mass spectrometry together with electrospray ionization and matrix-assisted laser desorption ionisation, with mass analysis such as quadruple and/or TOF (e.g., Quadrapole/TOF) or orbitrap mass analysers. Chromatographic methods are used for the separation of lipid mixtures such as gas chromatography, high pressure liquid chromatography (HPLC), ultra-high pressure liquid chromatography (UHPLC), capillary electrophoresis (CE). These may be used with mass spectrometry based detection systems or other detectors including optical detectors. Clinical mass spectrometry systems are used by clinical laboratories to provide lipid profiles and ratios upon request. Another suitable technique for quantitative lipid analysis is one or two dimensional nuclear magnetic resonance (NMR). Two dimensional techniques such as heteronuclear single quantum coherence (HSQC) are suitable for lipid profiling through the ability to elucidate C—H bonds within a structure. Any technique capable of identifying individual lipid species in the sample can be used for collecting information on the lipid species. Typically MS is used coupled to a separation method such as various forms of chromatography.

In one example, there is provided a high throughput lipidomics assay comprising:
obtaining a biological sample
separating the plasma sample analytes by liquid chromatographic separation
analysing the analytes using a mass spectrometer;
wherein the total run time is less than 15 minutes; and
wherein the resulting measured data can accurately assign individual analytes to a single lipid species.

In one example, at least two solvents are run.

In one example, the solvents are A) 50% H2O/30% acetonitrile/20% isopropanol (v/v/v) with 10 mM ammonium formate and B) 1% H2O/9% acetonitrile/90% isopropanol (v/v/v) with 10 mM ammonium formate.

In one example, the total run time is less than 10 minutes.

In one example, the total run time is less than 8 minutes.

In one example, the total run time is less than 7 minutes.

In one example, the total run time is not more than 5 minutes.

In one example, the biological sample is at least one of plasma, serum, dried blood spots, and dried plasma spots.

In one example, the plasma sample is extracted using a single-phase BuOH/MeOH.

In one example, the plasma sample is obtained from a subject.

In one example, the subject is a mammal.

In one example, the subject is a human.

### EXAMPLES

### Example 1: High Throughput Lipidomic Assay

A chromatographic gradient was devised to achieve the best possible separation of lipid species while maintaining a total runtime no greater than 5 minutes.

### Lipid Extraction

Aliquots of 10 uL plasma were mixed with 100 μL of butanol:methanol, 1:1, containing 10 mM ammonium formate, and a mixture of internal standards at known concentrations. Samples were vortexed thoroughly, bath sonicated for 1 hour at 25° C., then centrifuged at 14,000 rpm for 10 min at 20° C. A 90 μL aliquot was removed for analysis discarding any precipitate.

### Internal Standard Addition

To calculate lipid concentration within the sample, a mixture of internal standards is added to the extraction solution, such that it is included in each sample at known concentrations. Internal standards consist of stable-isotope-labelled and non-physiological species, with a total of 30 used. A mixture of these standards is prepared in-house from commercially available sources.

TABLE 1
Internal Standards

| Compound | pmol/sample (10 uL) |
|---|---|
| AcylCarnitine 16:0 d3 (IS) | 10 |
| CE 18:0-d6 (IS) | 1000 |
| Cer(d18:1-d7/18:0) (IS) | 50 |
| Cholic Acid d4 (IS) | 50 |
| COH-d7 (IS) | 10000 |
| DE(18:1) ester d6 (IS) | 100 |
| DG(15:0 18:1) d7 (IS) | 200 |
| FA(18:1) d9 (IS) | 200 |
| FA(20:4) d11 (IS) | 100 |
| FA(22:6) d5 (IS) | 100 |
| Hex2Cer(d18:1/15:0) d7 (IS) | 50 |
| Hex3Cer(d18:1/17:0) (IS) | 50 |
| HexCer(d18:1/15:0) d7 (IS) | 50 |
| LPC(18:1) d7 (IS) | 100 |
| LPE(18:1) d7 (IS) | 100 |
| LPI 13:0 (IS) | 20 |
| MG(18:1) d7 (IS) | 50 |
| PA(15:0_18:1) d7 (IS) | 50 |
| PC(15:0_18:1) d7 (IS) | 100 |
| PC(P-18:0/18:1) d9 (IS) | 100 |
| PE(15:0_18:1) d7 (IS) | 100 |
| PE(P-18:0/18:1) d9 (IS) | 100 |
| PG(15:0_18:1) d7 (IS) | 50 |
| PI(15:0_18:1) d7 (IS) | 50 |
| PS(15:0_18:1) d7 (IS) | 50 |
| S1P(d18:1) d7 (IS) | 40 |
| SHexCer(d18:1/12:0) (IS) | 10 |
| SM(d18:1/15:0) d9 (IS) | 100 |
| Sph(d17:1) (IS) | 20 |
| TG(48:1) [NL-18:1] d7 (IS) | 100 |

### Data Analysis

Peak area was calculated using Agilent's MassHunter Quant QQQ software package, via integration of the chromatogram. Lipid concentration was calculated using the ratio of peak area between analyte and assigned internal standard, along with the internal standard's known concentration.

### Research Protocol LC-MS/MS

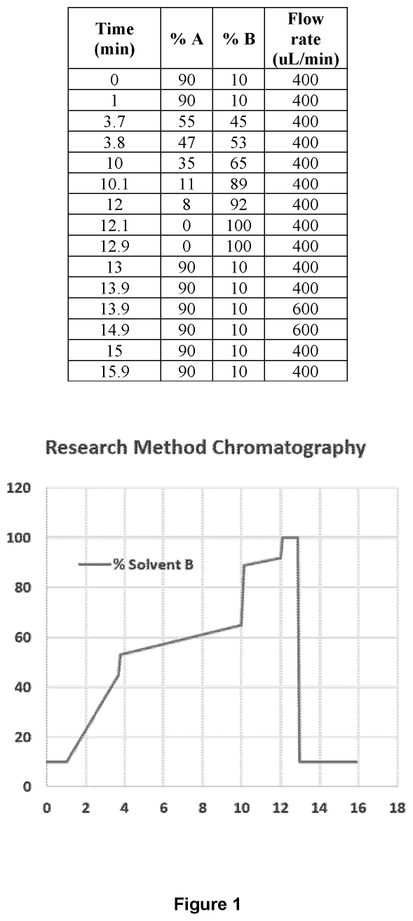

Extracted plasma samples were separated and analysed using an Agilent 1290 Infinity liquid chromatography (LC) system coupled to an Agilent 6495C QQQ mass spectrometer equipped with Jet Stream ionisation source. The LC system utilised a ZORBAX eclipse plus C18 column (2.1×100 mm 1.8 mm, Agilent), with the thermostat set at 45° C. The running solvents consisted of: A) 50% H2O/30% acetonitrile/20% isopropanol (v/v/v) with 10 mM ammonium formate and B) 1% H2O/9% acetonitrile/90% isopropanol (v/v/v) with 10 mM ammonium formate. A stepped, linear solvent gradient was used, detailed in FIG. 1.

The gradient started with a flow rate of 0.4 ml/minute at 10% B and increased to 45% B over 2.7 minutes, then to 53% over 0.1 minutes, to 65% over 6.2 minutes, to 89% over 0.1 minute, to 92% over 1.9 minutes and finally to 100% over 0.1 minute. The solvent was then held at 100% B for 0.8 minutes (total 11.9 minutes). For equilibration the solvent was decreased from 100% B to 10% B over 0.1 minute and held for an additional 0.9 minutes. Flow rate was then switched to 0.6 ml/minute for 1 minute before returning to 0.4 ml/minute over 0.1 minutes. Solvent B was held at 10% B for a further 0.9 minutes at 0.4 ml/minutes for a total cycle time of 15 minutes. Mass spectrometer conditions used were: gas temperature, 150° C.; gas flow rate 17 L/min; nebulizer 20 psi; sheath gas temperature 200° C.; capillary voltage 3500 V; and sheath gas flow 10 L/min.

### Clinical Protocol LC-MS/MS

Additional modifications were made to solvent flow rate, column temperature, column length and packing material, and MS source conditions. The gradient was shortened to 5 minutes, and the length and packing material of the C18 column was modified. The result was a chromatographic gradient lasting 5 minutes in total, consisting of 5 steps of variable length, and which achieved good separation across elution bands of high abundance analytes.

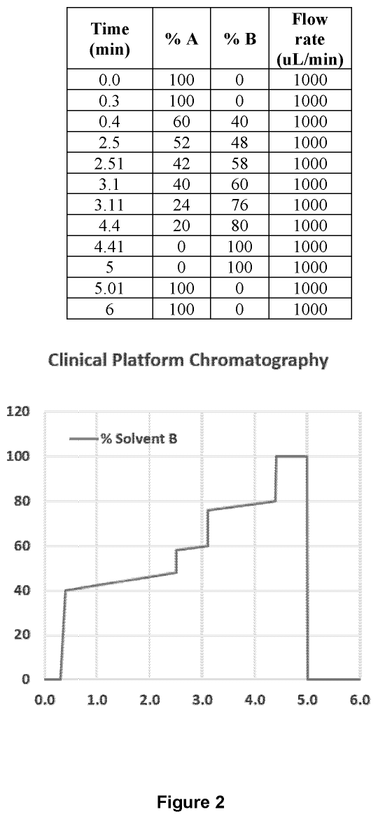

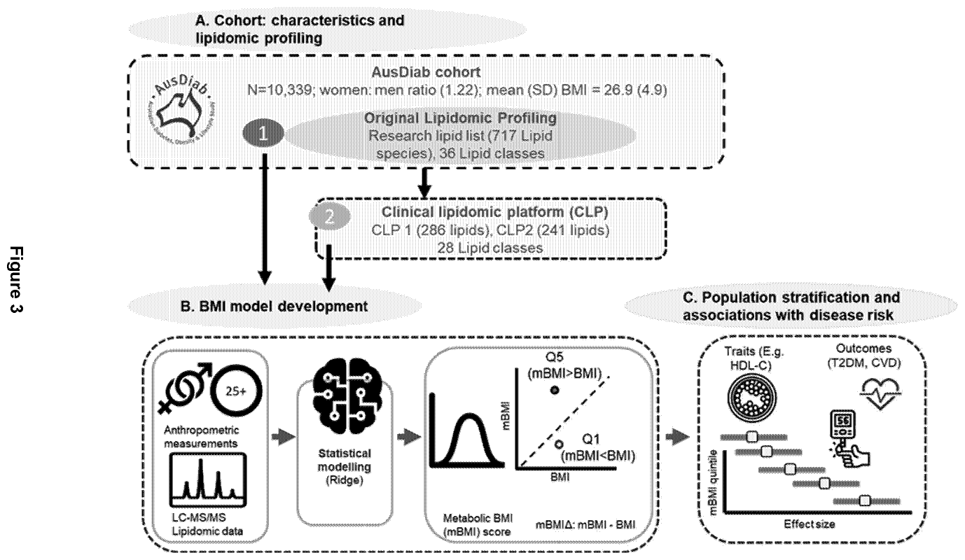

Extracted plasma samples were separated and analyzed using an Agilent 1290 Infinity II LC system coupled to an Agilent Ultivo QQQ mass spectrometer equipped with Jet Stream ionisation source. The LC system utilised an Agilent Poroshell 1290 EC-C18 column (2.1×30 mm 1.9 mm, Agilent), with the thermostat set at 40° C. The running solvents consisted of A) 50% H2O/30% acetonitrile/20% isopropanol (v/v/v) with 10 mM ammonium formate and B) 1% H2O/9% acetonitrile/90% isopropanol (v/v/v) with 10 mM ammonium formate. A stepped, linear solvent gradient was used, detailed in FIG. 2.

At 0 min, the gradient flow rate was initiated at 1 ml/minute, 0% B, maintained until 0.3 min, when B was increased to 40% over 0.1 min. B was further increased linearly to 48% B at 2.5 min, then stepped to 58% B at 2.51 min. B was increased linearly to 60% at 3.1 min, then stepped to 76% B at 3.11, before increasing linearly to 80% B at 4.4 min. B was increased at 4.41 minutes to 100%, maintained at 100% until 5 min, then immediately dropped to 0% B at 5.01 min, for equilibration at 0% B for 1 minute until 6 min. Total cycle time was 6 minutes; total analysis cycle time was 5 minutes. Mass spectrometer conditions used were: gas temperature 200° C.; gas flow rate 9 L/min; nebulizer 20 psi; sheath gas temperature 250° C.; capillary voltage 5500 V; and sheath gas flow 10 L/min.

Good separation of high abundance analytes was achieved for many lipid species. Where two or more dissimilar, low correlation lipid structures displayed the same transition measurement in the spectrometer, these species were removed from analysis, as the data regarding their measured concentration could not be accurately assigned to a single lipid species. Where the concentration of structurally similar co-eluting species (for example, isomeric and isobaric species) was observed to be highly correlated in human plasma, the species remained suitable for transition measurement despite containing multiple isomeric compounds. Such was the case, for example, for many sn1/sn2 positional isomers of glycerophospholipid structures.

### Example 2: Development of Clinical Lipidomics Platform (CLP) List

To develop the Clinical Lipidomics Platform 1 List (Table 4), a series of 791 mass transitions representing different lipid species were measured by mass spectrometer. These transitions were previously determined to be accurately quantifiable via the use of the Research Protocol LC-MS/MS totalling 15 minutes in length (described above). A series of experiments determined which of these species remain accurately quantifiable when the Clinical Protocol LC-MS/MS totalling 5 minutes in length (described above) is run to support high sample throughput. Analysed species (Table 2) which do not fit the criteria for accurate measurement following modifications to the chromatography conditions are discarded from the CLP1 List (Table 4). A summary of this process is provided below.

### Peak Overlap

A series of identical plasma extracts pooled from multiple individuals were analysed using the Research Protocol LC-MS/MS, then again using the Clinical Protocol LC-MS/MS Conditions. In both cases, lipids from the Research Lipid List (Table 2) were analysed. 30 internal standards were also measured (Table 3).

TABLE 2
Research Lipid List

| Lipid | | | Precursor | Product |
|---|---|---|---|---|
| Number | Lipid Class | Compound Name | (m/z) | (m/z) |
| 1 | AC | AC(10:0) | 316.3 | 85.1 |
| 2 | AC | AC(12:0) | 344.3 | 85.1 |
| 3 | AC | AC(12:1) | 342.3 | 85.1 |
| 4 | AC | AC(13:0) | 358.3 | 85.1 |
| 5 | AC | AC(14:0) | 372.3 | 85.1 |
| 6 | AC-OH | AC(14:0)-OH | 388.3 | 85.1 |
| 7 | AC | AC(14:1) | 370.3 | 85.1 |
| 8 | AC-OH | AC(14:1)-OH | 386.3 | 85.1 |
| 9 | AC | AC(14:2) | 368.3 | 85.1 |
| 10 | AC | AC(15:0) (a) | 386.3 | 85.1 |
| 11 | AC | AC(15:0) (b) | 386.3 | 85.1 |
| 12 | AC | AC(16:0) | 400.4 | 85.1 |
| 13 | AC-OH | AC(16:0)-OH | 416.4 | 85.1 |
| 14 | AC | AC(16:1) | 398.3 | 85.1 |
| 15 | AC-OH | AC(16:1)-OH | 414.3 | 85.1 |
| 16 | AC | AC(17:0) | 414.4 | 85.1 |
| 17 | AC | AC(18:0) | 428.4 | 85.1 |
| 18 | AC-OH | AC(18:0)-OH | 444.4 | 85.1 |
| 19 | AC | AC(18:1) | 426.4 | 85.1 |
| 20 | AC-OH | AC(18:1)-OH | 442.4 | 85.1 |
| 21 | AC | AC(18:2) | 424.3 | 85.1 |
| 22 | AC | AC(18:3) | 422.3 | 85.1 |
| 23 | AC | AC(20:3) (a) | 450.3 | 85.1 |
| 24 | AC | AC(20:3) (b) | 450.3 | 85.1 |
| 25 | AC-OH | AC(20:3)-OH | 466.3 | 85.1 |
| 26 | AC | AC(20:4) | 448.3 | 85.1 |
| 27 | AC | AC(20:5) | 446.3 | 85.1 |
| 28 | AC | AC(22:5) | 474.3 | 85.1 |
| 29 | AC-OH | AC(22:5)-OH | 490.3 | 85.1 |
| 30 | AC | AC(22:6) | 472.3 | 85.1 |
| 31 | AC | AC(24:0) | 512.3 | 85.1 |
| 32 | AC-OH | AC(24:0)-OH | 528.3 | 85.1 |
| 33 | AC | AC(24:1) (a) | 510.3 | 85.1 |
| 34 | AC | AC(24:1) (b) | 510.3 | 85.1 |
| 35 | AC-OH | AC(24:1)-OH | 526.3 | 85.1 |
| 36 | AC | AC(26:0) | 540.3 | 85.1 |
| 37 | AC | AC(26:1) | 538.3 | 85.1 |
| 38 | CE | CE(14:0) | 614.6 | 369.3 |
| 39 | CE | CE(15:0) | 628.6 | 369.3 |
| 40 | CE | CE(16:0) | 642.6 | 369.3 |
| 41 | CE | CE(16:1) | 640.6 | 369.3 |
| 42 | CE | CE(16:2) | 638.6 | 369.3 |
| 43 | CE | CE(17:0) | 656.6 | 369.3 |
| 44 | CE | CE(17:1) | 654.6 | 369.3 |
| 45 | CE | CE(18:0) | 670.7 | 369.3 |
| 46 | CE | CE(18:1) | 668.6 | 369.3 |
| 47 | CE | CE(18:2) | 666.6 | 369.3 |
| 48 | OxSpecies | CE(18:2) [+OH] | 682.6 | 369.3 |
| 49 | CE | CE(18:3) | 664.6 | 369.3 |
| 50 | CE | CE(20:0) | 698.7 | 369.3 |
| 51 | CE | CE(20:1) | 696.7 | 369.3 |
| 52 | CE | CE(20:2) | 694.7 | 369.3 |
| 53 | CE | CE(20:3) | 692.6 | 369.3 |
| 54 | CE | CE(20:4) | 690.6 | 369.3 |
| 55 | OxSpecies | CE(20:4) [+OH] | 706.6 | 369.3 |
| 56 | CE | CE(20:5) | 688.6 | 369.3 |
| 57 | CE | CE(22:0) | 726.7 | 369.3 |
| 58 | CE | CE(22:1) | 724.7 | 369.3 |
| 59 | CE | CE(22:4) | 718.7 | 369.3 |
| 60 | CE | CE(22:5) | 716.6 | 369.3 |
| 61 | CE | CE(22:6) | 714.6 | 369.3 |
| 62 | OxSpecies | CE(22:6) [+OH] | 730.6 | 369.3 |
| 63 | CE | CE(24:0) | 754.7 | 369.3 |
| 64 | CE | CE(24:1) | 752.7 | 369.3 |
| 65 | CE | CE(24:4) | 746.7 | 369.3 |
| 66 | CE | CE(24:5) | 744.7 | 369.3 |
| 67 | CE | CE(24:6) | 742.7 | 369.3 |
| 68 | Cer(d) | Cer(d16:1/16:0) | 510.6 | 236.3 |
| 69 | Cer(d) | Cer(d16:1/18:0) | 538.6 | 236.3 |
| 70 | Cer(d) | Cer(d16:1/20:0) | 566.6 | 236.3 |
| 71 | Cer(d) | Cer(d16:1/22:0) | 594.6 | 236.3 |
| 72 | Cer(d) | Cer(d16:1/23:0) | 608.6 | 236.3 |
| 73 | Cer(d) | Cer(d16:1/24:0) | 622.6 | 236.3 |
| 74 | Cer(d) | Cer(d16:1/24:1) | 620.6 | 236.3 |
| 75 | Cer(d) | Cer(d17:1/16:0) | 524.6 | 250.3 |
| 76 | Cer(d) | Cer(d17:1/18:0) | 552.6 | 250.3 |
| 77 | Cer(d) | Cer(d17:1/20:0) | 580.6 | 250.3 |
| 78 | Cer(d) | Cer(d17:1/22:0) | 608.6 | 250.3 |
| 79 | Cer(d) | Cer(d17:1/23:0) | 622.6 | 250.3 |
| 80 | Cer(d) | Cer(d17:1/24:0) | 636.6 | 250.3 |
| 81 | Cer(d) | Cer(d17:1/24:1) | 634.6 | 250.3 |
| 82 | Cer(d) | Cer(d18:1/14:0) | 510.5 | 264.3 |
| 83 | Cer(d) | Cer(d18:1/16:0) | 538.5 | 264.3 |
| 84 | Cer(d) | Cer(d18:1/18:0) | 566.6 | 264.3 |
| 85 | Cer(d) | Cer(d18:1/19:0) | 580.6 | 264.3 |
| 86 | Cer(d) | Cer(d18:1/20:0) | 594.6 | 264.3 |
| 87 | Cer(d) | Cer(d18:1/21:0) | 608.6 | 264.3 |
| 88 | Cer(d) | Cer(d18:1/22:0) | 622.6 | 264.3 |
| 89 | Cer(d) | Cer(d18:1/23:0) | 636.6 | 264.3 |
| 90 | Cer(d) | Cer(d18:1/24:0) | 650.6 | 264.3 |
| 91 | Cer(d) | Cer(d18:1/24:1) | 648.6 | 264.3 |
| 92 | Cer(d) | Cer(d18:1/26:0) | 678.6 | 264.3 |
| 93 | Cer(d) | Cer(d18:2/14:0) | 508.5 | 262.3 |
| 94 | Cer(d) | Cer(d18:2/16:0) | 536.5 | 262.3 |
| 95 | Cer(d) | Cer(d18:2/18:0) | 564.6 | 262.3 |
| 96 | Cer(d) | Cer(d18:2/20:0) | 592.6 | 262.3 |
| 97 | Cer(d) | Cer(d18:2/21:0) | 606.6 | 262.3 |
| 98 | Cer(d) | Cer(d18:2/22:0) | 620.6 | 262.3 |
| 99 | Cer(d) | Cer(d18:2/23:0) | 634.6 | 262.3 |
| 100 | Cer(d) | Cer(d18:2/24:0) | 648.6 | 262.3 |
| 101 | Cer(d) | Cer(d18:2/24:1) | 646.6 | 262.3 |
| 102 | Cer(d) | Cer(d18:2/26:0) | 676.6 | 262.3 |
| 103 | Cer(d) | Cer(d19:1/16:0) | 552.6 | 278.3 |
| 104 | Cer(d) | Cer(d19:1/18:0) | 580.6 | 278.3 |
| 105 | Cer(d) | Cer(d19:1/20:0) | 608.6 | 278.3 |
| 106 | Cer(d) | Cer(d19:1/22:0) | 636.6 | 278.3 |
| 107 | Cer(d) | Cer(d19:1/23:0) | 650.6 | 278.3 |
| 108 | Cer(d) | Cer(d19:1/24:0) | 664.6 | 278.3 |
| 109 | Cer(d) | Cer(d19:1/24:1) | 662.6 | 278.3 |
| 110 | Cer(d) | Cer(d19:1/26:0) | 692.6 | 278.3 |
| 111 | Cer(d) | Cer(d20:1/22:0) | 650.6 | 292.3 |
| 112 | Cer(d) | Cer(d20:1/23:0) | 664.6 | 292.3 |
| 113 | Cer(d) | Cer(d20:1/24:0) | 678.6 | 292.3 |
| 114 | Cer(d) | Cer(d20:1/24:1) | 676.6 | 292.3 |
| 115 | Cer(d) | Cer(d20:1/26:0) | 706.6 | 292.3 |
| 116 | Cer(m) | Cer(m18:0/20:0) | 580.6 | 268.4 |
| 117 | Cer(m) | Cer(m18:0/22:0) | 608.6 | 268.4 |
| 118 | Cer(m) | Cer(m18:0/23:0) | 622.6 | 268.4 |
| 119 | Cer(m) | Cer(m18:0/24:0) | 636.6 | 268.4 |
| 120 | Cer(m) | Cer(m18:0/24:1) | 634.6 | 268.4 |
| 121 | Cer(m) | Cer(m18:1/18:0) | 550.6 | 266.4 |
| 122 | Cer(m) | Cer(m18:1/20:0) | 578.6 | 266.4 |
| 123 | Cer(m) | Cer(m18:1/22:0) | 606.6 | 266.4 |
| 124 | Cer(m) | Cer(m18:1/23:0) | 620.6 | 266.4 |
| 125 | Cer(m) | Cer(m18:1/24:0) | 634.6 | 266.4 |
| 126 | Cer(m) | Cer(m18:1/24:1) | 632.6 | 266.4 |
| 127 | C1P | Cer1P(d18:1/16:0) | 618.4 | 264.3 |
| 128 | BA | CA | 426.3 | 355.3 |
| 129 | COH | COH | 369.4 | 161.2 |
| 130 | DE | DE(16:0) | 640.6 | 367.4 |
| 131 | DE | DE(18:1) | 666.6 | 367.4 |
| 132 | DE | DE(18:2) | 664.6 | 367.4 |
| 133 | DE | DE(20:4) | 688.6 | 367.4 |
| 134 | DE | DE(20:5) | 686.6 | 367.4 |
| 135 | DE | DE(22:6) | 712.6 | 367.4 |
| 136 | deDE | deDE(18:2) | 662.8 | 365.4 |
| 137 | deDE | deDE(20:4) | 686.8 | 365.4 |
| 138 | BA | dxCA | 410.3 | 357.3 |
| 139 | DG | DG(14:0_16:0) | 558.5 | 313.3 |
| 140 | DG | DG(16:0_16:0) | 586.5 | 313.2 |
| 141 | DG | DG(16:0_16:1) | 584.5 | 313.2 |
| 142 | DG | DG(14:0_18:2) | 582.5 | 285.2 |
| 143 | DG | DG(16:0_18:1) | 612.6 | 313.3 |
| 144 | DG | DG(16:1_18:1) | 610.5 | 339.2 |
| 145 | DG | DG(16:0_18:2) | 610.5 | 313.2 |
| 146 | DG | DG(18:0_18:1) | 640.6 | 341.3 |
| 147 | DG | DG(18:1_18:1) | 638.6 | 339.3 |
| 148 | DG | DG(18:0_18:2) | 638.6 | 341.3 |
| 149 | DG | DG(18:1_18:2) | 636.6 | 339.3 |
| 150 | DG | DG(18:2_18:2) | 634.5 | 337.2 |
| 151 | DG | DG(18:1_18:3) | 634.5 | 339.2 |
| 152 | DG | DG(16:0_20:4) | 634.5 | 313.2 |
| 153 | DG | DG(18:1_20:3) | 662.6 | 339.3 |
| 154 | DG | DG(18:0_20:4) | 662.6 | 341.3 |
| 155 | DG | DG(18:1_20:4) | 660.6 | 339.3 |
| 156 | DG | DG(16:0_22:5) | 660.6 | 313.3 |
| 157 | DG | DG(18:2_20:4) | 658.5 | 337.2 |
| 158 | DG | DG(16:0_22:6) | 658.5 | 313.2 |
| 159 | DG | DG(18:1_20:5) | 658.6 | 339.3 |
| 160 | DG | DG(18:1_22:5) | 686.6 | 339.3 |
| 161 | DG | DG(18:0_22:6) | 686.6 | 341.3 |
| 162 | DG | DG(18:1_22:6) | 684.6 | 339.3 |
| 163 | DG | DG(18:2_22:6) | 682.6 | 337.3 |
| 164 | dhCer | dhCer(d18:0/16:0) | 540.5 | 284.3 |
| 165 | dhCer | dhCer(d18:0/18:0) | 568.6 | 284.3 |
| 166 | dhCer | dhCer(d18:0/20:0) | 596.6 | 284.3 |
| 167 | dhCer | dhCer(d18:0/22:0) | 624.6 | 284.3 |
| 168 | dhCer | dhCer(d18:0/24:0) | 652.7 | 284.3 |
| 169 | dhCer | dhCer(d18:0/24:1) | 650.6 | 284.3 |
| 170 | ISTD | dhCer 8:0 (IS) | 428.4 | 284.3 |
| 171 | ISTD | dhCer(d18:0/13:0) d7 (IS) | 505.5 | 291.3 |
| 172 | dimethyl-CE | dimethyl-CE(18:1) | 696.6 | 397.3 |
| 173 | dimethyl-CE | dimethyl-CE(18:2) | 694.6 | 397.3 |
| 174 | dimethyl-CE | dimethyl-CE(20:4) | 718.6 | 397.3 |
| 175 | dimethyl-CE | dimethyl-CE(22:6) | 742.6 | 397.3 |
| 176 | FFA | FA(14:0) | 227.2 | 227.2 |
| 177 | FFA | FA(15:0) | 241.2 | 241.2 |
| 178 | FFA | FA(16:0) | 255.2 | 255.2 |
| 179 | FFA | FA(16:1) | 253.2 | 253.2 |
| 180 | FFA | FA(17:1) | 267.2 | 267.2 |
| 181 | FFA | FA(18:0) | 283.3 | 283.3 |
| 182 | FFA | FA(18:1) | 281.2 | 281.2 |
| 183 | FFA | FA(18:2) | 279.2 | 279.2 |
| 184 | FFA | FA(18:3) | 277.2 | 277.2 |
| 185 | FFA | FA(20:2) | 307.3 | 307.3 |
| 186 | FFA | FA(20:3) | 305.2 | 305.2 |
| 187 | FFA | FA(20:4) | 303.2 | 303.2 |
| 188 | FFA | FA(20:5) | 301.2 | 301.2 |
| 189 | FFA | FA(22:4) | 331.3 | 331.3 |
| 190 | FFA | FA(22:5) | 329.2 | 329.2 |
| 191 | FFA | FA(22:6) | 327.2 | 327.2 |
| 192 | GM1 | GM1(d18:1/16:0) | 760.1 | 366.2 |
| 193 | GM3 | GM3(d18:1/16:0) | 1153.7 | 264.3 |
| 194 | GM3 | GM3(d18:1/18:0) | 1181.8 | 264.3 |
| 195 | GM3 | GM3(d18:1/20:0) | 1209.8 | 264.3 |
| 196 | GM3 | GM3(d18:1/22:0) | 1237.8 | 264.3 |
| 197 | GM3 | GM3(d18:1/24:0) | 1265.8 | 264.3 |
| 198 | GM3 | GM3(d18:1/24:1) | 1263.8 | 264.3 |
| 199 | GM3 | GM3(d18:2/24:1) | 1261.8 | 262.3 |
| 200 | HexCer | HexCer(d16:1/18:0) | 700.6 | 236.3 |
| 201 | HexCer | HexCer(d16:1/20:0) | 728.6 | 236.3 |
| 202 | HexCer | HexCer(d16:1/22:0) | 756.7 | 236.3 |
| 203 | HexCer | HexCer(d16:1/24:0) | 784.7 | 236.3 |
| 204 | HexCer | HexCer(d18:1/16:0) | 700.6 | 264.3 |
| 205 | HexCer | HexCer(d18:1/18:0) | 728.6 | 264.3 |
| 206 | HexCer | HexCer(d18:1/20:0) | 756.6 | 264.3 |
| 207 | HexCer | HexCer(d18:1/22:0) | 784.7 | 264.3 |
| 208 | HexCer | HexCer(d18:1/24:0) | 812.7 | 264.3 |
| 209 | HexCer | HexCer(d18:1/24:1) | 810.7 | 264.3 |
| 210 | HexCer | HexCer(d18:2/18:0) | 726.6 | 262.3 |
| 211 | HexCer | HexCer(d18:2/20:0) | 754.6 | 262.3 |
| 212 | HexCer | HexCer(d18:2/22:0) | 782.7 | 262.3 |
| 213 | HexCer | HexCer(d18:2/24:0) | 810.7 | 262.3 |
| 214 | Hex2Cer | Hex2Cer(d16:1/16:0) | 834.6 | 236.3 |
| 215 | Hex2Cer | Hex2Cer(d16:1/24:1) | 944.7 | 236.3 |
| 216 | Hex2Cer | Hex2Cer(d18:1/16:0) | 862.6 | 264.3 |
| 217 | Hex2Cer | Hex2Cer(d18:1/18:0) | 890.7 | 264.3 |
| 218 | Hex2Cer | Hex2Cer(d18:1/20:0) | 918.7 | 264.3 |
| 219 | Hex2Cer | Hex2Cer(d18:1/22:0) | 946.7 | 264.3 |
| 220 | Hex2Cer | Hex2Cer(d18:1/24:0) | 974.8 | 264.3 |
| 221 | Hex2Cer | Hex2Cer(d18:1/24:1) | 972.7 | 264.3 |
| 222 | Hex2Cer | Hex2Cer(d18:2/16:0) | 860.6 | 262.3 |
| 223 | Hex2Cer | Hex2Cer(d18:2/18:0) | 888.7 | 262.3 |
| 224 | Hex2Cer | Hex2Cer(d18:2/24:1) | 970.7 | 262.3 |
| 225 | Hex3Cer | Hex3Cer(d18:1/16:0) | 1024.7 | 264.3 |
| 226 | Hex3Cer | Hex3Cer(d18:1/18:0) | 1052.7 | 264.3 |
| 227 | Hex3Cer | Hex3Cer(d18:1/20:0) | 1080.7 | 264.3 |
| 228 | Hex3Cer | Hex3Cer(d18:1/22:0) | 1108.8 | 264.3 |
| 229 | Hex3Cer | Hex3Cer(d18:1/24:0) | 1136.8 | 264.3 |
| 230 | Hex3Cer | Hex3Cer(d18:1/24:1) | 1134.8 | 264.3 |
| 231 | LPC | LPC(14:0) [sn2] | 468.3 | 184.1 |
| 232 | LPC | LPC(14:0) [sn1] | 468.3 | 184.1 |
| 233 | LPC | LPC(15:0) [sn2] | 482.3 | 184.1 |
| 234 | LPC | LPC(15:0) [sn1] | 482.3 | 184.1 |
| 235 | LPC | LPC(16:0) [sn2] | 496.3 | 184.1 |
| 236 | LPC | LPC(16:0) [sn1] | 496.3 | 184.1 |
| 237 | LPC | LPC(16:1) [sn2] | 494.3 | 184.1 |
| 238 | LPC | LPC(16:1) [sn1] | 494.3 | 184.1 |
| 239 | LPC | LPC(15-MHDA) [sn2] | 510.4 | 184.1 |
| 240 | LPC | LPC(15-MHDA) [sn1] & LPC(17:0) [sn2] | 510.4 | 184.1 |
| 241 | LPC | LPC(15-MHDA) [sn1] [104_sn1] | 510.4 | 104.1 |
| 242 | LPC | LPC(17:0) [sn1] | 510.4 | 184.1 |
| 243 | LPC | LPC(17:1) [sn2] (a) | 508.4 | 184.1 |
| 244 | LPC | LPC(17:1) [sn1] (a) & LPC(17:1) [sn2] (b) | 508.4 | 184.1 |
| 245 | LPC | LPC(17:1) (a) [sn1] [104_sn1] | 508.4 | 104.1 |
| 246 | LPC | LPC(17:1) [sn1] (b) | 508.4 | 184.1 |
| 247 | LPC | LPC(18:0) [sn2] | 524.4 | 184.1 |
| 248 | LPC | LPC(18:0) [sn1] | 524.4 | 184.1 |
| 249 | LPC | LPC(18:1) [sn2] | 522.4 | 184.1 |
| 250 | LPC | LPC(18:1) [sn1] | 522.4 | 184.1 |
| 251 | LPC | LPC(18:2) [sn2] | 520.3 | 184.1 |
| 252 | LPC | LPC(18:2) [sn1] | 520.3 | 184.1 |
| 253 | OxSpecies | LPC(18:2) [+OH] | 536.3 | 184.1 |
| 254 | LPC | LPC(18:3) [sn2] (a) | 518.3 | 184.1 |
| 255 | LPC | LPC(18:3) [sn1] (a) & LPC(18:3) [sn2] (b) | 518.3 | 184.1 |
| 256 | LPC | LPC(18:3) (a) [sn1] [104_sn1] | 518.3 | 104.1 |
| 257 | LPC | LPC(18:3) [sn1] (b) | 518.3 | 184.1 |
| 258 | LPC | LPC(19:0) [sn2] (a) | 538.4 | 184.1 |
| 259 | LPC | LPC(19:0) [sn1] (a) & LPC(19:0) [sn2] (b) | 538.4 | 184.1 |
| 260 | LPC | LPC(19:0) (a) [sn1] [104_sn1] | 538.4 | 104.1 |
| 261 | LPC | LPC(19:0) [sn1] (b) | 538.4 | 184.1 |
| 262 | LPC | LPC(19:1) (a) | 536.4 | 184.1 |
| 263 | LPC | LPC(19:1) (b) | 536.4 | 184.1 |
| 264 | LPC | LPC(19:1) (c) | 536.4 | 184.1 |
| 265 | LPC | LPC(20:0) [sn2] | 552.4 | 184.1 |
| 266 | LPC | LPC(20:0) [sn1] | 552.4 | 184.1 |
| 267 | LPC | LPC(20:1) [sn2] | 550.4 | 184.1 |
| 268 | LPC | LPC(20:1) [sn1] | 550.4 | 184.1 |
| 269 | LPC | LPC(20:2) [sn2] | 548.4 | 184.1 |
| 270 | LPC | LPC(20:2) [sn1] | 548.4 | 184.1 |
| 271 | LPC | LPC(20:3) [sn2] | 546.4 | 184.1 |
| 272 | LPC | LPC(20:3) [sn1] | 546.4 | 184.1 |
| 273 | LPC | LPC(20:4) [sn2] | 544.3 | 184.1 |
| 274 | LPC | LPC(20:4) [sn1] | 544.3 | 184.1 |
| 275 | OxSpecies | LPC(20:4) [+OH] | 560.3 | 184.1 |
| 276 | LPC | LPC(20:5) [sn2] | 542.3 | 184.1 |
| 277 | LPC | LPC(20:5) [sn1] | 542.3 | 184.1 |
| 278 | LPC | LPC(22:0) [sn2] | 580.4 | 184.1 |
| 279 | LPC | LPC(22:0) [sn1] | 580.4 | 184.1 |
| 280 | LPC | LPC(22:1) [sn2] | 578.4 | 184.1 |
| 281 | LPC | LPC(22:1) [sn1] | 578.4 | 184.1 |
| 282 | LPC | LPC(22:4) [sn2] | 572.4 | 184.1 |
| 283 | LPC | LPC(22:4) [sn1] | 572.4 | 184.1 |
| 284 | LPC | LPC(22:5) [sn2] (n3) | 570.4 | 184.1 |
| 285 | LPC | LPC(22:5) [sn1] (n3) & LPC(22:5) [sn2] (n6) | 570.4 | 184.1 |
| 286 | LPC | LPC(22:5) (n3) [sn1] [104_sn1] | 570.4 | 104.1 |
| 287 | LPC | LPC(22:5) [sn1] (n6) | 570.4 | 184.1 |
| 288 | LPC | LPC(22:6) [sn2] | 568.3 | 184.1 |
| 289 | LPC | LPC(22:6) [sn1] | 568.3 | 184.1 |
| 290 | OxSpecies | LPC(22:6) [+OH] | 584.3 | 184.1 |
| 291 | LPC | LPC(24:0) [sn2] | 608.5 | 184.1 |
| 292 | LPC | LPC(24:0) [sn1] | 608.5 | 184.1 |
| 293 | LPC | LPC(26:0) [sn2] | 636.5 | 184.1 |
| 294 | LPC | LPC(26:0) [sn1] | 636.5 | 184.1 |
| 295 | LPC(O) | LPC(O-16:0) | 482.4 | 104.1 |
| 296 | LPC(O) | LPC(O-18:0) | 510.4 | 104.1 |
| 297 | LPC(O) | LPC(O-18:1) | 508.4 | 104.1 |
| 298 | LPC(O) | LPC(O-20:0) | 538.4 | 104.1 |
| 299 | LPC(O) | LPC(O-20:1) | 536.4 | 104.1 |
| 300 | LPC(O) | LPC(O-22:0) | 566.5 | 104.1 |
| 301 | LPC(O) | LPC(O-22:1) | 564.4 | 104.1 |
| 302 | LPC(O) | LPC(O-24:0) | 594.5 | 104.1 |
| 303 | LPC(O) | LPC(O-24:1) | 592.5 | 104.1 |
| 304 | LPC(O) | LPC(O-24:2) | 590.5 | 104.1 |
| 305 | LPC(P) | LPC(P-16:0) | 480.3 | 104.1 |
| 306 | LPC(P) | LPC(P-17:0) (a) | 494.3 | 104.1 |
| 307 | LPC(P) | LPC(P-17:0) (b) | 494.3 | 104.1 |
| 308 | LPC(P) | LPC(P-18:0) | 508.3 | 104.1 |
| 309 | LPC(P) | LPC(P-18:1) | 506.3 | 104.1 |
| 310 | LPC(P) | LPC(P-20:0) | 536.3 | 104.1 |
| 311 | LPE | LPE(16:0) [sn2] | 454.3 | 313.3 |
| 312 | LPE | LPE(16:0) [sn1] | 454.3 | 313.3 |
| 313 | LPE | LPE(18:0) [sn2] | 482.3 | 341.3 |
| 314 | LPE | LPE(18:0) [sn1] | 482.3 | 341.3 |
| 315 | LPE | LPE(18:1) [sn2] | 480.3 | 339.3 |
| 316 | LPE | LPE(18:1) [sn1] | 480.3 | 339.3 |
| 317 | LPE | LPE(18:2) [sn2] | 478.3 | 337.3 |
| 318 | LPE | LPE(18:2) [sn1] | 478.3 | 337.3 |
| 319 | LPE | LPE(20:4) [sn2] | 502.3 | 361.3 |
| 320 | LPE | LPE(20:4) [sn1] | 502.3 | 361.3 |
| 321 | LPE | LPE(22:6) [sn2] | 526.3 | 385.3 |
| 322 | LPE | LPE(22:6) [sn1] | 526.3 | 385.3 |
| 323 | LPE(P) | LPE(P-16:0) | 438.3 | 266.4 |
| 324 | LPE(P) | LPE(P-18:0) | 466.3 | 294.4 |
| 325 | LPE(P) | LPE(P-18:1) | 464.3 | 292.4 |
| 326 | LPE(P) | LPE(P-20:0) | 494.3 | 322.4 |
| 327 | LPI | LPI(18:0) | 618.3 | 341.3 |
| 328 | LPI | LPI(18:1) [sn2] | 616.3 | 339.3 |
| 329 | LPI | LPI(18:1) [sn1] | 616.3 | 339.3 |
| 330 | LPI | LPI(18:2) [sn2] | 614.3 | 337.3 |
| 331 | LPI | LPI(18:2) [sn1] | 614.3 | 337.3 |
| 332 | LPI | LPI(20:4) [sn2] | 638.3 | 361.3 |
| 333 | LPI | LPI(20:4) [sn1] | 638.3 | 361.3 |
| 334 | methyl-CE | methyl-CE(18:0) | 684.6 | 383.3 |
| 335 | methyl-CE | methyl-CE(18:1) | 682.6 | 383.3 |
| 336 | methyl-CE | methyl-CE(18:2) | 680.6 | 383.3 |
| 337 | methyl-CE | methyl-CE(20:4) | 704.6 | 383.3 |
| 338 | methyl-CE | methyl-CE(22:6) | 728.6 | 383.3 |
| 339 | methyl-DE | methyl-DE(18:1) | 680.6 | 381.4 |
| 340 | methyl-DE | methyl-DE(18:2) | 678.6 | 381.4 |
| 341 | PA | PA(34:1) | 692.6 | 577.6 |
| 342 | PA | PA(36:1) | 720.6 | 605.6 |
| 343 | PA | PA(36:2) | 718.6 | 603.6 |
| 344 | PA | PA(36:3) | 716.6 | 601.6 |
| 345 | PA | PA(36:4) | 714.6 | 599.6 |
| 346 | PA | PA(40:6) | 766.6 | 651.6 |
| 347 | PC | PC(28:0) | 678.5 | 184.1 |
| 348 | PC | PC(14:0_16:0) | 706.5 | 184.1 |
| 349 | PC | PC(31:0) (a) | 720.6 | 184.1 |
| 350 | PC | PC(31:0) (b) | 720.6 | 184.1 |
| 351 | PC | PC(31:1) | 718.5 | 184.1 |
| 352 | PC | PC(16:0_16:0) | 734.6 | 184.1 |
| 353 | PC | PC(32:1) | 732.6 | 184.1 |
| 354 | PC | PC(32:2) | 730.5 | 184.1 |
| 355 | PC | PC(33:0) (a) | 748.6 | 184.1 |
| 356 | PC | PC(33:0) (b) | 748.6 | 184.1 |
| 357 | PC | PC(33:1) | 746.6 | 184.1 |
| 358 | PC | PC(33:2) | 744.6 | 184.1 |
| 359 | PC | PC(16:0_18:0) | 762.6 | 184.1 |
| 360 | PC | PC(16:0_18:1) | 760.6 | 184.1 |
| 361 | PC | PC(16:0_18:2) | 758.6 | 184.1 |
| 362 | OxSpecies | PC(34:2) [+OH] | 774.6 | 184.1 |
| 363 | PC | PC(16:1_18:2) | 756.6 | 184.1 |
| 364 | PC | PC(16:0_18:3) (a) | 756.6 | 184.1 |
| 365 | PC | PC(16:0_18:3) (b) | 756.6 | 184.1 |
| 366 | PC | PC(14:0_20:4) | 754.5 | 184.1 |
| 367 | PC | PC(34:5) | 752.6 | 184.1 |
| 368 | PC | PC(15-MHDA_18:1) | 774.6 | 184.1 |
| 369 | PC | PC(17:0_18:1) | 774.6 | 184.1 |
| 370 | PC | PC(15-MHDA_18:2) | 772.6 | 184.1 |
| 371 | PC | PC(17:0_18:2) | 772.6 | 184.1 |
| 372 | PC | PC(17:1_18:2) | 770.6 | 184.1 |
| 373 | PC | PC(15:0_20:3) | 770.6 | 184.1 |
| 374 | PC | PC(15:0_20:4) | 768.5 | 184.1 |
| 375 | PC | PC(35:5) | 766.5 | 184.1 |
| 376 | PC | PC(18:0_18:1) | 788.6 | 184.1 |
| 377 | PC | PC(18:1_18:1) | 786.6 | 184.1 |
| 378 | PC | PC(18:0_18:2) | 786.6 | 184.1 |
| 379 | PC | PC(18:1_18:2) | 784.6 | 184.1 |
| 380 | PC | PC(16:0_20:3) (a) | 784.6 | 184.1 |
| 381 | PC | PC(16:0_20:3) (b) | 784.6 | 184.1 |
| 382 | PC | PC(18:2_18:2) | 782.6 | 184.1 |
| 383 | OxSpecies | PC(36:4) [+OH] | 798.6 | 184.1 |
| 384 | PC | PC(16:0_20:4) | 782.6 | 184.1 |
| 385 | PC | PC(16:1_20:4) | 780.5 | 184.1 |
| 386 | PC | PC(16:0_20:5) | 780.5 | 184.1 |
| 387 | PC | PC(36:6) (a) | 778.5 | 184.1 |
| 388 | PC | PC(14:0_22:6) | 778.5 | 184.1 |
| 389 | PC | PC(15-MHDA_20:4) | 796.6 | 184.1 |
| 390 | PC | PC(17:0_20:4) | 796.6 | 184.1 |
| 391 | PC | PC(15:0_22:6) | 792.6 | 184.1 |
| 392 | PC | PC(38:2) | 814.6 | 184.1 |
| 393 | PC | PC(18:0_20:3) | 812.6 | 184.1 |
| 394 | PC | PC(18:1_20:3) | 810.6 | 184.1 |
| 395 | PC | PC(38:4) (b) | 810.6 | 184.1 |
| 396 | PC | PC(18:0_20:4) | 810.6 | 184.1 |
| 397 | PC | PC(38:5) (a) | 808.6 | 184.1 |
| 398 | PC | PC(38:5) (b) | 808.6 | 184.1 |
| 399 | PC | PC(38:6) (a) | 806.6 | 184.1 |
| 400 | OxSpecies | PC(38:6) [+OH] | 822.6 | 184.1 |
| 401 | PC | PC(16:0_22:6) | 806.6 | 184.1 |
| 402 | PC | PC(18:2_20:5) | 804.6 | 184.1 |
| 403 | PC | PC(16:1_22:6) | 804.6 | 184.1 |
| 404 | PC | PC(38:7) (c) | 804.6 | 184.1 |
| 405 | PC | PC(39:5) (a) | 822.6 | 184.1 |
| 406 | PC | PC(39:5) (b) | 822.6 | 184.1 |
| 407 | PC | PC(15-MHDA_22:6) | 820.6 | 184.1 |
| 408 | PC | PC(17:0_22:6) | 820.6 | 184.1 |
| 409 | PC | PC(18:0_22:4) | 838.6 | 184.1 |
| 410 | PC | PC(20:0_20:4) | 838.6 | 184.1 |
| 411 | PC | PC(18:0_22:5) (n3) & PC(20:1_20:4) | 836.6 | 184.1 |
| 412 | PC | PC(18:0_22:5) (n6) | 836.6 | 184.1 |
| 413 | PC | PC(18:0_22:6) | 834.6 | 184.1 |
| 414 | PC | PC(40:7) (a) | 832.6 | 184.1 |
| 415 | PC | PC(18:1_22:6) (a) | 832.6 | 184.1 |
| 416 | PC | PC(18:1_22:6) (b) | 832.6 | 184.1 |
| 417 | PC | PC(40:8) | 830.6 | 184.1 |
| 418 | PC | PC(42:10) | 854.5 | 184.1 |
| 419 | PC | PC(42:2) | 870.5 | 184.1 |
| 420 | PC | PC(42:3) | 868.5 | 184.1 |
| 421 | PC | PC(42:4) | 866.5 | 184.1 |
| 422 | PC | PC(42:5) (a) | 864.5 | 184.1 |
| 423 | PC | PC(42:5) (b) | 864.5 | 184.1 |
| 424 | PC | PC(42:6) (a) | 862.5 | 184.1 |
| 425 | PC | PC(42:6) (b) | 862.5 | 184.1 |
| 426 | PC | PC(42:7) | 860.5 | 184.1 |
| 427 | PC | PC(42:8) | 858.5 | 184.1 |
| 428 | PC | PC(42:9) | 856.5 | 184.1 |
| 429 | PC | PC(44:12) | 878.6 | 184.1 |
| 430 | PC | PC(44:4) (a) | 894.5 | 184.1 |
| 431 | PC | PC(44:4) (b) | 894.5 | 184.1 |
| 432 | PC | PC(44:5) | 892.5 | 184.1 |
| 433 | PC(O) | PC(O-16:0/16:0) | 720.6 | 184.1 |
| 434 | PC(O) | PC(O-32:1) | 718.5 | 184.1 |
| 435 | PC(O) | PC(O-32:2) | 716.6 | 184.1 |
| 436 | PC(O) | PC(O-34:1) | 746.6 | 184.1 |
| 437 | PC(O) | PC(O-34:2) | 744.6 | 184.1 |
| 438 | PC(O) | PC(O-34:4) | 740.6 | 184.1 |
| 439 | PC(O) | PC(O-36:0) | 776.6 | 184.1 |
| 440 | PC(O) | PC(O-18:0/18:1) | 774.6 | 184.1 |
| 441 | PC(O) | PC(O-18:1/18:1) | 772.6 | 184.1 |
| 442 | PC(O) | PC(O-18:0/18:2) | 772.6 | 184.1 |
| 443 | PC(O) | PC(O-18:1/18:2) | 770.6 | 184.1 |
| 444 | PC(O) | PC(O-16:0/20:3) | 770.6 | 184.1 |
| 445 | PC(O) | PC(O-16:0/20:4) | 768.5 | 184.1 |
| 446 | PC(O) | PC(O-36:5) | 766.5 | 184.1 |
| 447 | PC(O) | PC(O-18:0/20:4) | 796.6 | 184.1 |
| 448 | PC(O) | PC(O-38:5) | 794.6 | 184.1 |
| 449 | PC(O) | PC(O-16:0/22:6) | 792.6 | 184.1 |
| 450 | PC(O) | PC(O-40:5) | 822.6 | 184.1 |
| 451 | PC(O) | PC(O-18:0/22:6) | 820.6 | 184.1 |
| 452 | PC(O) | PC(O-40:7) | 818.6 | 184.1 |
| 453 | PC(O) | PC(O-42:4) (a) | 852.5 | 184.1 |
| 454 | PC(O) | PC(O-42:4) (b) | 852.5 | 184.1 |
| 455 | PC(O) | PC(O-42:5) (a) | 850.5 | 184.1 |
| 456 | PC(O) | PC(O-42:5) (b) | 850.5 | 184.1 |
| 457 | PC(O) | PC(O-42:6) | 848.5 | 184.1 |
| 458 | PC(O) | PC(O-42:7) | 846.5 | 184.1 |
| 459 | PC(O) | PC(O-42:8) | 844.5 | 184.1 |
| 460 | PC(O) | PC(O-44:6) | 876.5 | 184.1 |
| 461 | PC(O) | PC(O-44:7) | 874.5 | 184.1 |
| 462 | PC(O) | PC(O-46:7) (a) | 902.5 | 184.1 |
| 463 | PC(O) | PC(O-46:7) (b) | 902.5 | 184.1 |
| 464 | PC(O) | PC(O-46:8) | 900.5 | 184.1 |
| 465 | PC(P) | PC(P-16:0/14:0) | 690.4 | 184.1 |
| 466 | PC(P) | PC(P-16:0/16:0) | 718.5 | 184.1 |
| 467 | PC(P) | PC(P-16:0/16:1) | 716.6 | 184.1 |
| 468 | PC(P) | PC(P-16:0/18:0) | 746.6 | 184.1 |
| 469 | PC(P) | PC(P-16:0/18:1) | 744.6 | 184.1 |
| 470 | PC(P) | PC(P-16:0/18:2) | 742.5 | 184.1 |
| 471 | PC(P) | PC(P-16:0/18:3) | 740.6 | 184.1 |
| 472 | PC(P) | PC(P-35:2) (a) | 756.6 | 184.1 |
| 473 | PC(P) | PC(P-35:2) (b) | 756.6 | 184.1 |
| 474 | PC(P) | PC(P-15:0/20:4) (a) | 752.6 | 184.1 |
| 475 | PC(P) | PC(P-15:0/20:4) (b) | 752.6 | 184.1 |
| 476 | PC(P) | PC(P-18:1/18:1) | 770.6 | 184.1 |
| 477 | PC(P) | PC(P-18:0/18:2) | 770.6 | 184.1 |
| 478 | PC(P) | PC(P-36:3) | 768.5 | 184.1 |
| 479 | PC(P) | PC(P-16:0/20:4) | 766.5 | 184.1 |
| 480 | PC(P) | PC(P-16:0/20:5) | 764.6 | 184.1 |
| 481 | PC(P) | PC(P-17:0/20:4) (a) | 780.5 | 184.1 |
| 482 | PC(P) | PC(P-17:0/20:4) (b) | 780.5 | 184.1 |
| 483 | PC(P) | PC(P-18:0/20:4) | 794.6 | 184.1 |
| 484 | PC(P) | PC(P-38:5) (a) | 792.6 | 184.1 |
| 485 | PC(P) | PC(P-38:5) (b) | 792.6 | 184.1 |
| 486 | PC(P) | PC(P-16:0/22:6) | 790.6 | 184.1 |
| 487 | PC(P) | PC(P-20:0/20:4) | 822.6 | 184.1 |
| 488 | PC(P) | PC(P-18:0/22:5) | 820.6 | 184.1 |
| 489 | PC(P) | PC(P-18:0/22:6) | 818.6 | 184.1 |
| 490 | PC(P) | PC(P-18:1/22:6) | 816.6 | 184.1 |
| 491 | PC(P) | PC(P-42:5) | 848.5 | 184.1 |
| 492 | PC(P) | PC(P-44:5) | 876.5 | 184.1 |
| 493 | PC(P) | PC(P-46:8) | 898.5 | 184.1 |
| 494 | PE | PE(16:0_16:0) | 692.5 | 551.5 |
| 495 | PE | PE(16:0_16:1) | 690.5 | 549.5 |
| 496 | PE | PE(16:0_18:1) | 718.5 | 577.5 |
| 497 | PE | PE(16:0_18:2) | 716.5 | 575.5 |
| 498 | PE | PE(16:1_18:2) | 714.5 | 573.5 |
| 499 | PE | PE(16:0_18:3) (a) | 714.5 | 573.5 |
| 500 | PE | PE(16:0_18:3) (b) | 714.5 | 573.5 |
| 501 | PE | PE(15-MHDA_18:1) | 732.6 | 591.5 |
| 502 | PE | PE(17:0_18:1) | 732.6 | 591.5 |
| 503 | PE | PE(15-MHDA_18:2) | 730.5 | 589.5 |
| 504 | PE | PE(17:0_18:2) | 730.5 | 589.5 |
| 505 | PE | PE(36:0) | 748.6 | 607.6 |
| 506 | PE | PE(18:0_18:1) | 746.6 | 605.6 |
| 507 | PE | PE(18:1_18:1) | 744.6 | 603.5 |
| 508 | PE | PE(18:0_18:2) | 744.6 | 603.5 |
| 509 | PE | PE(18:1_18:2) | 742.5 | 601.5 |
| 510 | PE | PE(16:0_20:3) | 742.5 | 601.5 |
| 511 | PE | PE(16:0_20:4) | 740.5 | 599.5 |
| 512 | PE | PE(16:1_20:4) | 738.5 | 597.5 |
| 513 | PE | PE(16:0_20:5) | 738.5 | 597.5 |
| 514 | PE | PE(15-MHDA_20:4) | 754.6 | 613.5 |
| 515 | PE | PE(17:0_20:4) | 754.6 | 613.5 |
| 516 | PE | PE(18:0_20:3) (a) | 770.6 | 629.6 |
| 517 | PE | PE(18:0_20:3) (b) | 770.6 | 629.6 |
| 518 | PE | PE(18:0_20:4) | 768.6 | 627.5 |
| 519 | PE | PE(38:5) (a) | 766.5 | 625.5 |
| 520 | PE | PE(38:5) (b) | 766.5 | 625.5 |
| 521 | PE | PE(16:0_22:6) | 764.5 | 623.5 |
| 522 | PE | PE(15-MHDA_22:6) | 778.5 | 637.5 |
| 523 | PE | PE(17:0_22:6) | 778.5 | 637.5 |
| 524 | PE | PE(18:0_22:4) | 796.6 | 655.6 |
| 525 | PE | PE(20:0_20:4) | 796.6 | 655.6 |
| 526 | PE | PE(18:0_22:5) (n3) | 794.6 | 653.6 |
| 527 | PE | PE(18:0_22:5) (n6) | 794.6 | 653.6 |
| 528 | PE | PE(18:0_22:6) | 792.6 | 651.5 |
| 529 | PE | PE(18:1_22:6) (a) | 790.5 | 649.5 |
| 530 | PE | PE(18:1_22:6) (b) | 790.5 | 649.5 |
| 531 | PE(O) | PE(O-34:1) | 704.6 | 563.5 |
| 532 | PE(O) | PE(O-16:0/18:2) | 702.5 | 561.5 |
| 533 | PE(O) | PE(O-18:1/18:2) | 728.6 | 587.5 |
| 534 | PE(O) | PE(O-16:0/20:3) | 728.6 | 587.5 |
| 535 | PE(O) | PE(O-16:0/20:4) | 726.5 | 585.5 |
| 536 | PE(O) | PE(O-36:5) | 724.5 | 583.5 |
| 537 | PE(O) | PE(O-16:0/22:4) | 754.6 | 613.6 |
| 538 | PE(O) | PE(O-18:0/20:4) | 754.6 | 613.6 |
| 539 | PE(O) | PE(O-38:5) (a) | 752.6 | 611.5 |
| 540 | PE(O) | PE(O-38:5) (b) | 752.6 | 611.5 |
| 541 | PE(O) | PE(O-16:0/22:6) | 750.6 | 609.5 |
| 542 | PE(O) | PE(O-18:0/22:5) | 780.6 | 639.6 |
| 543 | PE(O) | PE(O-18:0/22:6) | 778.5 | 637.5 |
| 544 | PE(O) | PE(O-18:1/22:6) | 776.6 | 635.5 |
| 545 | PE(P) | PE(P-15:0/20:4) (a) | 710.5 | 361.3 |
| 546 | PE(P) | PE(P-15:0/20:4) (b) | 710.5 | 361.3 |
| 547 | PE(P) | PE(P-15:0/22:6) (a) | 734.5 | 385.3 |
| 548 | PE(P) | PE(P-15:0/22:6) (b) | 734.5 | 385.3 |
| 549 | PE(P) | PE(P-16:0/18:1) | 702.5 | 339.3 |
| 550 | PE(P) | PE(P-16:0/18:2) | 700.5 | 337.3 |
| 551 | PE(P) | PE(P-16:0/18:3) | 698.5 | 335.3 |
| 552 | PE(P) | PE(P-16:0/20:3) (a) | 726.5 | 363.3 |
| 553 | PE(P) | PE(P-16:0/20:3) (b) | 726.5 | 363.3 |
| 554 | PE(P) | PE(P-16:0/20:4) | 724.5 | 361.3 |
| 555 | PE(P) | PE(P-16:0/20:5) | 722.5 | 359.3 |
| 556 | PE(P) | PE(P-16:0/22:4) | 752.6 | 389.3 |
| 557 | PE(P) | PE(P-16:0/22:5) (n3) | 750.5 | 387.3 |
| 558 | PE(P) | PE(P-16:0/22:5) (n6) | 750.5 | 387.3 |
| 559 | PE(P) | PE(P-16:0/22:6) | 748.5 | 385.3 |
| 560 | PE(P) | PE(P-17:0/20:4) (a) | 738.6 | 361.3 |
| 561 | PE(P) | PE(P-17:0/20:4) (b) | 738.6 | 361.3 |
| 562 | PE(P) | PE(P-17:0/22:6) (a) | 762.6 | 385.3 |
| 563 | PE(P) | PE(P-17:0/22:6) (b) | 762.6 | 385.3 |
| 564 | PE(P) | PE(P-18:0/18:1) | 730.6 | 339.3 |
| 565 | PE(P) | PE(P-18:0/18:2) | 728.6 | 337.3 |
| 566 | PE(P) | PE(P-18:0/18:3) | 726.5 | 335.3 |
| 567 | PE(P) | PE(P-18:0/20:3) (a) | 754.5 | 363.3 |
| 568 | PE(P) | PE(P-18:0/20:3) (b) | 754.5 | 363.3 |
| 569 | PE(P) | PE(P-18:0/20:4) | 752.6 | 361.3 |
| 570 | PE(P) | PE(P-18:0/20:5) | 750.5 | 359.3 |
| 571 | PE(P) | PE(P-18:0/22:4) | 780.6 | 389.3 |
| 572 | PE(P) | PE(P-18:0/22:5) (n3) | 778.5 | 387.3 |
| 573 | PE(P) | PE(P-18:0/22:5) (n6) | 778.5 | 387.3 |
| 574 | PE(P) | PE(P-18:0/22:6) | 776.6 | 385.3 |
| 575 | PE(P) | PE(P-18:1/18:1) (a) | 728.6 | 339.3 |
| 576 | PE(P) | PE(P-18:1/18:1) (b) | 728.6 | 339.3 |
| 577 | PE(P) | PE(P-18:1/18:2) (a) | 726.5 | 337.3 |
| 578 | PE(P) | PE(P-18:1/18:2) (b) | 726.5 | 337.3 |
| 579 | PE(P) | PE(P-18:1/18:3) | 724.5 | 335.3 |
| 580 | PE(P) | PE(P-18:1/20:3) (a) | 752.5 | 363.3 |
| 581 | PE(P) | PE(P-18:1/20:3) (b) | 752.5 | 363.3 |
| 582 | PE(P) | PE(P-18:1/20:4) (a) | 750.5 | 361.3 |
| 583 | PE(P) | PE(P-18:1/20:4) (b) | 750.5 | 361.3 |
| 584 | PE(P) | PE(P-18:1/20:5) (a) | 748.5 | 359.3 |
| 585 | PE(P) | PE(P-18:1/20:5) (b) | 748.5 | 359.3 |
| 586 | PE(P) | PE(P-18:1/22:4) | 778.5 | 389.3 |
| 587 | PE(P) | PE(P-18:1/22:5) (a) | 776.6 | 387.3 |
| 588 | PE(P) | PE(P-18:1/22:5) (b) | 776.6 | 387.3 |
| 589 | PE(P) | PE(P-18:1/22:6) (a) | 774.5 | 385.3 |
| 590 | PE(P) | PE(P-18:1/22:6) (b) | 774.5 | 385.3 |
| 591 | PE(P) | PE(P-19:0/20:4) (a) | 766.6 | 361.3 |
| 592 | PE(P) | PE(P-19:0/20:4) (b) | 766.6 | 361.3 |
| 593 | PE(P) | PE(P-20:0/18:1) | 758.6 | 339.3 |
| 594 | PE(P) | PE(P-20:0/18:2) | 756.6 | 337.3 |
| 595 | PE(P) | PE(P-20:0/20:4) | 780.6 | 361.3 |
| 596 | PE(P) | PE(P-20:0/22:6) | 804.6 | 385.3 |
| 597 | PE(P) | PE(P-20:1/20:4) | 778.5 | 361.3 |
| 598 | PE(P) | PE(P-20:1/22:6) | 802.6 | 385.3 |
| 599 | PG | PG(34:1) | 766.6 | 577.5 |
| 600 | PG | PG(36:1) | 794.6 | 605.6 |
| 601 | PG | PG(36:2) | 792.6 | 603.5 |
| 602 | PI | PI(16:0/16:0) | 828.6 | 551.6 |
| 603 | PI | PI(16:0_16:1) | 826.5 | 549.5 |
| 604 | PI | PI(34:0) | 856.6 | 579.6 |
| 605 | PI | PI(34:1) | 854.6 | 577.6 |
| 606 | PI | PI(15-MHDA_18:1) & PI(17:0_18:1) | 868.6 | 591.6 |
| 607 | PI | PI(15-MHDA_18:2) & PI(17:0_18:2) | 866.6 | 589.6 |
| 608 | PI | PI(18:0_18:1) | 882.6 | 605.6 |
| 609 | PI | PI(36:2) | 880.6 | 603.6 |
| 610 | PI | PI(18:1_18:2) | 878.6 | 601.6 |
| 611 | PI | PI(16:0_20:3) (a) | 878.6 | 601.6 |
| 612 | PI | PI(16:0_20:3) (b) | 878.6 | 601.6 |
| 613 | PI | PI(16:0_20:4) | 876.6 | 599.6 |
| 614 | PI | PI(15-MHDA_20:4) & PI(17:0_20:4) | 890.6 | 613.6 |
| 615 | PI | PI(37:6) | 886.6 | 609.6 |
| 616 | PI | PI(18:0_20:2) | 908.6 | 631.6 |
| 617 | PI | PI(18:0_20:3) (a) | 906.6 | 629.6 |
| 618 | PI | PI(18:0_20:3) (b) | 906.6 | 629.6 |
| 619 | PI | PI(18:0_20:4) | 904.6 | 627.6 |
| 620 | PI | PI(38:5) (a) | 902.6 | 625.6 |
| 621 | PI | PI(38:5) (b) | 902.6 | 625.6 |
| 622 | PI | PI(38:6) | 900.6 | 623.6 |
| 623 | PI | PI(39:6) | 914.6 | 637.6 |
| 624 | PI | PI(18:0_22:4) | 932.6 | 655.6 |
| 625 | PI | PI(20:0_20:4) | 932.6 | 655.6 |
| 626 | PI | PI(18:0_22:5) (n3) | 930.6 | 653.6 |
| 627 | PI | PI(18:0_22:5) (n6) | 930.6 | 653.6 |
| 628 | PI | PI(18:0_22:6) | 928.6 | 651.6 |
| 629 | PIP1 | PIP1(38:4) | 984.7 | 627.7 |
| 630 | PS | PS(36:1) | 790.6 | 605.6 |
| 631 | PS | PS(36:2) | 788.5 | 603.5 |
| 632 | PS | PS(38:3) | 814.6 | 629.6 |
| 633 | PS | PS(38:4) | 812.5 | 627.5 |
| 634 | PS | PS(38:5) | 810.5 | 625.5 |
| 635 | PS | PS(40:5) | 838.6 | 653.6 |
| 636 | PS | PS(40:6) | 836.5 | 651.5 |
| 637 | S1P | S1P(d16:1) | 352.2 | 236.3 |
| 638 | S1P | S1P(d18:0) | 382.2 | 284.3 |
| 639 | S1P | S1P(d18:1) | 380.2 | 264.3 |
| 640 | S1P | S1P(d18:2) | 378.2 | 262.3 |
| 641 | SM | SM(d17:1/14:0) | 661.5 | 184.1 |
| 642 | SM | SM(d18:0/14:0) | 677.6 | 184.1 |
| 643 | SM | SM(d18:1/14:0) & SM(d16:1/16:0) | 675.5 | 184.1 |
| 644 | SM | SM(d18:2/14:0) | 673.5 | 184.1 |
| 645 | SM | SM(d17:1/16:0) | 689.6 | 184.1 |
| 646 | SM | SM(d18:0/16:0) | 705.6 | 184.1 |
| 647 | SM | SM(d18:1/16:0) | 703.6 | 184.1 |
| 648 | SM | SM(d18:2/16:0) | 701.6 | 184.1 |
| 649 | SM | SM(34:3) | 699.5 | 184.1 |
| 650 | SM | SM(d16:1/19:0) | 717.6 | 184.1 |
| 651 | SM | SM(d18:1/17:0) & SM(d17:1/18:0) | 717.6 | 184.1 |
| 652 | SM | SM(d18:2/17:0) | 715.6 | 184.1 |
| 653 | SM | SM(35:2) (b) | 715.6 | 184.1 |
| 654 | SM | SM(d18:1/18:0) & SM(d16:1/20:0) | 731.6 | 184.1 |
| 655 | SM | SM(36:2) (a) | 729.6 | 184.1 |
| 656 | SM | SM(36:2) (b) | 729.6 | 184.1 |
| 657 | SM | SM(d18:2/18:1) | 727.6 | 184.1 |
| 658 | SM | SM(37:1) | 745.6 | 184.1 |
| 659 | SM | SM(37:2) | 743.5 | 184.1 |
| 660 | SM | SM(d18:1/20:0) & SM(d16:1/22:0) | 759.6 | 184.1 |
| 661 | SM | SM(d18:2/20:0) | 757.6 | 184.1 |
| 662 | SM | SM(38:3) | 755.6 | 184.1 |
| 663 | SM | SM(d16:1/23:0) & SM(d17:1/22:0) | 773.7 | 184.1 |
| 664 | SM | SM(d18:0/22:0) | 789.7 | 184.1 |
| 665 | SM | SM(d18:1/22:0) & SM(d16:1/24:0) | 787.7 | 184.1 |
| 666 | SM | SM(d16:1/24:1) | 785.7 | 184.1 |
| 667 | SM | SM(d18:2/22:0) | 785.7 | 184.1 |
| 668 | SM | SM(40:3) (a) | 783.6 | 184.1 |
| 669 | SM | SM(40:3) (b) | 783.6 | 184.1 |
| 670 | SM | SM(40:4) | 781.5 | 184.1 |
| 671 | SM | SM(41:0) | 803.7 | 184.1 |
| 672 | SM | SM(41:1) (a) | 801.7 | 184.1 |
| 673 | SM | SM(d18:1/23:0) & SM(d17:1/24:0) | 801.7 | 184.1 |
| 674 | SM | SM(d17:1/24:1) | 799.7 | 184.1 |
| 675 | SM | SM(d18:2/23:0) | 799.7 | 184.1 |
| 676 | SM | SM(d18:1/24:0) | 815.7 | 184.1 |
| 677 | SM | SM(d18:1/24:1) | 813.7 | 184.1 |
| 678 | SM | SM(d18:2/24:0) | 813.7 | 184.1 |
| 679 | SM | SM(42:3) | 811.5 | 184.1 |
| 680 | SM | SM(42:4) | 809.5 | 184.1 |
| 681 | SM | SM(43:1) | 829.7 | 184.1 |
| 682 | SM | SM(d19:1/24:1) | 827.7 | 184.1 |
| 683 | SM | SM(43:2) (b) | 827.7 | 184.1 |
| 684 | SM | SM(43:2) (c) | 827.7 | 184.1 |
| 685 | SM | SM(44:1) | 843.6 | 184.1 |
| 686 | SM | SM(44:2) | 841.6 | 184.1 |
| 687 | SM | SM(44:3) | 839.6 | 184.1 |
| 688 | Sph | Sph(d18:1) | 300.3 | 282.3 |
| 689 | SHexCer | SHexCer(d18:1/16:0(OH)) | 796.8 | 264.3 |
| 690 | SHexCer | SHexCer(d18:1/16:0) | 780.8 | 264.3 |
| 691 | SHexCer | SHexCer(d18:1/24:0(OH)) | 908.8 | 264.3 |
| 692 | SHexCer | SHexCer(d18:1/24:0) | 892.8 | 264.3 |
| 693 | SHexCer | SHexCer(d18:1/24:1(OH)) | 906.8 | 264.3 |
| 694 | SHexCer | SHexCer(d18:1/24:1) | 890.8 | 264.3 |
| 695 | TG [NL] | TG(48:0) [NL-16:0] | 824.8 | 551.5 |
| 696 | TG [NL] | TG(48:0) [NL-18:0] | 824.8 | 523.5 |
| 697 | TG [NL] | TG(48:1) [NL-16:1] | 822.8 | 551.5 |
| 698 | TG [NL] | TG(48:1) [NL-18:1] | 822.8 | 523.5 |
| 699 | TG [NL] | TG(48:2) [NL-14:0] | 820.8 | 575.5 |
| 700 | TG [NL] | TG(48:2) [NL-14:1] | 820.8 | 577.5 |
| 701 | TG [NL] | TG(48:2) [NL-16:1] | 820.8 | 549.5 |
| 702 | TG [NL] | TG(48:2) [NL-18:2] | 820.8 | 523.5 |
| 703 | TG [NL] | TG(48:3) [NL-14:0] | 818.8 | 573.5 |
| 704 | TG [NL] | TG(48:3) [NL-16:1] | 818.8 | 547.5 |
| 705 | TG [NL] | TG(48:3) [NL-18:3] | 818.8 | 523.5 |
| 706 | TG [NL] | TG(49:1) [NL-16:1] | 836.8 | 565.5 |
| 707 | TG [NL] | TG(49:1) [NL-17:1] | 836.8 | 551.5 |
| 708 | TG [NL] | TG(50:0) [NL-18:0] | 852.8 | 551.5 |
| 709 | TG [NL] | TG(50:1) [NL-14:0] | 850.8 | 605.5 |
| 710 | TG [NL] | TG(50:1) [NL-16:0] | 850.8 | 577.5 |
| 711 | TG [NL] | TG(50:1) [NL-18:1] | 850.8 | 551.5 |
| 712 | TG [NL] | TG(50:2) [NL-14:0] | 848.8 | 603.5 |
| 713 | TG [NL] | TG(50:2) [NL-16:1] | 848.8 | 577.5 |
| 714 | TG [NL] | TG(50:2) [NL-18:1] | 848.8 | 549.5 |
| 715 | TG [NL] | TG(50:2) [NL-18:2] | 848.8 | 551.5 |
| 716 | TG [NL] | TG(50:3) [NL-14:0] | 846.8 | 601.5 |
| 717 | TG [NL] | TG(50:3) [NL-14:1] | 846.8 | 603.5 |
| 718 | TG [NL] | TG(50:3) [NL-16:1] | 846.8 | 575.5 |
| 719 | TG [NL] | TG(50:3) [NL-18:2] | 846.8 | 549.5 |
| 720 | TG [NL] | TG(50:3) [NL-18:3] | 846.8 | 551.5 |
| 721 | TG [NL] | TG(50:4) [NL-14:0] | 844.8 | 599.5 |
| 722 | TG [NL] | TG(50:4) [NL-18:3] | 844.8 | 549.5 |
| 723 | TG [NL] | TG(50:4) [NL-20:4] | 844.8 | 523.5 |
| 724 | TG [NL] | TG(51:0) [NL-16:0] | 866.7 | 593.4 |
| 725 | TG [NL] | TG(51:1) [NL-17:0] | 864.8 | 577.5 |
| 726 | TG [NL] | TG(51:2) [NL-15:0] | 862.8 | 603.5 |
| 727 | TG [NL] | TG(51:2) [NL-17:0] | 862.8 | 575.5 |
| 728 | TG [NL] | TG(51:2) [NL-17:1] | 862.8 | 577.5 |
| 729 | TG [NL] | TG(52:1) [NL-18:0] | 878.8 | 577.5 |
| 730 | TG [NL] | TG(52:1) [NL-18:1] | 878.8 | 579.5 |
| 731 | TG [NL] | TG(52:2) [NL-16:0] | 876.8 | 603.5 |
| 732 | TG [NL] | TG(52:2) [NL-18:2] | 876.8 | 579.5 |
| 733 | TG [NL] | TG(52:3) [NL-16:1] | 874.8 | 603.5 |
| 734 | TG [NL] | TG(52:3) [NL-18:2] | 874.8 | 577.5 |
| 735 | TG [NL] | TG(52:4) [NL-16:1] | 872.8 | 601.5 |
| 736 | TG [NL] | TG(52:4) [NL-18:2] | 872.8 | 575.5 |
| 737 | TG [NL] | TG(52:4) [NL-18:3] | 872.8 | 577.5 |
| 738 | TG [NL] | TG(52:5) [NL-18:3] | 870.8 | 575.5 |
| 739 | TG [NL] | TG(52:5) [NL-20:4] | 870.8 | 549.5 |
| 740 | TG [NL] | TG(52:5) [NL-20:5] | 870.8 | 551.5 |
| 741 | TG [NL] | TG(53:2) [NL-17:1] | 890.8 | 605.5 |
| 742 | TG [NL] | TG(53:2) [NL-18:1] | 890.8 | 591.5 |
| 743 | TG [NL] | TG(54:0) [NL-18:0] | 908.8 | 607.5 |
| 744 | TG [NL] | TG(54:1) [NL-18:1] | 906.8 | 607.5 |
| 745 | TG [NL] | TG(54:2) [NL-18:0] | 904.8 | 603.5 |
| 746 | TG [NL] | TG(54:2) [NL-20:1] | 904.8 | 577.5 |
| 747 | TG [NL] | TG(54:3) [NL-18:1] | 902.8 | 603.5 |
| 748 | TG [NL] | TG(54:3) [NL-18:2] | 902.8 | 605.5 |
| 749 | TG [NL] | TG(54:4) [NL-18:2] | 900.8 | 603.5 |
| 750 | TG [NL] | TG(54:4) [NL-20:3] | 900.8 | 577.5 |
| 751 | TG [NL] | TG(54:5) [NL-18:3] | 898.8 | 603.5 |
| 752 | TG [NL] | TG(54:5) [NL-20:4] | 898.8 | 577.5 |
| 753 | TG [NL] | TG(54:6) [NL-18:3] | 896.8 | 601.5 |
| 754 | TG [NL] | TG(54:6) [NL-20:4] | 896.8 | 575.5 |
| 755 | TG [NL] | TG(54:6) [NL-20:5] | 896.8 | 577.5 |
| 756 | TG [NL] | TG(54:6) [NL-22:6] | 896.8 | 551.5 |
| 757 | TG [NL] | TG(54:7) [NL-20:5] | 894.8 | 575.5 |
| 758 | TG [NL] | TG(54:7) [NL-22:6] | 894.8 | 549.5 |
| 759 | TG [NL] | TG(56:6) [NL-20:4] | 924.8 | 603.5 |
| 760 | TG [NL] | TG(56:6) [NL-22:5] | 924.8 | 577.5 |
| 761 | TG [NL] | TG(56:7) [NL-20:4] | 922.8 | 601.5 |
| 762 | TG [NL] | TG(56:7) [NL-20:5] | 922.8 | 603.5 |
| 763 | TG [NL] | TG(56:7) [NL-22:5] | 922.8 | 575.5 |
| 764 | TG [NL] | TG(56:7) [NL-22:6] | 922.8 | 577.5 |
| 765 | TG [NL] | TG(56:8) [NL-20:4] | 920.8 | 599.5 |
| 766 | TG [NL] | TG(56:8) [NL-20:5] | 920.8 | 601.5 |
| 767 | TG [NL] | TG(56:8) [NL-22:6] | 920.8 | 575.5 |
| 768 | TG [NL] | TG(56:9) [NL-22:6] | 918.8 | 573.5 |
| 769 | TG [NL] | TG(58:10) [NL-22:6] | 944.9 | 599.5 |
| 770 | TG [NL] | TG(58:8) [NL-22:6] | 948.8 | 603.5 |
| 771 | TG [NL] | TG(58:9) [NL-22:6] | 946.9 | 601.5 |
| 772 | TG(O) [NL] | TG(O-50:1) [NL-15:0] | 836.8 | 577.5 |
| 773 | TG(O) [NL] | TG(O-50:1) [NL-16:0] | 836.8 | 563.5 |
| 774 | TG(O) [NL] | TG(O-50:1) [NL-17:1] | 836.8 | 551.5 |
| 775 | TG(O) [NL] | TG(O-50:1) [NL-18:1] | 836.8 | 537.5 |
| 776 | TG(O) [NL] | TG(O-50:2) [NL-16:1] | 834.8 | 563.5 |
| 777 | TG(O) [NL] | TG(O-50:2) [NL-18:1] | 834.8 | 535.5 |
| 778 | TG(O) [NL] | TG(O-50:2) [NL-18:2] | 834.8 | 537.5 |
| 779 | TG(O) [NL] | TG(O-50:3) [NL-18:2] | 832.8 | 535.5 |
| 780 | TG(O) [NL] | TG(O-52:0) [NL-16:0] | 866.8 | 593.5 |
| 781 | TG(O) [NL] | TG(O-52:1) [NL-16:0] | 864.8 | 591.5 |
| 782 | TG(O) [NL] | TG(O-52:1) [NL-18:1] | 864.8 | 565.5 |
| 783 | TG(O) [NL] | TG(O-52:2) [NL-16:0] | 862.8 | 589.5 |
| 784 | TG(O) [NL] | TG(O-52:2) [NL-17:1] | 862.8 | 577.5 |
| 785 | TG(O) [NL] | TG(O-52:2) [NL-18:1] | 862.8 | 563.5 |
| 786 | TG(O) [NL] | TG(O-54:2) [NL-18:1] | 890.8 | 591.5 |
| 787 | TG(O) [NL] | TG(O-54:3) [NL-17:1] | 888.8 | 603.5 |
| 788 | TG(O) [NL] | TG(O-54:3) [NL-18:1] | 888.8 | 589.5 |
| 789 | TG(O) [NL] | TG(O-54:4) [NL-17:1] | 886.8 | 601.5 |
| 790 | TG(O) [NL] | TG(O-54:4) [NL-18:2] | 886.8 | 589.5 |
| 791 | Ubiquinone | Ubiquinone | 880.7 | 197 |

TABLE 3
Internal standards

| Lipid | | Precursor | Product |
|---|---|---|---|
| Number | Compound Name | (m/z) | (m/z) |
| S1 | AcylCarnitine 16:0 d3 (IS) | 403.4 | 85.1 |
| S2 | CE 18:0-d6 (IS) | 676.7 | 375.3 |
| S3 | Cer(d18:1-d7/18:0) (IS) | 573.6 | 271.4 |
| S4 | Cholic Acid d4 (IS) | 430.3 | 359.3 |
| S5 | COH-d7 (IS) | 376.4 | 161.2 |
| S6 | DE(18:1) ester d6 (IS) | 672.6 | 373.4 |
| S7 | DG(15:0 18:1) d7 (IS) | 605.5 | 299.5 |
| S8 | FA(18:1) d9 (IS) | 290.2 | 290.2 |
| S9 | FA(20:4) d11 (IS) | 314.2 | 314.2 |
| S10 | FA(22:6) d5 (IS) | 332.2 | 332.2 |
| S11 | Hex2Cer(d18:1/15:0) d7 (IS) | 855.6 | 271.3 |
| S12 | Hex3Cer(d18:1/17:0) (IS) | 1038.7 | 264.3 |
| S13 | HexCer(d18:1/15:0) d7 (IS) | 693.6 | 271.3 |
| S14 | LPC(18:1) d7 (IS) | 529.4 | 184.1 |
| S15 | LPE(18:1) d7 (IS) | 487.3 | 346.3 |
| S16 | LPI 13:0 (IS) | 548.3 | 271.3 |
| S17 | MG(18:1) d7 (IS) | 364.2 | 272.2 |
| S18 | PA(15:0_18:1) d7 (IS) | 685.6 | 570.6 |
| S19 | PC(15:0_18:1) d7 (IS) | 753.6 | 184.1 |
| S20 | PC(P-18:0/18:1) d9 (IS) | 781.6 | 184.1 |
| S21 | PE(15:0_18:1) d7 (IS) | 711.6 | 570.5 |
| S22 | PE(P-18:0/18:1) d9 (IS) | 739.5 | 348.3 |
| S23 | PG(15:0_18:1) d7 (IS) | 759.6 | 570.6 |
| S24 | PI(15:0_18:1) d7 (IS) | 847.6 | 570.6 |
| S25 | PS(15:0_18:1) d7 (IS) | 755.5 | 570.5 |
| S26 | S1P(d18:1) d7 (IS) | 387.2 | 271.3 |
| S27 | SM(d18:1/15:0) d9 (IS) | 698.6 | 193.1 |
| S28 | Sph(d17:1) (IS) | 286.3 | 268.3 |
| S29 | SHexCer(d18:1/12:0) (IS) | 724.8 | 264.3 |
| S30 | TG(48:1) [NL-18:1] d7 (IS) | 829.8 | 523.5 |

Comparison of the resulting extracted ion chromatograms was used to determine whether the peak of interest overlapped with any additional peaks after reducing the length of the chromatographic separation to 5 minutes for all 791 monitored transitions. A binary value was thus assigned to each lipid species (peak overlap=y/n).

Where peak overlap occurred between the peaks of highly correlated lipid species, such as the sn1 and sn2 positional isomers of glycerophospholipids, but no other species, these compounds were combined into a single compound and carried through to the next step of analysis. Correlation was determined through the use of a correlation matrix calculated using previously generated data resulting from a large cohort study.

### Additional Peak Parameters:

A series of 24 identical plasma extracts pooled from multiple individuals were analysed using the Clinical Protocol LC-MS/MS Conditions. The resulting dataset was used to determine the Mean Peak Area (the integrated chromatogram peak area for the target analyte) and the Measurement CV % (the peak area coefficient of variation across all replicate measurements) for each of the monitored transitions

### Lipid List Refinement

The following steps were undertaken to refine the lipid list:
1) From the list of 791 lipid transitions analysed, peaks determined to have merged with a neighbouring peak upon reduction in HPLC runtime were eliminated from further analysis unless they were highly correlated compounds, in which case they were combined and carried through to the next step.
2) Peaks that displayed a mean peak area <250 counts were eliminated from further analysis.
3) Peaks that displayed peak area CV %>20% were eliminated from further analysis.
4) Some minor additions were made to ensure good representation of all lipid classes.

This process of elimination resulted in the reduction of the 791 lipid species measured in the Research Lipid List (Table 2) to a list of 339 analytes (CLP1 list; Table 4) and 30 internal standards (Table 5). In order to utilise previously generated large cohort datasets, in the following examples a subset of CLP1 is sometimes used, termed CLP1s, which contains 292 of the above species (indicated ‘y’ in Table 4) which map directly on to older datasets.

TABLE 4
CLP1 List

| Lipid | | | Precursor | Product | |
|---|---|---|---|---|---|
| Number | Lipid Class | Compound Name | (m/z) | (m/z) | CLP1s |
| 1 | AC | AC(10:0) | 316.3 | 85.1 | |
| 2 | AC | AC(12:0) | 344.3 | 85.1 | y |
| 3 | AC | AC(12:1) | 342.3 | 85.1 | |
| 4 | AC | AC(13:0) | 358.3 | 85.1 | y |
| 5 | AC | AC(14:0) | 372.3 | 85.1 | y |
| 6 | AC-OH | AC(14:0)-OH | 388.3 | 85.1 | |
| 7 | AC | AC(14:1) | 370.3 | 85.1 | y |
| 8 | AC-OH | AC(14:1)-OH | 386.3 | 85.1 | |
| 9 | AC | AC(14:2) | 368.3 | 85.1 | y |
| 10 | AC | AC(16:0) | 400.4 | 85.1 | y |
| 11 | AC | AC(16:1) | 398.3 | 85.1 | y |
| 12 | AC | AC(18:1) | 426.4 | 85.1 | y |
| 13 | AC-OH | AC(18:1)-OH | 442.4 | 85.1 | |
| 14 | AC | AC(18:2) | 424.3 | 85.1 | y |
| 15 | AC | AC(24:0) | 512.3 | 85.1 | |
| 16 | AC | AC(26:0) | 540.3 | 85.1 | |
| 17 | AC | AC(26:1) | 538.3 | 85.1 | |
| 18 | CE | CE(14:0) | 614.6 | 369.3 | y |
| 19 | CE | CE(15:0) | 628.6 | 369.3 | y |
| 20 | CE | CE(16:0) | 642.6 | 369.3 | y |
| 21 | CE | CE(16:1) | 640.6 | 369.3 | y |
| 22 | CE | CE(16:2) | 638.6 | 369.3 | y |
| 23 | CE | CE(17:0) | 656.6 | 369.3 | y |
| 24 | CE | CE(18:0) | 670.7 | 369.3 | y |
| 25 | CE | CE(18:1) | 668.6 | 369.3 | y |
| 26 | CE | CE(18:2) | 666.6 | 369.3 | y |
| 27 | CE | CE(18:3) | 664.6 | 369.3 | y |
| 28 | CE | CE(20:0) | 698.7 | 369.3 | y |
| 29 | CE | CE(20:1) | 696.7 | 369.3 | y |
| 30 | CE | CE(20:2) | 694.7 | 369.3 | y |
| 31 | CE | CE(20:4) | 690.6 | 369.3 | y |
| 32 | CE | CE(20:5) | 688.6 | 369.3 | y |
| 33 | CE | CE(22:1) | 724.7 | 369.3 | y |
| 34 | CE | CE(22:6) | 714.6 | 369.3 | y |
| 35 | Cer(d) | Cer(d16:1/22:0) | 594.6 | 236.3 | y |
| 36 | Cer(d) | Cer(d16:1/24:0) | 622.6 | 236.3 | y |
| 37 | Cer(d) | Cer(d16:1/24:1) | 620.6 | 236.3 | y |
| 38 | Cer(d) | Cer(d17:1/24:0) | 636.6 | 250.3 | y |
| 39 | Cer(d) | Cer(d18:1/16:0) | 538.5 | 264.3 | y |
| 40 | Cer(d) | Cer(d18:1/20:0) | 594.6 | 264.3 | y |
| 41 | Cer(d) | Cer(d18:1/22:0) | 622.6 | 264.3 | y |
| 42 | Cer(d) | Cer(d18:1/23:0) | 636.6 | 264.3 | y |
| 43 | Cer(d) | Cer(d18:1/24:0) | 650.6 | 264.3 | y |
| 44 | Cer(d) | Cer(d18:1/24:1) | 648.6 | 264.3 | y |
| 45 | Cer(d) | Cer(d18:2/22:0) | 620.6 | 262.3 | y |
| 46 | Cer(d) | Cer(d18:2/23:0) | 634.6 | 262.3 | y |
| 47 | Cer(d) | Cer(d18:2/24:0) | 648.6 | 262.3 | y |
| 48 | Cer(d) | Cer(d18:2/24:1) | 646.6 | 262.3 | y |
| 49 | Cer(d) | Cer(d19:1/22:0) | 636.6 | 278.3 | y |
| 50 | Cer(d) | Cer(d19:1/24:0) | 664.6 | 278.3 | y |
| 51 | Cer(d) | Cer(d19:1/24:1) | 662.6 | 278.3 | y |
| 52 | Cer(m) | Cer(m18:0/22:0) | 608.6 | 268.4 | y |
| 53 | Cer(m) | Cer(m18:1/20:0) | 578.6 | 266.4 | y |
| 54 | Cer(m) | Cer(m18:1/22:0) | 606.6 | 266.4 | y |
| 55 | Cer(m) | Cer(m18:1/24:0) | 634.6 | 266.4 | y |
| 56 | Cer(m) | Cer(m18:1/24:1) | 632.6 | 266.4 | y |
| 57 | C1P | Cer1P(d18:1/16:0) | 618.4 | 264.3 | y |
| 58 | COH | COH | 369.4 | 161.2 | y |
| 59 | DE | DE(18:1) | 666.6 | 367.4 | y |
| 60 | DE | DE(18:2) | 664.6 | 367.4 | y |
| 61 | DE | DE(20:4) | 688.6 | 367.4 | y |
| 62 | DG | DG(16:0_16:0) | 586.5 | 313.2 | y |
| 63 | DG | DG(16:0_18:1) | 612.6 | 313.3 | y |
| 64 | DG | DG(16:0_18:2) | 610.5 | 313.2 | y |
| 65 | DG | DG(18:1_18:1) | 638.6 | 339.3 | y |
| 66 | DG | DG(18:1_18:2) | 636.6 | 339.3 | y |
| 67 | DG | DG(18:1_20:3) | 662.6 | 339.3 | y |
| 68 | DG | DG(18:2_18:2) | 634.5 | 337.2 | y |
| 69 | dimethyl-CE | dimethyl-CE(18:1) | 696.6 | 397.3 | |
| 70 | dimethyl-CE | dimethyl-CE(18:2) | 694.6 | 397.3 | |
| 71 | dimethyl-CE | dimethyl-CE(20:4) | 718.6 | 397.3 | |
| 72 | FFA | FA(14:0) | 227.2 | 227.2 | |
| 73 | FFA | FA(16:0) | 255.2 | 255.2 | |
| 74 | FFA | FA(16:1) | 253.2 | 253.2 | |
| 75 | FFA | FA(17:1) | 267.2 | 267.2 | |
| 76 | FFA | FA(18:1) | 281.3 | 281.2 | |
| 77 | FFA | FA(18:2) | 279.2 | 279.2 | |
| 78 | FFA | FA(18:3) | 277.2 | 277.2 | |
| 79 | FFA | FA(20:4) | 303.2 | 303.2 | |
| 80 | FFA | FA(22:4) | 331.3 | 331.3 | |
| 81 | FFA | FA(22:5) | 329.3 | 329.2 | |
| 82 | FFA | FA(22:6) | 327.2 | 327.2 | |
| 83 | GM3 | GM3(d18:1/16:0) | 1153.7 | 264.3 | y |
| 84 | GM3 | GM3(d18:1/18:0) | 1181.8 | 264.3 | y |
| 85 | GM3 | GM3(d18:1/22:0) | 1237.8 | 264.3 | y |
| 86 | GM3 | GM3(d18:1/24:1) | 1263.8 | 264.3 | y |
| 87 | Hex2Cer | Hex2Cer(d16:1/16:0) | 834.6 | 236.3 | y |
| 88 | Hex2Cer | Hex2Cer(d18:1/16:0) | 862.6 | 264.3 | y |
| 89 | Hex2Cer | Hex2Cer(d18:1/22:0) | 946.7 | 264.3 | y |
| 90 | Hex2Cer | Hex2Cer(d18:1/24:0) | 974.8 | 264.3 | y |
| 91 | Hex2Cer | Hex2Cer(d18:1/24:1) | 972.7 | 264.3 | y |
| 92 | Hex2Cer | Hex2Cer(d18:2/16:0) | 860.6 | 262.3 | y |
| 93 | Hex3Cer | Hex3Cer(d18:1/16:0) | 1024.7 | 264.3 | y |
| 94 | HexCer | HexCer(d18:1/16:0) | 700.6 | 264.3 | y |
| 95 | HexCer | HexCer(d18:1/18:0) | 728.6 | 264.3 | y |
| 96 | HexCer | HexCer(d18:1/22:0) | 784.7 | 264.3 | y |
| 97 | HexCer | HexCer(d18:1/24:0) | 812.7 | 264.3 | y |
| 98 | HexCer | HexCer(d18:1/24:1) | 810.7 | 264.3 | y |
| 99 | HexCer | HexCer(d18:2/24:0) | 810.7 | 262.3 | y |
| 100 | LPC | LPC(14:0) | 468.3 | 184.1 | y |
| 101 | LPC | LPC(17:0) | 510.4 | 184.1 | y |
| 102 | LPC | LPC(18:0) | 524.4 | 184.1 | y |
| 103 | LPC | LPC(18:1) | 522.4 | 184.1 | y |
| 104 | LPC | LPC(18:2) | 520.3 | 184.1 | y |
| 105 | LPC | LPC(18:3) | 518.3 | 184.1 | y |
| 106 | LPC | LPC(19:0) | 538.4 | 184.1 | y |
| 107 | LPC | LPC(19:1) | 536.4 | 184.1 | y |
| 108 | LPC | LPC(20:0) | 552.4 | 184.1 | y |
| 109 | LPC | LPC(20:4) | 544.3 | 184.1 | y |
| 110 | LPC | LPC(20:5) | 542.3 | 184.1 | y |
| 111 | LPC | LPC(22:0) | 580.4 | 184.1 | y |
| 112 | LPC | LPC(22:5) | 570.4 | 184.1 | y |
| 113 | LPC | LPC(22:6) | 568.3 | 184.1 | y |
| 114 | LPC | LPC(24:0) | 608.5 | 184.1 | y |
| 115 | LPC | LPC(26:0) | 636.5 | 184.1 | y |
| 116 | LPC(O) | LPC(O-20:0) | 538.4 | 104.1 | y |
| 117 | LPC(O) | LPC(O-22:0) | 566.5 | 104.1 | y |
| 118 | LPC(O) | LPC(O-22:1) | 564.4 | 104.1 | y |
| 119 | LPC(O) | LPC(O-24:0) | 594.5 | 104.1 | y |
| 120 | LPC(O) | LPC(O-24:2) | 590.5 | 104.1 | y |
| 121 | LPC(P) | LPC(P-16:0) | 480.3 | 104.1 | y |
| 122 | LPC(P) | LPC(P-17:0) | 494.3 | 104.1 | y |
| 123 | LPE | LPE(16:0) | 454.3 | 313.3 | y |
| 124 | LPE | LPE(18:0) | 482.3 | 341.3 | y |
| 125 | LPE | LPE(18:1) | 480.3 | 339.3 | y |
| 126 | LPE | LPE(18:2) | 478.3 | 337.3 | y |
| 127 | LPE | LPE(20:4) | 502.3 | 361.3 | y |
| 128 | LPE | LPE(22:6) | 526.3 | 385.3 | y |
| 129 | LPE(P) | LPE(P-16:0) | 438.3 | 266.4 | y |
| 130 | LPE(P) | LPE(P-18:0) | 466.3 | 294.4 | y |
| 131 | LPI | LPI(18:0) | 618.3 | 341.3 | y |
| 132 | methyl-CE | methyl-CE(18:0) | 684.6 | 383.3 | |
| 133 | methyl-CE | methyl-CE(18:2) | 680.6 | 383.3 | |
| 134 | methyl-CE | methyl-CE(20:4) | 704.6 | 383.3 | |
| 135 | methyl-DE | methyl-DE(18:1) | 680.6 | 381.4 | |
| 136 | methyl-DE | methyl-DE(18:2) | 678.6 | 381.4 | |
| 137 | PC | PC(14:0_20:4) | 754.5 | 184.1 | y |
| 138 | PC | PC(15:0_20:4) | 768.5 | 184.1 | y |
| 139 | PC | PC(15:0_22:6) | 792.6 | 184.1 | y |
| 140 | PC | PC(16:0_16:0) | 734.6 | 184.1 | y |
| 141 | PC | PC(16:0_18:0) | 762.6 | 184.1 | y |
| 142 | PC | PC(16:0_18:1) | 760.6 | 184.1 | y |
| 143 | PC | PC(16:0_18:2) | 758.6 | 184.1 | y |
| 144 | PE | PC(16:0_18:3) | 714.5 | 573.5 | y |
| 145 | PC | PC(16:0_20:4) | 782.6 | 184.1 | y |
| 146 | PC | PC(16:0_20:5) | 780.5 | 184.1 | y |
| 147 | PC | PC(16:0_22:6) | 806.6 | 184.1 | y |
| 148 | PC | PC(16:1_20:4) | 780.5 | 184.1 | y |
| 149 | PC | PC(18:0_18:1) | 788.6 | 184.1 | y |
| 150 | PC | PC(18:0_20:4) | 810.6 | 184.1 | y |
| 151 | PC | PC(18:0_22:6) | 834.6 | 184.1 | y |
| 152 | PC | PC(18:1_20:3) | 810.6 | 184.1 | y |
| 153 | PC | PC(18:2_18:2) | 782.6 | 184.1 | y |
| 154 | PC | PC(31:0) | 720.6 | 184.1 | y |
| 155 | PC | PC(31:1) | 718.5 | 184.1 | |
| 156 | PC | PC(32:1) | 732.6 | 184.1 | y |
| 157 | PC | PC(32:2) | 730.5 | 184.1 | y |
| 158 | PC | PC(33:1) | 746.6 | 184.1 | y |
| 159 | PC | PC(33:2) | 744.6 | 184.1 | y |
| 160 | OxSpecies | PC(34:2) [+OH] | 774.6 | 184.1 | |
| 161 | PC | PC(34:5) | 752.6 | 184.1 | y |
| 162 | PC | PC(35:5) | 766.5 | 184.1 | y |
| 163 | PC | PC(36:2) | 786.6 | 184.1 | y |
| 164 | PC | PC(36:4) | 782.6 | 184.1 | y |
| 165 | PC | PC(38:4) (b) | 810.6 | 184.1 | y |
| 166 | PC | PC(38:6) (a) | 806.6 | 184.1 | y |
| 167 | OxSpecies | PC(38:6) [+OH] | 822.6 | 184.1 | |
| 168 | PC | PC(40:8) | 830.6 | 184.1 | y |
| 169 | PC | PC(42:10) | 854.5 | 184.1 | |
| 170 | PC | PC(42:2) | 870.5 | 184.1 | |
| 171 | PC | PC(42:3) | 868.5 | 184.1 | |
| 172 | PC | PC(42:4) | 866.5 | 184.1 | |
| 173 | PC | PC(42:7) | 860.5 | 184.1 | |
| 174 | PC | PC(42:8) | 858.5 | 184.1 | |
| 175 | PC(O) | PC(O-16:0/16:0) | 720.6 | 184.1 | y |
| 176 | PC(O) | PC(O-34:2) | 744.6 | 184.1 | y |
| 177 | PC(O) | PC(O-36:0) | 776.6 | 184.1 | y |
| 178 | PC(O) | PC(O-36:5) | 766.5 | 184.1 | y |
| 179 | PC(O) | PC(O-42:8) | 844.5 | 184.1 | |
| 180 | PC(O) | PC(O-46:7) | 902.5 | 184.1 | |
| 181 | PC(P) | PC(P-16:0/14:0) | 690.4 | 184.1 | y |
| 182 | PC(P) | PC(P-16:0/16:0) | 718.5 | 184.1 | y |
| 183 | PC(P) | PC(P-16:0/18:1) | 744.6 | 184.1 | y |
| 184 | PC(P) | PC(P-16:0/18:2) | 742.5 | 184.1 | y |
| 185 | PC(P) | PC(P-16:0/20:4) | 766.5 | 184.1 | y |
| 186 | PC(P) | PC(P-16:0/20:5) | 764.6 | 184.1 | y |
| 187 | PC(P) | PC(P-17:0/20:4) | 780.5 | 184.1 | y |
| 188 | PC(P) | PC(P-46:8) | 898.5 | 184.1 | |
| 189 | PE | PE(16:0_16:1) | 690.5 | 549.5 | y |
| 190 | PE | PE(16:0_18:1) | 718.5 | 577.5 | y |
| 191 | PE | PE(16:0_18:2) | 716.5 | 575.5 | y |
| 192 | PE | PE(16:0_20:4) | 740.5 | 599.5 | y |
| 193 | PE | PE(16:0_22:6) | 764.5 | 623.5 | y |
| 194 | PE | PE(18:0_18:1) | 746.6 | 605.6 | y |
| 195 | PE | PE(18:0_20:4) | 768.6 | 627.5 | y |
| 196 | PE | PE(18:0_22:6) | 792.6 | 651.5 | y |
| 197 | PE | PE(36:2) | 744.6 | 603.5 | y |
| 198 | PE(O) | PE(O-16:0/18:2) | 702.5 | 561.5 | y |
| 199 | PE(O) | PE(O-16:0/20:4) | 726.5 | 585.5 | y |
| 200 | PE(O) | PE(O-34:1) | 704.6 | 563.5 | y |
| 201 | PE(P) | PE(P-16:0/18:1) | 702.5 | 339.3 | y |
| 202 | PE(P) | PE(P-16:0/18:2) | 700.5 | 337.3 | y |
| 203 | PE(P) | PE(P-16:0/20:4) | 724.5 | 361.3 | y |
| 204 | PE(P) | PE(P-16:0/20:5) | 722.5 | 359.3 | y |
| 205 | PE(P) | PE(P-16:0/22:4) | 752.6 | 389.3 | y |
| 206 | PE(P) | PE(P-16:0/22:6) | 748.5 | 385.3 | y |
| 207 | PE(P) | PE(P-17:0/20:4) | 738.6 | 361.3 | y |
| 208 | PE(P) | PE(P-17:0/22:6) | 762.6 | 385.3 | y |
| 209 | PE(P) | PE(P-18:0/18:1) | 730.6 | 339.3 | y |
| 210 | PE(P) | PE(P-18:0/18:2) | 728.6 | 337.3 | y |
| 211 | PE(P) | PE(P-18:0/20:4) | 752.6 | 361.3 | y |
| 212 | PE(P) | PE(P-18:0/20:5) | 750.5 | 359.3 | y |
| 213 | PE(P) | PE(P-18:0/22:4) | 780.6 | 389.3 | y |
| 214 | PE(P) | PE(P-18:0/22:6) | 776.6 | 385.3 | y |
| 215 | PE(P) | PE(P-18:1/20:4) | 750.5 | 361.3 | y |
| 216 | PE(P) | PE(P-18:1/20:5) | 748.5 | 359.3 | y |
| 217 | PE(P) | PE(P-20:0/18:2) | 756.6 | 337.3 | y |
| 218 | PE(P) | PE(P-20:0/20:4) | 780.6 | 361.3 | y |
| 219 | PI | PI(15-MHDA_20:4) & PI(17:0_20:4) | 890.6 | 613.6 | y |
| 220 | PI | PI(16:0_16:1) | 826.5 | 549.5 | y |
| 221 | PI | PI(16:0_20:4) | 876.6 | 599.6 | y |
| 222 | PI | PI(18:0_18:1) | 882.6 | 605.6 | y |
| 223 | PI | PI(18:0_20:3) | 906.6 | 629.6 | y |
| 224 | PI | PI(18:0_20:4) | 904.6 | 627.6 | y |
| 225 | PI | PI(18:0_22:6) | 928.6 | 651.6 | y |
| 226 | PI | PI(34:1) | 854.6 | 577.6 | y |
| 227 | PI | PI(36:2) | 880.6 | 603.6 | y |
| 228 | PI | PI(38:6) | 900.6 | 623.6 | y |
| 229 | PIP1 | PIP1(38:4) | 984.7 | 627.7 | |
| 230 | PS | PS(38:4) | 812.5 | 627.5 | y |
| 231 | S1P | S1P(d18:1) | 380.2 | 264.3 | y |
| 232 | SHexCer | SHexCer(d18:1/16:0(OH)) | 796.8 | 264.3 | y |
| 233 | SHexCer | SHexCer(d18:1/16:0) | 780.8 | 264.3 | y |
| 234 | SM | SM(34:3) | 699.5 | 184.1 | y |
| 235 | SM | SM(35:1) | 717.6 | 184.1 | y |
| 236 | SM | SM(35:2) | 715.6 | 184.1 | y |
| 237 | SM | SM(38:3) | 755.6 | 184.1 | y |
| 238 | SM | SM(40:4) | 781.5 | 184.1 | |
| 239 | SM | SM(41:1) | 801.7 | 184.1 | y |
| 240 | SM | SM(42:3) | 811.5 | 184.1 | |
| 241 | SM | SM(44:2) | 841.6 | 184.1 | y |
| 242 | SM | SM(44:3) | 839.6 | 184.1 | y |
| 243 | SM | SM(d17:1/14:0) | 661.5 | 184.1 | y |
| 244 | SM | SM(d17:1/16:0) | 689.6 | 184.1 | y |
| 245 | SM | SM(d18:1/14:0) & SM(d16:1/16:0) | 675.5 | 184.1 | y |
| 246 | SM | SM(d18:1/16:0) | 703.6 | 184.1 | y |
| 247 | SM | SM(d18:1/18:0) & SM(d16:1/20:0) | 731.6 | 184.1 | y |
| 248 | SM | SM(d18:1/20:0) & SM(d16:1/22:0) | 759.6 | 184.1 | y |
| 249 | SM | SM(d18:1/22:0) & SM(d16:1/24:0) | 787.7 | 184.1 | y |
| 250 | SM | SM(d18:2/14:0) | 673.5 | 184.1 | y |
| 251 | SM | SM(d18:2/16:0) | 701.6 | 184.1 | y |
| 252 | SM | SM(d18:2/18:1) | 727.6 | 184.1 | y |
| 253 | SM | SM(d18:2/20:0) | 757.6 | 184.1 | y |
| 254 | TG [NL] | TG(48:0) [NL-16:0] | 824.8 | 551.5 | y |
| 255 | TG [NL] | TG(48:1) [NL-16:1] | 822.8 | 551.5 | y |
| 256 | TG [NL] | TG(48:1) [NL-18:1] | 822.8 | 523.5 | y |
| 257 | TG [NL] | TG(48:2) [NL-14:0] | 820.8 | 575.5 | y |
| 258 | TG [NL] | TG(48:2) [NL-14:1] | 820.8 | 577.5 | y |
| 259 | TG [NL] | TG(48:2) [NL-16:1] | 820.8 | 549.5 | y |
| 260 | TG [NL] | TG(48:2) [NL-18:2] | 820.8 | 523.5 | y |
| 261 | TG [NL] | TG(48:3) [NL-14:0] | 818.8 | 573.5 | y |
| 262 | TG [NL] | TG(48:3) [NL-16:1] | 818.8 | 547.5 | y |
| 263 | TG [NL] | TG(48:3) [NL-18:3] | 818.8 | 523.5 | y |
| 264 | TG [NL] | TG(49:1) [NL-17:1] | 836.8 | 551.5 | y |
| 265 | TG [NL] | TG(50:0) [NL-18:0] | 852.8 | 551.5 | y |
| 266 | TG [NL] | TG(50:1) [NL-16:0] | 850.8 | 577.5 | y |
| 267 | TG [NL] | TG(50:1) [NL-18:1] | 850.8 | 551.5 | y |
| 268 | TG [NL] | TG(50:2) [NL-14:0] | 848.8 | 603.5 | y |
| 269 | TG [NL] | TG(50:2) [NL-16:1] | 848.8 | 577.5 | y |
| 270 | TG [NL] | TG(50:2) [NL-18:1] | 848.8 | 549.5 | y |
| 271 | TG [NL] | TG(50:2) [NL-18:2] | 848.8 | 551.5 | y |
| 272 | TG [NL] | TG(50:3) [NL-14:0] | 846.8 | 601.5 | y |
| 273 | TG [NL] | TG(50:3) [NL-14:1] | 846.8 | 603.5 | y |
| 274 | TG [NL] | TG(50:3) [NL-16:1] | 846.8 | 575.5 | y |
| 275 | TG [NL] | TG(50:3) [NL-18:2] | 846.8 | 549.5 | y |
| 276 | TG [NL] | TG(50:3) [NL-18:3] | 846.8 | 551.5 | y |
| 277 | TG [NL] | TG(50:4) [NL-14:0] | 844.8 | 599.5 | y |
| 278 | TG [NL] | TG(50:4) [NL-18:3] | 844.8 | 549.5 | y |
| 279 | TG [NL] | TG(50:4) [NL-20:4] | 844.8 | 523.5 | y |
| 280 | TG [NL] | TG(51:0) [NL-16:0] | 866.7 | 593.4 | y |
| 281 | TG [NL] | TG(51:1) [NL-17:0] | 864.8 | 577.5 | y |
| 282 | TG [NL] | TG(51:2) [NL-15:0] | 862.8 | 603.5 | y |
| 283 | TG [NL] | TG(51:2) [NL-17:0] | 862.8 | 575.5 | y |
| 284 | TG [NL] | TG(51:2) [NL-17:1] | 862.8 | 577.5 | y |
| 285 | TG [NL] | TG(52:1) [NL-18:0] | 878.8 | 577.5 | y |
| 286 | TG [NL] | TG(52:1) [NL-18:1] | 878.8 | 579.5 | y |
| 287 | TG [NL] | TG(52:2) [NL-16:0] | 876.8 | 603.5 | y |
| 288 | TG [NL] | TG(52:3) [NL-18:2] | 874.8 | 577.5 | y |
| 289 | TG [NL] | TG(52:4) [NL-16:1] | 872.8 | 601.5 | y |
| 290 | TG [NL] | TG(52:4) [NL-18:2] | 872.8 | 575.5 | y |
| 291 | TG [NL] | TG(52:4) [NL-18:3] | 872.8 | 577.5 | y |
| 292 | TG [NL] | TG(52:5) [NL-18:3] | 870.8 | 575.5 | y |
| 293 | TG [NL] | TG(52:5) [NL-20:4] | 870.8 | 549.5 | y |
| 294 | TG [NL] | TG(52:5) [NL-20:5] | 870.8 | 551.5 | y |
| 295 | TG [NL] | TG(53:2) [NL-17:1] | 890.8 | 605.5 | y |
| 296 | TG [NL] | TG(53:2) [NL-18:1] | 890.8 | 591.5 | y |
| 297 | TG [NL] | TG(54:2) [NL-18:0] | 904.8 | 603.5 | y |
| 298 | TG [NL] | TG(54:2) [NL-20:1] | 904.8 | 577.5 | y |
| 299 | TG [NL] | TG(54:3) [NL-18:1] | 902.8 | 603.5 | y |
| 300 | TG [NL] | TG(54:3) [NL-18:2] | 902.8 | 605.5 | y |
| 301 | TG [NL] | TG(54:4) [NL-18:2] | 900.8 | 603.5 | y |
| 302 | TG [NL] | TG(54:4) [NL-20:3] | 900.8 | 577.5 | y |
| 303 | TG [NL] | TG(54:5) [NL-18:3] | 898.8 | 603.5 | y |
| 304 | TG [NL] | TG(54:5) [NL-20:4] | 898.8 | 577.5 | y |
| 305 | TG [NL] | TG(54:6) [NL-18:3] | 896.8 | 601.5 | y |
| 306 | TG [NL] | TG(54:6) [NL-20:4] | 896.8 | 575.5 | y |
| 307 | TG [NL] | TG(54:6) [NL-20:5] | 896.8 | 577.5 | y |
| 308 | TG [NL] | TG(54:6) [NL-22:6] | 896.8 | 551.5 | y |
| 309 | TG [NL] | TG(54:7) [NL-20:5] | 894.8 | 575.5 | y |
| 310 | TG [NL] | TG(54:7) [NL-22:6] | 894.8 | 549.5 | y |
| 311 | TG [NL] | TG(56:6) [NL-20:4] | 924.8 | 603.5 | y |
| 312 | TG [NL] | TG(56:6) [NL-22:5] | 924.8 | 577.5 | y |
| 313 | TG [NL] | TG(56:7) [NL-20:4] | 922.8 | 601.5 | y |
| 314 | TG [NL] | TG(56:7) [NL-20:5] | 922.8 | 603.5 | y |
| 315 | TG [NL] | TG(56:7) [NL-22:5] | 922.8 | 575.5 | y |
| 316 | TG [NL] | TG(56:7) [NL-22:6] | 922.8 | 577.5 | y |
| 317 | TG [NL] | TG(56:8) [NL-20:4] | 920.8 | 599.5 | y |
| 318 | TG [NL] | TG(56:8) [NL-20:5] | 920.8 | 601.5 | y |
| 319 | TG [NL] | TG(56:8) [NL-22:6] | 920.8 | 575.5 | y |
| 320 | TG [NL] | TG(56:9) [NL-22:6] | 918.8 | 573.5 | y |
| 321 | TG [NL] | TG(58:10) [NL-22:6] | 944.9 | 599.5 | y |
| 322 | TG [NL] | TG(58:8) [NL-22:6] | 948.8 | 603.5 | y |
| 323 | TG [NL] | TG(58:9) [NL-22:6] | 946.9 | 601.5 | y |
| 324 | TG(O) [NL] | TG(O-50:1) [NL-15:0] | 836.8 | 577.5 | y |
| 325 | TG(O) [NL] | TG(O-50:1) [NL-16:0] | 836.8 | 563.5 | y |
| 326 | TG(O) [NL] | TG(O-50:1) [NL-17:1] | 836.8 | 551.5 | y |
| 327 | TG(O) [NL] | TG(O-50:1) [NL-18:1] | 836.8 | 537.5 | y |
| 328 | TG(O) [NL] | TG(O-50:2) [NL-16:1] | 834.8 | 563.5 | y |
| 329 | TG(O) [NL] | TG(O-50:2) [NL-18:1] | 834.8 | 535.5 | y |
| 330 | TG(O) [NL] | TG(O-50:2) [NL-18:2] | 834.8 | 537.5 | y |
| 331 | TG(O) [NL] | TG(O-52:0) [NL-16:0] | 866.8 | 593.5 | y |
| 332 | TG(O) [NL] | TG(O-52:2) [NL-16:0] | 862.8 | 589.5 | y |
| 333 | TG(O) [NL] | TG(O-52:2) [NL-17:1] | 862.8 | 577.5 | y |
| 334 | TG(O) [NL] | TG(O-52:2) [NL-18:1] | 862.8 | 563.5 | y |
| 335 | TG(O) [NL] | TG(O-54:3) [NL-17:1] | 888.8 | 603.5 | y |
| 336 | TG(O) [NL] | TG(O-54:3) [NL-18:1] | 888.8 | 589.5 | y |
| 337 | TG(O) [NL] | TG(O-54:4) [NL-17:1] | 886.8 | 601.5 | y |
| 338 | TG(O) [NL] | TG(O-54:4) [NL-18:2] | 886.8 | 589.5 | y |
| 339 | Ubiquinone | Ubiquinone | 880.7 | 197 | y |

TABLE 5
Internal standards

| Lipid | | Precursor | Product |
|---|---|---|---|
| Number | Compound Name | (m/z) | (m/z) |
| S1 | AcylCarnitine 16:0 d3 (IS) | 403.4 | 85.1 |
| S2 | CE 18:0-d6 (IS) | 676.7 | 375.3 |
| S3 | Cer(d18:1-d7/18:0) (IS) | 573.6 | 271.4 |
| S4 | Cholic Acid d4 (IS) | 430.3 | 359.3 |
| S5 | COH-d7 (IS) | 376.4 | 161.2 |
| S6 | DE(18:1) ester d6 (IS) | 672.6 | 373.4 |
| S7 | DG(15:0 18:1) d7 (IS) | 605.5 | 299.5 |
| S8 | FA(18:1) d9 (IS) | 290.3 | 290.2 |
| S9 | FA(20:4) d11 (IS) | 314.2 | 314.2 |
| S10 | FA(22:6) d5 (IS) | 332.2 | 332.2 |
| S11 | Hex2Cer(d18:1/15:0) d7 (IS) | 855.6 | 271.3 |
| S12 | Hex3Cer(d18:1/17:0) (IS) | 1038.7 | 264.3 |
| S13 | HexCer(d18:1/15:0) d7 (IS) | 693.6 | 271.3 |
| S14 | LPC(18:1) d7 (IS) | 529.4 | 184.1 |
| S15 | LPE(18:1) d7 (IS) | 487.3 | 346.3 |
| S16 | LPI 13:0 (IS) | 548.3 | 271.3 |
| S17 | MG(18:1) d7 (IS) | 364.2 | 272.2 |
| S18 | PA(15:0_18:1) d7 (IS) | 685.6 | 570.6 |
| S19 | PC(15:0_18:1) d7 (IS) | 753.6 | 184.1 |
| S20 | PC(P-18:0/18:1) d9 (IS) | 781.6 | 184.1 |
| S21 | PE(15:0_18:1) d7 (IS) | 711.6 | 570.5 |
| S22 | PE(P-18:0/18:1) d9 (IS) | 739.5 | 348.3 |
| S23 | PG(15:0_18:1) d7 (IS) | 759.6 | 570.6 |
| S24 | PI(15:0_18:1) d7 (IS) | 847.6 | 570.6 |
| S25 | PS(15:0_18:1) d7 (IS) | 755.5 | 570.5 |
| S26 | SIP(d18:1) d7 (IS) | 387.2 | 271.3 |
| S27 | SHexCer(d18:1/12:0) (IS) | 724.8 | 264.3 |
| S28 | SM(d18:1/15:0) d9 (IS) | 698.6 | 193.1 |
| S29 | Sph(d17:1) (IS) | 286.3 | 268.3 |
| S30 | TG(48:1) [NL-18:1] d7 (IS) | 829.8 | 523.5 |

### Example 3: Further Refinement to Generate Clinical Lipidomics Platform 2 (CLP2) List

To produce a list of lipid species in which the resulting chromatographic peaks of interest could be most readily integrated via software automation without the need for manual intervention, multiple reference peaks, or complex peak picking algorithms, the CLP1 list was further refined such that any lipid species for which the chromatographic peak of interest eluted within a 0.5 minute retention time window of any other chromatographic peaks sharing the same transition, were omitted from the CLP2 list (Table 6). This was achieved using the same dataset as above. The resulting CLP2 list contains 269 lipid species (Table 6) excluding 30 internal standards (Table 5).

TABLE 6
CLP2 Lipid List

| Lipid | Lipid | | Precursor | Product |
|---|---|---|---|---|
| Number | Class | Compound Name | (m/z) | (m/z) |
| 1 | AC | AC(10:0) | 316.3 | 85.1 |
| 2 | AC | AC(12:0) | 344.3 | 85.1 |
| 3 | AC | AC(12:1) | 342.3 | 85.1 |
| 4 | AC | AC(13:0) | 358.3 | 85.1 |
| 5 | AC | AC(14:0) | 372.3 | 85.1 |
| 6 | AC−OH | AC(14:0)-OH | 388.3 | 85.1 |
| 7 | AC | AC(14:1) | 370.3 | 85.1 |
| 8 | AC−OH | AC(14:1)-OH | 386.3 | 85.1 |
| 9 | AC | AC(14:2) | 368.3 | 85.1 |
| 10 | AC | AC(16:0) | 400.4 | 85.1 |
| 11 | AC | AC(16:1) | 398.3 | 85.1 |
| 12 | AC | AC(18:1) | 426.4 | 85.1 |
| 13 | AC | AC(18:2) | 424.3 | 85.1 |
| 14 | AC | AC(24:0) | 512.3 | 85.1 |
| 15 | AC | AC(26:0) | 540.3 | 85.1 |
| 16 | AC | AC(26:1) | 538.3 | 85.1 |
| 17 | CE | CE(14:0) | 614.6 | 369.3 |
| 18 | CE | CE(15:0) | 628.6 | 369.3 |
| 19 | CE | CE(16:0) | 642.6 | 369.3 |
| 20 | CE | CE(16:1) | 640.6 | 369.3 |
| 21 | CE | CE(16:2) | 638.6 | 369.3 |
| 22 | CE | CE(17:0) | 656.6 | 369.3 |
| 23 | CE | CE(18:2) | 666.6 | 369.3 |
| 24 | CE | CE(18:3) | 664.6 | 369.3 |
| 25 | CE | CE(20:0) | 698.7 | 369.3 |
| 26 | CE | CE(20:1) | 696.7 | 369.3 |
| 27 | CE | CE(20:4) | 690.6 | 369.3 |
| 28 | CE | CE(20:5) | 688.6 | 369.3 |
| 29 | CE | CE(22:1) | 724.7 | 369.3 |
| 30 | CE | CE(22:6) | 714.6 | 369.3 |
| 31 | Cer(d) | Cer(d16:1/22:0) | 594.6 | 236.3 |
| 32 | Cer(d) | Cer(d16:1/24:0) | 622.6 | 236.3 |
| 33 | Cer(d) | Cer(d16:1/24:1) | 620.6 | 236.3 |
| 34 | Cer(d) | Cer(d17:1/24:0) | 636.6 | 250.3 |
| 35 | Cer(d) | Cer(d18:1/16:0) | 538.5 | 264.3 |
| 36 | Cer(d) | Cer(d18:1/20:0) | 594.6 | 264.3 |
| 37 | Cer(d) | Cer(d18:1/22:0) | 622.6 | 264.3 |
| 38 | Cer(d) | Cer(d18:1/23:0) | 636.6 | 264.3 |
| 39 | Cer(d) | Cer(d18:1/24:0) | 650.6 | 264.3 |
| 40 | Cer(d) | Cer(d18:1/24:1) | 648.6 | 264.3 |
| 41 | Cer(d) | Cer(d18:2/22:0) | 620.6 | 262.3 |
| 42 | Cer(d) | Cer(d18:2/23:0) | 634.6 | 262.3 |
| 43 | Cer(d) | Cer(d18:2/24:0) | 648.6 | 262.3 |
| 44 | Cer(d) | Cer(d18:2/24:1) | 646.6 | 262.3 |
| 45 | Cer(d) | Cer(d19:1/22:0) | 636.6 | 278.3 |
| 46 | Cer(d) | Cer(d19:1/24:0) | 664.6 | 278.3 |
| 47 | Cer(d) | Cer(d19:1/24:1) | 662.6 | 278.3 |
| 48 | Cer(m) | Cer(m18:0/22:0) | 608.6 | 268.4 |
| 49 | Cer(m) | Cer(m18:1/20:0) | 578.6 | 266.4 |
| 50 | Cer(m) | Cer(m18:1/22:0) | 606.6 | 266.4 |
| 51 | Cer(m) | Cer(m18:1/24:0) | 634.6 | 266.4 |
| 52 | Cer(m) | Cer(m18:1/24:1) | 632.6 | 266.4 |
| 53 | C1P | Cer1P(d18:1/16:0) | 618.4 | 264.3 |
| 54 | COH | COH | 369.4 | 161.2 |
| 55 | DE | DE(18:1) | 666.6 | 367.4 |
| 56 | DE | DE(18:2) | 664.6 | 367.4 |
| 57 | DE | DE(20:4) | 688.6 | 367.4 |
| 58 | DG | DG(16:0_16:0) | 586.5 | 313.2 |
| 59 | DG | DG(16:0_18:1) | 612.6 | 313.3 |
| 60 | DG | DG(16:0_18:2) | 610.5 | 313.2 |
| 61 | DG | DG(18:1_18:1) | 638.6 | 339.3 |
| 62 | DG | DG(18:1_18:2) | 636.6 | 339.3 |
| 63 | DG | DG(18:1_20:3) | 662.6 | 339.3 |
| 64 | dimethyl-CE | dimethyl-CE(18:2) | 694.6 | 397.3 |
| 65 | dimethyl-CE | dimethyl-CE(20:4) | 718.6 | 397.3 |
| 66 | FFA | FA(14:0) | 227.2 | 227.2 |
| 67 | FFA | FA(16:0) | 255.2 | 255.2 |
| 68 | FFA | FA(16:1) | 253.2 | 253.2 |
| 69 | FFA | FA(17:1) | 267.2 | 267.2 |
| 70 | FFA | FA(18:1) | 281.3 | 281.2 |
| 71 | FFA | FA(18:2) | 279.2 | 279.2 |
| 72 | FFA | FA(18:3) | 277.2 | 277.2 |
| 73 | FFA | FA(20:4) | 303.2 | 303.2 |
| 74 | FFA | FA(22:4) | 331.3 | 331.3 |
| 75 | FFA | FA(22:5) | 329.3 | 329.2 |
| 76 | FFA | FA(22:6) | 327.2 | 327.2 |
| 77 | GM3 | GM3(d18:1/22:0) | 1237.8 | 264.3 |
| 78 | GM3 | GM3(d18:1/24:1) | 1263.8 | 264.3 |
| 79 | Hex2Cer | Hex2Cer(d16:1/16:0) | 834.6 | 236.3 |
| 80 | Hex2Cer | Hex2Cer(d18:1/16:0) | 862.6 | 264.3 |
| 81 | Hex2Cer | Hex2Cer(d18:1/22:0) | 946.7 | 264.3 |
| 82 | Hex2Cer | Hex2Cer(d18:1/24:1) | 972.7 | 264.3 |
| 83 | Hex3Cer | Hex3Cer(d18:1/16:0) | 1024.7 | 264.3 |
| 84 | HexCer | HexCer(d18:1/16:0) | 700.6 | 264.3 |
| 85 | HexCer | HexCer(d18:1/18:0) | 728.6 | 264.3 |
| 86 | HexCer | HexCer(d18:1/22:0) | 784.7 | 264.3 |
| 87 | HexCer | HexCer(d18:1/24:1) | 810.7 | 264.3 |
| 88 | HexCer | HexCer(d18:2/24:0) | 810.7 | 262.3 |
| 89 | LPC | LPC(14:0) | 468.3 | 184.1 |
| 90 | LPC | LPC(17:0) | 510.4 | 184.1 |
| 91 | LPC | LPC(18:0) | 524.4 | 184.1 |
| 92 | LPC | LPC(18:1) | 522.4 | 184.1 |
| 93 | LPC | LPC(18:2) | 520.3 | 184.1 |
| 94 | LPC | LPC(18:3) | 518.3 | 184.1 |
| 95 | LPC | LPC(19:0) | 538.4 | 184.1 |
| 96 | LPC | LPC(19:1) | 536.4 | 184.1 |
| 97 | LPC | LPC(20:4) | 544.3 | 184.1 |
| 98 | LPC | LPC(20:5) | 542.3 | 184.1 |
| 99 | LPC | LPC(22:0) | 580.4 | 184.1 |
| 100 | LPC | LPC(22:5) | 570.4 | 184.1 |
| 101 | LPC | LPC(22:6) | 568.3 | 184.1 |
| 102 | LPC | LPC(24:0) | 608.5 | 184.1 |
| 103 | LPC | LPC(26:0) | 636.5 | 184.1 |
| 104 | LPC(O) | LPC(O-22:0) | 566.5 | 104.1 |
| 105 | LPC(O) | LPC(O-22:1) | 564.4 | 104.1 |
| 106 | LPC(O) | LPC(O-24:0) | 594.5 | 104.1 |
| 107 | LPC(P) | LPC(P-16:0) | 480.3 | 104.1 |
| 108 | LPC(P) | LPC(P-17:0) | 494.3 | 104.1 |
| 109 | LPE | LPE(16:0) | 454.3 | 313.3 |
| 110 | LPE | LPE(18:0) | 482.3 | 341.3 |
| 111 | LPE | LPE(18:1) | 480.3 | 339.3 |
| 112 | LPE | LPE(18:2) | 478.3 | 337.3 |
| 113 | LPE | LPE(20:4) | 502.3 | 361.3 |
| 114 | LPE | LPE(22:6) | 526.3 | 385.3 |
| 115 | LPE(P) | LPE(P-16:0) | 438.3 | 266.4 |
| 116 | LPE(P) | LPE(P-18:0) | 466.3 | 294.4 |
| 117 | LPI | LPI(18:0) | 618.3 | 341.3 |
| 118 | methyl-CE | methyl-CE(18:0) | 684.6 | 383.3 |
| 119 | methyl-CE | methyl-CE(18:2) | 680.6 | 383.3 |
| 120 | methyl-CE | methyl-CE(20:4) | 704.6 | 383.3 |
| 121 | methyl-DE | methyl-DE(18:1) | 680.6 | 381.4 |
| 122 | methyl-DE | methyl-DE(18:2) | 678.6 | 381.4 |
| 123 | PC | PC(15:0_22:6) | 792.6 | 184.1 |
| 124 | PC | PC(16:0_16:0) | 734.6 | 184.1 |
| 125 | PC | PC(16:0_18:1) | 760.6 | 184.1 |
| 126 | PC | PC(16:0_18:2) | 758.6 | 184.1 |
| 127 | PC | PC(16:0_20:4) | 782.6 | 184.1 |
| 128 | PC | PC(16:0_20:5) | 780.5 | 184.1 |
| 129 | PC | PC(16:0_22:6) | 806.6 | 184.1 |
| 130 | PC | PC(16:1_20:4) | 780.5 | 184.1 |
| 131 | PC | PC(18:0_20:4) | 810.6 | 184.1 |
| 132 | PC | PC(18:0_22:6) | 834.6 | 184.1 |
| 133 | PC | PC(18:1_20:3) | 810.6 | 184.1 |
| 134 | PC | PC(18:2_18:2) | 782.6 | 184.1 |
| 135 | PC | PC(31:0) | 720.6 | 184.1 |
| 136 | PC | PC(31:1) | 718.5 | 184.1 |
| 137 | PC | PC(32:1) | 732.6 | 184.1 |
| 138 | PC | PC(32:2) | 730.5 | 184.1 |
| 139 | PC | PC(33:1) | 746.6 | 184.1 |
| 140 | PC | PC(33:2) | 744.6 | 184.1 |
| 141 | PC | PC(36:2) | 786.6 | 184.1 |
| 142 | PC | PC(36:4) | 782.6 | 184.1 |
| 143 | PC | PC(38:4) (b) | 810.6 | 184.1 |
| 144 | PC | PC(38:6) (a) | 806.6 | 184.1 |
| 145 | PC | PC(40:8) | 830.6 | 184.1 |
| 146 | PC | PC(42:10) | 854.5 | 184.1 |
| 147 | PC | PC(42:2) | 870.5 | 184.1 |
| 148 | PC | PC(42:3) | 868.5 | 184.1 |
| 149 | PC | PC(42:4) | 866.5 | 184.1 |
| 150 | PC | PC(42:7) | 860.5 | 184.1 |
| 151 | PC | PC(42:8) | 858.5 | 184.1 |
| 152 | PC(O) | PC(O-16:0/16:0) | 720.6 | 184.1 |
| 153 | PC(O) | PC(O-42:8) | 844.5 | 184.1 |
| 154 | PC(O) | PC(O-46:7) | 902.5 | 184.1 |
| 155 | PC(P) | PC(P-16:0/14:0) | 690.4 | 184.1 |
| 156 | PC(P) | PC(P-16:0/16:0) | 718.5 | 184.1 |
| 157 | PC(P) | PC(P-16:0/18:2) | 742.5 | 184.1 |
| 158 | PC(P) | PC(P-16:0/20:4) | 766.5 | 184.1 |
| 159 | PC(P) | PC(P-16:0/20:5) | 764.6 | 184.1 |
| 160 | PC(P) | PC(P-17:0/20:4) | 780.5 | 184.1 |
| 161 | PE | PE(16:0_16:1) | 690.5 | 549.5 |
| 162 | PE | PE(16:0_18:1) | 718.5 | 577.5 |
| 163 | PE | PE(16:0_18:2) | 716.5 | 575.5 |
| 164 | PE | PE(16:0_20:4) | 740.5 | 599.5 |
| 165 | PE | PE(16:0_22:6) | 764.5 | 623.5 |
| 166 | PE | PE(18:0_20:4) | 768.6 | 627.5 |
| 167 | PE | PE(18:0_22:6) | 792.6 | 651.5 |
| 168 | PE | PE(36:2) | 744.6 | 603.5 |
| 169 | PE(O) | PE(O-16:0/20:4) | 726.5 | 585.5 |
| 170 | PE(O) | PE(O-34:1) | 704.6 | 563.5 |
| 171 | PE(P) | PE(P-16:0/18:2) | 700.5 | 337.3 |
| 172 | PE(P) | PE(P-16:0/20:4) | 724.5 | 361.3 |
| 173 | PE(P) | PE(P-16:0/20:5) | 722.5 | 359.3 |
| 174 | PE(P) | PE(P-16:0/22:6) | 748.5 | 385.3 |
| 175 | PE(P) | PE(P-17:0/20:4) | 738.6 | 361.3 |
| 176 | PE(P) | PE(P-17:0/22:6) | 762.6 | 385.3 |
| 177 | PE(P) | PE(P-18:0/18:1) | 730.6 | 339.3 |
| 178 | PE(P) | PE(P-18:0/18:2) | 728.6 | 337.3 |
| 179 | PE(P) | PE(P-18:0/20:4) | 752.6 | 361.3 |
| 180 | PE(P) | PE(P-18:0/20:5) | 750.5 | 359.3 |
| 181 | PE(P) | PE(P-18:0/22:6) | 776.6 | 385.3 |
| 182 | PE(P) | PE(P-18:1/20:4) | 750.5 | 361.3 |
| 183 | PE(P) | PE(P-18:1/20:5) | 748.5 | 359.3 |
| 184 | PE(P) | PE(P-20:0/18:2) | 756.6 | 337.3 |
| 185 | PE(P) | PE(P-20:0/20:4) | 780.6 | 361.3 |
| 186 | PI | PI(15-MHDA_20:4) & | 890.6 | 613.6 |
| | | PI(17:0_20:4) | | |
| 187 | PI | PI(16:0_16:1) | 826.5 | 549.5 |
| 188 | PI | PI(16:0_20:4) | 876.6 | 599.6 |
| 189 | PI | PI(18:0_18:1) | 882.6 | 605.6 |
| 190 | PI | PI(18:0_20:4) | 904.6 | 627.6 |
| 191 | PI | PI(18:0_22:6) | 928.6 | 651.6 |
| 192 | PI | PI(34:1) | 854.6 | 577.6 |
| 193 | PI | PI(36:2) | 880.6 | 603.6 |
| 194 | PI | PI(38:6) | 900.6 | 623.6 |
| 195 | PIP1 | PIP1(38:4) | 984.7 | 627.7 |
| 196 | PS | PS(38:4) | 812.5 | 627.5 |
| 197 | S1P | S1P(d18:1) | 380.2 | 264.3 |
| 198 | SHexCer | SHexCer(d18:1/16:0(OH)) | 796.8 | 264.3 |
| 199 | SHexCer | SHexCer(d18:1/16:0) | 780.8 | 264.3 |
| 200 | SM | SM(34:3) | 699.5 | 184.1 |
| 201 | SM | SM(35:1) | 717.6 | 184.1 |
| 202 | SM | SM(35:2) | 715.6 | 184.1 |
| 203 | SM | SM(38:3) | 755.6 | 184.1 |
| 204 | SM | SM(41:1) | 801.7 | 184.1 |
| 205 | SM | SM(44:2) | 841.6 | 184.1 |
| 206 | SM | SM(d17:1/14:0) | 661.5 | 184.1 |
| 207 | SM | SM(d17:1/16:0) | 689.6 | 184.1 |
| 208 | SM | SM(d18:1/14:0) & | 675.5 | 184.1 |
| | | SM(d16:1/16:0) | | |
| 209 | SM | SM(d18:1/16:0) | 703.6 | 184.1 |
| 210 | SM | SM(d18:1/18:0) & | 731.6 | 184.1 |
| | | SM(d16:1/20:0) | | |
| 211 | SM | SM(d18:1/20:0) & | 759.6 | 184.1 |
| | | SM(d16:1/22:0) | | |
| 212 | SM | SM(d18:1/22:0) & | 787.7 | 184.1 |
| | | SM(d16:1/24:0) | | |
| 213 | SM | SM(d18:2/14:0) | 673.5 | 184.1 |
| 214 | SM | SM(d18:2/16:0) | 701.6 | 184.1 |
| 215 | SM | SM(d18:2/18:1) | 727.6 | 184.1 |
| 216 | SM | SM(d18:2/20:0) | 757.6 | 184.1 |
| 217 | TG [NL] | TG(48:1) [NL-18:1] | 822.8 | 523.5 |
| 218 | TG [NL] | TG(48:2) [NL-14:0] | 820.8 | 575.5 |
| 219 | TG [NL] | TG(48:2) [NL-14:1] | 820.8 | 577.5 |
| 220 | TG [NL] | TG(48:2) [NL-16:1] | 820.8 | 549.5 |
| 221 | TG [NL] | TG(48:2) [NL-18:2] | 820.8 | 523.5 |
| 222 | TG [NL] | TG(48:3) [NL-14:0] | 818.8 | 573.5 |
| 223 | TG [NL] | TG(48:3) [NL-16:1] | 818.8 | 547.5 |
| 224 | TG [NL] | TG(50:2) [NL-16:1] | 848.8 | 577.5 |
| 225 | TG [NL] | TG(50:2) [NL-18:1] | 848.8 | 549.5 |
| 226 | TG [NL] | TG(50:3) [NL-14:0] | 846.8 | 601.5 |
| 227 | TG [NL] | TG(50:3) [NL-14:1] | 846.8 | 603.5 |
| 228 | TG [NL] | TG(50:3) [NL-16:1] | 846.8 | 575.5 |
| 229 | TG [NL] | TG(50:3) [NL-18:2] | 846.8 | 549.5 |
| 230 | TG [NL] | TG(50:4) [NL-14:0] | 844.8 | 599.5 |
| 231 | TG [NL] | TG(50:4) [NL-18:3] | 844.8 | 549.5 |
| 232 | TG [NL] | TG(50:4) [NL-20:4] | 844.8 | 523.5 |
| 233 | TG [NL] | TG(51:2) [NL-17:0] | 862.8 | 575.5 |
| 234 | TG [NL] | TG(51:2) [NL-17:1] | 862.8 | 577.5 |
| 235 | TG [NL] | TG(52:2) [NL-16:0] | 876.8 | 603.5 |
| 236 | TG [NL] | TG(52:3) [NL-18:2] | 874.8 | 577.5 |
| 237 | TG [NL] | TG(52:4) [NL-16:1] | 872.8 | 601.5 |
| 238 | TG [NL] | TG(52:4) [NL-18:2] | 872.8 | 575.5 |
| 239 | TG [NL] | TG(52:4) [NL-18:3] | 872.8 | 577.5 |
| 240 | TG [NL] | TG(52:5) [NL-18:3] | 870.8 | 575.5 |
| 241 | TG [NL] | TG(52:5) [NL-20:4] | 870.8 | 549.5 |
| 242 | TG [NL] | TG(54:2) [NL-20:1] | 904.8 | 577.5 |
| 243 | TG [NL] | TG(54:3) [NL-18:1] | 902.8 | 603.5 |
| 244 | TG [NL] | TG(54:5) [NL-18:3] | 898.8 | 603.5 |
| 245 | TG [NL] | TG(54:5) [NL-20:4] | 898.8 | 577.5 |
| 246 | TG [NL] | TG(54:6) [NL-18:3] | 896.8 | 601.5 |
| 247 | TG [NL] | TG(54:6) [NL-20:4] | 896.8 | 575.5 |
| 248 | TG [NL] | TG(54:6) [NL-20:5] | 896.8 | 577.5 |
| 249 | TG [NL] | TG(54:6) [NL-22:6] | 896.8 | 551.5 |
| 250 | TG [NL] | TG(54:7) [NL-20:5] | 894.8 | 575.5 |
| 251 | TG [NL] | TG(54:7) [NL-22:6] | 894.8 | 549.5 |
| 252 | TG [NL] | TG(56:6) [NL-20:4] | 924.8 | 603.5 |
| 253 | TG [NL] | TG(56:6) [NL-22:5] | 924.8 | 577.5 |
| 254 | TG [NL] | TG(56:7) [NL-20:4] | 922.8 | 601.5 |
| 255 | TG [NL] | TG(56:7) [NL-20:5] | 922.8 | 603.5 |
| 256 | TG [NL] | TG(56:7) [NL-22:5] | 922.8 | 575.5 |
| 257 | TG [NL] | TG(56:7) [NL-22:6] | 922.8 | 577.5 |
| 258 | TG [NL] | TG(56:8) [NL-20:4] | 920.8 | 599.5 |
| 259 | TG [NL] | TG(56:8) [NL-20:5] | 920.8 | 601.5 |
| 260 | TG [NL] | TG(56:8) [NL-22:6] | 920.8 | 575.5 |
| 261 | TG [NL] | TG(56:9) [NL-22:6] | 918.8 | 573.5 |
| 262 | TG [NL] | TG(58:10) [NL-22:6] | 944.9 | 599.5 |
| 263 | TG [NL] | TG(58:8) [NL-22:6] | 948.8 | 603.5 |
| 264 | TG [NL] | TG(58:9) [NL-22:6] | 946.9 | 601.5 |
| 265 | TG(O) [NL] | TG(O-50:2) [NL-16:1] | 834.8 | 563.5 |
| 266 | TG(O) [NL] | TG(O-50:2) [NL-18:1] | 834.8 | 535.5 |
| 267 | TG(O) [NL] | TG(O-50:2) [NL-18:2] | 834.8 | 537.5 |
| 268 | TG(O) [NL] | TG(O-54:4) [NL-17:1] | 886.8 | 601.5 |
| 269 | Ubiquinone | Ubiquinone | 880.7 | 197 |

### Example 4: Development of mBMI

Statistical modelling was used to condense large volumes of lipidomic data into a single risk score, a Metabolic BMI (mBMI) score (FIG. 3), which can be easily interpreted by clinician and patient alike. Statistical modelling is used to predict an individual's mBMI from their lipid profile, with some additional transformation. mBMI was found to be a valuable indicator of an individual's cardiometabolic health, and a valuable predictive variable for disease risk and outcomes.

The development and subsequent calculation of mBMI was made using the Australian Diabetes, Obesity and Lifestyle Study (AusDiab; n=10,339), a large cohort dataset. In this cohort, 717 lipid species from the Research Lipid List (Table 2) were measured. Calculated values utilising all measured lipids from the Research Lipid List (717 species, Table 2) were compared to calculated values utilising lipids from the CLP1 list (298 species, CLP1s, Table 4) and/or calculated values utilising lipids from the CLP2 list (Table 6). Calculation of these risk scores was further demonstrated using lipids from a further, truncated list, the CLP2 list (Table 6), featuring only those lipids which can be easily integrated without the use of peak picking algorithms (269 species, Table 6; 235 lipid species, CLP2s, Table 6 (indicated by ‘y’)). A ridge regression model was used.

The downstream analyses, consisting of association of resulting mBMIΔ with cardio-metabolic traits/diseases were subsequently performed.

The following sections describe the development of metabolic BMI (mBMI) scores using the full lipidome (Research Lipid List, 717 lipid species, Table 2) and the CLP lipid lists (CLP1s, Table 4 and CLP2s, Table 6). The mBMI score, and a score derived from the difference between mBMI and measured or actual BMI (mBMIΔ), were examined for their association with cardio-metabolic traits, their variation depending on the lipid list used in their calculation, and their association with incident-cardiometabolic diseases (FIG. 3).

### The Performance of Different Regularised Linear Models to Predict BMI

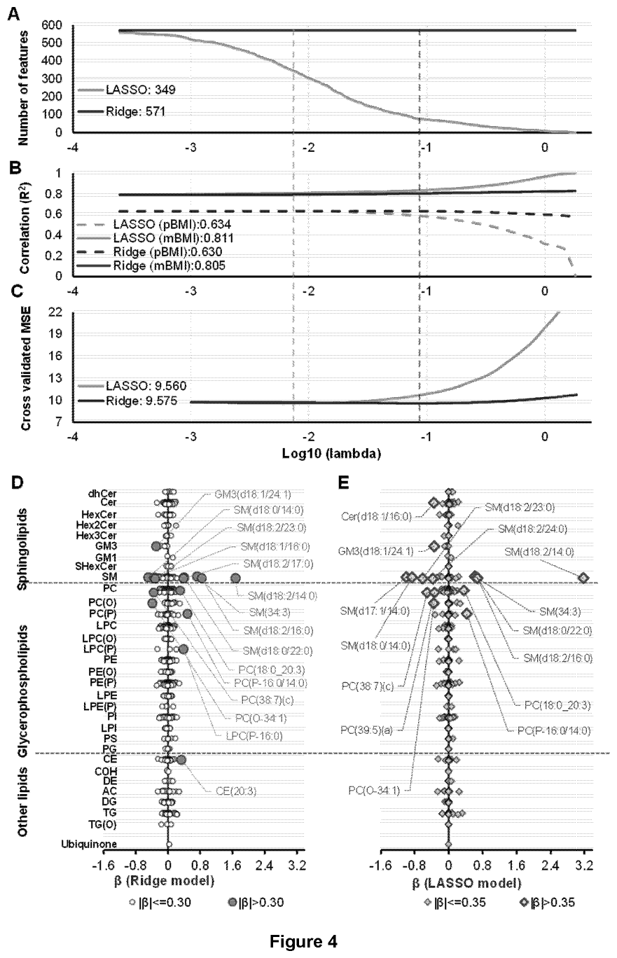

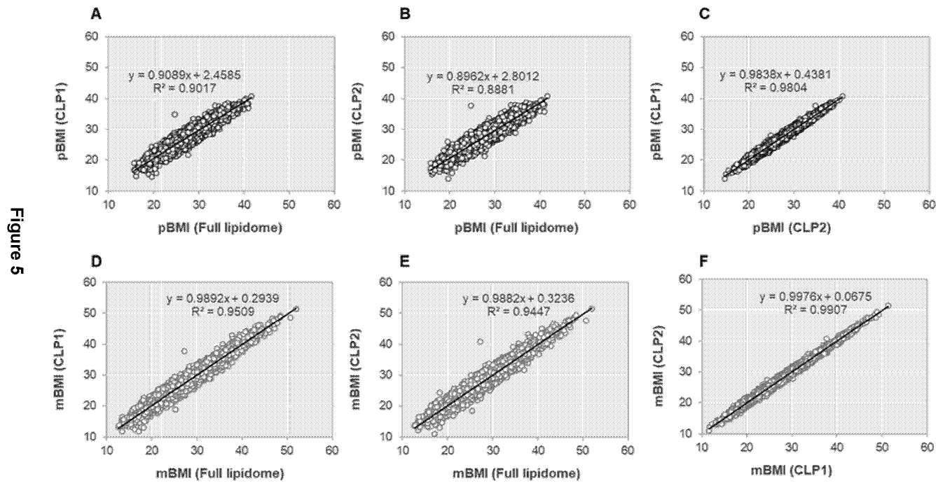

To assess the importance of the number of lipid species in the models, regularised linear models (ridge, elastic-net and LASSO) incorporating lipid species, age and sex, were compared for their ability to predict BMI in the AusDiab cohort and validated in the BHS. Using elastic-net (384 lipid species selected) and LASSO (349 lipid species selected) models, similar performance was observed as for the ridge model for the prediction of BMI (pBMI), with models explaining 60.8% to 60.9% of BMI variance in the AusDiab. To investigate how a further reduction in the number of lipid species in the model affected model performance, the regularisation parameter, lambda, was tuned in the LASSO models and in the ridge models for comparison, with log 10 lambda values between −4 and 0.2 (FIG. 4). As lambda was increased, the number of features selected into the LASSO model decreased until only 9 lipids were included in the model with a log 10 lambda of 0.

In the LASSO models, as lambda increased, the correlation (R2) between BMI and the pBMI decreased, while in the ridge models the R2 remained relatively stable (FIG. 4). The correlation (R2) between BMI and mBMI increased in the LASSO models reaching a R2 of 1.0 as the number of features incorporated into the LASSO models decreased to 0, but again showed little variation in the ridge models (FIG. 4). Optimisation of the lambda parameter by minimising the mean-squared error (MSE) using cv.glmnet showed the cross-validated MSE increasing in the LASSO models but again remaining relatively stable in the ridge models (FIG. 4). The optimum lambda used to model BMI for the ridge and LASSO models was defined by the lowest MSE. The beta-coefficients of the optimum ridge and LASSO models were then extracted: the lipid species showing the strongest contribution in the ridge and LASSO models were similar. SM(d18:2/14:0), displayed the strongest positive effect size in both models, β=1.677 (ridge) and β=3.172 (LASSO). FIG. 4 shows the beta coefficients from the ridge model and the LASSO model respectively.

Comparison of mBMI Scores Calculated Using the Research Lipid List Vs. The Clinical Lipidomics Platform lists (CLP1s and CLP2s) A Ridge regression was used to create a predictive model for BMI, with the inclusion of age and sex as covariates. To avoid overfitting, a 10-fold cross validation was employed in the AusDiab cohort (i.e. models were trained on the 9/10^{th} of the cohort, then used to predict BMI in the holdout 1/10^{th}). Using either the Research Lipid List (717 lipids, Table 2) or the CLP1s (298 lipids, Table 4) and CLP2s lists (235 lipids, Table 6), we observed similar performance for the prediction of BMI. The models provided predicted BMI (pBMI) values explaining 63.3%, 58.2% and 57.6% of the variance in BMI when either the Research Lipid List, CLP Is, or CLP2s lists were used, respectively (Table 7). The models were described as a list of the covariates and lipids used in each model together with their β-coefficients (Tables 8, 9, 10 and 11). The lipid species were ranked by the absolute value of their β-coefficients which defined the relative contribution they made to the model. LASSO models (using optimised lambda) were also developed with the CLP1s and CLP2s lipid lists and compared the number of lipids and performance of these models (Tables 8, 9, 10 and 11). It is recognised that the lipids at the top of these lists will contribute more to the model and so a subset of the lipids could be selected from the top 20 lipids, the top 50 lipids, the top 100 lipids, the top 200 lipids and used to make a model containing fewer lipids.

To standardise the pBMI to the population, the mBMI is derived as follows:

mBMI=BMI+(pBMI−pBMI value on the line of best fit between pBMI and BMI).

The mBMIΔ was then defined as the difference between BMI and mBMI. The correlation between BMI and mBMI was consistent across the lipid sets: R²=0.80.2, 80.3, and 80.2 when using the Research Lipid List, CLP1s and CLP2s respectively (Table 7).

TABLE 7
The predictive performances of ridge and
LASSO models for the prediction of BMI

| | Correlation (R²) (%) | |
|---|---|---|
| | BMI | BMI |
| Lipid set | versus pBMI | versus mBMI |
| Research Lipid List (ridge, | 63.3 | 80.2 |
| n = 717 species) | | |
| CLP1s List (ridge, n = 298 species) | 58.2 | 80.3 |
| CLP2s List (ridge, n = 235 species) | 57.6 | 80.1 |
| CLP1s List (LASSO, n = 151 species) | 58.3 | 80.0 |
| CLP2s List (LASSO, n = 112 species) | 57.5 | 80.0 |

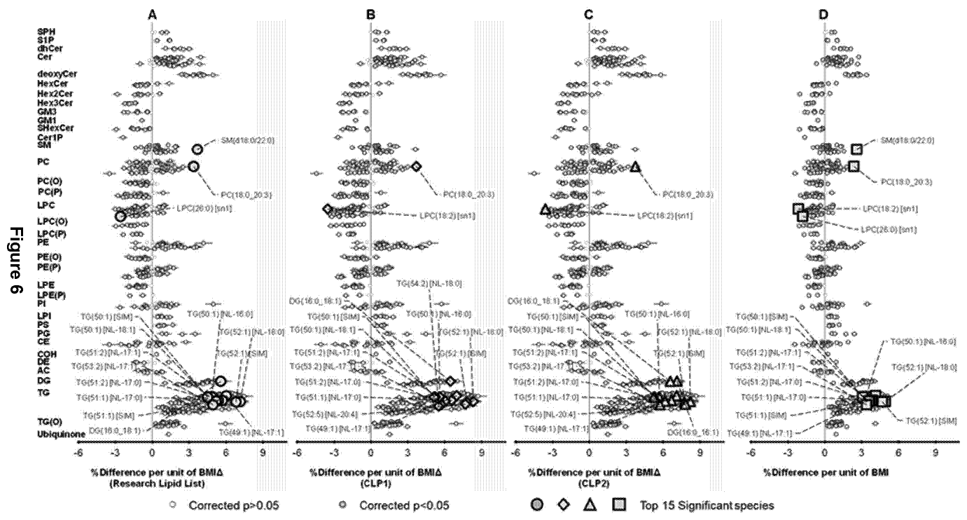

Similar values were achieved for pBMI and mBMI, whether the Research, CLP1s, or CLP2s lipid lists are used. The correlation (R²) between pBMI values calculated using the Research Lipid List, and pBMI values derived using CLP1s and CLP2s was 0.901 and 0.896 (FIG. 4) respectively. The correlation between pBMIs calculated using CLP2s and CLP1s was relatively higher: R²=0.980 (FIG. 5). Similar but tighter correlations among the mBMIs derived using the either the full lipidome, CLP2s or CLP1s were also evident (FIG. 4).

The Biological Signal Captured by mBMIΔ Generated Using the Research Lipid List, CLP1s Subset List and CLP2s Subset List Lipidome Strongly Align

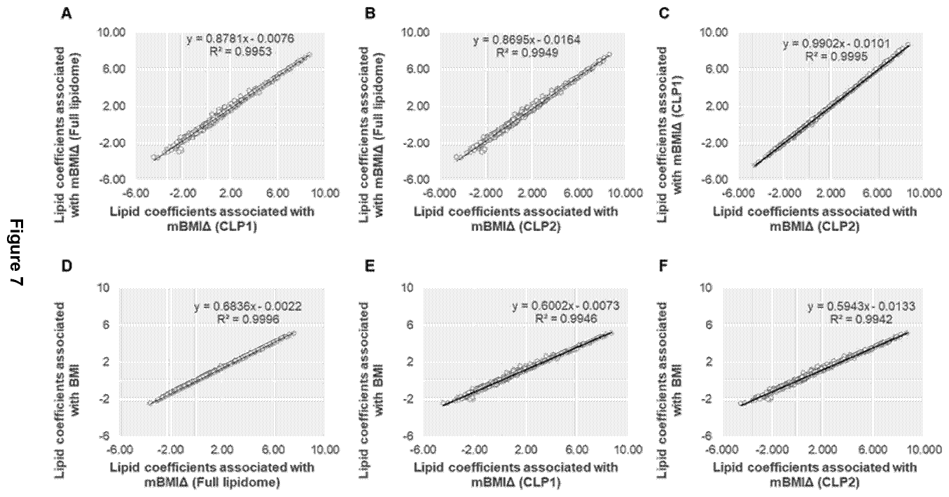

A regression analysis of lipid species was performed with BMI and mBMIΔ. In age and sex adjusted models, over 85% of the lipid species were observed to significantly associate with BMI and with mBMIΔ. Diacylglycerol, triacylglycerol and ceramide species showed a strong positive association, while most hexosylceramide, lyso and ether phospholipid species were negatively associated with both BMI and mBMIΔ (FIG. 5). The mBMIΔ calculated from the CLP 1 and the CLP2s show similar associations with lipids as the mBMIΔ calculated from the full lipidome (FIG. 6).

The beta coefficients of lipids associated with mBMIΔ (full lipidome, CLP1s or CLP2s) were compared with the coefficients associated with BMI. The correlation between effect sizes of each lipid associated with mBMIΔ (Research Lipoid List), mBMIΔ (CLP1s) and mBMIΔ (CLP2s) to each other and BMI resulted in R²>0.99 (FIG. 7). The biological signals captured by mBMIΔ derived using the CLP (both CLP1s and CLP2s) were similar to the signal captured by mBMIΔ calculated using the Research Lipid List and interestingly, with the signal captured by BMI.

Metabolic BMI (mBMI) Derived Using Either the Research Lipid List or CLP1s and CLP2s Lists Show Similar Associations with Cardiometabolic Risk Factors

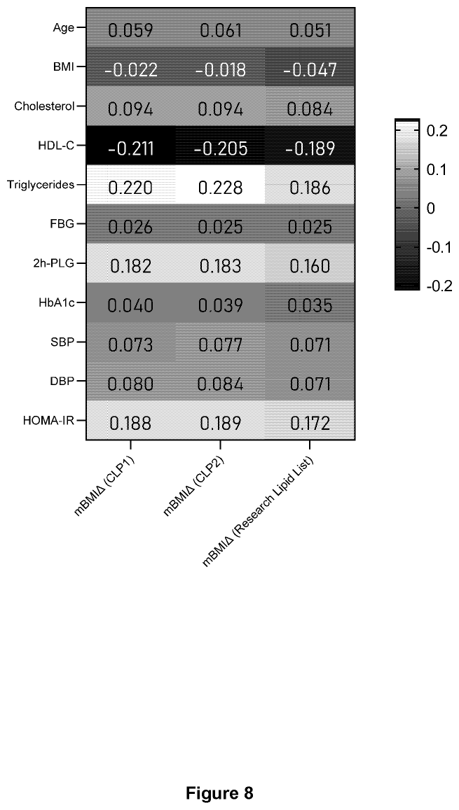

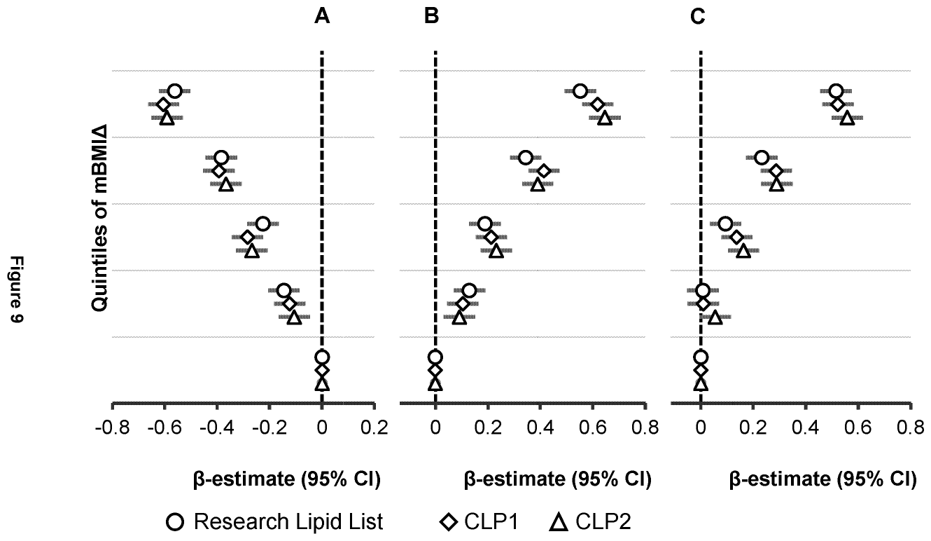

To check whether each of the mBMIΔ values calculated from the CLP1s, CLP2s and the Research Lipid List show the same association with disease risk, a correlation analysis against cardiometabolic risk factors was performed, and the Pearson's correlation coefficients were calculated. The correlation between mBMIΔ (CLP1s) or mBMIΔ (CLP2s) with cardiometabolic risk factors aligned closely with the correlation between mBMIΔ (Research Lipid List) and risk factors. Notably, mBMIΔ (CLP1s), mBMIΔ (CLP2s) and mBMIΔ (Research Lipid List) were all negatively correlated with HDL-C (r=−0.211, −0.205 and −0.189) (FIG. 8).

Next, the AusDiab participants were grouped into quintiles using mBMIΔ and a linear regression analysis was performed between selected cardio-metabolic risk factors (as outcome) and the quintiles of mBMIΔ (predictor) to assess the overall association. Individuals in the top quintile of mBMIΔ (Q5) had higher triglycerides (FIG. 9A), and lower HDL-C(FIG. 9B), as well as being more insulin resistant, and having higher HOMA-IR (FIG. 9C) compared to individuals in Q 1, regardless of the lipid set used to generated the mBMIΔ (FIG. 9).

The mBMI Scores Generated Using the Research Lipid List and CLP1s and CLP2s Lists Show Similar Associations with Cardio-Metabolic Diseases

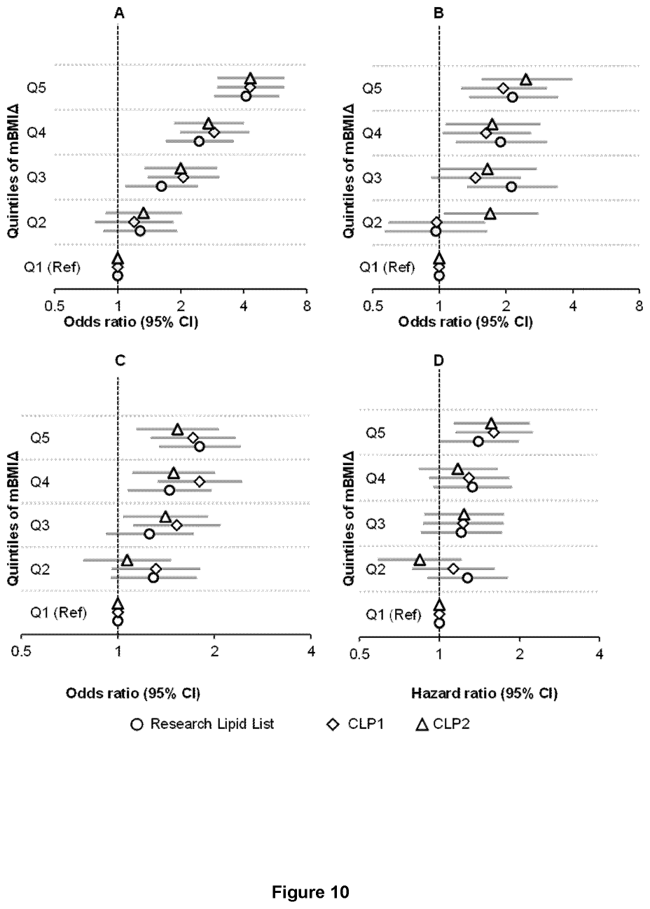

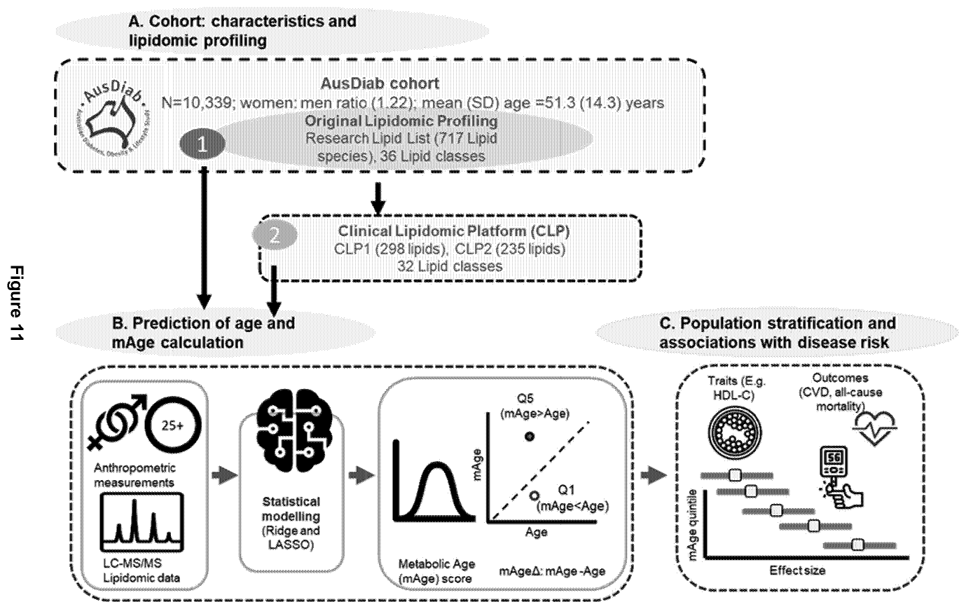

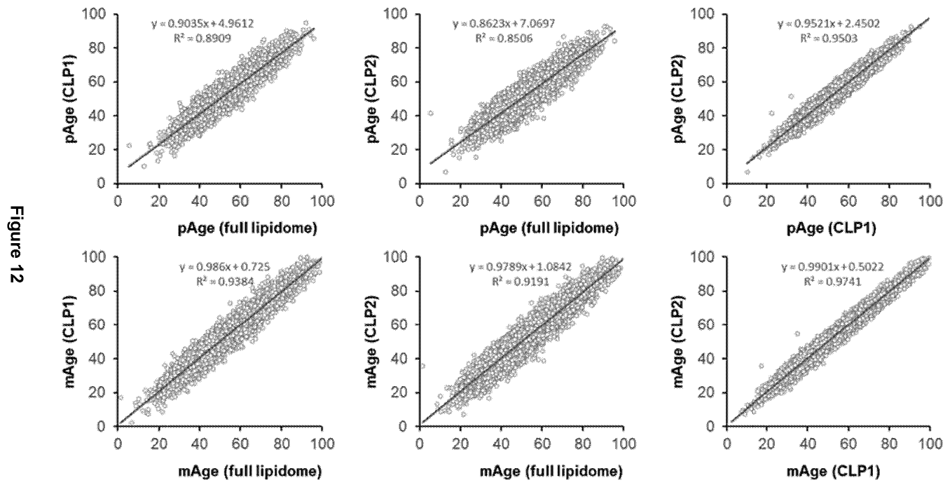

The associations between mBMIΔ scores (derived from the Research Lipid List, CLP1s and CLP 2) with type 2 diabetes mellitus (T2DM) were consistent across quintiles. However, the mBMIΔ derived form CLP1s showed a slightly better association with newly diagnosed T2DM and the 5-year incident T2DM compared to the mBMIΔ derived using the Research Lipid List and CLP2s (FIG. 10). Similarly, all the mBMIΔ scores regardless of the lipid set they were derived from showed a similar association with both prevalent CVD and incident CVE (FIG. 10).

TABLE 8
Predictive models of BMI developed using Ridge regression
incorporating age and sex together with CLP1s lipids.

| Rank # | Lipid # | Lipid/Covariate | Ridge | Ridge (ABS) |
|---|---|---|---|---|
| | | (Intercept) | 27.464 | 27.464 |
| | | drage_00 | −0.023 | 0.023 |
| | | drsex_00_n | 1.525 | 1.525 |
| 1 | 197 | SM 34:2 | 1.599 | 1.599 |
| 2 | 198 | SM 34:3 | 1.042 | 1.042 |
| 3 | 142 | PC(P-30:0) | 0.812 | 0.812 |
| 4 | 194 | SM 32:2 | 0.670 | 0.670 |
| 5 | 196 | SM 34:1 | −0.635 | 0.635 |
| 6 | 7 | AcylCarnitine 16:1 | 0.558 | 0.558 |
| 7 | 130 | PC 37:6 | −0.542 | 0.542 |
| 8 | 85 | LPC 19:1 | −0.442 | 0.442 |
| 9 | 1 | AcylCarnitine 12:0 | −0.437 | 0.437 |
| 10 | 3 | AcylCarnitine 14:0 | −0.430 | 0.430 |
| 11 | 180 | PI 32:1 | 0.422 | 0.422 |
| 12 | 44 | Cer(m18:0/22:0) | 0.417 | 0.417 |
| 13 | 41 | Cer(d19:1/22:0) | 0.398 | 0.398 |
| 14 | 195 | SM 33:1 | −0.388 | 0.388 |
| 15 | 64 | GM3(d18:1/24:1) | −0.387 | 0.387 |
| 16 | 80 | LPC 18:0 | 0.381 | 0.381 |
| 17 | 74 | Hex2Cer(d18:1/24:0) | −0.376 | 0.376 |
| 18 | 137 | PC 40:8 | −0.366 | 0.366 |
| 19 | 27 | Cer(d16:1/22:0) | 0.364 | 0.364 |
| 20 | 75 | Hex2Cer(d18:1/24:1) | 0.363 | 0.363 |
| 21 | 148 | PC(P-37:4) | −0.357 | 0.357 |
| 22 | 221 | TG(48:3) [NL-16:1] | −0.354 | 0.354 |
| 23 | 144 | PC(P-34:1) | −0.350 | 0.350 |
| 24 | 174 | PE(P-18:0/22:4) | −0.349 | 0.349 |
| 25 | 31 | Cer(d18:1/16:0) | −0.349 | 0.349 |
| 26 | 178 | PE(P-20:0/18:2) | −0.339 | 0.339 |
| 27 | 203 | SM 38:1 | −0.335 | 0.335 |
| 28 | 8 | AcylCarnitine 18:1 | 0.328 | 0.328 |
| 29 | 188 | PI 38:6 | −0.325 | 0.325 |
| 30 | 94 | LPC(O-20:0) | −0.319 | 0.319 |
| 31 | 199 | SM 35:1 | −0.318 | 0.318 |
| 32 | 209 | SM 44:3 | −0.314 | 0.314 |
| 33 | 10 | CE 14:0 | −0.308 | 0.308 |
| 34 | 271 | TG(56:6) [NL-22:5] | −0.301 | 0.301 |
| 35 | 13 | CE 16:1 | 0.298 | 0.298 |
| 36 | 79 | LPC 17:0 | −0.296 | 0.296 |
| 37 | 153 | PE 36:1 | 0.294 | 0.294 |
| 38 | 163 | PE(P-16:0/18:2) | 0.286 | 0.286 |
| 39 | 131 | PC 38:4(a) | 0.285 | 0.285 |
| 40 | 155 | PE 36:4 | −0.284 | 0.284 |
| 41 | 16 | CE 18:0 | 0.281 | 0.281 |
| 42 | 134 | PC 38:6(a) | −0.280 | 0.280 |
| 43 | 117 | PC 34:1 | −0.273 | 0.273 |
| 44 | 112 | PC 32:1 | 0.263 | 0.263 |
| 45 | 157 | PE 38:6 | −0.263 | 0.263 |
| 46 | 65 | Hex1Cer(d18:1/16:0) | 0.262 | 0.262 |
| 47 | 30 | Cer(d17:1/24:0) | −0.262 | 0.262 |
| 48 | 60 | DG 38:4 −(20:3) | −0.260 | 0.260 |
| 49 | 123 | PC 36:1 | −0.250 | 0.250 |
| 50 | 273 | TG(56:7) [NL-20:5] | 0.248 | 0.248 |
| 51 | 277 | TG(56:8) [NL-20:5] | 0.241 | 0.241 |
| 52 | 222 | TG(48:3) [NL-18:3] | −0.239 | 0.239 |
| 53 | 272 | TG(56:7) [NL-20:4] | 0.236 | 0.236 |
| 54 | 93 | LPC 26:0 | −0.232 | 0.232 |
| 55 | 135 | PC 38:6(b) | −0.232 | 0.232 |
| 56 | 33 | Cer(d18:1/22:0) | 0.230 | 0.230 |
| 57 | 293 | TG(O-52:2) [NL-18:1] | 0.228 | 0.228 |
| 58 | 289 | TG(O-50:2) [NL-18:2] | −0.228 | 0.228 |
| 59 | 145 | PC(P-34:2) | −0.225 | 0.225 |
| 60 | 71 | Hex2Cer(d16:1/16:0) | −0.223 | 0.223 |
| 61 | 171 | PE(P-18:0/18:2) | −0.221 | 0.221 |
| 62 | 56 | DG 34:2 −(18:2) | 0.220 | 0.220 |
| 63 | 83 | LPC 18:3 | 0.218 | 0.218 |
| 64 | 204 | SM 38:2 | 0.215 | 0.215 |
| 65 | 70 | Hex1Cer(d18:2/24:0) | −0.213 | 0.213 |
| 66 | 63 | GM3(d18:1/22:0) | −0.210 | 0.210 |
| 67 | 67 | Hex1Cer(d18:1/22:0) | −0.208 | 0.208 |
| 68 | 147 | PC(P-36:5) | 0.206 | 0.206 |
| 69 | 138 | PC(O-32:0) | 0.205 | 0.205 |
| 70 | 189 | PI 40:6 | −0.203 | 0.203 |
| 71 | 244 | TG(52:1) [NL-18:0] | 0.197 | 0.197 |
| 72 | 280 | TG(58:10) [NL-22:6] | −0.196 | 0.196 |
| 73 | 173 | PE(P-18:0/20:5) | −0.195 | 0.195 |
| 74 | 184 | PI 36:4 | 0.191 | 0.191 |
| 75 | 136 | PC 40:6 | 0.191 | 0.191 |
| 76 | 39 | Cer(d18:2/24:0) | −0.190 | 0.190 |
| 77 | 170 | PE(P-18:0/18:1) | 0.190 | 0.190 |
| 78 | 259 | TG(54:3) [NL-18:2] | −0.187 | 0.187 |
| 79 | 256 | TG(54:2) [NL-18:0] | 0.186 | 0.186 |
| 80 | 207 | SM 41:1 | 0.185 | 0.185 |
| 81 | 275 | TG(56:7) [NL-22:6] | 0.185 | 0.185 |
| 82 | 261 | TG(54:4) [NL-20:3] | 0.185 | 0.185 |
| 83 | 162 | PE(P-16:0/18:1) | 0.184 | 0.184 |
| 84 | 294 | TG(O-54:3) [NL-17:1] | −0.182 | 0.182 |
| 85 | 243 | TG(51:2) [NL-17:1] | 0.181 | 0.181 |
| 86 | 81 | LPC 18:1 | −0.180 | 0.180 |
| 87 | 114 | PC 33:1 | −0.180 | 0.180 |
| 88 | 242 | TG(51:2) [NL-17:0] | 0.176 | 0.176 |
| 89 | 62 | GM3(d18:1/18:0) | −0.173 | 0.173 |
| 90 | 92 | LPC 24:0 | −0.172 | 0.172 |
| 91 | 182 | PI 36:1 | 0.172 | 0.172 |
| 92 | 38 | Cer(d18:2/23:0) | −0.171 | 0.171 |
| 93 | 116 | PC 34:0 | 0.169 | 0.169 |
| 94 | 37 | Cer(d18:2/22:0) | 0.169 | 0.169 |
| 95 | 119 | PC 34:4 | 0.167 | 0.167 |
| 96 | 69 | Hex1Cer(d18:1/24:1) | 0.164 | 0.164 |
| 97 | 46 | Cer(m18:1/22:0) | 0.162 | 0.162 |
| 98 | 284 | TG(O-50:1) [NL-16:0] | −0.160 | 0.160 |
| 99 | 278 | TG(56:8) [NL-22:6] | 0.158 | 0.158 |
| 100 | 238 | TG(50:4) [NL-20:4] | −0.158 | 0.158 |
| 101 | 176 | PE(P-18:1/20:4) | 0.157 | 0.157 |
| 102 | 26 | CE 22:6 | 0.156 | 0.156 |
| 103 | 99 | LPC(P-16:0) | 0.156 | 0.156 |
| 104 | 22 | CE 20:2 | 0.155 | 0.155 |
| 105 | 192 | SM 31:1 | 0.152 | 0.152 |
| 106 | 132 | PC 38:4(b) | 0.152 | 0.152 |
| 107 | 166 | PE(P-16:0/22:4) | −0.148 | 0.148 |
| 108 | 191 | S1P(d18:1) | −0.147 | 0.147 |
| 109 | 230 | TG(50:2) [NL-18:2] | 0.147 | 0.147 |
| 110 | 100 | LPC(P-17:0) | 0.146 | 0.146 |
| 111 | 102 | LPE 18:0 | 0.146 | 0.146 |
| 112 | 283 | TG(O-50:1) [NL-15:0] | −0.145 | 0.145 |
| 113 | 219 | TG(48:2) [NL-18:2] | 0.144 | 0.144 |
| 114 | 118 | PC 34:2 | −0.142 | 0.142 |
| 115 | 232 | TG(50:3) [NL-14:1] | −0.140 | 0.140 |
| 116 | 257 | TG(54:2) [NL-20:1] | −0.136 | 0.136 |
| 117 | 158 | PE 40:6 | 0.135 | 0.135 |
| 118 | 206 | SM 40:1 | 0.135 | 0.135 |
| 119 | 213 | TG(48:0) [NL-16:0] | 0.135 | 0.135 |
| 120 | 108 | LPE(P-18:0) | −0.134 | 0.134 |
| 121 | 164 | PE(P-16:0/20:4) | 0.133 | 0.133 |
| 122 | 90 | LPC 22:5 | −0.131 | 0.131 |
| 123 | 285 | TG(O-50:1) [NL-17:1] | −0.131 | 0.131 |
| 124 | 101 | LPE 16:0 | −0.130 | 0.130 |
| 125 | 241 | TG(51:2) [NL-15:0] | −0.129 | 0.129 |
| 126 | 274 | TG(56:7) [NL-22:5] | −0.129 | 0.129 |
| 127 | 297 | TG(O-54:4) [NL-18:2] | 0.127 | 0.127 |
| 128 | 53 | DE(20:4) | 0.125 | 0.125 |
| 129 | 237 | TG(50:4) [NL-18:3] | −0.123 | 0.123 |
| 130 | 248 | TG(52:4) [NL-16:1] | 0.123 | 0.123 |
| 131 | 295 | TG(O-54:3) [NL-18:1] | 0.123 | 0.123 |
| 132 | 251 | TG(52:5) [NL-18:3] | −0.121 | 0.121 |
| 133 | 2 | AcylCarnitine 13:0 | 0.120 | 0.120 |
| 134 | 270 | TG(56:6) [NL-20:4] | 0.119 | 0.119 |
| 135 | 177 | PE(P-18:1/20:5) | 0.119 | 0.119 |
| 136 | 124 | PC 36:2 | −0.117 | 0.117 |
| 137 | 269 | TG(54:7) [NL-22:6] | −0.113 | 0.113 |
| 138 | 35 | Cer(d18:1/24:0) | −0.113 | 0.113 |
| 139 | 168 | PE(P-17:0/20:4) | 0.112 | 0.112 |
| 140 | 161 | PE(O-36:4) | −0.112 | 0.112 |
| 141 | 229 | TG(50:2) [NL-18:1] | 0.111 | 0.111 |
| 142 | 140 | PC(O-36:0) | 0.111 | 0.111 |
| 143 | 255 | TG(53:2) [NL-18:1] | −0.108 | 0.108 |
| 144 | 17 | CE 18:1 | −0.106 | 0.106 |
| 145 | 169 | PE(P-17:0/22:6) | 0.106 | 0.106 |
| 146 | 265 | TG(54:6) [NL-20:4] | 0.105 | 0.105 |
| 147 | 95 | LPC(O-22:0) | 0.104 | 0.104 |
| 148 | 151 | PE 34:2 | −0.104 | 0.104 |
| 149 | 15 | CE 17:0 | −0.104 | 0.104 |
| 150 | 11 | CE 15:0 | −0.103 | 0.103 |
| 151 | 23 | CE 20:4 | −0.103 | 0.103 |
| 152 | 68 | Hex1Cer(d18:1/24:0) | −0.102 | 0.102 |
| 153 | 217 | TG(48:2) [NL-14:1] | 0.100 | 0.100 |
| 154 | 52 | DE(18:2) | −0.098 | 0.098 |
| 155 | 160 | PE(O-34:2) | −0.097 | 0.097 |
| 156 | 288 | TG(O-50:2) [NL-18:1] | 0.097 | 0.097 |
| 157 | 107 | LPE(P-16:0) | 0.097 | 0.097 |
| 158 | 88 | LPC 20:5 | −0.094 | 0.094 |
| 159 | 128 | PC 36:5(a) | 0.094 | 0.094 |
| 160 | 97 | LPC(O-24:0) | 0.091 | 0.091 |
| 161 | 246 | TG(52:2) [NL-16:0] | −0.090 | 0.090 |
| 162 | 212 | Sulfatide (d18:1:/16:0) | −0.090 | 0.090 |
| 163 | 252 | TG(52:5) [NL-20:4] | 0.090 | 0.090 |
| 164 | 34 | Cer(d18:1/23:0) | −0.089 | 0.089 |
| 165 | 5 | AcylCarnitine 14:2 | −0.088 | 0.088 |
| 166 | 290 | TG(O-52:0) [NL-16:0] | 0.088 | 0.088 |
| 167 | 234 | TG(50:3) [NL-18:2] | 0.088 | 0.088 |
| 168 | 55 | DG 34:1 −(18:1) | 0.087 | 0.087 |
| 169 | 126 | PC 36:4(a) | −0.086 | 0.086 |
| 170 | 96 | LPC(O-22:1) | −0.086 | 0.086 |
| 171 | 239 | TG(51:0) [NL-16:0] | 0.086 | 0.086 |
| 172 | 125 | PC 36:4 | −0.085 | 0.085 |
| 173 | 235 | TG(50:3) [NL-18:3] | −0.084 | 0.084 |
| 174 | 51 | DE(18:1) | 0.084 | 0.084 |
| 175 | 202 | SM 36:3 | −0.083 | 0.083 |
| 176 | 260 | TG(54:4) [NL-18:2] | −0.082 | 0.082 |
| 177 | 190 | PS 38:4 | 0.082 | 0.082 |
| 178 | 214 | TG(48:1) [NL-16:1] | −0.080 | 0.080 |
| 179 | 28 | Cer(d16:1/24:0) | −0.080 | 0.080 |
| 180 | 122 | PC 35:5 | 0.079 | 0.079 |
| 181 | 29 | Cer(d16:1/24:1) | 0.078 | 0.078 |
| 182 | 121 | PC 35:4 | 0.078 | 0.078 |
| 183 | 24 | CE 20:5 | 0.077 | 0.077 |
| 184 | 84 | LPC 19:0 | −0.077 | 0.077 |
| 185 | 268 | TG(54:7) [NL-20:5] | 0.072 | 0.072 |
| 186 | 245 | TG(52:1) [NL-18:1] | 0.072 | 0.072 |
| 187 | 254 | TG(53:2) [NL-17:1] | 0.071 | 0.071 |
| 188 | 72 | Hex2Cer(d18:1/16:0) | 0.071 | 0.071 |
| 189 | 282 | TG(58:9) [NL-22:6] | 0.071 | 0.071 |
| 190 | 59 | DG 36:4 −(18:2) | −0.070 | 0.070 |
| 191 | 98 | LPC(O-24:2) | 0.070 | 0.070 |
| 192 | 25 | CE 22:1 | 0.070 | 0.070 |
| 193 | 66 | Hex1Cer(d18:1/18:0) | −0.069 | 0.069 |
| 194 | 6 | AcylCarnitine 16:0 | 0.069 | 0.069 |
| 195 | 250 | TG(52:4) [NL-18:3] | −0.068 | 0.068 |
| 196 | 287 | TG(O-50:2) [NL-16:1] | 0.068 | 0.068 |
| 197 | 224 | TG(50:0) [NL-18:0] | −0.066 | 0.066 |
| 198 | 32 | Cer(d18:1/20:0) | −0.063 | 0.063 |
| 199 | 172 | PE(P-18:0/20:4) | 0.062 | 0.062 |
| 200 | 129 | PC 36:5(b) | −0.060 | 0.060 |
| 201 | 216 | TG(48:2) [NL-14:0] | −0.059 | 0.059 |
| 202 | 36 | Cer(d18:1/24:1) | −0.058 | 0.058 |
| 203 | 156 | PE 38:4 | 0.057 | 0.057 |
| 204 | 49 | Cer1P(d18:1/16:0) | −0.056 | 0.056 |
| 205 | 231 | TG(50:3) [NL-14:0] | 0.055 | 0.055 |
| 206 | 45 | Cer(m18:1/20:0) | 0.055 | 0.055 |
| 207 | 43 | Cer(d19:1/24:1) | 0.055 | 0.055 |
| 208 | 286 | TG(O-50:1) [NL-18:1] | 0.055 | 0.055 |
| 209 | 175 | PE(P-18:0/22:6) | −0.053 | 0.053 |
| 210 | 165 | PE(P-16:0/20:5) | 0.053 | 0.053 |
| 211 | 291 | TG(O-52:2) [NL-16:0] | −0.052 | 0.052 |
| 212 | 40 | Cer(d18:2/24:1) | 0.052 | 0.052 |
| 213 | 225 | TG(50:1) [NL-16:0] | 0.052 | 0.052 |
| 214 | 82 | LPC 18:2 | −0.050 | 0.050 |
| 215 | 89 | LPC 22:0 | 0.049 | 0.049 |
| 216 | 211 | Sulfatide (d18:1:/16:0(OH)) | −0.049 | 0.049 |
| 217 | 152 | PE 34:3 | −0.048 | 0.048 |
| 218 | 106 | LPE 22:6 | −0.047 | 0.047 |
| 219 | 149 | PE 32:1 | −0.047 | 0.047 |
| 220 | 78 | LPC 14:0 | 0.044 | 0.044 |
| 221 | 167 | PE(P-16:0/22:6) | −0.044 | 0.044 |
| 222 | 20 | CE 20:0 | −0.042 | 0.042 |
| 223 | 279 | TG(56:9) [NL-22:6] | 0.042 | 0.042 |
| 224 | 185 | PI 37:4 | 0.040 | 0.040 |
| 225 | 236 | TG(50:4) [NL-14:0] | 0.040 | 0.040 |
| 226 | 205 | SM 38:3 | −0.040 | 0.040 |
| 227 | 266 | TG(54:6) [NL-20:5] | −0.039 | 0.039 |
| 228 | 113 | PC 32:2 | 0.039 | 0.039 |
| 229 | 267 | TG(54:6) [NL-22:6] | 0.038 | 0.038 |
| 230 | 87 | LPC 20:4 | 0.037 | 0.037 |
| 231 | 200 | SM 35:2 | 0.037 | 0.037 |
| 232 | 4 | AcylCarnitine 14:1 | 0.035 | 0.035 |
| 233 | 187 | PI 38:4 | −0.034 | 0.034 |
| 234 | 42 | Cer(d19:1/24:0) | 0.032 | 0.032 |
| 235 | 228 | TG(50:2) [NL-16:1] | −0.031 | 0.031 |
| 236 | 73 | Hex2Cer(d18:1/22:0) | 0.030 | 0.030 |
| 237 | 226 | TG(50:1) [NL-18:1] | 0.030 | 0.030 |
| 238 | 50 | COH (161) | −0.030 | 0.030 |
| 239 | 77 | Hex3Cer(d18:1/16:0) | −0.029 | 0.029 |
| 240 | 296 | TG(O-54:4) [NL-17:1] | −0.029 | 0.029 |
| 241 | 76 | Hex2Cer(d18:2/16:0) | −0.026 | 0.026 |
| 242 | 19 | CE 18:3 | 0.025 | 0.025 |
| 243 | 150 | PE 34:1 | 0.024 | 0.024 |
| 244 | 58 | DG 36:3 −(18:2) | 0.024 | 0.024 |
| 245 | 47 | Cer(m18:1/24:0) | 0.023 | 0.023 |
| 246 | 105 | LPE 20:4 | −0.022 | 0.022 |
| 247 | 227 | TG(50:2) [NL-14:0] | 0.022 | 0.022 |
| 248 | 233 | TG(50:3) [NL-16:1] | −0.022 | 0.022 |
| 249 | 91 | LPC 22:6 | 0.022 | 0.022 |
| 250 | 220 | TG(48:3) [NL-14:0] | −0.021 | 0.021 |
| 251 | 14 | CE 16:2 | −0.021 | 0.021 |
| 252 | 249 | TG(52:4) [NL-18:2] | 0.021 | 0.021 |
| 253 | 181 | PI 34:1 | 0.019 | 0.019 |
| 254 | 159 | PE(O-34:1) | −0.019 | 0.019 |
| 255 | 104 | LPE 18:2 | −0.019 | 0.019 |
| 256 | 183 | PI 36:2 | −0.019 | 0.019 |
| 257 | 109 | LPI 18:0 | −0.017 | 0.017 |
| 258 | 57 | DG 36:2 −(18:1) | 0.017 | 0.017 |
| 259 | 186 | PI 38:3 | 0.017 | 0.017 |
| 260 | 115 | PC 33:2 | 0.017 | 0.017 |
| 261 | 18 | CE 18:2 | −0.016 | 0.016 |
| 262 | 146 | PC(P-36:4) | −0.016 | 0.016 |
| 263 | 154 | PE 36:2 | −0.015 | 0.015 |
| 264 | 54 | DG 32:0 −(16:0) | 0.015 | 0.015 |
| 265 | 218 | TG(48:2) [NL-16:1] | −0.015 | 0.015 |
| 266 | 223 | TG(49:1) [NL-17:1] | 0.014 | 0.014 |
| 267 | 264 | TG(54:6) [NL-18:3] | −0.014 | 0.014 |
| 268 | 253 | TG(52:5) [NL-20:5] | 0.014 | 0.014 |
| 269 | 9 | AcylCarnitine 18:2 | −0.014 | 0.014 |
| 270 | 208 | SM 44:2 | 0.013 | 0.013 |
| 271 | 262 | TG(54:5) [NL-18:3] | −0.013 | 0.013 |
| 272 | 120 | PC 34:5 | 0.012 | 0.012 |
| 273 | 61 | GM3(d18:1/16:0) | 0.012 | 0.012 |
| 274 | 48 | Cer(m18:1/24:1) | 0.012 | 0.012 |
| 275 | 110 | PC 31:0 | 0.012 | 0.012 |
| 276 | 298 | Ubiquinone | −0.011 | 0.011 |
| 277 | 12 | CE 16:0 | 0.010 | 0.010 |
| 278 | 201 | SM 36:1 | 0.010 | 0.010 |
| 279 | 276 | TG(56:8) [NL-20:4] | −0.010 | 0.010 |
| 280 | 141 | PC(O-36:5) | −0.009 | 0.009 |
| 281 | 258 | TG(54:3) [NL-18:1] | −0.009 | 0.009 |
| 282 | 210 | Sph(d17:1) | 0.009 | 0.009 |
| 283 | 139 | PC(O-34:2) | 0.008 | 0.008 |
| 284 | 179 | PE(P-20:0/20:4) | −0.008 | 0.008 |
| 285 | 86 | LPC 20:0 | 0.008 | 0.008 |
| 286 | 133 | PC 38:4(c) | −0.007 | 0.007 |
| 287 | 263 | TG(54:5) [NL-20:4] | −0.007 | 0.007 |
| 288 | 111 | PC 32:0 | 0.007 | 0.007 |
| 289 | 21 | CE 20:1 | −0.007 | 0.007 |
| 290 | 215 | TG(48:1) [NL-18:1] | 0.006 | 0.006 |
| 291 | 292 | TG(O-52:2) [NL-17:1] | −0.006 | 0.006 |
| 292 | 127 | PC 36:4(b) | 0.006 | 0.006 |
| 293 | 281 | TG(58:8) [NL-22:6] | 0.006 | 0.006 |
| 294 | 240 | TG(51:1) [NL-17:0] | −0.005 | 0.005 |
| 295 | 103 | LPE 18:1 | 0.003 | 0.003 |
| 296 | 193 | SM 32:1 | 0.002 | 0.002 |
| 297 | 143 | PC(P-32:0) | 0.001 | 0.001 |
| 298 | 247 | TG(52:3) [NL-18:2] | 0.000 | 0.000 |

TABLE 9
Predictive models of BMI developed using LASSO regression
incorporating age and sex together with the CLP1s lipids

| | | | | LASSO |
|---|---|---|---|---|
| Rank # | Lipid # | Lipid/Covariate | LASSO | (ABS) |
| | | (Intercept) | 27.488 | 27.488 |
| | | drage_00 | −0.025 | 0.025 |
| | | drsex_00_n | 1.713 | 1.713 |
| 1 | 197 | SM 34:2 | 2.301 | 2.301 |
| 2 | 198 | SM 34:3 | 1.215 | 1.215 |
| 3 | 196 | SM 34:1 | −0.989 | 0.989 |
| 4 | 142 | PC(P-30:0) | 0.957 | 0.957 |
| 5 | 194 | SM 32:2 | 0.925 | 0.925 |
| 6 | 130 | PC 37:6 | −0.857 | 0.857 |
| 7 | 7 | AcylCarnitine 16:1 | 0.717 | 0.717 |
| 8 | 3 | AcylCarnitine 14:0 | −0.598 | 0.598 |
| 9 | 85 | LPC 19:1 | −0.579 | 0.579 |
| 10 | 44 | Cer(m18:0/22:0) | 0.572 | 0.572 |
| 11 | 195 | SM 33:1 | −0.546 | 0.546 |
| 12 | 80 | LPC 18:0 | 0.520 | 0.520 |
| 13 | 180 | PI 32:1 | 0.520 | 0.520 |
| 14 | 144 | PC(P-34:1) | −0.515 | 0.515 |
| 15 | 41 | Cer(d19:1/22:0) | 0.484 | 0.484 |
| 16 | 74 | Hex2Cer(d18:1/24:0) | −0.470 | 0.470 |
| 17 | 27 | Cer(d16:1/22:0) | 0.451 | 0.451 |
| 18 | 1 | AcylCarnitine 12:0 | −0.451 | 0.451 |
| 19 | 137 | PC 40:8 | −0.425 | 0.425 |
| 20 | 174 | PE(P-18:0/22:4) | −0.415 | 0.415 |
| 21 | 39 | Cer(d18:2/24:0) | −0.410 | 0.410 |
| 22 | 148 | PC(P-37:4) | −0.402 | 0.402 |
| 23 | 31 | Cer(d18:1/16:0) | −0.395 | 0.395 |
| 24 | 64 | GM3(d18:1/24:1) | −0.392 | 0.392 |
| 25 | 178 | PE(P-20:0/18:2) | −0.391 | 0.391 |
| 26 | 199 | SM 35:1 | −0.379 | 0.379 |
| 27 | 155 | PE 36:4 | −0.374 | 0.374 |
| 28 | 153 | PE 36:1 | 0.367 | 0.367 |
| 29 | 188 | PI 38:6 | −0.331 | 0.331 |
| 30 | 10 | CE 14:0 | −0.330 | 0.330 |
| 31 | 244 | TG(52:1) [NL-18:0] | 0.312 | 0.312 |
| 32 | 70 | Hex1Cer(d18:2/24:0) | −0.303 | 0.303 |
| 33 | 8 | AcylCarnitine 18:1 | 0.301 | 0.301 |
| 34 | 163 | PE(P-16:0/18:2) | 0.290 | 0.290 |
| 35 | 38 | Cer(d18:2/23:0) | −0.289 | 0.289 |
| 36 | 221 | TG(48:3) [NL-16:1] | −0.286 | 0.286 |
| 37 | 33 | Cer(d18:1/22:0) | 0.282 | 0.282 |
| 38 | 271 | TG(56:6) [NL-22:5] | −0.276 | 0.276 |
| 39 | 273 | TG(56:7) [NL-20:5] | 0.271 | 0.271 |
| 40 | 131 | PC 38:4(a) | 0.263 | 0.263 |
| 41 | 162 | PE(P-16:0/18:1) | 0.260 | 0.260 |
| 42 | 202 | SM 36:3 | −0.244 | 0.244 |
| 43 | 79 | LPC 17:0 | −0.243 | 0.243 |
| 44 | 219 | TG(48:2) [NL-18:2] | 0.230 | 0.230 |
| 45 | 136 | PC 40:6 | 0.226 | 0.226 |
| 46 | 272 | TG(56:7) [NL-20:4] | 0.222 | 0.222 |
| 47 | 92 | LPC 24:0 | −0.218 | 0.218 |
| 48 | 75 | Hex2Cer(d18:1/24:1) | 0.217 | 0.217 |
| 49 | 93 | LPC 26:0 | −0.217 | 0.217 |
| 50 | 71 | Hex2Cer(d16:1/16:0) | −0.212 | 0.212 |
| 51 | 289 | TG(O-50:2) [NL-18:2] | −0.210 | 0.210 |
| 52 | 13 | CE 16:1 | 0.207 | 0.207 |
| 53 | 209 | SM 44:3 | −0.196 | 0.196 |
| 54 | 207 | SM 41:1 | 0.194 | 0.194 |
| 55 | 121 | PC 35:4 | 0.193 | 0.193 |
| 56 | 16 | CE 18:0 | 0.184 | 0.184 |
| 57 | 176 | PE(P-18:1/20:4) | 0.181 | 0.181 |
| 58 | 126 | PC 36:4(a) | −0.178 | 0.178 |
| 59 | 118 | PC 34:2 | −0.175 | 0.175 |
| 60 | 62 | GM3(d18:1/18:0) | −0.175 | 0.175 |
| 61 | 117 | PC 34:1 | −0.169 | 0.169 |
| 62 | 30 | Cer(d17:1/24:0) | −0.169 | 0.169 |
| 63 | 251 | TG(52:5) [NL-18:3] | −0.168 | 0.168 |
| 64 | 65 | Hex1Cer(d18:1/16:0) | 0.167 | 0.167 |
| 65 | 230 | TG(50:2) [NL-18:2] | 0.167 | 0.167 |
| 66 | 2 | AcylCarnitine 13:0 | 0.166 | 0.166 |
| 67 | 157 | PE 38:6 | −0.162 | 0.162 |
| 68 | 147 | PC(P-36:5) | 0.153 | 0.153 |
| 69 | 168 | PE(P-17:0/20:4) | 0.151 | 0.151 |
| 70 | 275 | TG(56:7) [NL-22:6] | 0.149 | 0.149 |
| 71 | 138 | PC(O-32:0) | 0.147 | 0.147 |
| 72 | 94 | LPC(O-20:0) | −0.145 | 0.145 |
| 73 | 145 | PC(P-34:2) | −0.143 | 0.143 |
| 74 | 67 | Hex1Cer(d18:1/22:0) | −0.138 | 0.138 |
| 75 | 277 | TG(56:8) [NL-20:5] | 0.132 | 0.132 |
| 76 | 184 | PI 36:4 | 0.132 | 0.132 |
| 77 | 171 | PE(P-18:0/18:2) | −0.130 | 0.130 |
| 78 | 123 | PC 36:1 | −0.128 | 0.128 |
| 79 | 81 | LPC 18:1 | −0.125 | 0.125 |
| 80 | 203 | SM 38:1 | −0.123 | 0.123 |
| 81 | 69 | Hex1Cer(d18:1/24:1) | 0.122 | 0.122 |
| 82 | 100 | LPC(P-17:0) | 0.121 | 0.121 |
| 83 | 102 | LPE 18:0 | 0.111 | 0.111 |
| 84 | 191 | S1P(d18:1) | −0.107 | 0.107 |
| 85 | 63 | GM3(d18:1/22:0) | −0.107 | 0.107 |
| 86 | 294 | TG(O-54:3) [NL-17:1] | −0.104 | 0.104 |
| 87 | 243 | TG(51:2) [NL-17:1] | 0.101 | 0.101 |
| 88 | 116 | PC 34:0 | 0.098 | 0.098 |
| 89 | 295 | TG(O-54:3) [NL-18:1] | 0.096 | 0.096 |
| 90 | 56 | DG 34:2 −(18:2) | 0.095 | 0.095 |
| 91 | 46 | Cer(m18:1/22:0) | 0.094 | 0.094 |
| 92 | 26 | CE 22:6 | 0.093 | 0.093 |
| 93 | 189 | PI 40:6 | −0.091 | 0.091 |
| 94 | 260 | TG(54:4) [NL-18:2] | −0.090 | 0.090 |
| 95 | 140 | PC(O-36:0) | 0.086 | 0.086 |
| 96 | 53 | DE(20:4) | 0.083 | 0.083 |
| 97 | 259 | TG(54:3) [NL-18:2] | −0.083 | 0.083 |
| 98 | 264 | TG(54:6) [NL-18:3] | −0.082 | 0.082 |
| 99 | 160 | PE(O-34:2) | −0.082 | 0.082 |
| 100 | 297 | TG(O-54:4) [NL-18:2] | 0.080 | 0.080 |
| 101 | 22 | CE 20:2 | 0.080 | 0.080 |
| 102 | 83 | LPC 18:3 | 0.079 | 0.079 |
| 103 | 193 | SM 32:1 | −0.078 | 0.078 |
| 104 | 161 | PE(O-36:4) | −0.071 | 0.071 |
| 105 | 60 | DG 38:4 −(20:3) | −0.070 | 0.070 |
| 106 | 134 | PC 38:6(a) | −0.068 | 0.068 |
| 107 | 270 | TG(56:6) [NL-20:4] | 0.067 | 0.067 |
| 108 | 49 | Cer1P(d18:1/16:0) | −0.063 | 0.063 |
| 109 | 283 | TG(O-50:1) [NL-15:0] | −0.058 | 0.058 |
| 110 | 212 | Sulfatide (d18:1:/16:0) | −0.055 | 0.055 |
| 111 | 72 | Hex2Cer(d18:1/16:0) | 0.054 | 0.054 |
| 112 | 132 | PC 38:4(b) | 0.053 | 0.053 |
| 113 | 66 | Hex1Cer(d18:1/18:0) | −0.051 | 0.051 |
| 114 | 211 | Sulfatide (d18:1:/16:0(OH)) | −0.050 | 0.050 |
| 115 | 164 | PE(P-16:0/20:4) | 0.050 | 0.050 |
| 116 | 125 | PC 36:4 | −0.042 | 0.042 |
| 117 | 25 | CE 22:1 | 0.039 | 0.039 |
| 118 | 190 | PS 38:4 | 0.039 | 0.039 |
| 119 | 232 | TG(50:3) [NL-14:1] | −0.038 | 0.038 |
| 120 | 17 | CE 18:1 | −0.038 | 0.038 |
| 121 | 239 | TG(51:0) [NL-16:0] | 0.038 | 0.038 |
| 122 | 135 | PC 38:6(b) | −0.036 | 0.036 |
| 123 | 119 | PC 34:4 | 0.035 | 0.035 |
| 124 | 256 | TG(54:2) [NL-18:0] | 0.031 | 0.031 |
| 125 | 182 | PI 36:1 | 0.028 | 0.028 |
| 126 | 186 | PI 38:3 | 0.026 | 0.026 |
| 127 | 288 | TG(O-50:2) [NL-18:1] | 0.026 | 0.026 |
| 128 | 213 | TG(48:0) [NL-16:0] | 0.025 | 0.025 |
| 129 | 43 | Cer(d19:1/24:1) | 0.023 | 0.023 |
| 130 | 124 | PC 36:2 | −0.020 | 0.020 |
| 131 | 293 | TG(O-52:2) [NL-18:1] | 0.019 | 0.019 |
| 132 | 291 | TG(O-52:2) [NL-16:0] | −0.019 | 0.019 |
| 133 | 285 | TG(O-50:1) [NL-17:1] | −0.019 | 0.019 |
| 134 | 107 | LPE(P-16:0) | 0.018 | 0.018 |
| 135 | 51 | DE(18:1) | 0.014 | 0.014 |
| 136 | 242 | TG(51:2) [NL-17:0] | 0.010 | 0.010 |
| 137 | 210 | Sph(d17:1) | 0.009 | 0.009 |
| 138 | 170 | PE(P-18:0/18:1) | 0.007 | 0.007 |
| 139 | 29 | Cer(d16:1/24:1) | 0.006 | 0.006 |
| 140 | 166 | PE(P-16:0/22:4) | −0.006 | 0.006 |
| 141 | 173 | PE(P-18:0/20:5) | −0.004 | 0.004 |
| 142 | 159 | PE(O-34:1) | −0.004 | 0.004 |
| 143 | 50 | COH (161) | −0.004 | 0.004 |
| 144 | 179 | PE(P-20:0/20:4) | 0.004 | 0.004 |
| 145 | 185 | PI 37:4 | 0.003 | 0.003 |
| 146 | 252 | TG(52:5) [NL-20:4] | 0.002 | 0.002 |
| 147 | 21 | CE 20:1 | 0.001 | 0.001 |
| 148 | 101 | LPE 16:0 | −0.001 | 0.001 |
| 149 | 290 | TG(O-52:0) [NL-16:0] | 0.001 | 0.001 |
| 150 | 109 | LPI 18:0 | −0.001 | 0.001 |
| 151 | 250 | TG(52:4) [NL-18:3] | 0.000 | 0.000 |
| 152 | 4 | AcylCarnitine 14:1 | 0.000 | 0.000 |
| 153 | 5 | AcylCarnitine 14:2 | 0.000 | 0.000 |
| 154 | 6 | AcylCarnitine 16:0 | 0.000 | 0.000 |
| 155 | 9 | AcylCarnitine 18:2 | 0.000 | 0.000 |
| 156 | 11 | CE 15:0 | 0.000 | 0.000 |
| 157 | 12 | CE 16:0 | 0.000 | 0.000 |
| 158 | 14 | CE 16:2 | 0.000 | 0.000 |
| 159 | 15 | CE 17:0 | 0.000 | 0.000 |
| 160 | 18 | CE 18:2 | 0.000 | 0.000 |
| 161 | 19 | CE 18:3 | 0.000 | 0.000 |
| 162 | 20 | CE 20:0 | 0.000 | 0.000 |
| 163 | 23 | CE 20:4 | 0.000 | 0.000 |
| 164 | 24 | CE 20:5 | 0.000 | 0.000 |
| 165 | 28 | Cer(d16:1/24:0) | 0.000 | 0.000 |
| 166 | 32 | Cer(d18:1/20:0) | 0.000 | 0.000 |
| 167 | 34 | Cer(d18:1/23:0) | 0.000 | 0.000 |
| 168 | 35 | Cer(d18:1/24:0) | 0.000 | 0.000 |
| 169 | 36 | Cer(d18:1/24:1) | 0.000 | 0.000 |
| 170 | 37 | Cer(d18:2/22:0) | 0.000 | 0.000 |
| 171 | 40 | Cer(d18:2/24:1) | 0.000 | 0.000 |
| 172 | 42 | Cer(d19:1/24:0) | 0.000 | 0.000 |
| 173 | 45 | Cer(m18:1/20:0) | 0.000 | 0.000 |
| 174 | 47 | Cer(m18:1/24:0) | 0.000 | 0.000 |
| 175 | 48 | Cer(m18:1/24:1) | 0.000 | 0.000 |
| 176 | 52 | DE(18:2) | 0.000 | 0.000 |
| 177 | 54 | DG 32:0 −(16:0) | 0.000 | 0.000 |
| 178 | 55 | DG 34:1 −(18:1) | 0.000 | 0.000 |
| 179 | 57 | DG 36:2 −(18:1) | 0.000 | 0.000 |
| 180 | 58 | DG 36:3 −(18:2) | 0.000 | 0.000 |
| 181 | 59 | DG 36:4 −(18:2) | 0.000 | 0.000 |
| 182 | 61 | GM3(d18:1/16:0) | 0.000 | 0.000 |
| 183 | 68 | Hex1Cer(d18:1/24:0) | 0.000 | 0.000 |
| 184 | 73 | Hex2Cer(d18:1/22:0) | 0.000 | 0.000 |
| 185 | 76 | Hex2Cer(d18:2/16:0) | 0.000 | 0.000 |
| 186 | 77 | Hex3Cer(d18:1/16:0) | 0.000 | 0.000 |
| 187 | 78 | LPC 14:0 | 0.000 | 0.000 |
| 188 | 82 | LPC 18:2 | 0.000 | 0.000 |
| 189 | 84 | LPC 19:0 | 0.000 | 0.000 |
| 190 | 86 | LPC 20:0 | 0.000 | 0.000 |
| 191 | 87 | LPC 20:4 | 0.000 | 0.000 |
| 192 | 88 | LPC 20:5 | 0.000 | 0.000 |
| 193 | 89 | LPC 22:0 | 0.000 | 0.000 |
| 194 | 90 | LPC 22:5 | 0.000 | 0.000 |
| 195 | 91 | LPC 22:6 | 0.000 | 0.000 |
| 196 | 95 | LPC(O-22:0) | 0.000 | 0.000 |
| 197 | 96 | LPC(O-22:1) | 0.000 | 0.000 |
| 198 | 97 | LPC(O-24:0) | 0.000 | 0.000 |
| 199 | 98 | LPC(O-24:2) | 0.000 | 0.000 |
| 200 | 99 | LPC(P-16:0) | 0.000 | 0.000 |
| 201 | 103 | LPE 18:1 | 0.000 | 0.000 |
| 202 | 104 | LPE 18:2 | 0.000 | 0.000 |
| 203 | 105 | LPE 20:4 | 0.000 | 0.000 |
| 204 | 106 | LPE 22:6 | 0.000 | 0.000 |
| 205 | 108 | LPE(P-18:0) | 0.000 | 0.000 |
| 206 | 110 | PC 31:0 | 0.000 | 0.000 |
| 207 | 111 | PC 32:0 | 0.000 | 0.000 |
| 208 | 112 | PC 32:1 | 0.000 | 0.000 |
| 209 | 113 | PC 32:2 | 0.000 | 0.000 |
| 210 | 114 | PC 33:1 | 0.000 | 0.000 |
| 211 | 115 | PC 33:2 | 0.000 | 0.000 |
| 212 | 120 | PC 34:5 | 0.000 | 0.000 |
| 213 | 122 | PC 35:5 | 0.000 | 0.000 |
| 214 | 127 | PC 36:4(b) | 0.000 | 0.000 |
| 215 | 128 | PC 36:5(a) | 0.000 | 0.000 |
| 216 | 129 | PC 36:5(b) | 0.000 | 0.000 |
| 217 | 133 | PC 38:4(c) | 0.000 | 0.000 |
| 218 | 139 | PC(O-34:2) | 0.000 | 0.000 |
| 219 | 141 | PC(O-36:5) | 0.000 | 0.000 |
| 220 | 143 | PC(P-32:0) | 0.000 | 0.000 |
| 221 | 146 | PC(P-36:4) | 0.000 | 0.000 |
| 222 | 149 | PE 32:1 | 0.000 | 0.000 |
| 223 | 150 | PE 34:1 | 0.000 | 0.000 |
| 224 | 151 | PE 34:2 | 0.000 | 0.000 |
| 225 | 152 | PE 34:3 | 0.000 | 0.000 |
| 226 | 154 | PE 36:2 | 0.000 | 0.000 |
| 227 | 156 | PE 38:4 | 0.000 | 0.000 |
| 228 | 158 | PE 40:6 | 0.000 | 0.000 |
| 229 | 165 | PE(P-16:0/20:5) | 0.000 | 0.000 |
| 230 | 167 | PE(P-16:0/22:6) | 0.000 | 0.000 |
| 231 | 169 | PE(P-17:0/22:6) | 0.000 | 0.000 |
| 232 | 172 | PE(P-18:0/20:4) | 0.000 | 0.000 |
| 233 | 175 | PE(P-18:0/22:6) | 0.000 | 0.000 |
| 234 | 177 | PE(P-18:1/20:5) | 0.000 | 0.000 |
| 235 | 181 | PI 34:1 | 0.000 | 0.000 |
| 236 | 183 | PI 36:2 | 0.000 | 0.000 |
| 237 | 187 | PI 38:4 | 0.000 | 0.000 |
| 238 | 192 | SM 31:1 | 0.000 | 0.000 |
| 239 | 200 | SM 35:2 | 0.000 | 0.000 |
| 240 | 201 | SM 36:1 | 0.000 | 0.000 |
| 241 | 204 | SM 38:2 | 0.000 | 0.000 |
| 242 | 205 | SM 38:3 | 0.000 | 0.000 |
| 243 | 206 | SM 40:1 | 0.000 | 0.000 |
| 244 | 208 | SM 44:2 | 0.000 | 0.000 |
| 245 | 214 | TG(48:1) [NL-16:1] | 0.000 | 0.000 |
| 246 | 215 | TG(48:1) [NL-18:1] | 0.000 | 0.000 |
| 247 | 216 | TG(48:2) [NL-14:0] | 0.000 | 0.000 |
| 248 | 217 | TG(48:2) [NL-14:1] | 0.000 | 0.000 |
| 249 | 218 | TG(48:2) [NL-16:1] | 0.000 | 0.000 |
| 250 | 220 | TG(48:3) [NL-14:0] | 0.000 | 0.000 |
| 251 | 222 | TG(48:3) [NL-18:3] | 0.000 | 0.000 |
| 252 | 223 | TG(49:1) [NL-17:1] | 0.000 | 0.000 |
| 253 | 224 | TG(50:0) [NL-18:0] | 0.000 | 0.000 |
| 254 | 225 | TG(50:1) [NL-16:0] | 0.000 | 0.000 |
| 255 | 226 | TG(50:1) [NL-18:1] | 0.000 | 0.000 |
| 256 | 227 | TG(50:2) [NL-14:0] | 0.000 | 0.000 |
| 257 | 228 | TG(50:2) [NL-16:1] | 0.000 | 0.000 |
| 258 | 229 | TG(50:2) [NL-18:1] | 0.000 | 0.000 |
| 259 | 231 | TG(50:3) [NL-14:0] | 0.000 | 0.000 |
| 260 | 233 | TG(50:3) [NL-16:1] | 0.000 | 0.000 |
| 261 | 234 | TG(50:3) [NL-18:2] | 0.000 | 0.000 |
| 262 | 235 | TG(50:3) [NL-18:3] | 0.000 | 0.000 |
| 263 | 236 | TG(50:4) [NL-14:0] | 0.000 | 0.000 |
| 264 | 237 | TG(50:4) [NL-18:3] | 0.000 | 0.000 |
| 265 | 238 | TG(50:4) [NL-20:4] | 0.000 | 0.000 |
| 266 | 240 | TG(51:1) [NL-17:0] | 0.000 | 0.000 |
| 267 | 241 | TG(51:2) [NL-15:0] | 0.000 | 0.000 |
| 268 | 245 | TG(52:1) [NL-18:1] | 0.000 | 0.000 |
| 269 | 246 | TG(52:2) [NL-16:0] | 0.000 | 0.000 |
| 270 | 247 | TG(52:3) [NL-18:2] | 0.000 | 0.000 |
| 271 | 248 | TG(52:4) [NL-16:1] | 0.000 | 0.000 |
| 272 | 249 | TG(52:4) [NL-18:2] | 0.000 | 0.000 |
| 273 | 253 | TG(52:5) [NL-20:5] | 0.000 | 0.000 |
| 274 | 254 | TG(53:2) [NL-17:1] | 0.000 | 0.000 |
| 275 | 255 | TG(53:2) [NL-18:1] | 0.000 | 0.000 |
| 276 | 257 | TG(54:2) [NL-20:1] | 0.000 | 0.000 |
| 277 | 258 | TG(54:3) [NL-18:1] | 0.000 | 0.000 |
| 278 | 261 | TG(54:4) [NL-20:3] | 0.000 | 0.000 |
| 279 | 262 | TG(54:5) [NL-18:3] | 0.000 | 0.000 |
| 280 | 263 | TG(54:5) [NL-20:4] | 0.000 | 0.000 |
| 281 | 265 | TG(54:6) [NL-20:4] | 0.000 | 0.000 |
| 282 | 266 | TG(54:6) [NL-20:5] | 0.000 | 0.000 |
| 283 | 267 | TG(54:6) [NL-22:6] | 0.000 | 0.000 |
| 284 | 268 | TG(54:7) [NL-20:5] | 0.000 | 0.000 |
| 285 | 269 | TG(54:7) [NL-22:6] | 0.000 | 0.000 |
| 286 | 274 | TG(56:7) [NL-22:5] | 0.000 | 0.000 |
| 287 | 276 | TG(56:8) [NL-20:4] | 0.000 | 0.000 |
| 288 | 278 | TG(56:8) [NL-22:6] | 0.000 | 0.000 |
| 289 | 279 | TG(56:9) [NL-22:6] | 0.000 | 0.000 |
| 290 | 280 | TG(58:10) [NL-22:6] | 0.000 | 0.000 |
| 291 | 281 | TG(58:8) [NL-22:6] | 0.000 | 0.000 |
| 292 | 282 | TG(58:9) [NL-22:6] | 0.000 | 0.000 |
| 293 | 284 | TG(O-50:1) [NL-16:0] | 0.000 | 0.000 |
| 294 | 286 | TG(O-50:1) [NL-18:1] | 0.000 | 0.000 |
| 295 | 287 | TG(O-50:2) [NL-16:1] | 0.000 | 0.000 |
| 296 | 292 | TG(O-52:2) [NL-17:1] | 0.000 | 0.000 |
| 297 | 296 | TG(O-54:4) [NL-17:1] | 0.000 | 0.000 |
| 298 | 298 | Ubiquinone | 0.000 | 0.000 |

TABLE 10
Predictive models of BMI developed using
Ridge regression incorporating age and
sex together with the CLP2s lipidsRank

| | | | | Ridge |
|---|---|---|---|---|
| # | Lipid # | Lipid/Covariate | Ridge | (ABS) |
| | | (Intercept) | 27.460 | |
| | | drage_00 | −0.023 | 0.023 |
| | | drsex_00_n | 1.450 | 1.450 |
| 1 | 168 | SM 34:2 | 1.696 | 1.696 |
| 2 | 169 | SM 34:3 | 1.115 | 1.115 |
| 3 | 121 | PC(P-30:0) | 0.811 | 0.811 |
| 4 | 167 | SM 34:1 | −0.746 | 0.746 |
| 5 | 165 | SM 32:2 | 0.704 | 0.704 |
| 6 | 7 | AcylCarnitine 16:1 | 0.588 | 0.588 |
| 7 | 112 | PC 37:6 | −0.567 | 0.567 |
| 8 | 76 | LPC 19:1 | −0.535 | 0.535 |
| 9 | 3 | AcylCarnitine 14:0 | −0.522 | 0.522 |
| 10 | 70 | LPC 17:0 | −0.507 | 0.507 |
| 11 | 1 | AcylCarnitine 12:0 | −0.472 | 0.472 |
| 12 | 58 | GM3(d18:1/24:1) | −0.470 | 0.470 |
| 13 | 189 | TG(48:3) [NL-16:1] | −0.462 | 0.462 |
| 14 | 24 | Cer(d16:1/22:0) | 0.452 | 0.452 |
| 15 | 41 | Cer(m18:0/22:0) | 0.440 | 0.440 |
| 16 | 170 | SM 35:1 | −0.430 | 0.430 |
| 17 | 38 | Cer(d19:1/22:0) | 0.428 | 0.428 |
| 18 | 123 | PC(P-34:2) | −0.423 | 0.423 |
| 19 | 152 | PI 32:1 | 0.406 | 0.406 |
| 20 | 126 | PC(P-37:4) | −0.405 | 0.405 |
| 21 | 166 | SM 33:1 | −0.400 | 0.400 |
| 22 | 28 | Cer(d18:1/16:0) | −0.399 | 0.399 |
| 23 | 119 | PC 40:8 | −0.397 | 0.397 |
| 24 | 71 | LPC 18:0 | 0.396 | 0.396 |
| 25 | 125 | PC(P-36:5) | 0.392 | 0.392 |
| 26 | 219 | TG(56:6) [NL-22:5] | −0.380 | 0.380 |
| 27 | 8 | AcylCarnitine 18:1 | 0.378 | 0.378 |
| 28 | 159 | PI 38:6 | −0.372 | 0.372 |
| 29 | 118 | PC 40:6 | 0.361 | 0.361 |
| 30 | 150 | PE(P-20:0/18:2) | −0.361 | 0.361 |
| 31 | 137 | PE(P-16:0/18:2) | 0.353 | 0.353 |
| 32 | 131 | PE 36:4 | −0.334 | 0.334 |
| 33 | 10 | CE 14:0 | −0.327 | 0.327 |
| 34 | 133 | PE 38:6 | −0.323 | 0.323 |
| 35 | 174 | SM 38:1 | −0.321 | 0.321 |
| 36 | 104 | PC 34:1 | −0.320 | 0.320 |
| 37 | 30 | Cer(d18:1/22:0) | 0.317 | 0.317 |
| 38 | 113 | PC 38:4(a) | 0.314 | 0.314 |
| 39 | 116 | PC 38:6(a) | −0.302 | 0.302 |
| 40 | 100 | PC 32:1 | 0.300 | 0.300 |
| 41 | 72 | LPC 18:1 | −0.288 | 0.288 |
| 42 | 67 | Hex2Cer(d18:1/24:1) | 0.285 | 0.285 |
| 43 | 27 | Cer(d17:1/24:0) | −0.283 | 0.283 |
| 44 | 221 | TG(56:7) [NL-20:5] | 0.278 | 0.278 |
| 45 | 187 | TG(48:2) [NL-18:2] | 0.272 | 0.272 |
| 46 | 59 | Hex1Cer(d18:1/16:0) | 0.272 | 0.272 |
| 47 | 63 | Hex1Cer(d18:2/24:0) | −0.271 | 0.271 |
| 48 | 56 | DG 38:4 -(20:3) | −0.270 | 0.270 |
| 49 | 178 | SM 41:1 | 0.267 | 0.267 |
| 50 | 146 | PE(P-18:0/20:5) | −0.267 | 0.267 |
| 51 | 228 | TG(58:10) [NL-22:6] | −0.265 | 0.265 |
| 52 | 223 | TG(56:7) [NL-22:6] | 0.258 | 0.258 |
| 53 | 23 | CE 22:6 | 0.258 | 0.258 |
| 54 | 200 | TG(51:2) [NL-17:1] | 0.257 | 0.257 |
| 55 | 226 | TG(56:8) [NL-22:6] | 0.248 | 0.248 |
| 56 | 199 | TG(51:2) [NL-17:0] | 0.245 | 0.245 |
| 57 | 13 | CE 16:1 | 0.245 | 0.245 |
| 58 | 154 | PI 36:1 | 0.244 | 0.244 |
| 59 | 64 | Hex2Cer(d16:1/16:0) | −0.241 | 0.241 |
| 60 | 83 | LPC 26:0 | −0.240 | 0.240 |
| 61 | 61 | Hex1Cer(d18:1/22:0) | −0.240 | 0.240 |
| 62 | 102 | PC 33:1 | −0.240 | 0.240 |
| 63 | 117 | PC 38:6(b) | −0.235 | 0.235 |
| 64 | 197 | TG(50:4) [NL-18:3] | −0.233 | 0.233 |
| 65 | 156 | PI 36:4 | 0.231 | 0.231 |
| 66 | 134 | PE 40:6 | 0.230 | 0.230 |
| 67 | 20 | CE 20:4 | −0.229 | 0.229 |
| 68 | 220 | TG(56:7) [NL-20:4] | 0.226 | 0.226 |
| 69 | 53 | DG 34:2 -(18:2) | 0.220 | 0.220 |
| 70 | 233 | TG(O-50:2) [NL-18:2] | −0.212 | 0.212 |
| 71 | 160 | PI 40:6 | −0.211 | 0.211 |
| 72 | 225 | TG(56:8) [NL-20:5] | 0.208 | 0.208 |
| 73 | 138 | PE(P-16:0/20:4) | 0.207 | 0.207 |
| 74 | 177 | SM 40:1 | 0.206 | 0.206 |
| 75 | 74 | LPC 18:3 | 0.204 | 0.204 |
| 76 | 36 | Cer(d18:2/24:0) | −0.204 | 0.204 |
| 77 | 96 | LPE(P-18:0) | −0.203 | 0.203 |
| 78 | 43 | Cer(m18:1/22:0) | 0.193 | 0.193 |
| 79 | 85 | LPC(O-22:1) | −0.192 | 0.192 |
| 80 | 86 | LPC(O-24:0) | 0.189 | 0.189 |
| 81 | 89 | LPE 16:0 | −0.187 | 0.187 |
| 82 | 35 | Cer(d18:2/23:0) | −0.184 | 0.184 |
| 83 | 140 | PE(P-16:0/22:6) | −0.180 | 0.180 |
| 84 | 57 | GM3(d18:1/22:0) | −0.180 | 0.180 |
| 85 | 106 | PC 36:2 | −0.178 | 0.178 |
| 86 | 162 | S1P(d18:1) | −0.168 | 0.168 |
| 87 | 173 | SM 36:3 | −0.167 | 0.167 |
| 88 | 2 | AcylCarnitine 13:0 | 0.163 | 0.163 |
| 89 | 105 | PC 34:2 | −0.162 | 0.162 |
| 90 | 114 | PC 38:4(b) | 0.158 | 0.158 |
| 91 | 179 | SM 44:2 | −0.157 | 0.157 |
| 92 | 87 | LPC(P-16:0) | 0.157 | 0.157 |
| 93 | 91 | LPE 18:1 | 0.155 | 0.155 |
| 94 | 80 | LPC 22:5 | −0.152 | 0.152 |
| 95 | 32 | Cer(d18:1/24:0) | −0.152 | 0.152 |
| 96 | 144 | PE(P-18:0/18:2) | −0.151 | 0.151 |
| 97 | 90 | LPE 18:0 | 0.151 | 0.151 |
| 98 | 143 | PE(P-18:0/18:1) | 0.147 | 0.147 |
| 99 | 149 | PE(P-18:1/20:5) | 0.146 | 0.146 |
| 100 | 198 | TG(50:4) [NL-20:4] | −0.145 | 0.145 |
| 101 | 213 | TG(54:6) [NL-20:4] | 0.140 | 0.140 |
| 102 | 206 | TG(52:5) [NL-18:3] | −0.136 | 0.136 |
| 103 | 217 | TG(54:7) [NL-22:6] | −0.135 | 0.135 |
| 104 | 82 | LPC 24:0 | −0.134 | 0.134 |
| 105 | 136 | PE(O-36:4) | −0.134 | 0.134 |
| 106 | 208 | TG(54:2) [NL-20:1] | −0.132 | 0.132 |
| 107 | 163 | SM 31:1 | 0.132 | 0.132 |
| 108 | 218 | TG(56:6) [NL-20:4] | 0.131 | 0.131 |
| 109 | 50 | DE(20:4) | 0.131 | 0.131 |
| 110 | 232 | TG(O-50:2) [NL-18:1] | 0.130 | 0.130 |
| 111 | 120 | PC(O-32:0) | 0.128 | 0.128 |
| 112 | 99 | PC 32:0 | 0.127 | 0.127 |
| 113 | 11 | CE 15:0 | −0.126 | 0.126 |
| 114 | 195 | TG(50:3) [NL-18:2] | 0.125 | 0.125 |
| 115 | 109 | PC 36:4(b) | 0.124 | 0.124 |
| 116 | 52 | DG 34:1 -(18:1) | 0.122 | 0.122 |
| 117 | 62 | Hex1Cer(d18:1/24:1) | 0.120 | 0.120 |
| 118 | 222 | TG(56:7) [NL-22:5] | −0.116 | 0.116 |
| 119 | 193 | TG(50:3) [NL-14:1] | −0.114 | 0.114 |
| 120 | 207 | TG(52:5) [NL-20:4] | 0.114 | 0.114 |
| 121 | 141 | PE(P-17:0/20:4) | 0.113 | 0.113 |
| 122 | 60 | Hex1Cer(d18:1/18:0) | −0.111 | 0.111 |
| 123 | 175 | SM 38:2 | 0.110 | 0.110 |
| 124 | 147 | PE(P-18:0/22:6) | −0.108 | 0.108 |
| 125 | 34 | Cer(d18:2/22:0) | 0.107 | 0.107 |
| 126 | 46 | Cer1P(d18:1/16:0) | −0.104 | 0.104 |
| 127 | 129 | PE 34:2 | −0.102 | 0.102 |
| 128 | 227 | TG(56:9) [NL-22:6] | 0.102 | 0.102 |
| 129 | 101 | PC 32:2 | 0.102 | 0.102 |
| 130 | 148 | PE(P-18:1/20:4) | 0.102 | 0.102 |
| 131 | 40 | Cer(d19:1/24:1) | 0.101 | 0.101 |
| 132 | 191 | TG(50:2) [NL-18:1] | 0.101 | 0.101 |
| 133 | 88 | LPC(P-17:0) | 0.100 | 0.100 |
| 134 | 25 | Cer(d16:1/24:0) | −0.097 | 0.097 |
| 135 | 75 | LPC 19:0 | −0.097 | 0.097 |
| 136 | 215 | TG(54:6) [NL-22:6] | 0.096 | 0.096 |
| 137 | 132 | PE 38:4 | 0.096 | 0.096 |
| 138 | 5 | AcylCarnitine 14:2 | −0.094 | 0.094 |
| 139 | 26 | Cer(d16:1/24:1) | 0.094 | 0.094 |
| 140 | 183 | TG(48:1) [NL-18:1] | 0.092 | 0.092 |
| 141 | 48 | DE(18:1) | 0.088 | 0.088 |
| 142 | 212 | TG(54:6) [NL-18:3] | −0.088 | 0.088 |
| 143 | 124 | PC(P-36:4) | −0.087 | 0.087 |
| 144 | 182 | Sulfatide (d18:1:/16:0) | −0.085 | 0.085 |
| 145 | 184 | TG(48:2) [NL-14:0] | −0.084 | 0.084 |
| 146 | 69 | LPC 14:0 | 0.083 | 0.083 |
| 147 | 122 | PC(P-32:0) | −0.083 | 0.083 |
| 148 | 19 | CE 20:1 | 0.082 | 0.082 |
| 149 | 95 | LPE(P-16:0) | 0.082 | 0.082 |
| 150 | 142 | PE(P-17:0/22:6) | 0.082 | 0.082 |
| 151 | 51 | DG 32:0 -(16:0) | 0.081 | 0.081 |
| 152 | 4 | AcylCarnitine 14:1 | 0.080 | 0.080 |
| 153 | 201 | TG(52:2) [NL-16:0] | −0.080 | 0.080 |
| 154 | 188 | TG(48:3) [NL-14:0] | −0.080 | 0.080 |
| 155 | 145 | PE(P-18:0/20:4) | 0.079 | 0.079 |
| 156 | 185 | TG(48:2) [NL-14:1] | 0.079 | 0.079 |
| 157 | 211 | TG(54:5) [NL-20:4] | 0.078 | 0.078 |
| 158 | 110 | PC 36:5(a) | 0.077 | 0.077 |
| 159 | 31 | Cer(d18:1/23:0) | −0.077 | 0.077 |
| 160 | 216 | TG(54:7) [NL-20:5] | 0.077 | 0.077 |
| 161 | 205 | TG(52:4) [NL-18:3] | −0.075 | 0.075 |
| 162 | 204 | TG(52:4) [NL-18:2] | 0.075 | 0.075 |
| 163 | 139 | PE(P-16:0/20:5) | 0.074 | 0.074 |
| 164 | 33 | Cer(d18:1/24:1) | −0.072 | 0.072 |
| 165 | 130 | PE 36:2 | 0.069 | 0.069 |
| 166 | 6 | AcylCarnitine 16:0 | 0.067 | 0.067 |
| 167 | 128 | PE 34:1 | 0.067 | 0.067 |
| 168 | 77 | LPC 20:4 | 0.066 | 0.066 |
| 169 | 115 | PC 38:4(c) | −0.065 | 0.065 |
| 170 | 21 | CE 20:5 | 0.064 | 0.064 |
| 171 | 55 | DG 36:3 -(18:2) | −0.064 | 0.064 |
| 172 | 81 | LPC 22:6 | 0.060 | 0.060 |
| 173 | 127 | PE 32:1 | 0.058 | 0.058 |
| 174 | 234 | TG(O-54:4) [NL-17:1] | −0.058 | 0.058 |
| 175 | 176 | SM 38:3 | −0.058 | 0.058 |
| 176 | 231 | TG(O-50:2) [NL-16:1] | −0.057 | 0.057 |
| 177 | 108 | PC 36:4(a) | −0.056 | 0.056 |
| 178 | 12 | CE 16:0 | 0.055 | 0.055 |
| 179 | 107 | PC 36:4 | −0.054 | 0.054 |
| 180 | 66 | Hex2Cer(d18:1/22:0) | −0.054 | 0.054 |
| 181 | 84 | LPC(O-22:0) | −0.053 | 0.053 |
| 182 | 135 | PE(O-34:1) | −0.052 | 0.052 |
| 183 | 16 | CE 18:2 | 0.051 | 0.051 |
| 184 | 9 | AcylCarnitine 18:2 | −0.047 | 0.047 |
| 185 | 224 | TG(56:8) [NL-20:4] | −0.047 | 0.047 |
| 186 | 78 | LPC 20:5 | −0.045 | 0.045 |
| 187 | 14 | CE 16:2 | −0.045 | 0.045 |
| 188 | 65 | Hex2Cer(d18:1/16:0) | 0.045 | 0.045 |
| 189 | 98 | PC 31:0 | 0.044 | 0.044 |
| 190 | 203 | TG(52:4) [NL-16:1] | 0.044 | 0.044 |
| 191 | 42 | Cer(m18:1/20:0) | 0.043 | 0.043 |
| 192 | 49 | DE(18:2) | −0.043 | 0.043 |
| 193 | 190 | TG(50:2) [NL-16:1] | −0.042 | 0.042 |
| 194 | 181 | Sulfatide (d18:1:/16:0(OH)) | −0.038 | 0.038 |
| 195 | 29 | Cer(d18:1/20:0) | −0.037 | 0.037 |
| 196 | 194 | TG(50:3) [NL-16:1] | −0.031 | 0.031 |
| 197 | 186 | TG(48:2) [NL-16:1] | 0.030 | 0.030 |
| 198 | 209 | TG(54:3) [NL-18:1] | −0.029 | 0.029 |
| 199 | 22 | CE 22:1 | 0.027 | 0.027 |
| 200 | 172 | SM 36:1 | 0.027 | 0.027 |
| 201 | 171 | SM 35:2 | 0.027 | 0.027 |
| 202 | 44 | Cer(m18:1/24:0) | 0.026 | 0.026 |
| 203 | 93 | LPE 20:4 | −0.024 | 0.024 |
| 204 | 73 | LPC 18:2 | −0.023 | 0.023 |
| 205 | 180 | Sph(d17:1) | 0.023 | 0.023 |
| 206 | 17 | CE 18:3 | −0.023 | 0.023 |
| 207 | 92 | LPE 18:2 | 0.022 | 0.022 |
| 208 | 54 | DG 36:2 -(18:1) | 0.019 | 0.019 |
| 209 | 230 | TG(58:9) [NL-22:6] | 0.019 | 0.019 |
| 210 | 15 | CE 17:0 | 0.019 | 0.019 |
| 211 | 214 | TG(54:6) [NL-20:5] | −0.018 | 0.018 |
| 212 | 94 | LPE 22:6 | −0.016 | 0.016 |
| 213 | 103 | PC 33:2 | −0.014 | 0.014 |
| 214 | 97 | LPI 18:0 | −0.013 | 0.013 |
| 215 | 235 | Ubiquinone | 0.012 | 0.012 |
| 216 | 68 | Hex3Cer(d18:1/16:0) | −0.012 | 0.012 |
| 217 | 39 | Cer(d19:1/24:0) | 0.011 | 0.011 |
| 218 | 37 | Cer(d18:2/24:1) | −0.011 | 0.011 |
| 219 | 157 | PI 37:4 | −0.010 | 0.010 |
| 220 | 202 | TG(52:3) [NL-18:2] | 0.010 | 0.010 |
| 221 | 111 | PC 36:5(b) | −0.009 | 0.009 |
| 222 | 45 | Cer(m18:1/24:1) | −0.008 | 0.008 |
| 223 | 151 | PE(P-20:0/20:4) | 0.007 | 0.007 |
| 224 | 155 | PI 36:2 | 0.007 | 0.007 |
| 225 | 161 | PS 38:4 | −0.007 | 0.007 |
| 226 | 229 | TG(58:8) [NL-22:6] | −0.006 | 0.006 |
| 227 | 192 | TG(50:3) [NL-14:0] | −0.005 | 0.005 |
| 228 | 18 | CE 20:0 | 0.004 | 0.004 |
| 229 | 210 | TG(54:5) [NL-18:3] | −0.003 | 0.003 |
| 230 | 79 | LPC 22:0 | −0.003 | 0.003 |
| 231 | 158 | PI 38:4 | 0.003 | 0.003 |
| 232 | 47 | COH (161) | 0.002 | 0.002 |
| 233 | 153 | PI 34:1 | 0.001 | 0.001 |
| 234 | 196 | TG(50:4) [NL-14:0] | −0.001 | 0.001 |
| 235 | 164 | SM 32:1 | 0.001 | 0.001 |

TABLE 11
Predictive models of BMI developed using LASSO regression
incorporating age and sex together with the CLP2s lipids

| | | | | LASSO |
|---|---|---|---|---|
| Rank # | Lipid # | Lipid/Covariate | LASSO | (ABS) |
| | | (Intercept) | 27.521 | |
| | | drage_00 | −0.026 | 0.026 |
| | | drsex_00_n | 1.641 | 1.641 |
| 1 | 168 | SM 34:2 | 2.302 | 2.302 |
| 2 | 169 | SM 34:3 | 1.075 | 1.075 |
| 3 | 167 | SM 34:1 | −1.072 | 1.072 |
| 4 | 165 | SM 32:2 | 0.803 | 0.803 |
| 5 | 112 | PC 37:6 | −0.782 | 0.782 |
| 6 | 121 | PC(P-30:0) | 0.744 | 0.744 |
| 7 | 76 | LPC 19:1 | −0.712 | 0.712 |
| 8 | 7 | AcylCarnitine 16:1 | 0.669 | 0.669 |
| 9 | 41 | Cer(m18:0/22:0) | 0.586 | 0.586 |
| 10 | 38 | Cer(d19:1/22:0) | 0.559 | 0.559 |
| 11 | 3 | AcylCarnitine 14:0 | −0.554 | 0.554 |
| 12 | 152 | PI 32:1 | 0.531 | 0.531 |
| 13 | 71 | LPC 18:0 | 0.526 | 0.526 |
| 14 | 123 | PC(P-34:2) | −0.517 | 0.517 |
| 15 | 166 | SM 33:1 | −0.510 | 0.510 |
| 16 | 70 | LPC 17:0 | −0.504 | 0.504 |
| 17 | 119 | PC 40:8 | −0.495 | 0.495 |
| 18 | 118 | PC 40:6 | 0.465 | 0.465 |
| 19 | 36 | Cer(d18:2/24:0) | −0.442 | 0.442 |
| 20 | 58 | GM3(d18:1/24:1) | −0.440 | 0.440 |
| 21 | 1 | AcylCarnitine 12:0 | −0.436 | 0.436 |
| 22 | 170 | SM 35:1 | −0.436 | 0.436 |
| 23 | 24 | Cer(d16:1/22:0) | 0.424 | 0.424 |
| 24 | 63 | Hex1Cer(d18:2/24:0) | −0.418 | 0.418 |
| 25 | 189 | TG(48:3) [NL-16:1] | −0.401 | 0.401 |
| 26 | 150 | PE(P-20:0/18:2) | −0.378 | 0.378 |
| 27 | 200 | TG(51:2) [NL-17:1] | 0.365 | 0.365 |
| 28 | 28 | Cer(d18:1/16:0) | −0.354 | 0.354 |
| 29 | 221 | TG(56:7) [NL-20:5] | 0.341 | 0.341 |
| 30 | 83 | LPC 26:0 | −0.318 | 0.318 |
| 31 | 125 | PC(P-36:5) | 0.313 | 0.313 |
| 32 | 137 | PE(P-16:0/18:2) | 0.312 | 0.312 |
| 33 | 131 | PE 36:4 | −0.310 | 0.310 |
| 34 | 187 | TG(48:2) [NL-18:2] | 0.309 | 0.309 |
| 35 | 219 | TG(56:6) [NL-22:5] | −0.304 | 0.304 |
| 36 | 126 | PC(P-37:4) | −0.280 | 0.280 |
| 37 | 159 | PI 38:6 | −0.270 | 0.270 |
| 38 | 30 | Cer(d18:1/22:0) | 0.263 | 0.263 |
| 39 | 51 | DG 32:0 -(16:0) | 0.249 | 0.249 |
| 40 | 113 | PC 38:4(a) | 0.244 | 0.244 |
| 41 | 59 | Hex1Cer(d18:1/16:0) | 0.239 | 0.239 |
| 42 | 35 | Cer(d18:2/23:0) | −0.217 | 0.217 |
| 43 | 8 | AcylCarnitine 18:1 | 0.214 | 0.214 |
| 44 | 13 | CE 16:1 | 0.209 | 0.209 |
| 45 | 104 | PC 34:1 | −0.206 | 0.206 |
| 46 | 72 | LPC 18:1 | −0.205 | 0.205 |
| 47 | 10 | CE 14:0 | −0.199 | 0.199 |
| 48 | 91 | LPE 18:1 | 0.181 | 0.181 |
| 49 | 138 | PE(P-16:0/20:4) | 0.175 | 0.175 |
| 50 | 160 | PI 40:6 | −0.173 | 0.173 |
| 51 | 57 | GM3(d18:1/22:0) | −0.164 | 0.164 |
| 52 | 64 | Hex2Cer(d16:1/16:0) | −0.150 | 0.150 |
| 53 | 233 | TG(O-50:2) [NL-18:2] | −0.146 | 0.146 |
| 54 | 67 | Hex2Cer(d18:1/24:1) | 0.144 | 0.144 |
| 55 | 220 | TG(56:7) [NL-20:4] | 0.133 | 0.133 |
| 56 | 105 | PC 34:2 | −0.133 | 0.133 |
| 57 | 173 | SM 36:3 | −0.131 | 0.131 |
| 58 | 178 | SM 41:1 | 0.131 | 0.131 |
| 59 | 56 | DG 38:4 -(20:3) | −0.131 | 0.131 |
| 60 | 27 | Cer(d17:1/24:0) | −0.130 | 0.130 |
| 61 | 107 | PC 36:4 | −0.126 | 0.126 |
| 62 | 82 | LPC 24:0 | −0.123 | 0.123 |
| 63 | 215 | TG(54:6) [NL-22:6] | 0.123 | 0.123 |
| 64 | 156 | PI 36:4 | 0.121 | 0.121 |
| 65 | 2 | AcylCarnitine 13:0 | 0.118 | 0.118 |
| 66 | 43 | Cer(m18:1/22:0) | 0.117 | 0.117 |
| 67 | 133 | PE 38:6 | −0.117 | 0.117 |
| 68 | 212 | TG(54:6) [NL-18:3] | −0.117 | 0.117 |
| 69 | 90 | LPE 18:0 | 0.114 | 0.114 |
| 70 | 74 | LPC 18:3 | 0.111 | 0.111 |
| 71 | 136 | PE(O-36:4) | −0.109 | 0.109 |
| 72 | 147 | PE(P-18:0/22:6) | −0.102 | 0.102 |
| 73 | 213 | TG(54:6) [NL-20:4] | 0.099 | 0.099 |
| 74 | 85 | LPC(O-22:1) | −0.091 | 0.091 |
| 75 | 162 | S1P(d18:1) | −0.087 | 0.087 |
| 76 | 53 | DG 34:2 -(18:2) | 0.086 | 0.086 |
| 77 | 46 | Cer1P(d18:1/16:0) | −0.077 | 0.077 |
| 78 | 154 | PI 36:1 | 0.076 | 0.076 |
| 79 | 50 | DE(20:4) | 0.075 | 0.075 |
| 80 | 211 | TG(54:5) [NL-20:4] | 0.075 | 0.075 |
| 81 | 96 | LPE(P-18:0) | −0.070 | 0.070 |
| 82 | 12 | CE 16:0 | 0.069 | 0.069 |
| 83 | 106 | PC 36:2 | −0.065 | 0.065 |
| 84 | 60 | Hex1Cer(d18:1/18:0) | −0.064 | 0.064 |
| 85 | 11 | CE 15:0 | −0.064 | 0.064 |
| 86 | 232 | TG(O-50:2) [NL-18:1] | 0.059 | 0.059 |
| 87 | 174 | SM 38:1 | −0.058 | 0.058 |
| 88 | 148 | PE(P-18:1/20:4) | 0.056 | 0.056 |
| 89 | 75 | LPC 19:0 | −0.050 | 0.050 |
| 90 | 182 | Sulfatide (d18:1:/16:0) | −0.049 | 0.049 |
| 91 | 22 | CE 22:1 | 0.047 | 0.047 |
| 92 | 88 | LPC(P-17:0) | 0.047 | 0.047 |
| 93 | 19 | CE 20:1 | 0.045 | 0.045 |
| 94 | 181 | Sulfatide (d18:1:/16:0(OH)) | −0.040 | 0.040 |
| 95 | 231 | TG(O-50:2) [NL-16:1] | −0.032 | 0.032 |
| 96 | 48 | DE(18:1) | 0.031 | 0.031 |
| 97 | 234 | TG(O-54:4) [NL-17:1] | −0.026 | 0.026 |
| 98 | 120 | PC(O-32:0) | 0.025 | 0.025 |
| 99 | 218 | TG(56:6) [NL-20:4] | 0.023 | 0.023 |
| 100 | 65 | Hex2Cer(d18:1/16:0) | 0.022 | 0.022 |
| 101 | 206 | TG(52:5) [NL-18:3] | −0.022 | 0.022 |
| 102 | 149 | PE(P-18:1/20:5) | 0.019 | 0.019 |
| 103 | 193 | TG(50:3) [NL-14:1] | −0.015 | 0.015 |
| 104 | 199 | TG(51:2) [NL-17:0] | 0.015 | 0.015 |
| 105 | 108 | PC 36:4(a) | −0.012 | 0.012 |
| 106 | 95 | LPE(P-16:0) | 0.004 | 0.004 |
| 107 | 62 | Hex1Cer(d18:1/24:1) | 0.001 | 0.001 |
| 108 | 201 | TG(52:2) [NL-16:0] | −0.001 | 0.001 |
| 109 | 144 | PE(P-18:0/18:2) | 0.000 | 0.000 |
| 110 | 207 | TG(52:5) [NL-20:4] | 0.000 | 0.000 |
| 111 | 61 | Hex1Cer(d18:1/22:0) | 0.000 | 0.000 |
| 112 | 179 | SM 44:2 | 0.000 | 0.000 |
| 113 | 4 | AcylCarnitine 14:1 | 0.000 | 0.000 |
| 114 | 5 | AcylCarnitine 14:2 | 0.000 | 0.000 |
| 115 | 6 | AcylCarnitine 16:0 | 0.000 | 0.000 |
| 116 | 9 | AcylCarnitine 18:2 | 0.000 | 0.000 |
| 117 | 14 | CE 16:2 | 0.000 | 0.000 |
| 118 | 15 | CE 17:0 | 0.000 | 0.000 |
| 119 | 16 | CE 18:2 | 0.000 | 0.000 |
| 120 | 17 | CE 18:3 | 0.000 | 0.000 |
| 121 | 18 | CE 20:0 | 0.000 | 0.000 |
| 122 | 20 | CE 20:4 | 0.000 | 0.000 |
| 123 | 21 | CE 20:5 | 0.000 | 0.000 |
| 124 | 23 | CE 22:6 | 0.000 | 0.000 |
| 125 | 25 | Cer(d16:1/24:0) | 0.000 | 0.000 |
| 126 | 26 | Cer(d16:1/24:1) | 0.000 | 0.000 |
| 127 | 29 | Cer(d18:1/20:0) | 0.000 | 0.000 |
| 128 | 31 | Cer(d18:1/23:0) | 0.000 | 0.000 |
| 129 | 32 | Cer(d18:1/24:0) | 0.000 | 0.000 |
| 130 | 33 | Cer(d18:1/24:1) | 0.000 | 0.000 |
| 131 | 34 | Cer(d18:2/22:0) | 0.000 | 0.000 |
| 132 | 37 | Cer(d18:2/24:1) | 0.000 | 0.000 |
| 133 | 39 | Cer(d19:1/24:0) | 0.000 | 0.000 |
| 134 | 40 | Cer(d19:1/24:1) | 0.000 | 0.000 |
| 135 | 42 | Cer(m18:1/20:0) | 0.000 | 0.000 |
| 136 | 44 | Cer(m18:1/24:0) | 0.000 | 0.000 |
| 137 | 45 | Cer(m18:1/24:1) | 0.000 | 0.000 |
| 138 | 47 | COH (161) | 0.000 | 0.000 |
| 139 | 49 | DE(18:2) | 0.000 | 0.000 |
| 140 | 52 | DG 34:1 -(18:1) | 0.000 | 0.000 |
| 141 | 54 | DG 36:2 -(18:1) | 0.000 | 0.000 |
| 142 | 55 | DG 36:3 -(18:2) | 0.000 | 0.000 |
| 143 | 66 | Hex2Cer(d18:1/22:0) | 0.000 | 0.000 |
| 144 | 68 | Hex3Cer(d18:1/16:0) | 0.000 | 0.000 |
| 145 | 69 | LPC 14:0 | 0.000 | 0.000 |
| 146 | 73 | LPC 18:2 | 0.000 | 0.000 |
| 147 | 77 | LPC 20:4 | 0.000 | 0.000 |
| 148 | 78 | LPC 20:5 | 0.000 | 0.000 |
| 149 | 79 | LPC 22:0 | 0.000 | 0.000 |
| 150 | 80 | LPC 22:5 | 0.000 | 0.000 |
| 151 | 81 | LPC 22:6 | 0.000 | 0.000 |
| 152 | 84 | LPC(O-22:0) | 0.000 | 0.000 |
| 153 | 86 | LPC(O-24:0) | 0.000 | 0.000 |
| 154 | 87 | LPC(P-16:0) | 0.000 | 0.000 |
| 155 | 89 | LPE 16:0 | 0.000 | 0.000 |
| 156 | 92 | LPE 18:2 | 0.000 | 0.000 |
| 157 | 93 | LPE 20:4 | 0.000 | 0.000 |
| 158 | 94 | LPE 22:6 | 0.000 | 0.000 |
| 159 | 97 | LPI 18:0 | 0.000 | 0.000 |
| 160 | 98 | PC 31:0 | 0.000 | 0.000 |
| 161 | 99 | PC 32:0 | 0.000 | 0.000 |
| 162 | 100 | PC 32:1 | 0.000 | 0.000 |
| 163 | 101 | PC 32:2 | 0.000 | 0.000 |
| 164 | 102 | PC 33:1 | 0.000 | 0.000 |
| 165 | 103 | PC 33:2 | 0.000 | 0.000 |
| 166 | 109 | PC 36:4(b) | 0.000 | 0.000 |
| 167 | 110 | PC 36:5(a) | 0.000 | 0.000 |
| 168 | 111 | PC 36:5(b) | 0.000 | 0.000 |
| 169 | 114 | PC 38:4(b) | 0.000 | 0.000 |
| 170 | 115 | PC 38:4(c) | 0.000 | 0.000 |
| 171 | 116 | PC 38:6(a) | 0.000 | 0.000 |
| 172 | 117 | PC 38:6(b) | 0.000 | 0.000 |
| 173 | 122 | PC(P-32:0) | 0.000 | 0.000 |
| 174 | 124 | PC(P-36:4) | 0.000 | 0.000 |
| 175 | 127 | PE 32:1 | 0.000 | 0.000 |
| 176 | 128 | PE 34:1 | 0.000 | 0.000 |
| 177 | 129 | PE 34:2 | 0.000 | 0.000 |
| 178 | 130 | PE 36:2 | 0.000 | 0.000 |
| 179 | 132 | PE 38:4 | 0.000 | 0.000 |
| 180 | 134 | PE 40:6 | 0.000 | 0.000 |
| 181 | 135 | PE(O-34:1) | 0.000 | 0.000 |
| 182 | 139 | PE(P-16:0/20:5) | 0.000 | 0.000 |
| 183 | 140 | PE(P-16:0/22:6) | 0.000 | 0.000 |
| 184 | 141 | PE(P-17:0/20:4) | 0.000 | 0.000 |
| 185 | 142 | PE(P-17:0/22:6) | 0.000 | 0.000 |
| 186 | 143 | PE(P-18:0/18:1) | 0.000 | 0.000 |
| 187 | 145 | PE(P-18:0/20:4) | 0.000 | 0.000 |
| 188 | 146 | PE(P-18:0/20:5) | 0.000 | 0.000 |
| 189 | 151 | PE(P-20:0/20:4) | 0.000 | 0.000 |
| 190 | 153 | PI 34:1 | 0.000 | 0.000 |
| 191 | 155 | PI 36:2 | 0.000 | 0.000 |
| 192 | 157 | PI 37:4 | 0.000 | 0.000 |
| 193 | 158 | PI 38:4 | 0.000 | 0.000 |
| 194 | 161 | PS 38:4 | 0.000 | 0.000 |
| 195 | 163 | SM 31:1 | 0.000 | 0.000 |
| 196 | 164 | SM 32:1 | 0.000 | 0.000 |
| 197 | 171 | SM 35:2 | 0.000 | 0.000 |
| 198 | 172 | SM 36:1 | 0.000 | 0.000 |
| 199 | 175 | SM 38:2 | 0.000 | 0.000 |
| 200 | 176 | SM 38:3 | 0.000 | 0.000 |
| 201 | 177 | SM 40:1 | 0.000 | 0.000 |
| 202 | 180 | Sph(d17:1) | 0.000 | 0.000 |
| 203 | 183 | TG(48:1) [NL-18:1] | 0.000 | 0.000 |
| 204 | 184 | TG(48:2) [NL-14:0] | 0.000 | 0.000 |
| 205 | 185 | TG(48:2) [NL-14:1] | 0.000 | 0.000 |
| 206 | 186 | TG(48:2) [NL-16:1] | 0.000 | 0.000 |
| 207 | 188 | TG(48:3) [NL-14:0] | 0.000 | 0.000 |
| 208 | 190 | TG(50:2) [NL-16:1] | 0.000 | 0.000 |
| 209 | 191 | TG(50:2) [NL-18:1] | 0.000 | 0.000 |
| 210 | 192 | TG(50:3) [NL-14:0] | 0.000 | 0.000 |
| 211 | 194 | TG(50:3) [NL-16:1] | 0.000 | 0.000 |
| 212 | 195 | TG(50:3) [NL-18:2] | 0.000 | 0.000 |
| 213 | 196 | TG(50:4) [NL-14:0] | 0.000 | 0.000 |
| 214 | 197 | TG(50:4) [NL-18:3] | 0.000 | 0.000 |
| 215 | 198 | TG(50:4) [NL-20:4] | 0.000 | 0.000 |
| 216 | 202 | TG(52:3) [NL-18:2] | 0.000 | 0.000 |
| 217 | 203 | TG(52:4) [NL-16:1] | 0.000 | 0.000 |
| 218 | 204 | TG(52:4) [NL-18:2] | 0.000 | 0.000 |
| 219 | 205 | TG(52:4) [NL-18:3] | 0.000 | 0.000 |
| 220 | 208 | TG(54:2) [NL-20:1] | 0.000 | 0.000 |
| 221 | 209 | TG(54:3) [NL-18:1] | 0.000 | 0.000 |
| 222 | 210 | TG(54:5) [NL-18:3] | 0.000 | 0.000 |
| 223 | 214 | TG(54:6) [NL-20:5] | 0.000 | 0.000 |
| 224 | 216 | TG(54:7) [NL-20:5] | 0.000 | 0.000 |
| 225 | 217 | TG(54:7) [NL-22:6] | 0.000 | 0.000 |
| 226 | 222 | TG(56:7) [NL-22:5] | 0.000 | 0.000 |
| 227 | 223 | TG(56:7) [NL-22:6] | 0.000 | 0.000 |
| 228 | 224 | TG(56:8) [NL-20:4] | 0.000 | 0.000 |
| 229 | 225 | TG(56:8) [NL-20:5] | 0.000 | 0.000 |
| 230 | 226 | TG(56:8) [NL-22:6] | 0.000 | 0.000 |
| 231 | 227 | TG(56:9) [NL-22:6] | 0.000 | 0.000 |
| 232 | 228 | TG(58:10) [NL-22:6] | 0.000 | 0.000 |
| 233 | 229 | TG(58:8) [NL-22:6] | 0.000 | 0.000 |
| 234 | 230 | TG(58:9) [NL-22:6] | 0.000 | 0.000 |
| 235 | 235 | Ubiquinone | 0.000 | 0.000 |

### Example 5: Development of a Metabolic Age Score (mAge)

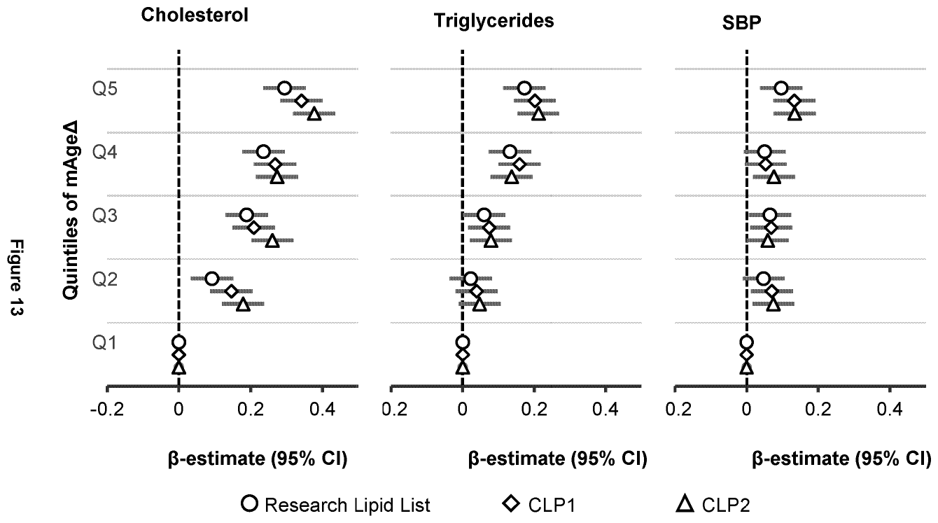

Statistical modelling was used to condense large volumes of lipidomic data into a single risk score, a Metabolic Age (mAge) score (FIG. 11), which can be easily interpreted by clinician and patient alike. Statistical modelling is used to predict an individual's mAge from their lipid profile, with some additional transformation. mAge was found to be a valuable indicator of an individual's cardiometabolic health, and a valuable predictive variable for disease risk and outcomes.

The development and subsequent calculation of mAge was made using the Australian Diabetes, Obesity and Lifestyle Study (AusDiab; n=10,339), a large cohort dataset. In this cohort, 717 lipid species from the Research Lipid List (Table 2) were measured. Calculated values utilising all measured lipids from the Research Lipid List (717 species, Table 2) were compared to calculated values utilising lipids from the CLP1 list (310 species, CLP Is, Table 4) and/or calculated values utilising lipids from the CLP2 list (Table 6). Calculation of these risk scores was further demonstrated using lipids from a further, truncated list, the CLP2 list (Table 6), featuring only those lipids which can be easily integrated without the use of peak picking algorithms, to support automated data processing (235 lipid species, CLP2s, Table 4). A ridge regression model was used.

The following sections describe the development of metabolic age (mAge) scores using the full lipidome (n=717 lipid species) and the CLP lipid lists (CLP1s and CLP2s). The mAge score, and a derived score from the difference between mAge and the chronological age (mAgeΔ), were examined for their association with cardio-metabolic traits, the lipids used to generate the scores and with prevalent-, and incident-cardiometabolic diseases (FIG. 11).

Prediction of mAge Scores Using the Research Lipid List and the CLP1s and CLP2s Lists

A ridge regression was used to create a predictive model for age including sex and BMI as covariates. To avoid overfitting, a 10-fold cross validation was employed in the AusDiab cohort (i.e. models were trained on 9/10^{th} of the cohort and used to predict BMI in the holdout 1/10^{th}). Using either the Research Lipid List (717 lipids, Table 2) or the CLP1s (310 lipids, Table 4) and CLP2s (254 lipids, Table 6) lists, similar performance for the prediction of age was observed. The models provided predicted age (pAge) values explaining 70.35%, 64.04% and 61.62% of the variance in age when either the Research Lipid List, CLP1s, or CLP2s lists were used, respectively (Table 12).

The models were described as a list of the covariates and lipids used in each model together with their β-coefficients (Tables 13, 14, 15 and 16). The lipid species were ranked by the absolute value of their β-coefficients which defined the relative contribution they made to the model. LASSO models (using optimised lambda) were also developed with the CLP1s and CLP2s lipid lists and the number of lipids and performance of these models was compared (Tables 13, 14, 15 and 16). It is recognised that the lipids at the top of these lists will contribute more to the model and so a subset of the lipids could be selected from the top 20 lipids, the top 50 lipids, the top 100 lipids, the top 200 lipids and used to make a model containing fewer lipids.

To standardise the pAge to the population, the mAge was then derived from the pAge scores as follows:

mAge=age+(pAge−(pAge value on the line of best fit between pAge and actual age))

The mAgeΔ was then defined as the difference between age and mAge. The correlation between age and mAge was consistent across the lipid sets: R²=81.83, 80.7, and 80.2 using the full lipidome, CLP1s and CLP2s respectively (Ridge) (Table 12).

TABLE 12
The predictive performances of ridge
model for the prediction of age

| | Correlation (R²) (%) | |
|---|---|---|
| | Age versus | Age versus |
| Lipid set | pAge | mAge |
| Research Lipid List (ridge, n = 717 | 70.35 | 81.83 |
| species) | | |
| CLP1s List (ridge, n = 298 species) | 64.4 | 80.7 |
| CLP2s List (ridge, n = 235 species) | 61.4 | 80.2 |
| CLP1s List (LASSO, n = 208 species) | 64.2 | 80.7 |
| CLP2s List (LASSO, n = 170) | 61.6 | 80.3 |

Similar values were demonstrated and achieved for pAge and mAge, whether the Research Lipid List or high-throughput CLP1s or CLP2s lipid lists are used. The correlation (R²) for the pAge using the Research Lipid List against the pAge derived using CLP1s, and CLP2s was 0.891 and 0.851 respectively. The correlation of pAges using CLP1s and CLP2s is relatively higher: R²=0.95. Similar but tighter correlations among the mAges derived using the Research Lipid List and CLP2s or CLP1s were also evident (FIG. 12).

Metabolic Age Derived Using the CLP and the Lipidome Show Similar Associations with Cardio-Metabolic Risk Factors

AusDiab participants were stratified into quintiles of mAgeΔ (defined as the difference between the participants' mAge and chronological age). A linear regression analysis between selected cardio-metabolic risk factors (as outcome) and the quintiles of mAgeΔ (as predictor), was performed to assess the overall association. Individuals in the top quintile of mAgeΔ (Q5) had higher triglycerides, cholesterol and SBP compared to individuals in Q1 regardless of the lipid set used to generate the mAgeΔ (FIG. 13).

The mAge Scores Generated Using the Full Lipidome and CLP Showed Similar Associations with Cardio-Metabolic Diseases

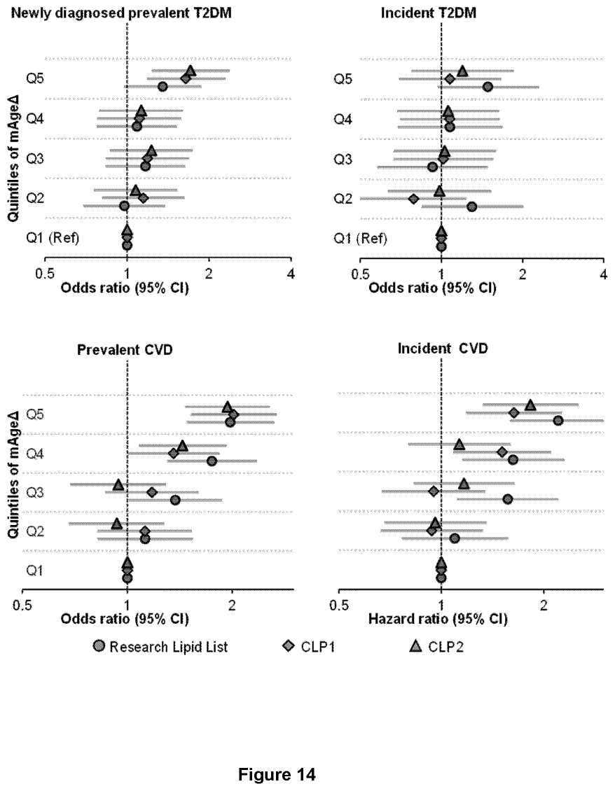

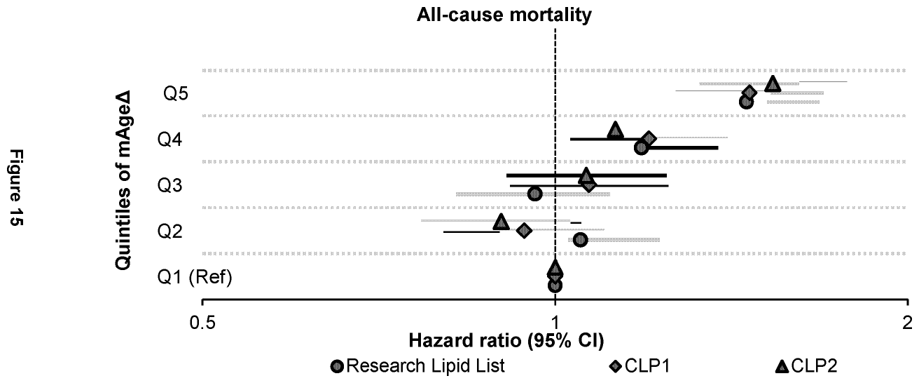

The associations between the mAgeΔ quintiles (derived from the Research Lipid List, CLP1s and CLP 2) with T2DM were consistent across quintiles. Similarly, all the mAgeΔ, regardless of the lipids set they are derived, from showed a similar association with both prevalent CVD and incident CVE (FIG. 14).

The Older mAge Group in Stratified Populations are Under Higher Risk of all-Cause Mortality

To examine whether mAge is a risk factors for all-cause mortality, independent of chronological age, a Cox regression was performed based on proportional hazard assumptions during 17 years follow-up (FIG. 15). The hazard ratios for risk of all-cause mortality (Q5/Q4 relative to Q1) were significant regardless of which lipid set was used to generate mAgeΔ (FIG. 15).

TABLE 13
Predictive models of age developed using Ridge regression
incorporating BMI and sex together with the CLP1s lipids.

| | | | | Ridge |
|---|---|---|---|---|
| Rank # | Lipid # | Lipid/Covariate | Ridge | (ABS) |
| | | (Intercept) | 55.022 | |
| | | drsex_00_n | 3.777 | 3.777 |
| | | bmi_00 | −0.200 | 0.200 |
| 1 | 145 | PC(P-34:2) | −4.372 | 4.372 |
| 2 | 4 | AcylCarnitine 14:1 | −4.109 | 4.109 |
| 3 | 7 | AcylCarnitine 16:1 | 3.572 | 3.572 |
| 4 | 77 | Hex3Cer(d18:1/16:0) | −3.166 | 3.166 |
| 5 | 195 | SM 33:1 | 2.915 | 2.915 |
| 6 | 136 | PC 40:6 | 2.803 | 2.803 |
| 7 | 197 | SM 34:2 | 2.737 | 2.737 |
| 8 | 6 | AcylCarnitine 16:0 | −2.336 | 2.336 |
| 9 | 144 | PC(P-34:1) | 2.332 | 2.332 |
| 10 | 122 | PC 35:5 | 2.326 | 2.326 |
| 11 | 34 | Cer(d18:1/23:0) | 2.131 | 2.131 |
| 12 | 72 | Hex2Cer(d18:1/16:0) | −1.997 | 1.997 |
| 13 | 123 | PC 36:1 | −1.893 | 1.893 |
| 14 | 151 | PE 34:2 | 1.869 | 1.869 |
| 15 | 171 | PE(P-18:0/18:2) | 1.845 | 1.845 |
| 16 | 188 | PI 38:6 | −1.829 | 1.829 |
| 17 | 162 | PE(P-16:0/18:1) | −1.779 | 1.779 |
| 18 | 94 | LPC(O-20:0) | −1.770 | 1.770 |
| 19 | 8 | AcylCarnitine 18:1 | 1.671 | 1.671 |
| 20 | 82 | LPC 18:2 | −1.665 | 1.665 |
| 21 | 129 | PC 36:5(b) | 1.647 | 1.647 |
| 22 | 166 | PE(P-16:0/22:4) | −1.595 | 1.595 |
| 23 | 153 | PE 36:1 | −1.589 | 1.589 |
| 24 | 172 | PE(P-18:0/20:4) | 1.580 | 1.580 |
| 25 | 209 | SM 44:3 | 1.573 | 1.573 |
| 26 | 130 | PC 37:6 | −1.569 | 1.569 |
| 27 | 265 | TG(54:6) [NL-20:4] | 1.566 | 1.566 |
| 28 | 74 | Hex2Cer(d18:1/24:0) | 1.554 | 1.554 |
| 29 | 39 | Cer(d18:2/24:0) | −1.536 | 1.536 |
| 30 | 40 | Cer(d18:2/24:1) | −1.531 | 1.531 |
| 31 | 201 | SM 36:1 | −1.485 | 1.485 |
| 32 | 5 | AcylCarnitine 14:2 | 1.485 | 1.485 |
| 33 | 113 | PC 32:2 | −1.453 | 1.453 |
| 34 | 193 | SM 32:1 | −1.451 | 1.451 |
| 35 | 173 | PE(P-18:0/20:5) | −1.446 | 1.446 |
| 36 | 231 | TG(50:3) [NL-14:0] | 1.445 | 1.445 |
| 37 | 15 | CE 17:0 | −1.429 | 1.429 |
| 38 | 17 | CE 18:1 | −1.413 | 1.413 |
| 39 | 47 | Cer(m18:1/24:0) | 1.399 | 1.399 |
| 40 | 219 | TG(48:2) [NL-18:2] | 1.378 | 1.378 |
| 41 | 255 | TG(53:2) [NL-18:1] | 1.337 | 1.337 |
| 42 | 235 | TG(50:3) [NL-18:3] | −1.334 | 1.334 |
| 43 | 85 | LPC 19:1 | −1.304 | 1.304 |
| 44 | 3 | AcylCarnitine 14:0 | 1.300 | 1.300 |
| 45 | 116 | PC 34:0 | 1.294 | 1.294 |
| 46 | 1 | AcylCarnitine 12:0 | 1.292 | 1.292 |
| 47 | 198 | SM 34:3 | 1.274 | 1.274 |
| 48 | 202 | SM 36:3 | −1.257 | 1.257 |
| 49 | 139 | PC(O-34:2) | 1.247 | 1.247 |
| 50 | 134 | PC 38:6(a) | −1.244 | 1.244 |
| 51 | 284 | TG(O-50:1) [NL-16:0] | 1.216 | 1.216 |
| 52 | 98 | LPC(O-24:2) | 1.214 | 1.214 |
| 53 | 203 | SM 38:1 | −1.211 | 1.211 |
| 54 | 99 | LPC(P-16:0) | 1.199 | 1.199 |
| 55 | 87 | LPC 20:4 | −1.192 | 1.192 |
| 56 | 111 | PC 32:0 | 1.150 | 1.150 |
| 57 | 102 | LPE 18:0 | 1.148 | 1.148 |
| 58 | 135 | PC 38:6(b) | −1.141 | 1.141 |
| 59 | 175 | PE(P-18:0/22:6) | 1.134 | 1.134 |
| 60 | 138 | PC(O-32:0) | −1.108 | 1.108 |
| 61 | 79 | LPC 17:0 | −1.106 | 1.106 |
| 62 | 44 | Cer(m18:0/22:0) | 1.101 | 1.101 |
| 63 | 220 | TG(48:3) [NL-14:0] | −1.099 | 1.099 |
| 64 | 78 | LPC 14:0 | −1.098 | 1.098 |
| 65 | 149 | PE 32:1 | −1.098 | 1.098 |
| 66 | 196 | SM 34:1 | 1.083 | 1.083 |
| 67 | 207 | SM 41:1 | −1.070 | 1.070 |
| 68 | 206 | SM 40:1 | −1.055 | 1.055 |
| 69 | 277 | TG(56:8) [NL-20:5] | −1.050 | 1.050 |
| 70 | 282 | TG(58:9) [NL-22:6] | −1.026 | 1.026 |
| 71 | 140 | PC(O-36:0) | 1.021 | 1.021 |
| 72 | 191 | S1P(d18:1) | −1.015 | 1.015 |
| 73 | 22 | CE 20:2 | 1.007 | 1.007 |
| 74 | 32 | Cer(d18:1/20:0) | 0.996 | 0.996 |
| 75 | 230 | TG(50:2) [NL-18:2] | −0.995 | 0.995 |
| 76 | 55 | DG 34:1 -(18:1) | −0.984 | 0.984 |
| 77 | 233 | TG(50:3) [NL-16:1] | −0.968 | 0.968 |
| 78 | 211 | Sulfatide (d18:1:/16:0(OH)) | −0.960 | 0.960 |
| 79 | 76 | Hex2Cer(d18:2/16:0) | 0.930 | 0.930 |
| 80 | 70 | Hex1Cer(d18:2/24:0) | 0.924 | 0.924 |
| 81 | 182 | PI 36:1 | −0.912 | 0.912 |
| 82 | 57 | DG 36:2 -(18:1) | −0.898 | 0.898 |
| 83 | 238 | TG(50:4) [NL-20:4] | 0.896 | 0.896 |
| 84 | 189 | PI 40:6 | −0.893 | 0.893 |
| 85 | 264 | TG(54:6) [NL-18:3] | 0.893 | 0.893 |
| 86 | 11 | CE 15:0 | 0.888 | 0.888 |
| 87 | 174 | PE(P-18:0/22:4) | −0.873 | 0.873 |
| 88 | 168 | PE(P-17:0/20:4) | −0.865 | 0.865 |
| 89 | 141 | PC(O-36:5) | −0.862 | 0.862 |
| 90 | 58 | DG 36:3 -(18:2) | 0.859 | 0.859 |
| 91 | 2 | AcylCarnitine 13:0 | 0.851 | 0.851 |
| 92 | 104 | LPE 18:2 | 0.849 | 0.849 |
| 93 | 262 | TG(54:5) [NL-18:3] | 0.842 | 0.842 |
| 94 | 232 | TG(50:3) [NL-14:1] | −0.841 | 0.841 |
| 95 | 286 | TG(O-50:1) [NL-18:1] | −0.840 | 0.840 |
| 96 | 263 | TG(54:5) [NL-20:4] | 0.830 | 0.830 |
| 97 | 158 | PE 40:6 | 0.829 | 0.829 |
| 98 | 161 | PE(O-36:4) | −0.828 | 0.828 |
| 99 | 9 | AcylCarnitine 18:2 | −0.826 | 0.826 |
| 100 | 38 | Cer(d18:2/23:0) | 0.821 | 0.821 |
| 101 | 21 | CE 20:1 | −0.805 | 0.805 |
| 102 | 45 | Cer(m18:1/20:0) | 0.800 | 0.800 |
| 103 | 12 | CE 16:0 | 0.792 | 0.792 |
| 104 | 81 | LPC 18:1 | 0.790 | 0.790 |
| 105 | 244 | TG(52:1) [NL-18:0] | 0.787 | 0.787 |
| 106 | 65 | Hex1Cer(d18:1/16:0) | −0.773 | 0.773 |
| 107 | 67 | Hex1Cer(d18:1/22:0) | 0.763 | 0.763 |
| 108 | 16 | CE 18:0 | −0.752 | 0.752 |
| 109 | 147 | PC(P-36:5) | 0.748 | 0.748 |
| 110 | 101 | LPE 16:0 | 0.747 | 0.747 |
| 111 | 69 | Hex1Cer(d18:1/24:1) | −0.745 | 0.745 |
| 112 | 227 | TG(50:2) [NL-14:0] | 0.744 | 0.744 |
| 113 | 280 | TG(58:10) [NL-22:6] | −0.742 | 0.742 |
| 114 | 276 | TG(56:8) [NL-20:4] | 0.734 | 0.734 |
| 115 | 33 | Cer(d18:1/22:0) | 0.723 | 0.723 |
| 116 | 71 | Hex2Cer(d16:1/16:0) | 0.705 | 0.705 |
| 117 | 142 | PC(P-30:0) | 0.703 | 0.703 |
| 118 | 118 | PC 34:2 | 0.702 | 0.702 |
| 119 | 270 | TG(56:6) [NL-20:4] | 0.701 | 0.701 |
| 120 | 214 | TG(48:1) [NL-16:1] | −0.694 | 0.694 |
| 121 | 283 | TG(O-50:1) [NL-15:0] | −0.693 | 0.693 |
| 122 | 186 | PI 38:3 | 0.693 | 0.693 |
| 123 | 36 | Cer(d18:1/24:1) | 0.691 | 0.691 |
| 124 | 221 | TG(48:3) [NL-16:1] | −0.691 | 0.691 |
| 125 | 92 | LPC 24:0 | 0.687 | 0.687 |
| 126 | 234 | TG(50:3) [NL-18:2] | −0.683 | 0.683 |
| 127 | 35 | Cer(d18:1/24:0) | −0.676 | 0.676 |
| 128 | 13 | CE 16:1 | 0.661 | 0.661 |
| 129 | 208 | SM 44:2 | 0.658 | 0.658 |
| 130 | 164 | PE(P-16:0/20:4) | 0.645 | 0.645 |
| 131 | 289 | TG(O-50:2) [NL-18:2] | −0.630 | 0.630 |
| 132 | 46 | Cer(m18:1/22:0) | 0.618 | 0.618 |
| 133 | 156 | PE 38:4 | −0.595 | 0.595 |
| 134 | 28 | Cer(d16:1/24:0) | −0.584 | 0.584 |
| 135 | 95 | LPC(O-22:0) | 0.580 | 0.580 |
| 136 | 252 | TG(52:5) [NL-20:4] | −0.576 | 0.576 |
| 137 | 61 | GM3(d18:1/16:0) | −0.575 | 0.575 |
| 138 | 157 | PE 38:6 | −0.572 | 0.572 |
| 139 | 181 | PI 34:1 | 0.567 | 0.567 |
| 140 | 31 | Cer(d18:1/16:0) | 0.566 | 0.566 |
| 141 | 248 | TG(52:4) [NL-16:1] | 0.560 | 0.560 |
| 142 | 159 | PE(O-34:1) | −0.556 | 0.556 |
| 143 | 267 | TG(54:6) [NL-22:6] | −0.552 | 0.552 |
| 144 | 160 | PE(O-34:2) | 0.548 | 0.548 |
| 145 | 146 | PC(P-36:4) | 0.547 | 0.547 |
| 146 | 215 | TG(48:1) [NL-18:1] | 0.544 | 0.544 |
| 147 | 64 | GM3(d18:1/24:1) | 0.543 | 0.543 |
| 148 | 204 | SM 38:2 | 0.543 | 0.543 |
| 149 | 210 | Sph(d17:1) | −0.541 | 0.541 |
| 150 | 30 | Cer(d17:1/24:0) | 0.541 | 0.541 |
| 151 | 27 | Cer(d16:1/22:0) | 0.526 | 0.526 |
| 152 | 68 | Hex1Cer(d18:1/24:0) | −0.519 | 0.519 |
| 153 | 251 | TG(52:5) [NL-18:3] | −0.516 | 0.516 |
| 154 | 298 | Ubiquinone | 0.501 | 0.501 |
| 155 | 199 | SM 35:1 | 0.499 | 0.499 |
| 156 | 23 | CE 20:4 | 0.485 | 0.485 |
| 157 | 287 | TG(O-50:2) [NL-16:1] | −0.480 | 0.480 |
| 158 | 275 | TG(56:7) [NL-22:6] | −0.472 | 0.472 |
| 159 | 285 | TG(O-50:1) [NL-17:1] | 0.465 | 0.465 |
| 160 | 105 | LPE 20:4 | −0.462 | 0.462 |
| 161 | 18 | CE 18:2 | −0.459 | 0.459 |
| 162 | 103 | LPE 18:1 | 0.459 | 0.459 |
| 163 | 10 | CE 14:0 | 0.456 | 0.456 |
| 164 | 19 | CE 18:3 | 0.455 | 0.455 |
| 165 | 62 | GM3(d18:1/18:0) | −0.448 | 0.448 |
| 166 | 212 | Sulfatide (d18:1:/16:0) | 0.446 | 0.446 |
| 167 | 165 | PE(P-16:0/20:5) | −0.433 | 0.433 |
| 168 | 217 | TG(48:2) [NL-14:1] | −0.433 | 0.433 |
| 169 | 24 | CE 20:5 | 0.428 | 0.428 |
| 170 | 253 | TG(52:5) [NL-20:5] | −0.425 | 0.425 |
| 171 | 131 | PC 38:4(a) | −0.419 | 0.419 |
| 172 | 37 | Cer(d18:2/22:0) | −0.415 | 0.415 |
| 173 | 124 | PC 36:2 | 0.413 | 0.413 |
| 174 | 163 | PE(P-16:0/18:2) | 0.412 | 0.412 |
| 175 | 127 | PC 36:4(b) | −0.412 | 0.412 |
| 176 | 254 | TG(53:2) [NL-17:1] | 0.408 | 0.408 |
| 177 | 273 | TG(56:7) [NL-20:5] | 0.407 | 0.407 |
| 178 | 274 | TG(56:7) [NL-22:5] | 0.407 | 0.407 |
| 179 | 169 | PE(P-17:0/22:6) | 0.407 | 0.407 |
| 180 | 96 | LPC(O-22:1) | 0.405 | 0.405 |
| 181 | 120 | PC 34:5 | −0.404 | 0.404 |
| 182 | 152 | PE 34:3 | 0.402 | 0.402 |
| 183 | 222 | TG(48:3) [NL-18:3] | 0.399 | 0.399 |
| 184 | 183 | PI 36:2 | 0.397 | 0.397 |
| 185 | 240 | TG(51:1) [NL-17:0] | 0.396 | 0.396 |
| 186 | 154 | PE 36:2 | −0.393 | 0.393 |
| 187 | 213 | TG(48:0) [NL-16:0] | 0.390 | 0.390 |
| 188 | 119 | PC 34:4 | −0.382 | 0.382 |
| 189 | 179 | PE(P-20:0/20:4) | 0.376 | 0.376 |
| 190 | 194 | SM 32:2 | −0.376 | 0.376 |
| 191 | 187 | PI 38:4 | −0.374 | 0.374 |
| 192 | 83 | LPC 18:3 | 0.368 | 0.368 |
| 193 | 292 | TG(O-52:2) [NL-17:1] | 0.365 | 0.365 |
| 194 | 115 | PC 33:2 | −0.361 | 0.361 |
| 195 | 148 | PC(P-37:4) | −0.360 | 0.360 |
| 196 | 246 | TG(52:2) [NL-16:0] | −0.357 | 0.357 |
| 197 | 66 | Hex1Cer(d18:1/18:0) | 0.354 | 0.354 |
| 198 | 218 | TG(48:2) [NL-16:1] | 0.348 | 0.348 |
| 199 | 271 | TG(56:6) [NL-22:5] | −0.343 | 0.343 |
| 200 | 266 | TG(54:6) [NL-20:5] | −0.341 | 0.341 |
| 201 | 93 | LPC 26:0 | −0.340 | 0.340 |
| 202 | 63 | GM3(d18:1/22:0) | 0.339 | 0.339 |
| 203 | 132 | PC 38:4(b) | 0.323 | 0.323 |
| 204 | 73 | Hex2Cer(d18:1/22:0) | 0.316 | 0.316 |
| 205 | 52 | DE(18:2) | −0.312 | 0.312 |
| 206 | 88 | LPC 20:5 | 0.309 | 0.309 |
| 207 | 200 | SM 35:2 | −0.308 | 0.308 |
| 208 | 241 | TG(51:2) [NL-15:0] | −0.305 | 0.305 |
| 209 | 260 | TG(54:4) [NL-18:2] | −0.304 | 0.304 |
| 210 | 43 | Cer(d19:1/24:1) | 0.294 | 0.294 |
| 211 | 184 | PI 36:4 | 0.293 | 0.293 |
| 212 | 49 | Cer1P(d18:1/16:0) | −0.282 | 0.282 |
| 213 | 51 | DE(18:1) | −0.279 | 0.279 |
| 214 | 117 | PC 34:1 | −0.277 | 0.277 |
| 215 | 290 | TG(O-52:0) [NL-16:0] | −0.276 | 0.276 |
| 216 | 107 | LPE(P-16:0) | 0.275 | 0.275 |
| 217 | 75 | Hex2Cer(d18:1/24:1) | −0.273 | 0.273 |
| 218 | 26 | CE 22:6 | 0.265 | 0.265 |
| 219 | 291 | TG(O-52:2) [NL-16:0] | 0.259 | 0.259 |
| 220 | 137 | PC 40:8 | 0.259 | 0.259 |
| 221 | 224 | TG(50:0) [NL-18:0] | −0.255 | 0.255 |
| 222 | 259 | TG(54:3) [NL-18:2] | 0.249 | 0.249 |
| 223 | 89 | LPC 22:0 | 0.240 | 0.240 |
| 224 | 180 | PI 32:1 | 0.236 | 0.236 |
| 225 | 296 | TG(O-54:4) [NL-17:1] | 0.235 | 0.235 |
| 226 | 86 | LPC 20:0 | −0.223 | 0.223 |
| 227 | 25 | CE 22:1 | −0.220 | 0.220 |
| 228 | 170 | PE(P-18:0/18:1) | 0.219 | 0.219 |
| 229 | 110 | PC 31:0 | 0.216 | 0.216 |
| 230 | 133 | PC 38:4(c) | 0.215 | 0.215 |
| 231 | 205 | SM 38:3 | −0.214 | 0.214 |
| 232 | 229 | TG(50:2) [NL-18:1] | −0.210 | 0.210 |
| 233 | 60 | DG 38:4 -(20:3) | −0.207 | 0.207 |
| 234 | 278 | TG(56:8) [NL-22:6] | −0.204 | 0.204 |
| 235 | 114 | PC 33:1 | −0.199 | 0.199 |
| 236 | 50 | COH (161) | −0.198 | 0.198 |
| 237 | 167 | PE(P-16:0/22:6) | −0.196 | 0.196 |
| 238 | 178 | PE(P-20:0/18:2) | 0.186 | 0.186 |
| 239 | 112 | PC 32:1 | 0.185 | 0.185 |
| 240 | 272 | TG(56:7) [NL-20:4] | −0.182 | 0.182 |
| 241 | 90 | LPC 22:5 | 0.180 | 0.180 |
| 242 | 279 | TG(56:9) [NL-22:6] | −0.177 | 0.177 |
| 243 | 29 | Cer(d16:1/24:1) | −0.169 | 0.169 |
| 244 | 245 | TG(52:1) [NL-18:1] | 0.162 | 0.162 |
| 245 | 261 | TG(54:4) [NL-20:3] | 0.162 | 0.162 |
| 246 | 237 | TG(50:4) [NL-18:3] | −0.150 | 0.150 |
| 247 | 53 | DE(20:4) | 0.143 | 0.143 |
| 248 | 48 | Cer(m18:1/24:1) | −0.138 | 0.138 |
| 249 | 250 | TG(52:4) [NL-18:3] | −0.137 | 0.137 |
| 250 | 97 | LPC(O-24:0) | −0.135 | 0.135 |
| 251 | 236 | TG(50:4) [NL-14:0] | −0.132 | 0.132 |
| 252 | 176 | PE(P-18:1/20:4) | 0.127 | 0.127 |
| 253 | 20 | CE 20:0 | 0.126 | 0.126 |
| 254 | 121 | PC 35:4 | −0.123 | 0.123 |
| 255 | 128 | PC 36:5(a) | 0.121 | 0.121 |
| 256 | 249 | TG(52:4) [NL-18:2] | −0.120 | 0.120 |
| 257 | 91 | LPC 22:6 | 0.111 | 0.111 |
| 258 | 56 | DG 34:2 -(18:2) | −0.108 | 0.108 |
| 259 | 294 | TG(O-54:3) [NL-17:1] | 0.099 | 0.099 |
| 260 | 225 | TG(50:1) [NL-16:0] | 0.095 | 0.095 |
| 261 | 109 | LPI 18:0 | 0.092 | 0.092 |
| 262 | 54 | DG 32:0 -(16:0) | 0.091 | 0.091 |
| 263 | 228 | TG(50:2) [NL-16:1] | 0.088 | 0.088 |
| 264 | 297 | TG(O-54:4) [NL-18:2] | −0.087 | 0.087 |
| 265 | 216 | TG(48:2) [NL-14:0] | −0.085 | 0.085 |
| 266 | 155 | PE 36:4 | 0.079 | 0.079 |
| 267 | 80 | LPC 18:0 | 0.077 | 0.077 |
| 268 | 100 | LPC(P-17:0) | 0.076 | 0.076 |
| 269 | 192 | SM 31:1 | −0.072 | 0.072 |
| 270 | 59 | DG 36:4 -(18:2) | 0.072 | 0.072 |
| 271 | 84 | LPC 19:0 | −0.071 | 0.071 |
| 272 | 126 | PC 36:4(a) | −0.062 | 0.062 |
| 273 | 185 | PI 37:4 | 0.062 | 0.062 |
| 274 | 288 | TG(O-50:2) [NL-18:1] | 0.061 | 0.061 |
| 275 | 268 | TG(54:7) [NL-20:5] | 0.058 | 0.058 |
| 276 | 177 | PE(P-18:1/20:5) | −0.057 | 0.057 |
| 277 | 293 | TG(O-52:2) [NL-18:1] | 0.053 | 0.053 |
| 278 | 281 | TG(58:8) [NL-22:6] | 0.053 | 0.053 |
| 279 | 108 | LPE(P-18:0) | 0.048 | 0.048 |
| 280 | 150 | PE 34:1 | 0.046 | 0.046 |
| 281 | 295 | TG(O-54:3) [NL-18:1] | 0.043 | 0.043 |
| 282 | 247 | TG(52:3) [NL-18:2] | 0.042 | 0.042 |
| 283 | 125 | PC 36:4 | −0.040 | 0.040 |
| 284 | 269 | TG(54:7) [NL-22:6] | 0.039 | 0.039 |
| 285 | 106 | LPE 22:6 | −0.036 | 0.036 |
| 286 | 143 | PC(P-32:0) | 0.036 | 0.036 |
| 287 | 190 | PS 38:4 | 0.035 | 0.035 |
| 288 | 243 | TG(51:2) [NL-17:1] | 0.032 | 0.032 |
| 289 | 242 | TG(51:2) [NL-17:0] | 0.025 | 0.025 |
| 290 | 239 | TG(51:0) [NL-16:0] | −0.023 | 0.023 |
| 291 | 256 | TG(54:2) [NL-18:0] | 0.014 | 0.014 |
| 292 | 41 | Cer(d19:1/22:0) | −0.014 | 0.014 |
| 293 | 257 | TG(54:2) [NL-20:1] | 0.013 | 0.013 |
| 294 | 223 | TG(49:1) [NL-17:1] | −0.011 | 0.011 |
| 295 | 14 | CE 16:2 | 0.010 | 0.010 |
| 296 | 42 | Cer(d19:1/24:0) | −0.009 | 0.009 |
| 297 | 226 | TG(50:1) [NL-18:1] | 0.007 | 0.007 |
| 298 | 258 | TG(54:3) [NL-18:1] | −0.006 | 0.006 |

TABLE 14
Predictive models of age developed using LASSO regression
incorporating BMI and sex together with the CLP1s lipids.

| | | | | LASSO |
|---|---|---|---|---|
| Rank # | Lipid # | Lipid/Covariate | LASSO | (ABS) |
| | | (Intercept) | 54.682 | |
| | | drsex_00_n | 3.965 | 3.965 |
| | | bmi_00 | −0.192 | 0.192 |
| 1 | 4 | AcylCarnitine 14:1 | −6.870 | 6.870 |
| 2 | 145 | PC(P-34:2) | −4.935 | 4.935 |
| 3 | 7 | AcylCarnitine 16:1 | 4.897 | 4.897 |
| 4 | 77 | Hex3Cer(d18:1/16:0) | −4.026 | 4.026 |
| 5 | 195 | SM 33:1 | 3.186 | 3.186 |
| 6 | 136 | PC 40:6 | 3.074 | 3.074 |
| 7 | 5 | AcylCarnitine 14:2 | 3.000 | 3.000 |
| 8 | 197 | SM 34:2 | 2.972 | 2.972 |
| 9 | 6 | AcylCarnitine 16:0 | −2.949 | 2.949 |
| 10 | 144 | PC(P-34:1) | 2.898 | 2.898 |
| 11 | 122 | PC 35:5 | 2.686 | 2.686 |
| 12 | 34 | Cer(d18:1/23:0) | 2.333 | 2.333 |
| 13 | 171 | PE(P-18:0/18:2) | 2.305 | 2.305 |
| 14 | 130 | PC 37:6 | −2.272 | 2.272 |
| 15 | 151 | PE 34:2 | 2.113 | 2.113 |
| 16 | 72 | Hex2Cer(d18:1/16:0) | −2.080 | 2.080 |
| 17 | 1 | AcylCarnitine 12:0 | 1.914 | 1.914 |
| 18 | 123 | PC 36:1 | −1.802 | 1.802 |
| 19 | 172 | PE(P-18:0/20:4) | 1.755 | 1.755 |
| 20 | 162 | PE(P-16:0/18:1) | −1.728 | 1.728 |
| 21 | 15 | CE 17:0 | −1.701 | 1.701 |
| 22 | 166 | PE(P-16:0/22:4) | −1.696 | 1.696 |
| 23 | 188 | PI 38:6 | −1.649 | 1.649 |
| 24 | 235 | TG(50:3) [NL-18:3] | −1.624 | 1.624 |
| 25 | 98 | LPC(O-24:2) | 1.620 | 1.620 |
| 26 | 265 | TG(54:6) [NL-20:4] | 1.616 | 1.616 |
| 27 | 39 | Cer(d18:2/24:0) | −1.604 | 1.604 |
| 28 | 47 | Cer(m18:1/24:0) | 1.586 | 1.586 |
| 29 | 99 | LPC(P-16:0) | 1.561 | 1.561 |
| 30 | 8 | AcylCarnitine 18:1 | 1.554 | 1.554 |
| 31 | 40 | Cer(d18:2/24:1) | −1.550 | 1.550 |
| 32 | 153 | PE 36:1 | −1.540 | 1.540 |
| 33 | 193 | SM 32:1 | −1.516 | 1.516 |
| 34 | 94 | LPC(O-20:0) | −1.445 | 1.445 |
| 35 | 173 | PE(P-18:0/20:5) | −1.440 | 1.440 |
| 36 | 201 | SM 36:1 | −1.395 | 1.395 |
| 37 | 116 | PC 34:0 | 1.378 | 1.378 |
| 38 | 203 | SM 38:1 | −1.314 | 1.314 |
| 39 | 209 | SM 44:3 | 1.313 | 1.313 |
| 40 | 82 | LPC 18:2 | −1.307 | 1.307 |
| 41 | 202 | SM 36:3 | −1.300 | 1.300 |
| 42 | 149 | PE 32:1 | −1.299 | 1.299 |
| 43 | 255 | TG(53:2) [NL-18:1] | 1.290 | 1.290 |
| 44 | 231 | TG(50:3) [NL-14:0] | 1.265 | 1.265 |
| 45 | 74 | Hex2Cer(d18:1/24:0) | 1.262 | 1.262 |
| 46 | 139 | PC(O-34:2) | 1.238 | 1.238 |
| 47 | 129 | PC 36:5(b) | 1.221 | 1.221 |
| 48 | 111 | PC 32:0 | 1.217 | 1.217 |
| 49 | 284 | TG(O-50:1) [NL-16:0] | 1.210 | 1.210 |
| 50 | 17 | CE 18:1 | −1.207 | 1.207 |
| 51 | 3 | AcylCarnitine 14:0 | 1.135 | 1.135 |
| 52 | 220 | TG(48:3) [NL-14:0] | −1.111 | 1.111 |
| 53 | 32 | Cer(d18:1/20:0) | 1.095 | 1.095 |
| 54 | 44 | Cer(m18:0/22:0) | 1.091 | 1.091 |
| 55 | 233 | TG(50:3) [NL-16:1] | −1.078 | 1.078 |
| 56 | 206 | SM 40:1 | −1.075 | 1.075 |
| 57 | 219 | TG(48:2) [NL-18:2] | 1.072 | 1.072 |
| 58 | 138 | PC(O-32:0) | −1.067 | 1.067 |
| 59 | 22 | CE 20:2 | 1.035 | 1.035 |
| 60 | 196 | SM 34:1 | 1.028 | 1.028 |
| 61 | 11 | CE 15:0 | 1.024 | 1.024 |
| 62 | 134 | PC 38:6(a) | −1.014 | 1.014 |
| 63 | 79 | LPC 17:0 | −1.008 | 1.008 |
| 64 | 102 | LPE 18:0 | 0.972 | 0.972 |
| 65 | 191 | S1P(d18:1) | −0.970 | 0.970 |
| 66 | 70 | Hex1Cer(d18:2/24:0) | 0.959 | 0.959 |
| 67 | 2 | AcylCarnitine 13:0 | 0.939 | 0.939 |
| 68 | 211 | Sulfatide (d18:1:/16:0(OH)) | −0.937 | 0.937 |
| 69 | 113 | PC 32:2 | −0.913 | 0.913 |
| 70 | 140 | PC(O-36:0) | 0.904 | 0.904 |
| 71 | 85 | LPC 19:1 | −0.890 | 0.890 |
| 72 | 55 | DG 34:1 -(18:1) | −0.881 | 0.881 |
| 73 | 282 | TG(58:9) [NL-22:6] | −0.853 | 0.853 |
| 74 | 87 | LPC 20:4 | −0.851 | 0.851 |
| 75 | 45 | Cer(m18:1/20:0) | 0.850 | 0.850 |
| 76 | 147 | PC(P-36:5) | 0.850 | 0.850 |
| 77 | 275 | TG(56:7) [NL-22:6] | −0.849 | 0.849 |
| 78 | 141 | PC(O-36:5) | −0.828 | 0.828 |
| 79 | 198 | SM 34:3 | 0.810 | 0.810 |
| 80 | 161 | PE(O-36:4) | −0.807 | 0.807 |
| 81 | 227 | TG(50:2) [NL-14:0] | 0.787 | 0.787 |
| 82 | 135 | PC 38:6(b) | −0.779 | 0.779 |
| 83 | 175 | PE(P-18:0/22:6) | 0.773 | 0.773 |
| 84 | 277 | TG(56:8) [NL-20:5] | −0.766 | 0.766 |
| 85 | 9 | AcylCarnitine 18:2 | −0.764 | 0.764 |
| 86 | 81 | LPC 18:1 | 0.756 | 0.756 |
| 87 | 21 | CE 20:1 | −0.748 | 0.748 |
| 88 | 232 | TG(50:3) [NL-14:1] | −0.746 | 0.746 |
| 89 | 174 | PE(P-18:0/22:4) | −0.725 | 0.725 |
| 90 | 263 | TG(54:5) [NL-20:4] | 0.723 | 0.723 |
| 91 | 280 | TG(58:10) [NL-22:6] | −0.710 | 0.710 |
| 92 | 283 | TG(O-50:1) [NL-15:0] | −0.696 | 0.696 |
| 93 | 230 | TG(50:2) [NL-18:2] | −0.695 | 0.695 |
| 94 | 270 | TG(56:6) [NL-20:4] | 0.666 | 0.666 |
| 95 | 69 | Hex1Cer(d18:1/24:1) | −0.661 | 0.661 |
| 96 | 65 | Hex1Cer(d18:1/16:0) | −0.653 | 0.653 |
| 97 | 12 | CE 16:0 | 0.645 | 0.645 |
| 98 | 16 | CE 18:0 | −0.644 | 0.644 |
| 99 | 262 | TG(54:5) [NL-18:3] | 0.644 | 0.644 |
| 100 | 164 | PE(P-16:0/20:4) | 0.638 | 0.638 |
| 101 | 189 | PI 40:6 | −0.631 | 0.631 |
| 102 | 36 | Cer(d18:1/24:1) | 0.626 | 0.626 |
| 103 | 64 | GM3(d18:1/24:1) | 0.625 | 0.625 |
| 104 | 104 | LPE 18:2 | 0.624 | 0.624 |
| 105 | 101 | LPE 16:0 | 0.600 | 0.600 |
| 106 | 289 | TG(O-50:2) [NL-18:2] | −0.593 | 0.593 |
| 107 | 71 | Hex2Cer(d16:1/16:0) | 0.591 | 0.591 |
| 108 | 221 | TG(48:3) [NL-16:1] | −0.573 | 0.573 |
| 109 | 244 | TG(52:1) [NL-18:0] | 0.570 | 0.570 |
| 110 | 120 | PC 34:5 | −0.563 | 0.563 |
| 111 | 210 | Sph(d17:1) | −0.554 | 0.554 |
| 112 | 38 | Cer(d18:2/23:0) | 0.539 | 0.539 |
| 113 | 33 | Cer(d18:1/22:0) | 0.532 | 0.532 |
| 114 | 186 | PI 38:3 | 0.526 | 0.526 |
| 115 | 10 | CE 14:0 | 0.512 | 0.512 |
| 116 | 67 | Hex1Cer(d18:1/22:0) | 0.506 | 0.506 |
| 117 | 215 | TG(48:1) [NL-18:1] | 0.499 | 0.499 |
| 118 | 31 | Cer(d18:1/16:0) | 0.498 | 0.498 |
| 119 | 286 | TG(O-50:1) [NL-18:1] | −0.488 | 0.488 |
| 120 | 234 | TG(50:3) [NL-18:2] | −0.475 | 0.475 |
| 121 | 76 | Hex2Cer(d18:2/16:0) | 0.472 | 0.472 |
| 122 | 208 | SM 44:2 | 0.470 | 0.470 |
| 123 | 118 | PC 34:2 | 0.464 | 0.464 |
| 124 | 62 | GM3(d18:1/18:0) | −0.462 | 0.462 |
| 125 | 61 | GM3(d18:1/16:0) | −0.460 | 0.460 |
| 126 | 19 | CE 18:3 | 0.458 | 0.458 |
| 127 | 182 | PI 36:1 | −0.457 | 0.457 |
| 128 | 92 | LPC 24:0 | 0.454 | 0.454 |
| 129 | 207 | SM 41:1 | −0.438 | 0.438 |
| 130 | 212 | Sulfatide (d18:1:/16:0) | 0.422 | 0.422 |
| 131 | 95 | LPC(O-22:0) | 0.408 | 0.408 |
| 132 | 264 | TG(54:6) [NL-18:3] | 0.402 | 0.402 |
| 133 | 26 | CE 22:6 | 0.393 | 0.393 |
| 134 | 168 | PE(P-17:0/20:4) | −0.377 | 0.377 |
| 135 | 18 | CE 18:2 | −0.373 | 0.373 |
| 136 | 287 | TG(O-50:2) [NL-16:1] | −0.370 | 0.370 |
| 137 | 159 | PE(O-34:1) | −0.367 | 0.367 |
| 138 | 57 | DG 36:2 -(18:1) | −0.364 | 0.364 |
| 139 | 298 | Ubiquinone | 0.360 | 0.360 |
| 140 | 156 | PE 38:4 | −0.359 | 0.359 |
| 141 | 204 | SM 38:2 | 0.354 | 0.354 |
| 142 | 267 | TG(54:6) [NL-22:6] | −0.352 | 0.352 |
| 143 | 142 | PC(P-30:0) | 0.340 | 0.340 |
| 144 | 292 | TG(O-52:2) [NL-17:1] | 0.320 | 0.320 |
| 145 | 51 | DE(18:1) | −0.307 | 0.307 |
| 146 | 241 | TG(51:2) [NL-15:0] | −0.306 | 0.306 |
| 147 | 78 | LPC 14:0 | −0.302 | 0.302 |
| 148 | 148 | PC(P-37:4) | −0.289 | 0.289 |
| 149 | 68 | Hex1Cer(d18:1/24:0) | −0.283 | 0.283 |
| 150 | 49 | Cer1P(d18:1/16:0) | −0.277 | 0.277 |
| 151 | 160 | PE(O-34:2) | 0.276 | 0.276 |
| 152 | 259 | TG(54:3) [NL-18:2] | 0.269 | 0.269 |
| 153 | 105 | LPE 20:4 | −0.258 | 0.258 |
| 154 | 23 | CE 20:4 | 0.256 | 0.256 |
| 155 | 285 | TG(O-50:1) [NL-17:1] | 0.254 | 0.254 |
| 156 | 107 | LPE(P-16:0) | 0.250 | 0.250 |
| 157 | 163 | PE(P-16:0/18:2) | 0.245 | 0.245 |
| 158 | 63 | GM3(d18:1/22:0) | 0.244 | 0.244 |
| 159 | 178 | PE(P-20:0/18:2) | 0.225 | 0.225 |
| 160 | 276 | TG(56:8) [NL-20:4] | 0.218 | 0.218 |
| 161 | 89 | LPC 22:0 | 0.216 | 0.216 |
| 162 | 66 | Hex1Cer(d18:1/18:0) | 0.216 | 0.216 |
| 163 | 93 | LPC 26:0 | −0.207 | 0.207 |
| 164 | 46 | Cer(m18:1/22:0) | 0.200 | 0.200 |
| 165 | 13 | CE 16:1 | 0.199 | 0.199 |
| 166 | 294 | TG(O-54:3) [NL-17:1] | 0.197 | 0.197 |
| 167 | 119 | PC 34:4 | −0.194 | 0.194 |
| 168 | 214 | TG(48:1) [NL-16:1] | −0.192 | 0.192 |
| 169 | 296 | TG(O-54:4) [NL-17:1] | 0.187 | 0.187 |
| 170 | 179 | PE(P-20:0/20:4) | 0.184 | 0.184 |
| 171 | 181 | PI 34:1 | 0.178 | 0.178 |
| 172 | 28 | Cer(d16:1/24:0) | −0.171 | 0.171 |
| 173 | 146 | PC(P-36:4) | 0.171 | 0.171 |
| 174 | 24 | CE 20:5 | 0.169 | 0.169 |
| 175 | 184 | PI 36:4 | 0.163 | 0.163 |
| 176 | 194 | SM 32:2 | −0.163 | 0.163 |
| 177 | 50 | COH (161) | −0.154 | 0.154 |
| 178 | 124 | PC 36:2 | 0.154 | 0.154 |
| 179 | 96 | LPC(O-22:1) | 0.153 | 0.153 |
| 180 | 109 | LPI 18:0 | 0.152 | 0.152 |
| 181 | 133 | PC 38:4(c) | −0.145 | 0.145 |
| 182 | 240 | TG(51:1) [NL-17:0] | 0.133 | 0.133 |
| 183 | 152 | PE 34:3 | 0.131 | 0.131 |
| 184 | 169 | PE(P-17:0/22:6) | 0.123 | 0.123 |
| 185 | 52 | DE(18:2) | −0.120 | 0.120 |
| 186 | 103 | LPE 18:1 | 0.113 | 0.113 |
| 187 | 246 | TG(52:2) [NL-16:0] | −0.110 | 0.110 |
| 188 | 199 | SM 35:1 | 0.108 | 0.108 |
| 189 | 187 | PI 38:4 | −0.107 | 0.107 |
| 190 | 291 | TG(O-52:2) [NL-16:0] | 0.097 | 0.097 |
| 191 | 131 | PC 38:4(a) | −0.089 | 0.089 |
| 192 | 205 | SM 38:3 | −0.068 | 0.068 |
| 193 | 25 | CE 22:1 | −0.065 | 0.065 |
| 194 | 35 | Cer(d18:1/24:0) | −0.057 | 0.057 |
| 195 | 43 | Cer(d19:1/24:1) | 0.048 | 0.048 |
| 196 | 192 | SM 31:1 | −0.041 | 0.041 |
| 197 | 108 | LPE(P-18:0) | 0.024 | 0.024 |
| 198 | 180 | PI 32:1 | 0.021 | 0.021 |
| 199 | 53 | DE(20:4) | 0.014 | 0.014 |
| 200 | 273 | TG(56:7) [NL-20:5] | 0.013 | 0.013 |
| 201 | 115 | PC 33:2 | −0.011 | 0.011 |
| 202 | 260 | TG(54:4) [NL-18:2] | −0.006 | 0.006 |
| 203 | 143 | PC(P-32:0) | 0.003 | 0.003 |
| 204 | 165 | PE(P-16:0/20:5) | −0.003 | 0.003 |
| 205 | 238 | TG(50:4) [NL-20:4] | 0.000 | 0.000 |
| 206 | 127 | PC 36:4(b) | 0.000 | 0.000 |
| 207 | 14 | CE 16:2 | 0.000 | 0.000 |
| 208 | 20 | CE 20:0 | 0.000 | 0.000 |
| 209 | 27 | Cer(d16:1/22:0) | 0.000 | 0.000 |
| 210 | 29 | Cer(d16:1/24:1) | 0.000 | 0.000 |
| 211 | 30 | Cer(d17:1/24:0) | 0.000 | 0.000 |
| 212 | 37 | Cer(d18:2/22:0) | 0.000 | 0.000 |
| 213 | 41 | Cer(d19:1/22:0) | 0.000 | 0.000 |
| 214 | 42 | Cer(d19:1/24:0) | 0.000 | 0.000 |
| 215 | 48 | Cer(m18:1/24:1) | 0.000 | 0.000 |
| 216 | 54 | DG 32:0 -(16:0) | 0.000 | 0.000 |
| 217 | 56 | DG 34:2 -(18:2) | 0.000 | 0.000 |
| 218 | 58 | DG 36:3 -(18:2) | 0.000 | 0.000 |
| 219 | 59 | DG 36:4 -(18:2) | 0.000 | 0.000 |
| 220 | 60 | DG 38:4 -(20:3) | 0.000 | 0.000 |
| 221 | 73 | Hex2Cer(d18:1/22:0) | 0.000 | 0.000 |
| 222 | 75 | Hex2Cer(d18:1/24:1) | 0.000 | 0.000 |
| 223 | 80 | LPC 18:0 | 0.000 | 0.000 |
| 224 | 83 | LPC 18:3 | 0.000 | 0.000 |
| 225 | 84 | LPC 19:0 | 0.000 | 0.000 |
| 226 | 86 | LPC 20:0 | 0.000 | 0.000 |
| 227 | 88 | LPC 20:5 | 0.000 | 0.000 |
| 228 | 90 | LPC 22:5 | 0.000 | 0.000 |
| 229 | 91 | LPC 22:6 | 0.000 | 0.000 |
| 230 | 97 | LPC(O-24:0) | 0.000 | 0.000 |
| 231 | 100 | LPC(P-17:0) | 0.000 | 0.000 |
| 232 | 106 | LPE 22:6 | 0.000 | 0.000 |
| 233 | 110 | PC 31:0 | 0.000 | 0.000 |
| 234 | 112 | PC 32:1 | 0.000 | 0.000 |
| 235 | 114 | PC 33:1 | 0.000 | 0.000 |
| 236 | 117 | PC 34:1 | 0.000 | 0.000 |
| 237 | 121 | PC 35:4 | 0.000 | 0.000 |
| 238 | 125 | PC 36:4 | 0.000 | 0.000 |
| 239 | 126 | PC 36:4(a) | 0.000 | 0.000 |
| 240 | 128 | PC 36:5(a) | 0.000 | 0.000 |
| 241 | 132 | PC 38:4(b) | 0.000 | 0.000 |
| 242 | 137 | PC 40:8 | 0.000 | 0.000 |
| 243 | 150 | PE 34:1 | 0.000 | 0.000 |
| 244 | 154 | PE 36:2 | 0.000 | 0.000 |
| 245 | 155 | PE 36:4 | 0.000 | 0.000 |
| 246 | 157 | PE 38:6 | 0.000 | 0.000 |
| 247 | 158 | PE 40:6 | 0.000 | 0.000 |
| 248 | 167 | PE(P-16:0/22:6) | 0.000 | 0.000 |
| 249 | 170 | PE(P-18:0/18:1) | 0.000 | 0.000 |
| 250 | 176 | PE(P-18:1/20:4) | 0.000 | 0.000 |
| 251 | 177 | PE(P-18:1/20:5) | 0.000 | 0.000 |
| 252 | 183 | PI 36:2 | 0.000 | 0.000 |
| 253 | 185 | PI 37:4 | 0.000 | 0.000 |
| 254 | 190 | PS 38:4 | 0.000 | 0.000 |
| 255 | 200 | SM 35:2 | 0.000 | 0.000 |
| 256 | 213 | TG(48:0) [NL-16:0] | 0.000 | 0.000 |
| 257 | 216 | TG(48:2) [NL-14:0] | 0.000 | 0.000 |
| 258 | 217 | TG(48:2) [NL-14:1] | 0.000 | 0.000 |
| 259 | 218 | TG(48:2) [NL-16:1] | 0.000 | 0.000 |
| 260 | 222 | TG(48:3) [NL-18:3] | 0.000 | 0.000 |
| 261 | 223 | TG(49:1) [NL-17:1] | 0.000 | 0.000 |
| 262 | 224 | TG(50:0) [NL-18:0] | 0.000 | 0.000 |
| 263 | 225 | TG(50:1) [NL-16:0] | 0.000 | 0.000 |
| 264 | 226 | TG(50:1) [NL-18:1] | 0.000 | 0.000 |
| 265 | 228 | TG(50:2) [NL-16:1] | 0.000 | 0.000 |
| 266 | 229 | TG(50:2) [NL-18:1] | 0.000 | 0.000 |
| 267 | 236 | TG(50:4) [NL-14:0] | 0.000 | 0.000 |
| 268 | 237 | TG(50:4) [NL-18:3] | 0.000 | 0.000 |
| 269 | 239 | TG(51:0) [NL-16:0] | 0.000 | 0.000 |
| 270 | 242 | TG(51:2) [NL-17:0] | 0.000 | 0.000 |
| 271 | 243 | TG(51:2) [NL-17:1] | 0.000 | 0.000 |
| 272 | 245 | TG(52:1) [NL-18:1] | 0.000 | 0.000 |
| 273 | 247 | TG(52:3) [NL-18:2] | 0.000 | 0.000 |
| 274 | 248 | TG(52:4) [NL-16:1] | 0.000 | 0.000 |
| 275 | 249 | TG(52:4) [NL-18:2] | 0.000 | 0.000 |
| 276 | 250 | TG(52:4) [NL-18:3] | 0.000 | 0.000 |
| 277 | 251 | TG(52:5) [NL-18:3] | 0.000 | 0.000 |
| 278 | 252 | TG(52:5) [NL-20:4] | 0.000 | 0.000 |
| 279 | 253 | TG(52:5) [NL-20:5] | 0.000 | 0.000 |
| 280 | 254 | TG(53:2) [NL-17:1] | 0.000 | 0.000 |
| 281 | 256 | TG(54:2) [NL-18:0] | 0.000 | 0.000 |
| 282 | 257 | TG(54:2) [NL-20:1] | 0.000 | 0.000 |
| 283 | 258 | TG(54:3) [NL-18:1] | 0.000 | 0.000 |
| 284 | 261 | TG(54:4) [NL-20:3] | 0.000 | 0.000 |
| 285 | 266 | TG(54:6) [NL-20:5] | 0.000 | 0.000 |
| 286 | 268 | TG(54:7) [NL-20:5] | 0.000 | 0.000 |
| 287 | 269 | TG(54:7) [NL-22:6] | 0.000 | 0.000 |
| 288 | 271 | TG(56:6) [NL-22:5] | 0.000 | 0.000 |
| 289 | 272 | TG(56:7) [NL-20:4] | 0.000 | 0.000 |
| 290 | 274 | TG(56:7) [NL-22:5] | 0.000 | 0.000 |
| 291 | 278 | TG(56:8) [NL-22:6] | 0.000 | 0.000 |
| 292 | 279 | TG(56:9) [NL-22:6] | 0.000 | 0.000 |
| 293 | 281 | TG(58:8) [NL-22:6] | 0.000 | 0.000 |
| 294 | 288 | TG(O-50:2) [NL-18:1] | 0.000 | 0.000 |
| 295 | 290 | TG(O-52:0) [NL-16:0] | 0.000 | 0.000 |
| 296 | 293 | TG(O-52:2) [NL-18:1] | 0.000 | 0.000 |
| 297 | 295 | TG(O-54:3) [NL-18:1] | 0.000 | 0.000 |
| 298 | 297 | TG(O-54:4) [NL-18:2] | 0.000 | 0.000 |

TABLE 15
Predictive models of age developed using Ridge regression
incorporating BMI and sex together with the CLP2s lipids

| Rank # | Lipid # | Lipid/Covariate | Ridge | Ridge (ABS) |
|---|---|---|---|---|
| | | (Intercept) | 55.227 | |
| | | drsex_00_n | 3.045 | 3.045 |
| | | bmi_00 | −0.197 | 0.197 |
| 1 | 7 | AcylCarnitine 16:1 | 3.490 | 3.490 |
| 2 | 123 | PC(P-34:2) | −3.259 | 3.259 |
| 3 | 4 | AcylCarnitine 14:1 | −3.227 | 3.227 |
| 4 | 118 | PC 40:6 | 3.070 | 3.070 |
| 5 | 68 | Hex3Cer(d18:1/16:0) | −2.749 | 2.749 |
| 6 | 168 | SM 34:2 | 2.713 | 2.713 |
| 7 | 166 | SM 33:1 | 2.703 | 2.703 |
| 8 | 6 | AcylCarnitine 16:0 | −2.546 | 2.546 |
| 9 | 31 | Cer(d18:1/23:0) | 2.354 | 2.354 |
| 10 | 65 | Hex2Cer(d18:1/16:0) | −2.071 | 2.071 |
| 11 | 111 | PC 36:5(b) | 2.048 | 2.048 |
| 12 | 144 | PE(P-18:0/18:2) | 2.018 | 2.018 |
| 13 | 159 | PI 38:6 | −1.998 | 1.998 |
| 14 | 15 | CE 17:0 | −1.937 | 1.937 |
| 15 | 99 | PC 32:0 | 1.889 | 1.889 |
| 16 | 76 | LPC 19:1 | −1.821 | 1.821 |
| 17 | 129 | PE 34:2 | 1.804 | 1.804 |
| 18 | 172 | SM 36:1 | −1.724 | 1.724 |
| 19 | 192 | TG(50:3) [NL-14:0] | 1.705 | 1.705 |
| 20 | 73 | LPC 18:2 | −1.699 | 1.699 |
| 21 | 44 | Cer(m18:1/24:0) | 1.698 | 1.698 |
| 22 | 179 | SM 44:2 | 1.559 | 1.559 |
| 23 | 36 | Cer(d18:2/24:0) | −1.505 | 1.505 |
| 24 | 90 | LPE 18:0 | 1.405 | 1.405 |
| 25 | 164 | SM 32:1 | −1.384 | 1.384 |
| 26 | 120 | PC(O-32:0) | −1.370 | 1.370 |
| 27 | 169 | SM 34:3 | 1.366 | 1.366 |
| 28 | 8 | AcylCarnitine 18:1 | 1.359 | 1.359 |
| 29 | 37 | Cer(d18:2/24:1) | −1.324 | 1.324 |
| 30 | 178 | SM 41:1 | −1.255 | 1.255 |
| 31 | 213 | TG(54:6) [NL-20:4] | 1.243 | 1.243 |
| 32 | 173 | SM 36:3 | −1.236 | 1.236 |
| 33 | 77 | LPC 20:4 | −1.217 | 1.217 |
| 34 | 215 | TG(54:6) [NL-22:6] | −1.214 | 1.214 |
| 35 | 5 | AcylCarnitine 14:2 | 1.206 | 1.206 |
| 36 | 125 | PC(P-36:5) | 1.186 | 1.186 |
| 37 | 174 | SM 38:1 | −1.185 | 1.185 |
| 38 | 188 | TG(48:3) [NL-14:0] | −1.172 | 1.172 |
| 39 | 146 | PE(P-18:0/20:5) | −1.163 | 1.163 |
| 40 | 140 | PE(P-16:0/22:6) | −1.146 | 1.146 |
| 41 | 139 | PE(P-16:0/20:5) | −1.138 | 1.138 |
| 42 | 177 | SM 40:1 | −1.137 | 1.137 |
| 43 | 145 | PE(P-18:0/20:4) | 1.133 | 1.133 |
| 44 | 52 | DG 34:1 -(18:1) | −1.129 | 1.129 |
| 45 | 143 | PE(P-18:0/18:1) | −1.111 | 1.111 |
| 46 | 87 | LPC(P-16:0) | 1.085 | 1.085 |
| 47 | 167 | SM 34:1 | 1.076 | 1.076 |
| 48 | 1 | AcylCarnitine 12:0 | 1.075 | 1.075 |
| 49 | 181 | Sulfatide (d18:1:/16:0(OH)) | −1.065 | 1.065 |
| 50 | 101 | PC 32:2 | −1.047 | 1.047 |
| 51 | 127 | PE 32:1 | −1.043 | 1.043 |
| 52 | 3 | AcylCarnitine 14:0 | 1.037 | 1.037 |
| 53 | 194 | TG(50:3) [NL-16:1] | −1.033 | 1.033 |
| 54 | 162 | S1P(d18:1) | −1.020 | 1.020 |
| 55 | 63 | Hex1Cer(d18:2/24:0) | 1.012 | 1.012 |
| 56 | 29 | Cer(d18:1/20:0) | 1.003 | 1.003 |
| 57 | 160 | PI 40:6 | −0.998 | 0.998 |
| 58 | 116 | PC 38:6(a) | −0.998 | 0.998 |
| 59 | 19 | CE 20:1 | −0.976 | 0.976 |
| 60 | 11 | CE 15:0 | 0.936 | 0.936 |
| 61 | 132 | PE 38:4 | −0.935 | 0.935 |
| 62 | 62 | Hex1Cer(d18:1/24:1) | −0.917 | 0.917 |
| 63 | 54 | DG 36:2 -(18:1) | −0.911 | 0.911 |
| 64 | 89 | LPE 16:0 | 0.899 | 0.899 |
| 65 | 112 | PC 37:6 | −0.898 | 0.898 |
| 66 | 208 | TG(54:2) [NL-20:1] | 0.893 | 0.893 |
| 67 | 230 | TG(58:9) [NL-22:6] | −0.880 | 0.880 |
| 68 | 212 | TG(54:6) [NL-18:3] | 0.871 | 0.871 |
| 69 | 224 | TG(56:8) [NL-20:4] | 0.870 | 0.870 |
| 70 | 122 | PC(P-32:0) | 0.861 | 0.861 |
| 71 | 135 | PE(O-34:1) | −0.855 | 0.855 |
| 72 | 42 | Cer(m18:1/20:0) | 0.854 | 0.854 |
| 73 | 82 | LPC 24:0 | 0.853 | 0.853 |
| 74 | 154 | PI 36:1 | −0.831 | 0.831 |
| 75 | 92 | LPE 18:2 | 0.827 | 0.827 |
| 76 | 41 | Cer(m18:0/22:0) | 0.825 | 0.825 |
| 77 | 59 | Hex1Cer(d18:1/16:0) | −0.820 | 0.820 |
| 78 | 23 | CE 22:6 | 0.800 | 0.800 |
| 79 | 228 | TG(58:10) [NL-22:6] | −0.797 | 0.797 |
| 80 | 72 | LPC 18:1 | 0.784 | 0.784 |
| 81 | 183 | TG(48:1) [NL-18:1] | 0.774 | 0.774 |
| 82 | 203 | TG(52:4) [NL-16:1] | 0.773 | 0.773 |
| 83 | 91 | LPE 18:1 | 0.770 | 0.770 |
| 84 | 225 | TG(56:8) [NL-20:5] | −0.767 | 0.767 |
| 85 | 64 | Hex2Cer(d16:1/16:0) | 0.762 | 0.762 |
| 86 | 74 | LPC 18:3 | 0.756 | 0.756 |
| 87 | 104 | PC 34:1 | −0.755 | 0.755 |
| 88 | 35 | Cer(d18:2/23:0) | 0.754 | 0.754 |
| 89 | 121 | PC(P-30:0) | 0.754 | 0.754 |
| 90 | 2 | AcylCarnitine 13:0 | 0.748 | 0.748 |
| 91 | 85 | LPC(O-22:1) | 0.724 | 0.724 |
| 92 | 28 | Cer(d18:1/16:0) | 0.724 | 0.724 |
| 93 | 43 | Cer(m18:1/22:0) | 0.720 | 0.720 |
| 94 | 147 | PE(P-18:0/22:6) | 0.719 | 0.719 |
| 95 | 25 | Cer(d16:1/24:0) | −0.705 | 0.705 |
| 96 | 170 | SM 35:1 | 0.704 | 0.704 |
| 97 | 33 | Cer(d18:1/24:1) | 0.697 | 0.697 |
| 98 | 12 | CE 16:0 | 0.694 | 0.694 |
| 99 | 21 | CE 20:5 | 0.690 | 0.690 |
| 100 | 231 | TG(O-50:2) [NL-16:1] | −0.684 | 0.684 |
| 101 | 210 | TG(54:5) [NL-18:3] | 0.681 | 0.681 |
| 102 | 141 | PE(P-17:0/20:4) | −0.679 | 0.679 |
| 103 | 206 | TG(52:5) [NL-18:3] | −0.667 | 0.667 |
| 104 | 69 | LPC 14:0 | −0.662 | 0.662 |
| 105 | 195 | TG(50:3) [NL-18:2] | −0.655 | 0.655 |
| 106 | 117 | PC 38:6(b) | −0.654 | 0.654 |
| 107 | 32 | Cer(d18:1/24:0) | −0.640 | 0.640 |
| 108 | 182 | Sulfatide (d18:1:/16:0) | 0.633 | 0.633 |
| 109 | 55 | DG 36:3 -(18:2) | 0.626 | 0.626 |
| 110 | 211 | TG(54:5) [NL-20:4] | 0.624 | 0.624 |
| 111 | 218 | TG(56:6) [NL-20:4] | 0.624 | 0.624 |
| 112 | 66 | Hex2Cer(d18:1/22:0) | 0.617 | 0.617 |
| 113 | 27 | Cer(d17:1/24:0) | 0.609 | 0.609 |
| 114 | 115 | PC 38:4(c) | −0.605 | 0.605 |
| 115 | 198 | TG(50:4) [NL-20:4] | 0.603 | 0.603 |
| 116 | 175 | SM 38:2 | 0.595 | 0.595 |
| 117 | 53 | DG 34:2 -(18:2) | −0.580 | 0.580 |
| 118 | 40 | Cer(d19:1/24:1) | 0.577 | 0.577 |
| 119 | 126 | PC(P-37:4) | −0.569 | 0.569 |
| 120 | 106 | PC 36:2 | 0.569 | 0.569 |
| 121 | 155 | PI 36:2 | 0.553 | 0.553 |
| 122 | 48 | DE(18:1) | −0.541 | 0.541 |
| 123 | 189 | TG(48:3) [NL-16:1] | −0.537 | 0.537 |
| 124 | 10 | CE 14:0 | 0.532 | 0.532 |
| 125 | 58 | GM3(d18:1/24:1) | 0.527 | 0.527 |
| 126 | 187 | TG(48:2) [NL-18:2] | 0.517 | 0.517 |
| 127 | 109 | PC 36:4(b) | −0.515 | 0.515 |
| 128 | 131 | PE 36:4 | −0.505 | 0.505 |
| 129 | 34 | Cer(d18:2/22:0) | −0.504 | 0.504 |
| 130 | 180 | Sph(d17:1) | −0.501 | 0.501 |
| 131 | 78 | LPC 20:5 | 0.494 | 0.494 |
| 132 | 223 | TG(56:7) [NL-22:6] | −0.493 | 0.493 |
| 133 | 207 | TG(52:5) [NL-20:4] | −0.490 | 0.490 |
| 134 | 95 | LPE(P-16:0) | 0.489 | 0.489 |
| 135 | 134 | PE 40:6 | 0.480 | 0.480 |
| 136 | 199 | TG(51:2) [NL-17:0] | 0.478 | 0.478 |
| 137 | 200 | TG(51:2) [NL-17:1] | 0.475 | 0.475 |
| 138 | 157 | PI 37:4 | 0.465 | 0.465 |
| 139 | 193 | TG(50:3) [NL-14:1] | −0.463 | 0.463 |
| 140 | 161 | PS 38:4 | −0.456 | 0.456 |
| 141 | 98 | PC 31:0 | 0.450 | 0.450 |
| 142 | 234 | TG(O-54:4) [NL-17:1] | 0.440 | 0.440 |
| 143 | 205 | TG(52:4) [NL-18:3] | −0.429 | 0.429 |
| 144 | 130 | PE 36:2 | −0.429 | 0.429 |
| 145 | 100 | PC 32:1 | −0.427 | 0.427 |
| 146 | 221 | TG(56:7) [NL-20:5] | 0.420 | 0.420 |
| 147 | 67 | Hex2Cer(d18:1/24:1) | −0.418 | 0.418 |
| 148 | 30 | Cer(d18:1/22:0) | 0.418 | 0.418 |
| 149 | 16 | CE 18:2 | −0.416 | 0.416 |
| 150 | 105 | PC 34:2 | 0.411 | 0.411 |
| 151 | 60 | Hex1Cer(d18:1/18:0) | 0.408 | 0.408 |
| 152 | 201 | TG(52:2) [NL-16:0] | 0.405 | 0.405 |
| 153 | 235 | Ubiquinone | 0.403 | 0.403 |
| 154 | 149 | PE(P-18:1/20:5) | 0.392 | 0.392 |
| 155 | 79 | LPC 22:0 | 0.391 | 0.391 |
| 156 | 24 | Cer(d16:1/22:0) | 0.389 | 0.389 |
| 157 | 214 | TG(54:6) [NL-20:5] | −0.374 | 0.374 |
| 158 | 150 | PE(P-20:0/18:2) | 0.368 | 0.368 |
| 159 | 222 | TG(56:7) [NL-22:5] | 0.367 | 0.367 |
| 160 | 197 | TG(50:4) [NL-18:3] | −0.365 | 0.365 |
| 161 | 61 | Hex1Cer(d18:1/22:0) | 0.356 | 0.356 |
| 162 | 148 | PE(P-18:1/20:4) | 0.355 | 0.355 |
| 163 | 136 | PE(O-36:4) | −0.350 | 0.350 |
| 164 | 113 | PC 38:4(a) | 0.349 | 0.349 |
| 165 | 185 | TG(48:2) [NL-14:1] | −0.341 | 0.341 |
| 166 | 93 | LPE 20:4 | −0.326 | 0.326 |
| 167 | 96 | LPE(P-18:0) | 0.313 | 0.313 |
| 168 | 26 | Cer(d16:1/24:1) | −0.309 | 0.309 |
| 169 | 14 | CE 16:2 | −0.300 | 0.300 |
| 170 | 47 | COH (161) | −0.297 | 0.297 |
| 171 | 232 | TG(O-50:2) [NL-18:1] | 0.292 | 0.292 |
| 172 | 50 | DE(20:4) | 0.285 | 0.285 |
| 173 | 186 | TG(48:2) [NL-16:1] | 0.277 | 0.277 |
| 174 | 80 | LPC 22:5 | −0.273 | 0.273 |
| 175 | 49 | DE(18:2) | −0.270 | 0.270 |
| 176 | 56 | DG 38:4 -(20:3) | 0.268 | 0.268 |
| 177 | 138 | PE(P-16:0/20:4) | 0.253 | 0.253 |
| 178 | 71 | LPC 18:0 | −0.247 | 0.247 |
| 179 | 83 | LPC 26:0 | −0.240 | 0.240 |
| 180 | 219 | TG(56:6) [NL-22:5] | −0.229 | 0.229 |
| 181 | 39 | Cer(d19:1/24:0) | −0.222 | 0.222 |
| 182 | 227 | TG(56:9) [NL-22:6] | −0.221 | 0.221 |
| 183 | 137 | PE(P-16:0/18:2) | 0.218 | 0.218 |
| 184 | 13 | CE 16:1 | 0.213 | 0.213 |
| 185 | 94 | LPE 22:6 | 0.197 | 0.197 |
| 186 | 51 | DG 32:0 -(16:0) | −0.191 | 0.191 |
| 187 | 46 | Cer1P(d18:1/16:0) | −0.189 | 0.189 |
| 188 | 233 | TG(O-50:2) [NL-18:2] | −0.189 | 0.189 |
| 189 | 103 | PC 33:2 | 0.182 | 0.182 |
| 190 | 75 | LPC 19:0 | 0.172 | 0.172 |
| 191 | 81 | LPC 22:6 | 0.172 | 0.172 |
| 192 | 18 | CE 20:0 | −0.167 | 0.167 |
| 193 | 97 | LPI 18:0 | 0.164 | 0.164 |
| 194 | 190 | TG(50:2) [NL-16:1] | 0.161 | 0.161 |
| 195 | 153 | PI 34:1 | 0.160 | 0.160 |
| 196 | 45 | Cer(m18:1/24:1) | −0.156 | 0.156 |
| 197 | 158 | PI 38:4 | −0.155 | 0.155 |
| 198 | 202 | TG(52:3) [NL-18:2] | −0.142 | 0.142 |
| 199 | 114 | PC 38:4(b) | −0.129 | 0.129 |
| 200 | 9 | AcylCarnitine 18:2 | −0.129 | 0.129 |
| 201 | 151 | PE(P-20:0/20:4) | 0.124 | 0.124 |
| 202 | 184 | TG(48:2) [NL-14:0] | −0.122 | 0.122 |
| 203 | 88 | LPC(P-17:0) | −0.121 | 0.121 |
| 204 | 57 | GM3(d18:1/22:0) | 0.112 | 0.112 |
| 205 | 20 | CE 20:4 | 0.112 | 0.112 |
| 206 | 22 | CE 22:1 | −0.110 | 0.110 |
| 207 | 216 | TG(54:7) [NL-20:5] | −0.106 | 0.106 |
| 208 | 102 | PC 33:1 | −0.095 | 0.095 |
| 209 | 165 | SM 32:2 | −0.088 | 0.088 |
| 210 | 156 | PI 36:4 | 0.088 | 0.088 |
| 211 | 171 | SM 35:2 | 0.086 | 0.086 |
| 212 | 229 | TG(58:8) [NL-22:6] | 0.078 | 0.078 |
| 213 | 196 | TG(50:4) [NL-14:0] | 0.077 | 0.077 |
| 214 | 209 | TG(54:3) [NL-18:1] | 0.073 | 0.073 |
| 215 | 226 | TG(56:8) [NL-22:6] | 0.066 | 0.066 |
| 216 | 142 | PE(P-17:0/22:6) | 0.059 | 0.059 |
| 217 | 124 | PC(P-36:4) | −0.056 | 0.056 |
| 218 | 84 | LPC(O-22:0) | 0.056 | 0.056 |
| 219 | 217 | TG(54:7) [NL-22:6] | 0.055 | 0.055 |
| 220 | 191 | TG(50:2) [NL-18:1] | −0.049 | 0.049 |
| 221 | 176 | SM 38:3 | 0.048 | 0.048 |
| 222 | 128 | PE 34:1 | 0.045 | 0.045 |
| 223 | 110 | PC 36:5(a) | 0.043 | 0.043 |
| 224 | 204 | TG(52:4) [NL-18:2] | −0.043 | 0.043 |
| 225 | 119 | PC 40:8 | 0.042 | 0.042 |
| 226 | 70 | LPC 17:0 | −0.040 | 0.040 |
| 227 | 86 | LPC(O-24:0) | −0.032 | 0.032 |
| 228 | 38 | Cer(d19:1/22:0) | 0.030 | 0.030 |
| 229 | 220 | TG(56:7) [NL-20:4] | 0.028 | 0.028 |
| 230 | 163 | SM 31:1 | 0.026 | 0.026 |
| 231 | 108 | PC 36:4(a) | −0.020 | 0.020 |
| 232 | 17 | CE 18:3 | 0.017 | 0.017 |
| 233 | 152 | PI 32:1 | 0.010 | 0.010 |
| 234 | 133 | PE 38:6 | 0.009 | 0.009 |
| 235 | 107 | PC 36:4 | −0.009 | 0.009 |

TABLE 16
Predictive models of age developed using LASSO regression
incorporating BMI and sex together with the CLP2s lipids

| | | | | LASSO |
|---|---|---|---|---|
| Rank # | Lipid # | Lipid/Covariate | LASSO | (ABS) |
| | | (Intercept) | 54.974 | |
| | | drsex_00_n | 2.883 | 2.883 |
| | | bmi_00 | −0.184 | 0.184 |
| 1 | 4 | AcylCarnitine 14:1 | −6.496 | 6.496 |
| 2 | 7 | AcylCarnitine 16:1 | 5.290 | 5.290 |
| 3 | 68 | Hex3Cer(d18:1/16:0) | −3.818 | 3.818 |
| 4 | 123 | PC(P-34:2) | −3.686 | 3.686 |
| 5 | 166 | SM 33:1 | 3.576 | 3.576 |
| 6 | 118 | PC 40:6 | 3.382 | 3.382 |
| 7 | 6 | AcylCarnitine 16:0 | −3.215 | 3.215 |
| 8 | 168 | SM 34:2 | 3.174 | 3.174 |
| 9 | 192 | TG(50:3) [NL-14:0] | 2.819 | 2.819 |
| 10 | 5 | AcylCarnitine 14:2 | 2.749 | 2.749 |
| 11 | 31 | Cer(d18:1/23:0) | 2.617 | 2.617 |
| 12 | 144 | PE(P-18:0/18:2) | 2.503 | 2.503 |
| 13 | 159 | PI 38:6 | −2.201 | 2.201 |
| 14 | 99 | PC 32:0 | 2.173 | 2.173 |
| 15 | 129 | PE 34:2 | 2.123 | 2.123 |
| 16 | 44 | Cer(m18:1/24:0) | 2.105 | 2.105 |
| 17 | 215 | TG(54:6) [NL-22:6] | −2.030 | 2.030 |
| 18 | 65 | Hex2Cer(d18:1/16:0) | −2.007 | 2.007 |
| 19 | 76 | LPC 19:1 | −2.004 | 2.004 |
| 20 | 15 | CE 17:0 | −1.973 | 1.973 |
| 21 | 188 | TG(48:3) [NL-14:0] | −1.947 | 1.947 |
| 22 | 111 | PC 36:5(b) | 1.940 | 1.940 |
| 23 | 164 | SM 32:1 | −1.938 | 1.938 |
| 24 | 36 | Cer(d18:2/24:0) | −1.772 | 1.772 |
| 25 | 1 | AcylCarnitine 12:0 | 1.761 | 1.761 |
| 26 | 172 | SM 36:1 | −1.604 | 1.604 |
| 27 | 52 | DG 34:1 -(18:1) | −1.599 | 1.599 |
| 28 | 179 | SM 44:2 | 1.523 | 1.523 |
| 29 | 127 | PE 32:1 | −1.464 | 1.464 |
| 30 | 120 | PC(O-32:0) | −1.403 | 1.403 |
| 31 | 8 | AcylCarnitine 18:1 | 1.378 | 1.378 |
| 32 | 87 | LPC(P-16:0) | 1.375 | 1.375 |
| 33 | 213 | TG(54:6) [NL-20:4] | 1.362 | 1.362 |
| 34 | 90 | LPE 18:0 | 1.356 | 1.356 |
| 35 | 37 | Cer(d18:2/24:1) | −1.351 | 1.351 |
| 36 | 174 | SM 38:1 | −1.212 | 1.212 |
| 37 | 173 | SM 36:3 | −1.197 | 1.197 |
| 38 | 77 | LPC 20:4 | −1.194 | 1.194 |
| 39 | 194 | TG(50:3) [NL-16:1] | −1.173 | 1.173 |
| 40 | 178 | SM 41:1 | −1.101 | 1.101 |
| 41 | 115 | PC 38:4(c) | −1.100 | 1.100 |
| 42 | 169 | SM 34:3 | 1.100 | 1.100 |
| 43 | 101 | PC 32:2 | −1.087 | 1.087 |
| 44 | 177 | SM 40:1 | −1.077 | 1.077 |
| 45 | 125 | PC(P-36:5) | 1.074 | 1.074 |
| 46 | 181 | Sulfatide (d18:1:/16:0(OH)) | −1.062 | 1.062 |
| 47 | 145 | PE(P-18:0/20:4) | 1.040 | 1.040 |
| 48 | 3 | AcylCarnitine 14:0 | 1.037 | 1.037 |
| 49 | 89 | LPE 16:0 | 1.034 | 1.034 |
| 50 | 29 | Cer(d18:1/20:0) | 1.028 | 1.028 |
| 51 | 132 | PE 38:4 | −1.018 | 1.018 |
| 52 | 162 | S1P(d18:1) | −0.996 | 0.996 |
| 53 | 2 | AcylCarnitine 13:0 | 0.990 | 0.990 |
| 54 | 143 | PE(P-18:0/18:1) | −0.962 | 0.962 |
| 55 | 195 | TG(50:3) [NL-18:2] | −0.918 | 0.918 |
| 56 | 63 | Hex1Cer(d18:2/24:0) | 0.910 | 0.910 |
| 57 | 11 | CE 15:0 | 0.907 | 0.907 |
| 58 | 59 | Hex1Cer(d18:1/16:0) | −0.903 | 0.903 |
| 59 | 146 | PE(P-18:0/20:5) | −0.893 | 0.893 |
| 60 | 42 | Cer(m18:1/20:0) | 0.866 | 0.866 |
| 61 | 19 | CE 20:1 | −0.861 | 0.861 |
| 62 | 116 | PC 38:6(a) | −0.847 | 0.847 |
| 63 | 135 | PE(O-34:1) | −0.777 | 0.777 |
| 64 | 122 | PC(P-32:0) | 0.764 | 0.764 |
| 65 | 121 | PC(P-30:0) | 0.756 | 0.756 |
| 66 | 183 | TG(48:1) [NL-18:1] | 0.756 | 0.756 |
| 67 | 117 | PC 38:6(b) | −0.745 | 0.745 |
| 68 | 41 | Cer(m18:0/22:0) | 0.731 | 0.731 |
| 69 | 12 | CE 16:0 | 0.726 | 0.726 |
| 70 | 140 | PE(P-16:0/22:6) | −0.712 | 0.712 |
| 71 | 91 | LPE 18:1 | 0.711 | 0.711 |
| 72 | 208 | TG(54:2) [NL-20:1] | 0.702 | 0.702 |
| 73 | 218 | TG(56:6) [NL-20:4] | 0.684 | 0.684 |
| 74 | 21 | CE 20:5 | 0.683 | 0.683 |
| 75 | 160 | PI 40:6 | −0.627 | 0.627 |
| 76 | 58 | GM3(d18:1/24:1) | 0.620 | 0.620 |
| 77 | 82 | LPC 24:0 | 0.619 | 0.619 |
| 78 | 231 | TG(O-50:2) [NL-16:1] | −0.612 | 0.612 |
| 79 | 92 | LPE 18:2 | 0.603 | 0.603 |
| 80 | 230 | TG(58:9) [NL-22:6] | −0.601 | 0.601 |
| 81 | 33 | Cer(d18:1/24:1) | 0.601 | 0.601 |
| 82 | 167 | SM 34:1 | 0.588 | 0.588 |
| 83 | 64 | Hex2Cer(d16:1/16:0) | 0.574 | 0.574 |
| 84 | 73 | LPC 18:2 | −0.571 | 0.571 |
| 85 | 139 | PE(P-16:0/20:5) | −0.567 | 0.567 |
| 86 | 182 | Sulfatide (d18:1:/16:0) | 0.565 | 0.565 |
| 87 | 126 | PC(P-37:4) | −0.564 | 0.564 |
| 88 | 28 | Cer(d18:1/16:0) | 0.555 | 0.555 |
| 89 | 62 | Hex1Cer(d18:1/24:1) | −0.551 | 0.551 |
| 90 | 72 | LPC 18:1 | 0.547 | 0.547 |
| 91 | 228 | TG(58:10) [NL-22:6] | −0.529 | 0.529 |
| 92 | 141 | PE(P-17:0/20:4) | −0.525 | 0.525 |
| 93 | 104 | PC 34:1 | −0.524 | 0.524 |
| 94 | 10 | CE 14:0 | 0.522 | 0.522 |
| 95 | 180 | Sph(d17:1) | −0.496 | 0.496 |
| 96 | 23 | CE 22:6 | 0.485 | 0.485 |
| 97 | 210 | TG(54:5) [NL-18:3] | 0.475 | 0.475 |
| 98 | 48 | DE(18:1) | −0.470 | 0.470 |
| 99 | 154 | PI 36:1 | −0.461 | 0.461 |
| 100 | 175 | SM 38:2 | 0.430 | 0.430 |
| 101 | 95 | LPE(P-16:0) | 0.423 | 0.423 |
| 102 | 199 | TG(51:2) [NL-17:0] | 0.420 | 0.420 |
| 103 | 105 | PC 34:2 | 0.417 | 0.417 |
| 104 | 161 | PS 38:4 | −0.415 | 0.415 |
| 105 | 234 | TG(O-54:4) [NL-17:1] | 0.411 | 0.411 |
| 106 | 206 | TG(52:5) [NL-18:3] | −0.407 | 0.407 |
| 107 | 9 | AcylCarnitine 18:2 | −0.407 | 0.407 |
| 108 | 16 | CE 18:2 | −0.405 | 0.405 |
| 109 | 75 | LPC 19:0 | 0.402 | 0.402 |
| 110 | 170 | SM 35:1 | 0.395 | 0.395 |
| 111 | 106 | PC 36:2 | 0.382 | 0.382 |
| 112 | 136 | PE(O-36:4) | −0.380 | 0.380 |
| 113 | 148 | PE(P-18:1/20:4) | 0.363 | 0.363 |
| 114 | 54 | DG 36:2 -(18:1) | −0.361 | 0.361 |
| 115 | 211 | TG(54:5) [NL-20:4] | 0.349 | 0.349 |
| 116 | 60 | Hex1Cer(d18:1/18:0) | 0.311 | 0.311 |
| 117 | 85 | LPC(O-22:1) | 0.310 | 0.310 |
| 118 | 235 | Ubiquinone | 0.300 | 0.300 |
| 119 | 43 | Cer(m18:1/22:0) | 0.293 | 0.293 |
| 120 | 61 | Hex1Cer(d18:1/22:0) | 0.290 | 0.290 |
| 121 | 157 | PI 37:4 | 0.285 | 0.285 |
| 122 | 147 | PE(P-18:0/22:6) | 0.270 | 0.270 |
| 123 | 47 | COH (161) | −0.268 | 0.268 |
| 124 | 150 | PE(P-20:0/18:2) | 0.262 | 0.262 |
| 125 | 30 | Cer(d18:1/22:0) | 0.260 | 0.260 |
| 126 | 223 | TG(56:7) [NL-22:6] | −0.253 | 0.253 |
| 127 | 35 | Cer(d18:2/23:0) | 0.244 | 0.244 |
| 128 | 96 | LPE(P-18:0) | 0.238 | 0.238 |
| 129 | 201 | TG(52:2) [NL-16:0] | −0.222 | 0.222 |
| 130 | 46 | Cer1P(d18:1/16:0) | −0.209 | 0.209 |
| 131 | 113 | PC 38:4(a) | 0.204 | 0.204 |
| 132 | 109 | PC 36:4(b) | −0.201 | 0.201 |
| 133 | 50 | DE(20:4) | 0.192 | 0.192 |
| 134 | 189 | TG(48:3) [NL-16:1] | −0.179 | 0.179 |
| 135 | 40 | Cer(d19:1/24:1) | 0.166 | 0.166 |
| 136 | 212 | TG(54:6) [NL-18:3] | 0.164 | 0.164 |
| 137 | 130 | PE 36:2 | −0.164 | 0.164 |
| 138 | 187 | TG(48:2) [NL-18:2] | 0.149 | 0.149 |
| 139 | 49 | DE(18:2) | −0.145 | 0.145 |
| 140 | 221 | TG(56:7) [NL-20:5] | 0.142 | 0.142 |
| 141 | 22 | CE 22:1 | −0.140 | 0.140 |
| 142 | 98 | PC 31:0 | 0.140 | 0.140 |
| 143 | 200 | TG(51:2) [NL-17:1] | 0.137 | 0.137 |
| 144 | 232 | TG(O-50:2) [NL-18:1] | 0.136 | 0.136 |
| 145 | 79 | LPC 22:0 | 0.127 | 0.127 |
| 146 | 18 | CE 20:0 | −0.106 | 0.106 |
| 147 | 112 | PC 37:6 | −0.100 | 0.100 |
| 148 | 155 | PI 36:2 | 0.095 | 0.095 |
| 149 | 151 | PE(P-20:0/20:4) | 0.093 | 0.093 |
| 150 | 193 | TG(50:3) [NL-14:1] | −0.087 | 0.087 |
| 151 | 138 | PE(P-16:0/20:4) | 0.080 | 0.080 |
| 152 | 14 | CE 16:2 | −0.062 | 0.062 |
| 153 | 25 | Cer(d16:1/24:0) | −0.061 | 0.061 |
| 154 | 32 | Cer(d18:1/24:0) | −0.056 | 0.056 |
| 155 | 97 | LPI 18:0 | 0.054 | 0.054 |
| 156 | 176 | SM 38:3 | 0.045 | 0.045 |
| 157 | 220 | TG(56:7) [NL-20:4] | 0.038 | 0.038 |
| 158 | 84 | LPC(O-22:0) | 0.028 | 0.028 |
| 159 | 57 | GM3(d18:1/22:0) | 0.028 | 0.028 |
| 160 | 158 | PI 38:4 | −0.019 | 0.019 |
| 161 | 94 | LPE 22:6 | 0.007 | 0.007 |
| 162 | 209 | TG(54:3) [NL-18:1] | 0.005 | 0.005 |
| 163 | 27 | Cer(d17:1/24:0) | 0.003 | 0.003 |
| 164 | 83 | LPC 26:0 | −0.003 | 0.003 |
| 165 | 203 | TG(52:4) [NL-16:1] | 0.003 | 0.003 |
| 166 | 100 | PC 32:1 | −0.002 | 0.002 |
| 167 | 93 | LPE 20:4 | 0.000 | 0.000 |
| 168 | 197 | TG(50:4) [NL-18:3] | 0.000 | 0.000 |
| 169 | 13 | CE 16:1 | 0.000 | 0.000 |
| 170 | 17 | CE 18:3 | 0.000 | 0.000 |
| 171 | 20 | CE 20:4 | 0.000 | 0.000 |
| 172 | 24 | Cer(d16:1/22:0) | 0.000 | 0.000 |
| 173 | 26 | Cer(d16:1/24:1) | 0.000 | 0.000 |
| 174 | 34 | Cer(d18:2/22:0) | 0.000 | 0.000 |
| 175 | 38 | Cer(d19:1/22:0) | 0.000 | 0.000 |
| 176 | 39 | Cer(d19:1/24:0) | 0.000 | 0.000 |
| 177 | 45 | Cer(m18:1/24:1) | 0.000 | 0.000 |
| 178 | 51 | DG 32:0 -(16:0) | 0.000 | 0.000 |
| 179 | 53 | DG 34:2 -(18:2) | 0.000 | 0.000 |
| 180 | 55 | DG 36:3 -(18:2) | 0.000 | 0.000 |
| 181 | 56 | DG 38:4 -(20:3) | 0.000 | 0.000 |
| 182 | 66 | Hex2Cer(d18:1/22:0) | 0.000 | 0.000 |
| 183 | 67 | Hex2Cer(d18:1/24:1) | 0.000 | 0.000 |
| 184 | 69 | LPC 14:0 | 0.000 | 0.000 |
| 185 | 70 | LPC 17:0 | 0.000 | 0.000 |
| 186 | 71 | LPC 18:0 | 0.000 | 0.000 |
| 187 | 74 | LPC 18:3 | 0.000 | 0.000 |
| 188 | 78 | LPC 20:5 | 0.000 | 0.000 |
| 189 | 80 | LPC 22:5 | 0.000 | 0.000 |
| 190 | 81 | LPC 22:6 | 0.000 | 0.000 |
| 191 | 86 | LPC(O-24:0) | 0.000 | 0.000 |
| 192 | 88 | LPC(P-17:0) | 0.000 | 0.000 |
| 193 | 102 | PC 33:1 | 0.000 | 0.000 |
| 194 | 103 | PC 33:2 | 0.000 | 0.000 |
| 195 | 107 | PC 36:4 | 0.000 | 0.000 |
| 196 | 108 | PC 36:4(a) | 0.000 | 0.000 |
| 197 | 110 | PC 36:5(a) | 0.000 | 0.000 |
| 198 | 114 | PC 38:4(b) | 0.000 | 0.000 |
| 199 | 119 | PC 40:8 | 0.000 | 0.000 |
| 200 | 124 | PC(P-36:4) | 0.000 | 0.000 |
| 201 | 128 | PE 34:1 | 0.000 | 0.000 |
| 202 | 131 | PE 36:4 | 0.000 | 0.000 |
| 203 | 133 | PE 38:6 | 0.000 | 0.000 |
| 204 | 134 | PE 40:6 | 0.000 | 0.000 |
| 205 | 137 | PE(P-16:0/18:2) | 0.000 | 0.000 |
| 206 | 142 | PE(P-17:0/22:6) | 0.000 | 0.000 |
| 207 | 149 | PE(P-18:1/20:5) | 0.000 | 0.000 |
| 208 | 152 | PI 32:1 | 0.000 | 0.000 |
| 209 | 153 | PI 34:1 | 0.000 | 0.000 |
| 210 | 156 | PI 36:4 | 0.000 | 0.000 |
| 211 | 163 | SM 31:1 | 0.000 | 0.000 |
| 212 | 165 | SM 32:2 | 0.000 | 0.000 |
| 213 | 171 | SM 35:2 | 0.000 | 0.000 |
| 214 | 184 | TG(48:2) [NL-14:0] | 0.000 | 0.000 |
| 215 | 185 | TG(48:2) [NL-14:1] | 0.000 | 0.000 |
| 216 | 186 | TG(48:2) [NL-16:1] | 0.000 | 0.000 |
| 217 | 190 | TG(50:2) [NL-16:1] | 0.000 | 0.000 |
| 218 | 191 | TG(50:2) [NL-18:1] | 0.000 | 0.000 |
| 219 | 196 | TG(50:4) [NL-14:0] | 0.000 | 0.000 |
| 220 | 198 | TG(50:4) [NL-20:4] | 0.000 | 0.000 |
| 221 | 202 | TG(52:3) [NL-18:2] | 0.000 | 0.000 |
| 222 | 204 | TG(52:4) [NL-18:2] | 0.000 | 0.000 |
| 223 | 205 | TG(52:4) [NL-18:3] | 0.000 | 0.000 |
| 224 | 207 | TG(52:5) [NL-20:4] | 0.000 | 0.000 |
| 225 | 214 | TG(54:6) [NL-20:5] | 0.000 | 0.000 |
| 226 | 216 | TG(54:7) [NL-20:5] | 0.000 | 0.000 |
| 227 | 217 | TG(54:7) [NL-22:6] | 0.000 | 0.000 |
| 228 | 219 | TG(56:6) [NL-22:5] | 0.000 | 0.000 |
| 229 | 222 | TG(56:7) [NL-22:5] | 0.000 | 0.000 |
| 230 | 224 | TG(56:8) [NL-20:4] | 0.000 | 0.000 |
| 231 | 225 | TG(56:8) [NL-20:5] | 0.000 | 0.000 |
| 232 | 226 | TG(56:8) [NL-22:6] | 0.000 | 0.000 |
| 233 | 227 | TG(56:9) [NL-22:6] | 0.000 | 0.000 |
| 234 | 229 | TG(58:8) [NL-22:6] | 0.000 | 0.000 |
| 235 | 233 | TG(O-50:2) [NL-18:2] | 0.000 | 0.000 |

### Example 6: Development of a Plasmalogen Score

Statistical modelling approaches were used to generate portable metabolic health scores using the CLP lists (described above) to determine a patient's overall metabolic health. Plasmalogens were demonstrated to be strongly associated with metabolic health. Phosphatidylethanolamine plasmalogens (alkenylphosphatidylethanolamine) species showed opposing associations to phosphatidylethanolamine species. It was demonstrated that the two lipid classes exist in an equilibrium where the level of one counterbalances the level of the other. These two lipid classes were combined into a single score (the plasmalogen score) to better reflect not only a patient's level of plasmalogens but also the relative levels of both phosphatidylethanolamine plasmalogens (PE(P) species and phosphatidylethanolamine (PE) species.

Plasmalogen scores were generated using the entire Research Lipid List (whole lipidome, 717 lipids, Table 2) and the CLP lipid lists (described above, Tables 4 and 6) and demonstrated to associate with cardio-metabolic risk factors and/or diseases in the Australian Diabetes, Obesity and Lifestyle Study (AusDiab; n=10,339).

### Generation and Comparison of Plasmalogen Scores

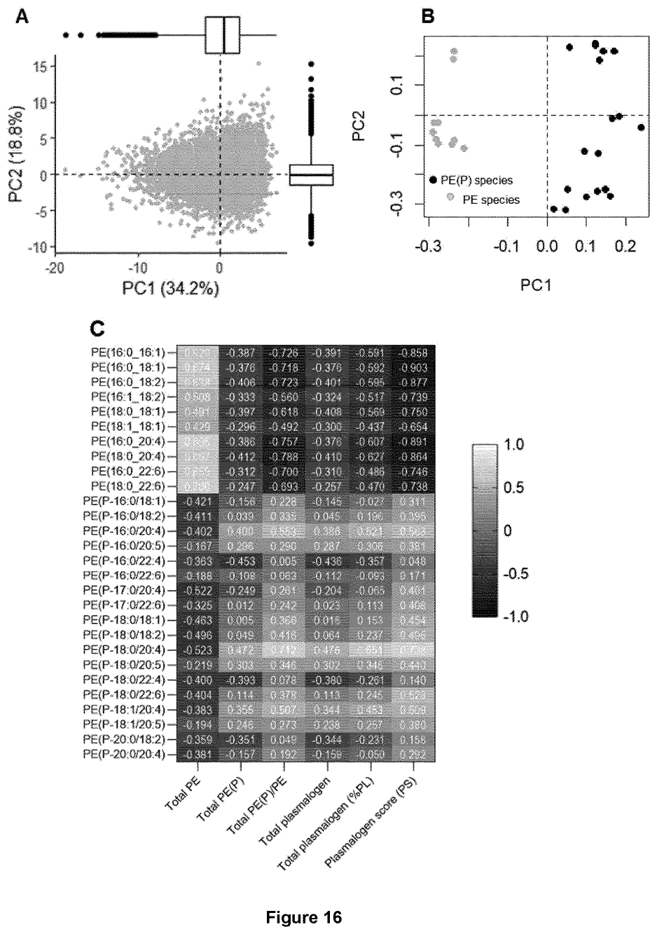

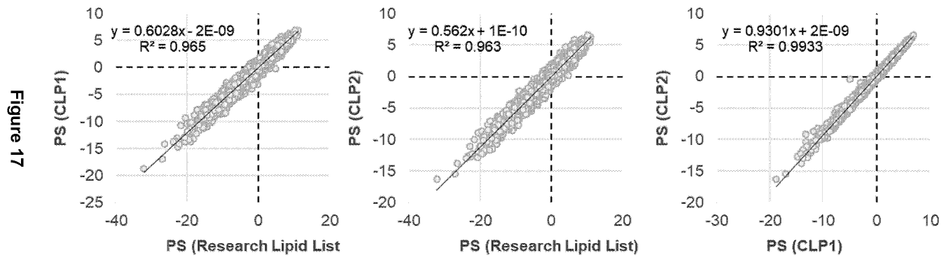

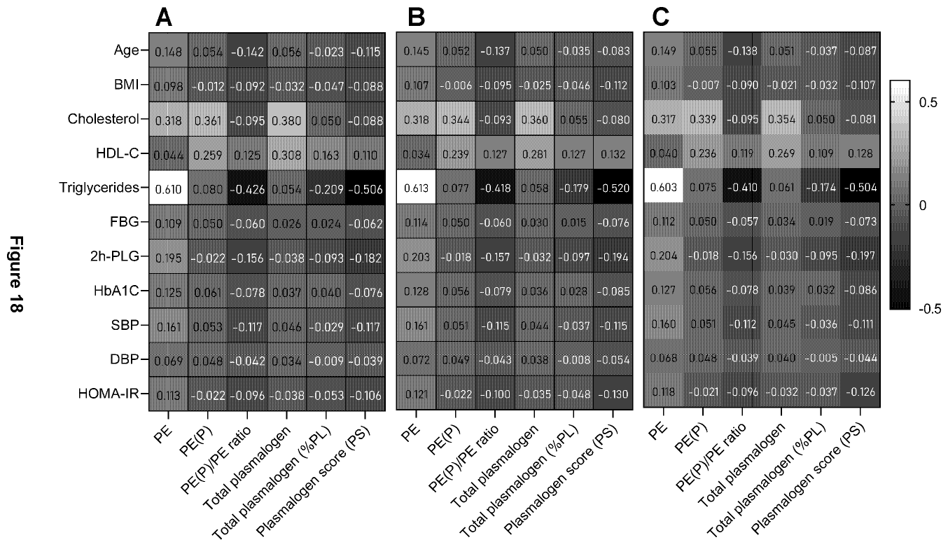

The 298 plasma lipid species in the CLP1s (Table 4), represent 32 lipid classes/subclasses, of which phospholipids account for 100 species, including 10 PE species, and 18 PE(P) species. A principal component analysis (PCA) performed on only the PE and PE(P) compositional data showed the large spread of the population within the first two principle components, with the first PC1 explaining 34.2% of the variance, and the PC2 explaining 18.8% of the variance (FIG. 16). The PCA loadings plot showed a clear separation between the PE and PE(P) species; where PC1 is positively correlated with PE(P) species but negatively correlated with PE species (FIG. 16B). The correlation between the individual lipid species and the Plasmalogen Score as well as several other plasmalogen measures is shown in FIG. 16C. The PC1 value is defined as the Plasmalogen Score (PS), a measure which best captures the counterbalance that exists between the PE and PE(P) lipid species.

The relationship between the Plasmalogen Score calculated using the 91 PE and PE(P) species in the Research Lipid List, the 28 PE and PE(P) species in the CLP1s lipid list, 23 species in the CLP2s lipid list was further assessed for correlation between these scores. The correlation (R²) for the Plasmalogen Score created with the full lipid list against the Plasmalogen Score derived using CLP1s and CLP2S was 0.965 and 0.963 respectively (FIG. 17). The weight of the individual PE and PE(P) species within the plasmalogen scores derived using CLP1s and CLP2s is shown in Table 17.

Plasmalogen Score (PS) Derived Using the Research Lipid List and CLP1s and CLP2s Lists Show Similar Correlations with Cardio-Metabolic Risk Factors

To assess whether a PS using the CLPs and the Research Lipid List, showed the same association with disease risk, a correlation analysis against cardio-metabolic risk factors was performed and the Pearson's correlation coefficients were calculated. The correlations between a PS (CLP1 and CLP2) with cardo-metabolic risk factors aligned closely with the correlation between PS (Research Lipid List or full lipidome) and the same risk factors (FIG. 18).

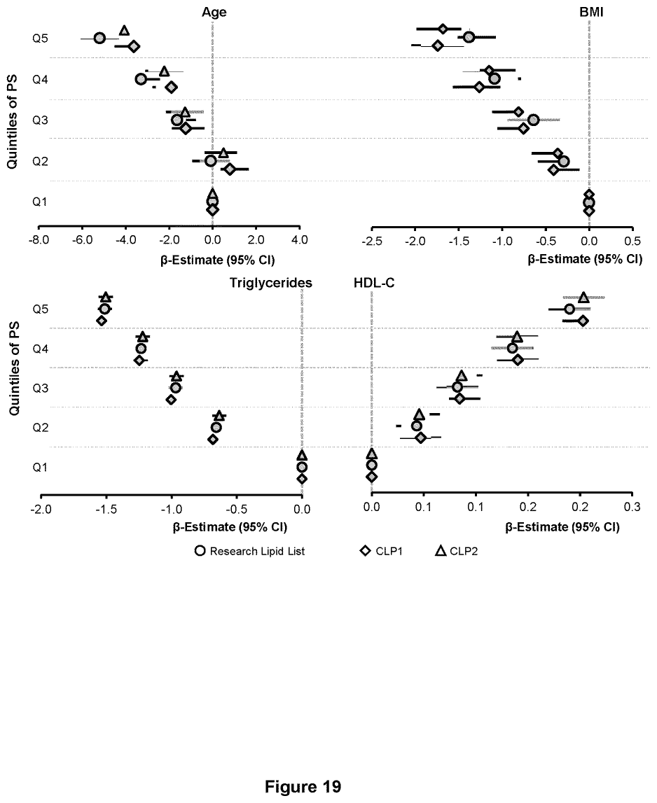

The AusDiab participants were then into quintiles of the PS, and a linear regression analysis between selected cardio-metabolic risk factors (as outcome) and the quintiles of PS (predictor) was performed to assess the overall association. An increase from Q5 to Q1 of the plasmalogen score was associated with a decrease by 5 years or, 1.8 kg/m² lower BMI and 1.5 mmol/L lower triglycerides, and 0.2 mM higher HDL-C using the original sets of PE and PE(P) species (FIG. 19, circles). Using the CLP1 and CLP2 similar effect sizes were observed (FIG. 19, diamonds and triangle).

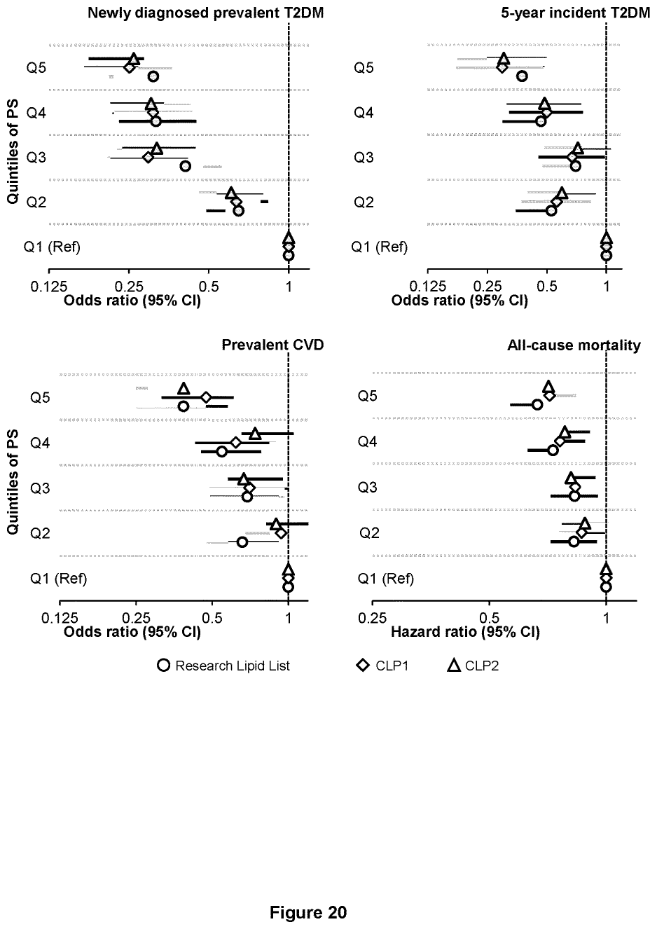

These associations between the PS (derived from the full lipidome, and the CLPs) with T2DM (both prevalent and incident) were consistent across quintiles (FIG. 20). Similar associations between the PS (derived from the full lipidome, and the CLP1 and CLP2) with CVD and all-cause mortality (FIG. 20) were also observed.

The plasmalogen score derived using the CLP1s lipid lists provided similar associations with disease risk factors and disease outcomes as the scores derived using the entire lipidome. The plasmalogen score represents anew risk marker that specifically identified people with low plasmalogen levels and particularly with low phosphatidylethanolamine plasmalogen levels relative to phosphatidylethanolamine. The plasmalogen score as measured on the CLP1s and other relevant lipid lists is a useful marker of metabolic health.

TABLE 17
Weights for the PE and PE(P) species in the Plasmalogen
Scores derived from CLP1s and CLP2s lipid lists

| | PS based in CLP1s | | | | PS based in CLP2s | | | |
|---|---|---|---|---|---|---|---|---|
| | | | | Loadings | | | | Loadings |
| Rank | Lipid | | Loadings | (PS) | Lipid | | Loadings | (PS) |
| # | # | Lipid | (PS) | (ABS) | # | Lipid | (PS) | (ABS) |
| 1 | 2 | PE(16:0_18:1) | −0.292 | 0.292 | 5 | PE(16:0_20:4) | −0.312 | 0.312 |
| 2 | 7 | PE(16:0_20:4) | −0.288 | 0.288 | 2 | PE(16:0_18:1) | −0.307 | 0.307 |
| 3 | 3 | PE(16:0_18:2) | −0.283 | 0.283 | 6 | PE(18:0_20:4) | −0.305 | 0.305 |
| 4 | 8 | PE(18:0_20:4) | −0.279 | 0.279 | 3 | PE(16:0_18:2) | −0.301 | 0.301 |
| 5 | 1 | PE(16:0_16:1) | −0.277 | 0.277 | 1 | PE(16:0_16:1) | −0.291 | 0.291 |
| 6 | 5 | PE(18:0_18:1) | −0.242 | 0.242 | 7 | PE(16:0_22:6) | −0.268 | 0.268 |
| 7 | 9 | PE(16:0_22:6) | −0.241 | 0.241 | 8 | PE(18:0_22:6) | −0.264 | 0.264 |
| 8 | 4 | PE(16:1_18:2) | −0.239 | 0.239 | 17 | PE(P-18:0/20:4) | 0.256 | 0.256 |
| 9 | 10 | PE(18:0_22:6) | −0.239 | 0.239 | 4 | PE(18:1_18:1) | −0.214 | 0.214 |
| 10 | 21 | PE(P-18:0/20:4) | 0.238 | 0.238 | 10 | PE(P-16:0/20:4) | 0.194 | 0.194 |
| 11 | 6 | PE(18:1_18:1) | −0.211 | 0.211 | 19 | PE(P-18:0/22:6) | 0.181 | 0.181 |
| 12 | 13 | PE(P-16:0/20:4) | 0.182 | 0.182 | 20 | PE(P-18:1/20:4) | 0.174 | 0.174 |
| 13 | 24 | PE(P-18:0/22:6) | 0.171 | 0.171 | 16 | PE(P-18:0/18:2) | 0.170 | 0.170 |
| 14 | 25 | PE(P-18:1/20:4) | 0.165 | 0.165 | 18 | PE(P-18:0/20:5) | 0.159 | 0.159 |
| 15 | 20 | PE(P-18:0/18:2) | 0.160 | 0.160 | 15 | PE(P-18:0/18:1) | 0.154 | 0.154 |
| 16 | 19 | PE(P-18:0/18:1) | 0.147 | 0.147 | 14 | PE(P-17:0/22:6) | 0.140 | 0.140 |
| 17 | 22 | PE(P-18:0/20:5) | 0.142 | 0.142 | 11 | PE(P-16:0/20:5) | 0.138 | 0.138 |
| 18 | 18 | PE(P-17:0/22:6) | 0.132 | 0.132 | 21 | PE(P-18:1/20:5) | 0.137 | 0.137 |
| 19 | 17 | PE(P-17:0/20:4) | 0.130 | 0.130 | 13 | PE(P-17:0/20:4) | 0.135 | 0.135 |
| 20 | 12 | PE(P-16:0/18:2) | 0.128 | 0.128 | 9 | PE(P-16:0/18:2) | 0.135 | 0.135 |
| 21 | 14 | PE(P-16:0/20:5) | 0.123 | 0.123 | 23 | PE(P-20:0/20:4) | 0.095 | 0.095 |
| 22 | 26 | PE(P-18:1/20:5) | 0.123 | 0.123 | 12 | PE(P-16:0/22:6) | 0.054 | 0.054 |
| 23 | 11 | PE(P-16:0/18:1) | 0.101 | 0.101 | 22 | PE(P-20:0/18:2) | 0.052 | 0.052 |
| 24 | 28 | PE(P-20:0/20:4) | 0.094 | 0.094 | | | | |
| 25 | 16 | PE(P-16:0/22:6) | 0.055 | 0.055 | | | | |
| 26 | 27 | PE(P-20:0/18:2) | 0.051 | 0.051 | | | | |
| 27 | 23 | PE(P-18:0/22:4) | 0.045 | 0.045 | | | | |
| 28 | 15 | PE(P-16:0/22:4) | 0.016 | 0.016 | | | | |

### Example 7: Cardiovascular Disease (CVD) Risk Score

Using a machine learning workflow, a risk score which predicts a patient's risk for developing future cardiovascular disease (CVD) in ten years was developed. This risk score, termed the CVD lipidomic-augmented risk score (LARS) was calculated using the concentration of various lipids in a given patient's plasma. The CVD LARS was particularly effective at predicting the incident CVD for intermediate-risk individuals, regardless of whether the Research List (referred to as CLP0, Table 2), CLP1 List (Table 4), CLP2 List (Table 6), or other variants of these lists derived by excluding specific lipid species, for example CLP3, CLP4, and CLP5 (Table 30), were used to train the model. The CVD LARS was created by using a large cohort observational dataset (AusDiab) containing detailed lipidomic data and incident CVD outcomes. This dataset was used to train a Ridge regression model with each lipid concentration as predictor and the outcome set to incident CVD events, followed by further transformation outlined in the next section.

### Development of a CVD Risk Score

A machine learning workflow was used to develop the LARS model through internal validation (10-fold cross validation) in Ausdiab. For data pre-processing, each continuous variable was standardised (centre and scaled) and each lipid concentration was log-transformed and then standardised prior to being used as the predictors (independent variables), with the outcome set as incident CVD event (dependent variable). Different penalising strategies were investigated by optimising the alpha parameter (alpha=0 for ridge, alpha=1 for lasso, and alpha between 0 and 1 for elastic net) with values ranging from 0 to 1, but no major difference in model performance across different alpha values was observed. Secondly, for the Ridge regression, the lambda value was tuned by examining a wide range of lambda values (with log 10 values ranging from −1.2 to −4.5) for model screening with AUC used as the evaluation metric for selecting the optimal parameter. The model was constructed in the following three steps. First, the ridge regression was performed using the R package glmnet based on the following formula (1) with alpha set to 0 and 1, respectively and n referred to the total number of the lipids.

$$FRS ∼ μ + {\sum }_{i = 1}^{n} {\hat{β}}_{i} \times {lipid}_{i} + {\hat{β}}_{age} \times age \qquad ( 1 )$$

Second, a term ‘FRSΔ’ was introduced, which was calculated as the residuals of pFRS and the best fit of line between pFRS and FRS:

$$pFRS ∼ μ + \hat{β} \times FRS \qquad ( 2 )$$

The residual FRSΔ was calculated by subtracting the predicted value of (2) from the predicted value of (1). The residue adjusted FRS (raFRS) was then obtained by adding the FRSΔ to FRS. Finally, the derived raFRS along with age were used to derive the LRS for the prediction of the incident CVD.

The ridge and LASSO models developed using CLP1s and CLP2s lipid lists were described as a list of the covariates and lipids used in each model together with their β-coefficients (Tables 19, 20, 21 and 22).

Evaluation of LARS Derived from Whole Lipidome and Clinical Lipid Platforms

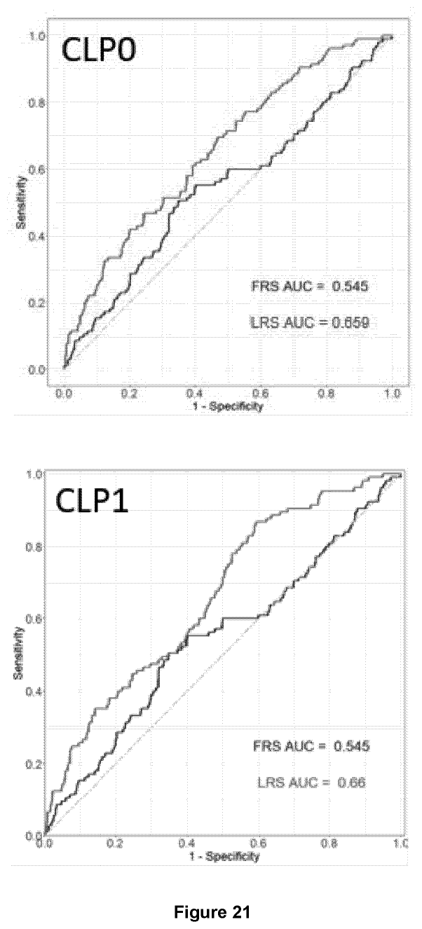

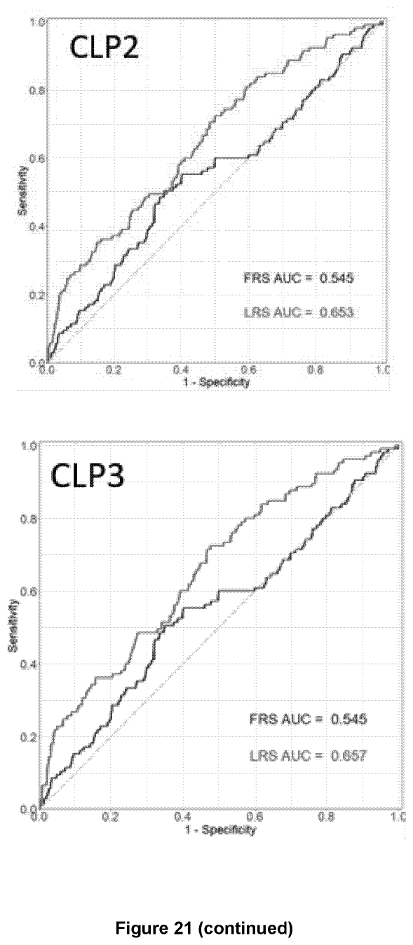

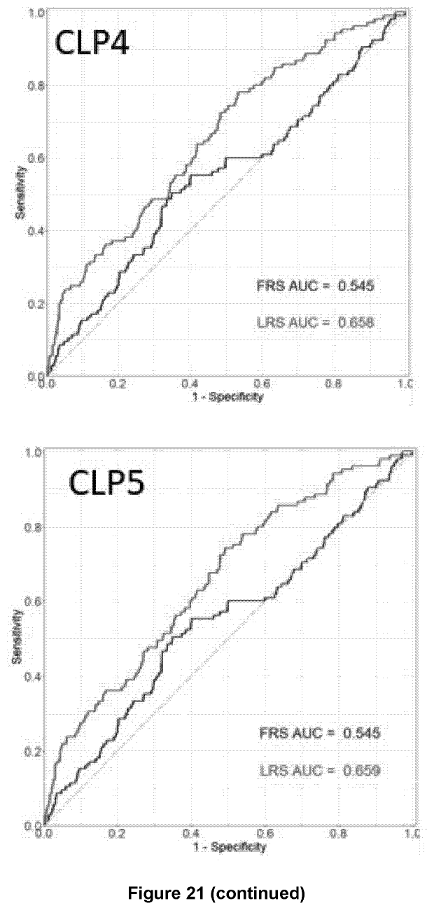

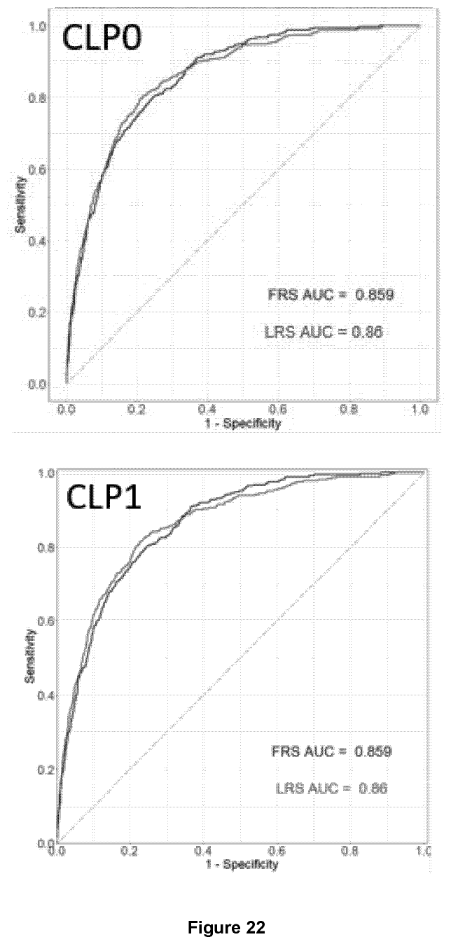

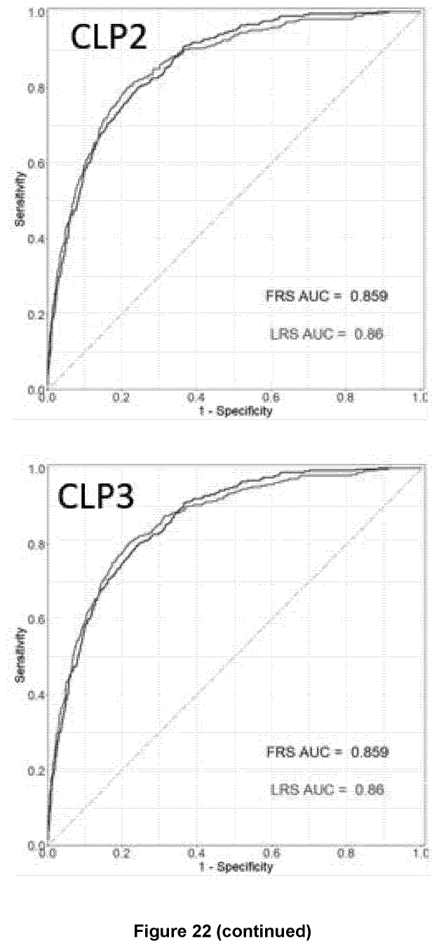

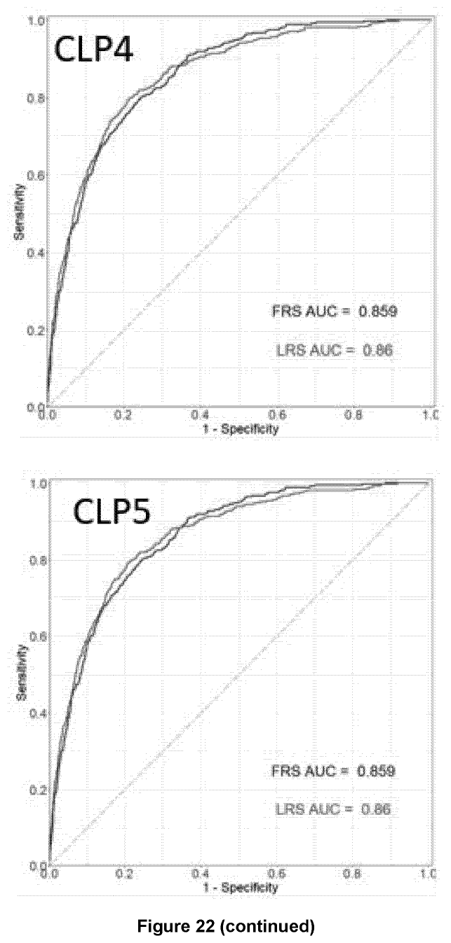

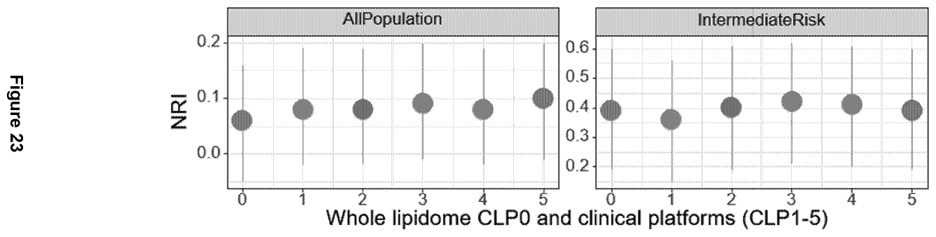

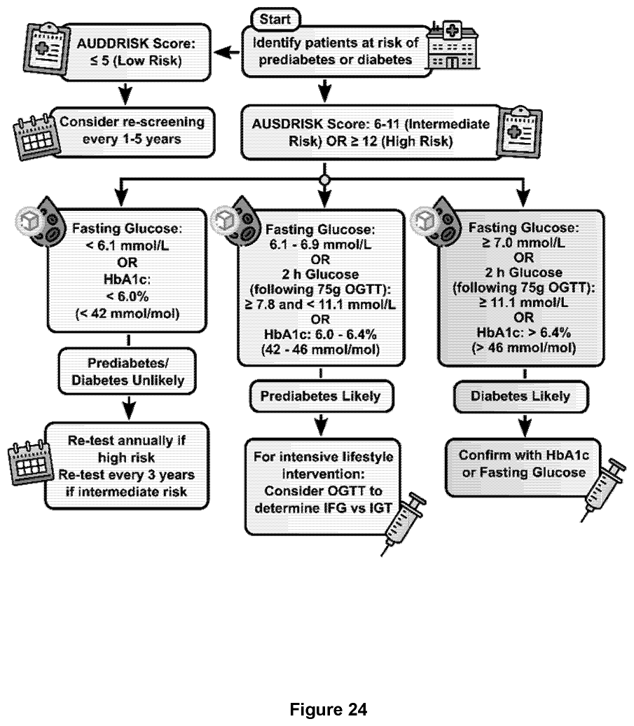

For evaluating the model performance, predictions were generated for all individuals in the validation dataset. To assess the discrimination of the models, two metrics: the receiver operating characteristic (area under the curve (ROC-AUC)) and the net reclassification improvement (NRI) were used. Table 18 lists the performance metrics of the LARS model (derived from the whole lipids panel and clinical platforms 1 to 5) which was 10-fold cross validated in Ausdiab. FIGS. 21 and 22 show the AUC plots for the intermediate-risk group and the whole population in Ausdiab, respectively. FIG. 23 shows the NRI metric for the intermediate-risk group and the whole population in Ausdiab. In summary, for LARS derived either from the whole lipidome or one of the 5 clinical lipid lists, the metrics of AUC and NRI were significantly improved as compared to the FRS in the intermediate-risk subgroup of AusDiab.

TABLE 18
The performance metrics in Ausdiab for LARS models derived
from whole lipids panel and clinical platforms 1 to 5.

| CLP | | LARS | | p | | NRI p |
|---|---|---|---|---|---|---|
| list¹ | Group² | AUC | delta AUC CI³ | value | NRI⁴ | value |
| 0 | LowRisk | 0.792 | −0.004(−0.027-0.018) | 0.685 | −0.22 (−0.38-−0.06) | 0.01 |
| 0 | IntermediateRisk | 0.659 | 0.114(0.036-0.192) | 0.004 | 0.39 (0.19-0.6) | 0 |
| 0 | HighRisk | 0.641 | 0.023(−0.038-0.082) | 0.467 | 0.3 (0.08-0.52) | 0.01 |
| 0 | AllPopulation | 0.86 | 0.001(−0.009-0.011) | 0.808 | 0.06 (−0.05-0.16) | 0.29 |
| 1 | LowRisk | 0.787 | −0.009(−0.034-0.014) | 0.433 | −0.17 (−0.33-−0.01) | 0.03 |
| 1 | IntermediateRisk | 0.66 | 0.115(0.039-0.192) | 0.003 | 0.36 (0.15-0.56) | 0 |
| 1 | HighRisk | 0.647 | 0.03(−0.028-0.086) | 0.321 | 0.33 (0.11-0.55) | 0 |
| 1 | AllPopulation | 0.86 | 0.001(−0.009-0.011) | 0.895 | 0.08 (−0.02-0.19) | 0.11 |
| 2 | LowRisk | 0.789 | −0.007(−0.03-0.016) | 0.531 | −0.25 (−0.41-−0.09) | 0 |
| 2 | IntermediateRisk | 0.653 | 0.107(0.03-0.184) | 0.006 | 0.4 (0.19-0.61) | 0 |
| 2 | HighRisk | 0.653 | 0.035(−0.026-0.094) | 0.27 | 0.41 (0.2-0.63) | 0 |
| 2 | AllPopulation | 0.86 | 0.001(−0.009-0.01) | 0.927 | 0.08 (−0.02-0.19) | 0.12 |
| 3 | LowRisk | 0.789 | −0.007(−0.03-0.015) | 0.524 | −0.26 (−0.42-−0.1) | 0 |
| 3 | IntermediateRisk | 0.657 | 0.112(0.035-0.189) | 0.004 | 0.42 (0.21-0.62) | 0 |
| 3 | HighRisk | 0.654 | 0.037(−0.024-0.095) | 0.243 | 0.33 (0.11-0.55) | 0 |
| 3 | AllPopulation | 0.86 | 0.001(−0.009-0.01) | 0.898 | 0.09 (−0.01-0.2) | 0.08 |
| 4 | LowRisk | 0.79 | −0.006(−0.029-0.016) | 0.567 | −0.21 (−0.37-−0.05) | 0.01 |
| 4 | IntermediateRisk | 0.658 | 0.113(0.036-0.189) | 0.004 | 0.41 (0.2-0.61) | 0 |
| 4 | HighRisk | 0.652 | 0.034(−0.025-0.091) | 0.266 | 0.39 (0.17-0.6) | 0 |
| 4 | AllPopulation | 0.86 | 0.001(−0.009-0.011) | 0.834 | 0.08 (−0.02-0.19) | 0.12 |
| 5 | LowRisk | 0.79 | −0.007(−0.03-0.016) | 0.559 | −0.21 (−0.37-−0.05) | 0.01 |
| 5 | IntermediateRisk | 0.659 | 0.114(0.038-0.19) | 0.003 | 0.39 (0.19-0.6) | 0 |
| 5 | HighRisk | 0.652 | 0.034(−0.025-0.091) | 0.269 | 0.35 (0.13-0.57) | 0 |
| 5 | AllPopulation | 0.86 | 0.001(−0.009-0.011) | 0.846 | 0.1 (−0.01-0.2) | 0.07 |

¹CLP list 0-5: 0 refers to the Research Lipid list; 1-5 refers to CLP1-5.
²Group: the whole population and the stratified risk groups using calibrated FRS; low risk (FRS < 0.1), intermediate risk (0.1 ≤ FRS < 0.2), and high risk (FRS ≥ 0.2).
³Delta AUC CI: The AUC difference (LARS AUC − FRS AUC); CI refers to the confidence interval.
⁴NRI: net reclassification improvement.

The LARS model derived from the CLP lists predicted incident CVD as accurately as the LARS derived from the Research Lipid List whole lipidome. In particular, for the intermediate risk group, the LARS derived from the CLP achieved the same level of improved performance as the LARS derived from whole lipidome when compared to the original FRS. The CLP list have demonstrated advantage in clinical translation of lipidomic data, and this LARS model implemented with a simple, fast, and clinically viable blood test has demonstrated advantage, for example, used for early detection of CVD.

TABLE 19
Predictive models of cardiovascular disease events developed
using Ridge or LASSO regression with the CLP1s lipids.

| Rank | Lipid | | | Ridge |
|---|---|---|---|---|
| # | # | Lipid/Covariate | Ridge | (ABS) |
| | | drage_00 | 0.0056 | 0.0056 |
| | | intercept | −0.1678 | 0.1678 |
| 1 | 190 | SM 31:1 | −0.0093 | 0.0093 |
| 2 | 135 | PC 40:8 | −0.0089 | 0.0089 |
| 3 | 1 | AcylCarnitine 12:0 | 0.0078 | 0.0078 |
| 4 | 47 | Cer(m18:1/24:0) | 0.0077 | 0.0077 |
| 5 | 156 | PE 40:6 | −0.0075 | 0.0075 |
| 6 | 134 | PC 40:6 | 0.0073 | 0.0073 |
| 7 | 97 | LPC(O-24:0) | −0.0073 | 0.0073 |
| 8 | 45 | Cer(m18:1/20:0) | 0.0071 | 0.0071 |
| 9 | 52 | DE(18:2) | 0.0068 | 0.0068 |
| 10 | 100 | LPC(P-17:0) | 0.0066 | 0.0066 |
| 11 | 78 | LPC 14:0 | −0.0065 | 0.0065 |
| 12 | 169 | PE(P-18:0/18:2) | 0.0063 | 0.0063 |
| 13 | 143 | PC(P-34:2) | −0.0063 | 0.0063 |
| 14 | 16 | CE 18:0 | 0.0060 | 0.0060 |
| 15 | 140 | PC(P-30:0) | 0.0060 | 0.0060 |
| 16 | 109 | PC 32:0 | 0.0058 | 0.0058 |
| 17 | 129 | PC 38:4(a) | −0.0058 | 0.0058 |
| 18 | 98 | LPC(O-24:2) | 0.0057 | 0.0057 |
| 19 | 142 | PC(P-34:1) | −0.0057 | 0.0057 |
| 20 | 291 | TG(O-52:2) [NL-18:1] | 0.0057 | 0.0057 |
| 21 | 55 | DG 34:1 -(18:1) | −0.0056 | 0.0056 |
| 22 | 194 | SM 34:1 | 0.0056 | 0.0056 |
| 23 | 12 | CE 16:0 | 0.0055 | 0.0055 |
| 24 | 200 | SM 36:3 | −0.0055 | 0.0055 |
| 25 | 259 | TG(54:4) [NL-20:3] | 0.0054 | 0.0054 |
| 26 | 203 | SM 38:3 | −0.0054 | 0.0054 |
| 27 | 130 | PC 38:4(b) | 0.0054 | 0.0054 |
| 28 | 217 | TG(48:2) [NL-18:2] | 0.0053 | 0.0053 |
| 29 | 195 | SM 34:2 | −0.0052 | 0.0052 |
| 30 | 23 | CE 20:4 | −0.0052 | 0.0052 |
| 31 | 148 | PE 34:1 | 0.0052 | 0.0052 |
| 32 | 41 | Cer(d19:1/22:0) | −0.0050 | 0.0050 |
| 33 | 250 | TG(52:5) [NL-20:4] | −0.0050 | 0.0050 |
| 34 | 77 | Hex3Cer(d18:1/16:0) | −0.0048 | 0.0048 |
| 35 | 274 | TG(56:8) [NL-20:4] | 0.0047 | 0.0047 |
| 36 | 147 | PE 32:1 | −0.0047 | 0.0047 |
| 37 | 262 | TG(54:6) [NL-18:3] | 0.0046 | 0.0046 |
| 38 | 94 | LPC(O-20:0) | −0.0046 | 0.0046 |
| 39 | 160 | PE(P-16:0/18:1) | −0.0045 | 0.0045 |
| 40 | 85 | LPC 19:1 | −0.0044 | 0.0044 |
| 41 | 157 | PE(O-34:1) | −0.0044 | 0.0044 |
| 42 | 103 | LPE 18:1 | 0.0044 | 0.0044 |
| 43 | 181 | PI 36:2 | −0.0043 | 0.0043 |
| 44 | 57 | DG 36:2 -(18:1) | −0.0043 | 0.0043 |
| 45 | 266 | TG(54:7) [NL-20:5] | 0.0042 | 0.0042 |
| 46 | 81 | LPC 18:1 | 0.0040 | 0.0040 |
| 47 | 173 | PE(P-18:0/22:6) | −0.0040 | 0.0040 |
| 48 | 35 | Cer(d18:1/24:0) | 0.0040 | 0.0040 |
| 49 | 15 | CE 17:0 | 0.0039 | 0.0039 |
| 50 | 90 | LPC 22:5 | −0.0039 | 0.0039 |
| 51 | 273 | TG(56:7) [NL-22:6] | −0.0039 | 0.0039 |
| 52 | 43 | Cer(d19:1/24:1) | 0.0038 | 0.0038 |
| 53 | 272 | TG(56:7) [NL-22:5] | 0.0038 | 0.0038 |
| 54 | 121 | PC 36:1 | −0.0038 | 0.0038 |
| 55 | 82 | LPC 18:2 | −0.0037 | 0.0037 |
| 56 | 178 | PI 32:1 | −0.0037 | 0.0037 |
| 57 | 168 | PE(P-18:0/18:1) | 0.0037 | 0.0037 |
| 58 | 29 | Cer(d16:1/24:1) | 0.0037 | 0.0037 |
| 59 | 208 | Sph(d17:1) | −0.0036 | 0.0036 |
| 60 | 192 | SM 32:2 | −0.0035 | 0.0035 |
| 61 | 153 | PE 36:4 | 0.0035 | 0.0035 |
| 62 | 116 | PC 34:2 | 0.0035 | 0.0035 |
| 63 | 58 | DG 36:3 -(18:2) | 0.0034 | 0.0034 |
| 64 | 3 | AcylCarnitine 14:0 | 0.0034 | 0.0034 |
| 65 | 61 | GM3(d18:1/16:0) | 0.0034 | 0.0034 |
| 66 | 111 | PC 32:2 | −0.0034 | 0.0034 |
| 67 | 26 | CE 22:6 | −0.0034 | 0.0034 |
| 68 | 39 | Cer(d18:2/24:0) | −0.0033 | 0.0033 |
| 69 | 132 | PC 38:6(a) | −0.0033 | 0.0033 |
| 70 | 14 | CE 16:2 | −0.0033 | 0.0033 |
| 71 | 117 | PC 34:4 | −0.0033 | 0.0033 |
| 72 | 28 | Cer(d16:1/24:0) | −0.0033 | 0.0033 |
| 73 | 264 | TG(54:6) [NL-20:5] | −0.0033 | 0.0033 |
| 74 | 36 | Cer(d18:1/24:1) | 0.0032 | 0.0032 |
| 75 | 201 | SM 38:1 | 0.0031 | 0.0031 |
| 76 | 255 | TG(54:2) [NL-20:1] | 0.0031 | 0.0031 |
| 77 | 180 | PI 36:1 | 0.0031 | 0.0031 |
| 78 | 280 | TG(58:9) [NL-22:6] | 0.0031 | 0.0031 |
| 79 | 245 | TG(52:3) [NL-18:2] | −0.0031 | 0.0031 |
| 80 | 138 | PC(O-36:0) | 0.0030 | 0.0030 |
| 81 | 278 | TG(58:10) [NL-22:6] | 0.0030 | 0.0030 |
| 82 | 75 | Hex2Cer(d18:1/24:1) | 0.0030 | 0.0030 |
| 83 | 60 | DG 38:4 -(20:3) | −0.0030 | 0.0030 |
| 84 | 141 | PC(P-32:0) | 0.0029 | 0.0029 |
| 85 | 158 | PE(O-34:2) | −0.0028 | 0.0028 |
| 86 | 105 | LPE 20:4 | 0.0028 | 0.0028 |
| 87 | 126 | PC 36:5(a) | −0.0027 | 0.0027 |
| 88 | 40 | Cer(d18:2/24:1) | 0.0027 | 0.0027 |
| 89 | 197 | SM 35:1 | 0.0027 | 0.0027 |
| 90 | 290 | TG(O-52:2) [NL-17:1] | 0.0027 | 0.0027 |
| 91 | 283 | TG(O-50:1) [NL-17:1] | −0.0027 | 0.0027 |
| 92 | 32 | Cer(d18:1/20:0) | 0.0027 | 0.0027 |
| 93 | 269 | TG(56:6) [NL-22:5] | −0.0026 | 0.0026 |
| 94 | 63 | GM3(d18:1/22:0) | −0.0026 | 0.0026 |
| 95 | 196 | SM 34:3 | −0.0026 | 0.0026 |
| 96 | 184 | PI 38:3 | −0.0026 | 0.0026 |
| 97 | 241 | TG(51:2) [NL-17:1] | −0.0026 | 0.0026 |
| 98 | 68 | Hex1Cer(d18:1/24:0) | 0.0026 | 0.0026 |
| 99 | 128 | PC 37:6 | −0.0026 | 0.0026 |
| 100 | 76 | Hex2Cer(d18:2/16:0) | 0.0026 | 0.0026 |
| 101 | 119 | PC 35:4 | −0.0025 | 0.0025 |
| 102 | 240 | TG(51:2) [NL-17:0] | −0.0025 | 0.0025 |
| 103 | 42 | Cer(d19:1/24:0) | 0.0025 | 0.0025 |
| 104 | 56 | DG 34:2 -(18:2) | 0.0025 | 0.0025 |
| 105 | 38 | Cer(d18:2/23:0) | 0.0025 | 0.0025 |
| 106 | 96 | LPC(O-22:1) | −0.0025 | 0.0025 |
| 107 | 239 | TG(51:2) [NL-15:0] | 0.0025 | 0.0025 |
| 108 | 247 | TG(52:4) [NL-18:2] | 0.0025 | 0.0025 |
| 109 | 86 | LPC 20:0 | 0.0024 | 0.0024 |
| 110 | 288 | TG(O-52:0) [NL-16:0] | 0.0024 | 0.0024 |
| 111 | 233 | TG(50:3) [NL-18:3] | 0.0024 | 0.0024 |
| 112 | 120 | PC 35:5 | 0.0024 | 0.0024 |
| 113 | 11 | CE 15:0 | −0.0024 | 0.0024 |
| 114 | 287 | TG(O-50:2) [NL-18:2] | −0.0024 | 0.0024 |
| 115 | 170 | PE(P-18:0/20:4) | −0.0024 | 0.0024 |
| 116 | 8 | AcylCarnitine 18:1 | 0.0024 | 0.0024 |
| 117 | 257 | TG(54:3) [NL-18:2] | −0.0024 | 0.0024 |
| 118 | 37 | Cer(d18:2/22:0) | 0.0024 | 0.0024 |
| 119 | 154 | PE 38:4 | −0.0024 | 0.0024 |
| 120 | 164 | PE(P-16:0/22:4) | 0.0024 | 0.0024 |
| 121 | 172 | PE(P-18:0/22:4) | 0.0023 | 0.0023 |
| 122 | 110 | PC 32:1 | 0.0023 | 0.0023 |
| 123 | 7 | AcylCarnitine 16:1 | −0.0023 | 0.0023 |
| 124 | 225 | TG(50:2) [NL-14:0] | 0.0023 | 0.0023 |
| 125 | 149 | PE 34:2 | 0.0023 | 0.0023 |
| 126 | 179 | PI 34:1 | 0.0023 | 0.0023 |
| 127 | 171 | PE(P-18:0/20:5) | −0.0023 | 0.0023 |
| 128 | 212 | TG(48:1) [NL-16:1] | −0.0022 | 0.0022 |
| 129 | 186 | PI 38:6 | 0.0022 | 0.0022 |
| 130 | 289 | TG(O-52:2) [NL-16:0] | −0.0022 | 0.0022 |
| 131 | 27 | Cer(d16:1/22:0) | 0.0022 | 0.0022 |
| 132 | 114 | PC 34:0 | 0.0022 | 0.0022 |
| 133 | 244 | TG(52:2) [NL-16:0] | −0.0022 | 0.0022 |
| 134 | 281 | TG(O-50:1) [NL-15:0] | 0.0021 | 0.0021 |
| 135 | 155 | PE 38:6 | −0.0021 | 0.0021 |
| 136 | 276 | TG(56:8) [NL-22:6] | 0.0020 | 0.0020 |
| 137 | 33 | Cer(d18:1/22:0) | −0.0020 | 0.0020 |
| 138 | 236 | TG(50:4) [NL-20:4] | −0.0020 | 0.0020 |
| 139 | 189 | S1P(d18:1) | −0.0020 | 0.0020 |
| 140 | 34 | Cer(d18:1/23:0) | −0.0019 | 0.0019 |
| 141 | 202 | SM 38:2 | −0.0019 | 0.0019 |
| 142 | 223 | TG(50:1) [NL-16:0] | 0.0019 | 0.0019 |
| 143 | 193 | SM 33:1 | 0.0019 | 0.0019 |
| 144 | 124 | PC 36:4(a) | −0.0019 | 0.0019 |
| 145 | 123 | PC 36:4 | −0.0018 | 0.0018 |
| 146 | 2 | AcylCarnitine 13:0 | −0.0018 | 0.0018 |
| 147 | 174 | PE(P-18:1/20:4) | −0.0018 | 0.0018 |
| 148 | 224 | TG(50:1) [NL-18:1] | −0.0018 | 0.0018 |
| 149 | 166 | PE(P-17:0/20:4) | 0.0018 | 0.0018 |
| 150 | 228 | TG(50:2) [NL-18:2] | −0.0018 | 0.0018 |
| 151 | 234 | TG(50:4) [NL-14:0] | 0.0017 | 0.0017 |
| 152 | 243 | TG(52:1) [NL-18:1] | −0.0017 | 0.0017 |
| 153 | 235 | TG(50:4) [NL-18:3] | −0.0017 | 0.0017 |
| 154 | 112 | PC 33:1 | 0.0016 | 0.0016 |
| 155 | 219 | TG(48:3) [NL-16:1] | −0.0016 | 0.0016 |
| 156 | 249 | TG(52:5) [NL-18:3] | 0.0016 | 0.0016 |
| 157 | 268 | TG(56:6) [NL-20:4] | −0.0016 | 0.0016 |
| 158 | 177 | PE(P-20:0/20:4) | −0.0016 | 0.0016 |
| 159 | 187 | PI 40:6 | −0.0016 | 0.0016 |
| 160 | 237 | TG(51:0) [NL-16:0] | 0.0016 | 0.0016 |
| 161 | 95 | LPC(O-22:0) | −0.0016 | 0.0016 |
| 162 | 175 | PE(P-18:1/20:5) | 0.0016 | 0.0016 |
| 163 | 10 | CE 14:0 | −0.0016 | 0.0016 |
| 164 | 182 | PI 36:4 | −0.0015 | 0.0015 |
| 165 | 232 | TG(50:3) [NL-18:2] | 0.0015 | 0.0015 |
| 166 | 167 | PE(P-17:0/22:6) | 0.0015 | 0.0015 |
| 167 | 198 | SM 35:2 | −0.0015 | 0.0015 |
| 168 | 48 | Cer(m18:1/24:1) | −0.0015 | 0.0015 |
| 169 | 252 | TG(53:2) [NL-17:1] | 0.0015 | 0.0015 |
| 170 | 25 | CE 22:1 | 0.0015 | 0.0015 |
| 171 | 9 | AcylCarnitine 18:2 | −0.0015 | 0.0015 |
| 172 | 205 | SM 41:1 | −0.0015 | 0.0015 |
| 173 | 261 | TG(54:5) [NL-20:4] | −0.0014 | 0.0014 |
| 174 | 214 | TG(48:2) [NL-14:0] | 0.0014 | 0.0014 |
| 175 | 91 | LPC 22:6 | 0.0014 | 0.0014 |
| 176 | 87 | LPC 20:4 | −0.0014 | 0.0014 |
| 177 | 188 | PS 38:4 | −0.0014 | 0.0014 |
| 178 | 74 | Hex2Cer(d18:1/24:0) | 0.0014 | 0.0014 |
| 179 | 106 | LPE 22:6 | −0.0014 | 0.0014 |
| 180 | 4 | AcylCarnitine 14:1 | −0.0013 | 0.0013 |
| 181 | 65 | Hex1Cer(d18:1/16:0) | −0.0013 | 0.0013 |
| 182 | 131 | PC 38:4(c) | 0.0013 | 0.0013 |
| 183 | 84 | LPC 19:0 | −0.0013 | 0.0013 |
| 184 | 293 | TG(O-54:3) [NL-18:1] | −0.0013 | 0.0013 |
| 185 | 231 | TG(50:3) [NL-16:1] | 0.0013 | 0.0013 |
| 186 | 229 | TG(50:3) [NL-14:0] | 0.0013 | 0.0013 |
| 187 | 248 | TG(52:4) [NL-18:3] | −0.0013 | 0.0013 |
| 188 | 263 | TG(54:6) [NL-20:4] | −0.0012 | 0.0012 |
| 189 | 285 | TG(O-50:2) [NL-16:1] | −0.0012 | 0.0012 |
| 190 | 50 | COH (161) | 0.0012 | 0.0012 |
| 191 | 6 | AcylCarnitine 16:0 | 0.0012 | 0.0012 |
| 192 | 115 | PC 34:1 | −0.0012 | 0.0012 |
| 193 | 227 | TG(50:2) [NL-18:1] | −0.0012 | 0.0012 |
| 194 | 51 | DE(18:1) | 0.0011 | 0.0011 |
| 195 | 66 | Hex1Cer(d18:1/18:0) | −0.0011 | 0.0011 |
| 196 | 101 | LPE 16:0 | 0.0011 | 0.0011 |
| 197 | 298 | LPE(P-18:0) | 0.0011 | 0.0011 |
| 198 | 83 | LPC 18:3 | 0.0011 | 0.0011 |
| 199 | 49 | Cer1P(d18:1/16:0) | −0.0011 | 0.0011 |
| 200 | 221 | TG(49:1) [NL-17:1] | 0.0011 | 0.0011 |
| 201 | 92 | LPC 24:0 | −0.0010 | 0.0010 |
| 202 | 210 | Sulfatide (d18:1:/16:0) | 0.0010 | 0.0010 |
| 203 | 20 | CE 20:0 | 0.0010 | 0.0010 |
| 204 | 136 | PC(O-32:0) | −0.0010 | 0.0010 |
| 205 | 204 | SM 40:1 | 0.0010 | 0.0010 |
| 206 | 122 | PC 36:2 | 0.0010 | 0.0010 |
| 207 | 62 | GM3(d18:1/18:0) | 0.0010 | 0.0010 |
| 208 | 296 | Ubiquinone | 0.0009 | 0.0009 |
| 209 | 144 | PC(P-36:4) | −0.0009 | 0.0009 |
| 210 | 271 | TG(56:7) [NL-20:5] | −0.0009 | 0.0009 |
| 211 | 185 | PI 38:4 | −0.0009 | 0.0009 |
| 212 | 251 | TG(52:5) [NL-20:5] | −0.0009 | 0.0009 |
| 213 | 161 | PE(P-16:0/18:2) | 0.0009 | 0.0009 |
| 214 | 215 | TG(48:2) [NL-14:1] | 0.0009 | 0.0009 |
| 215 | 5 | AcylCarnitine 14:2 | −0.0009 | 0.0009 |
| 216 | 297 | LPE(P-16:0) | 0.0009 | 0.0009 |
| 217 | 275 | TG(56:8) [NL-20:5] | 0.0009 | 0.0009 |
| 218 | 295 | TG(O-54:4) [NL-18:2] | −0.0009 | 0.0009 |
| 219 | 213 | TG(48:1) [NL-18:1] | 0.0009 | 0.0009 |
| 220 | 226 | TG(50:2) [NL-16:1] | −0.0008 | 0.0008 |
| 221 | 159 | PE(O-36:4) | −0.0008 | 0.0008 |
| 222 | 277 | TG(56:9) [NL-22:6] | 0.0008 | 0.0008 |
| 223 | 93 | LPC 26:0 | −0.0008 | 0.0008 |
| 224 | 206 | SM 44:2 | 0.0008 | 0.0008 |
| 225 | 18 | CE 18:2 | −0.0008 | 0.0008 |
| 226 | 13 | CE 16:1 | 0.0008 | 0.0008 |
| 227 | 163 | PE(P-16:0/20:5) | 0.0008 | 0.0008 |
| 228 | 246 | TG(52:4) [NL-16:1] | −0.0007 | 0.0007 |
| 229 | 256 | TG(54:3) [NL-18:1] | 0.0007 | 0.0007 |
| 230 | 73 | Hex2Cer(d18:1/22:0) | 0.0007 | 0.0007 |
| 231 | 69 | Hex1Cer(d18:1/24:1) | −0.0007 | 0.0007 |
| 232 | 230 | TG(50:3) [NL-14:1] | −0.0007 | 0.0007 |
| 233 | 222 | TG(50:0) [NL-18:0] | −0.0007 | 0.0007 |
| 234 | 107 | LPI 18:0 | −0.0007 | 0.0007 |
| 235 | 279 | TG(58:8) [NL-22:6] | 0.0007 | 0.0007 |
| 236 | 270 | TG(56:7) [NL-20:4] | −0.0007 | 0.0007 |
| 237 | 22 | CE 20:2 | 0.0007 | 0.0007 |
| 238 | 127 | PC 36:5(b) | −0.0007 | 0.0007 |
| 239 | 118 | PC 34:5 | −0.0007 | 0.0007 |
| 240 | 265 | TG(54:6) [NL-22:6] | 0.0006 | 0.0006 |
| 241 | 150 | PE 34:3 | 0.0006 | 0.0006 |
| 242 | 59 | DG 36:4 -(18:2) | 0.0006 | 0.0006 |
| 243 | 71 | Hex2Cer(d16:1/16:0) | −0.0006 | 0.0006 |
| 244 | 218 | TG(48:3) [NL-14:0] | −0.0006 | 0.0006 |
| 245 | 207 | SM 44:3 | 0.0006 | 0.0006 |
| 246 | 139 | PC(O-36:5) | 0.0006 | 0.0006 |
| 247 | 253 | TG(53:2) [NL-18:1] | 0.0006 | 0.0006 |
| 248 | 53 | DE(20:4) | 0.0006 | 0.0006 |
| 249 | 80 | LPC 18:0 | 0.0005 | 0.0005 |
| 250 | 267 | TG(54:7) [NL-22:6] | 0.0005 | 0.0005 |
| 251 | 282 | TG(O-50:1) [NL-16:0] | 0.0005 | 0.0005 |
| 252 | 220 | TG(48:3) [NL-18:3] | 0.0005 | 0.0005 |
| 253 | 183 | PI 37:4 | 0.0005 | 0.0005 |
| 254 | 30 | Cer(d17:1/24:0) | 0.0005 | 0.0005 |
| 255 | 292 | TG(O-54:3) [NL-17:1] | 0.0005 | 0.0005 |
| 256 | 162 | PE(P-16:0/20:4) | −0.0005 | 0.0005 |
| 257 | 113 | PC 33:2 | 0.0005 | 0.0005 |
| 258 | 64 | GM3(d18:1/24:1) | 0.0005 | 0.0005 |
| 259 | 209 | Sulfatide (d18:1:/16:0(OH)) | 0.0005 | 0.0005 |
| 260 | 70 | Hex1Cer(d18:2/24:0) | −0.0005 | 0.0005 |
| 261 | 21 | CE 20:1 | 0.0005 | 0.0005 |
| 262 | 133 | PC 38:6(b) | 0.0005 | 0.0005 |
| 263 | 46 | Cer(m18:1/22:0) | 0.0005 | 0.0005 |
| 264 | 102 | LPE 18:0 | −0.0005 | 0.0005 |
| 265 | 125 | PC 36:4(b) | −0.0004 | 0.0004 |
| 266 | 19 | CE 18:3 | −0.0004 | 0.0004 |
| 267 | 152 | PE 36:2 | −0.0004 | 0.0004 |
| 268 | 211 | TG(48:0) [NL-16:0] | −0.0004 | 0.0004 |
| 269 | 260 | TG(54:5) [NL-18:3] | 0.0004 | 0.0004 |
| 270 | 67 | Hex1Cer(d18:1/22:0) | −0.0004 | 0.0004 |
| 271 | 145 | PC(P-36:5) | 0.0003 | 0.0003 |
| 272 | 216 | TG(48:2) [NL-16:1] | −0.0003 | 0.0003 |
| 273 | 24 | CE 20:5 | 0.0003 | 0.0003 |
| 274 | 242 | TG(52:1) [NL-18:0] | 0.0003 | 0.0003 |
| 275 | 137 | PC(O-34:2) | 0.0003 | 0.0003 |
| 276 | 72 | Hex2Cer(d18:1/16:0) | −0.0003 | 0.0003 |
| 277 | 238 | TG(51:1) [NL-17:0] | 0.0003 | 0.0003 |
| 278 | 88 | LPC 20:5 | 0.0003 | 0.0003 |
| 279 | 79 | LPC 17:0 | −0.0003 | 0.0003 |
| 280 | 104 | LPE 18:2 | 0.0003 | 0.0003 |
| 281 | 31 | Cer(d18:1/16:0) | −0.0003 | 0.0003 |
| 282 | 146 | PC(P-37:4) | 0.0003 | 0.0003 |
| 283 | 176 | PE(P-20:0/18:2) | 0.0002 | 0.0002 |
| 284 | 165 | PE(P-16:0/22:6) | −0.0002 | 0.0002 |
| 285 | 254 | TG(54:2) [NL-18:0] | −0.0002 | 0.0002 |
| 286 | 89 | LPC 22:0 | −0.0002 | 0.0002 |
| 287 | 54 | DG 32:0 -(16:0) | −0.0002 | 0.0002 |
| 288 | 99 | LPC(P-16:0) | 0.0001 | 0.0001 |
| 289 | 258 | TG(54:4) [NL-18:2] | −0.0001 | 0.0001 |
| 290 | 191 | SM 32:1 | −0.0001 | 0.0001 |
| 291 | 294 | TG(O-54:4) [NL-17:1] | 0.0001 | 0.0001 |
| 292 | 44 | Cer(m18:0/22:0) | 0.0001 | 0.0001 |
| 293 | 284 | TG(O-50:1) [NL-18:1] | −0.0001 | 0.0001 |
| 294 | 17 | CE 18:1 | −0.0001 | 0.0001 |
| 295 | 199 | SM 36:1 | 0.0001 | 0.0001 |
| 296 | 151 | PE 36:1 | −0.0001 | 0.0001 |
| 297 | 108 | PC 31:0 | 0.0000 | 0.0000 |
| 298 | 286 | TG(O-50:2) [NL-18:1] | 0.0000 | 0.0000 |

TABLE 20
Predictive models of cardiovascular disease
events developed using with the CLP1s lipids.

| Rank | | | | LASSO |
|---|---|---|---|---|
| # | Lipid # | Lipid/Covariate | LASSO | (ABS) |
| | | drage_00 | 0.0062 | 0.0062 |
| | | intercept | −0.1998 | 0.1998 |
| 1 | 190 | SM 31:1 | −0.0110 | 0.0110 |
| 2 | 135 | PC 40:8 | −0.0102 | 0.0102 |
| 3 | 195 | SM 34:2 | −0.0098 | 0.0098 |
| 4 | 97 | LPC(O-24:0) | −0.0086 | 0.0086 |
| 5 | 16 | CE 18:0 | 0.0085 | 0.0085 |
| 6 | 47 | Cer(m18:1/24:0) | 0.0074 | 0.0074 |
| 7 | 142 | PC(P-34:1) | −0.0068 | 0.0068 |
| 8 | 126 | PC 36:5(a) | −0.0064 | 0.0064 |
| 9 | 1 | AcylCarnitine 12:0 | 0.0056 | 0.0056 |
| 10 | 109 | PC 32:0 | 0.0056 | 0.0056 |
| 11 | 156 | PE 40:6 | −0.0053 | 0.0053 |
| 12 | 45 | Cer(m18:1/20:0) | 0.0052 | 0.0052 |
| 13 | 23 | CE 20:4 | −0.0052 | 0.0052 |
| 14 | 15 | CE 17:0 | 0.0051 | 0.0051 |
| 15 | 194 | SM 34:1 | 0.0050 | 0.0050 |
| 16 | 200 | SM 36:3 | −0.0048 | 0.0048 |
| 17 | 12 | CE 16:0 | 0.0048 | 0.0048 |
| 18 | 100 | LPC(P-17:0) | 0.0044 | 0.0044 |
| 19 | 98 | LPC(O-24:2) | 0.0043 | 0.0043 |
| 20 | 234 | TG(50:4) [NL-14:0] | 0.0043 | 0.0043 |
| 21 | 52 | DE(18:2) | 0.0039 | 0.0039 |
| 22 | 103 | LPE 18:1 | 0.0038 | 0.0038 |
| 23 | 157 | PE(O-34:1) | −0.0035 | 0.0035 |
| 24 | 141 | PC(P-32:0) | 0.0035 | 0.0035 |
| 25 | 90 | LPC 22:5 | −0.0034 | 0.0034 |
| 26 | 203 | SM 38:3 | −0.0033 | 0.0033 |
| 27 | 14 | CE 16:2 | −0.0033 | 0.0033 |
| 28 | 36 | Cer(d18:1/24:1) | 0.0032 | 0.0032 |
| 29 | 278 | TG(58:10) [NL-22:6] | 0.0030 | 0.0030 |
| 30 | 129 | PC 38:4(a) | −0.0028 | 0.0028 |
| 31 | 180 | PI 36:1 | 0.0028 | 0.0028 |
| 32 | 280 | TG(58:9) [NL-22:6] | 0.0027 | 0.0027 |
| 33 | 208 | Sph(d17:1) | −0.0027 | 0.0027 |
| 34 | 262 | TG(54:6) [NL-18:3] | 0.0025 | 0.0025 |
| 35 | 143 | PC(P-34:2) | −0.0025 | 0.0025 |
| 36 | 138 | PC(O-36:0) | 0.0024 | 0.0024 |
| 37 | 132 | PC 38:6(a) | −0.0024 | 0.0024 |
| 38 | 29 | Cer(d16:1/24:1) | 0.0023 | 0.0023 |
| 39 | 140 | PC(P-30:0) | 0.0023 | 0.0023 |
| 40 | 25 | CE 22:1 | 0.0022 | 0.0022 |
| 41 | 43 | Cer(d19:1/24:1) | 0.0022 | 0.0022 |
| 42 | 168 | PE(P-18:0/18:1) | 0.0022 | 0.0022 |
| 43 | 255 | TG(54:2) [NL-20:1] | 0.0021 | 0.0021 |
| 44 | 61 | GM3(d18:1/16:0) | 0.0020 | 0.0020 |
| 45 | 38 | Cer(d18:2/23:0) | −0.0019 | 0.0019 |
| 46 | 181 | PI 36:2 | −0.0019 | 0.0019 |
| 47 | 78 | LPC 14:0 | −0.0019 | 0.0019 |
| 48 | 158 | PE(O-34:2) | −0.0019 | 0.0019 |
| 49 | 172 | PE(P-18:0/22:4) | 0.0016 | 0.0016 |
| 50 | 159 | PE(O-36:4) | −0.0015 | 0.0015 |
| 51 | 94 | LPC(O-20:0) | −0.0014 | 0.0014 |
| 52 | 105 | LPE 20:4 | 0.0012 | 0.0012 |
| 53 | 95 | LPC(O-22:0) | −0.0012 | 0.0012 |
| 54 | 184 | PI 38:3 | −0.0011 | 0.0011 |
| 55 | 233 | TG(50:3) [NL-18:3] | 0.0011 | 0.0011 |
| 56 | 266 | TG(54:7) [NL-20:5] | 0.0011 | 0.0011 |
| 57 | 130 | PC 38:4(b) | 0.0010 | 0.0010 |
| 58 | 50 | COH (161) | 0.0010 | 0.0010 |
| 59 | 51 | DE(18:1) | 0.0009 | 0.0009 |
| 60 | 26 | CE 22:6 | −0.0009 | 0.0009 |
| 61 | 21 | CE 20:1 | 0.0009 | 0.0009 |
| 62 | 259 | TG(54:4) [NL-20:3] | 0.0008 | 0.0008 |
| 63 | 173 | PE(P-18:0/22:6) | −0.0008 | 0.0008 |
| 64 | 296 | Ubiquinone | 0.0008 | 0.0008 |
| 65 | 237 | TG(51:0) [NL-16:0] | 0.0007 | 0.0007 |
| 66 | 177 | PE(P-20:0/20:4) | −0.0007 | 0.0007 |
| 67 | 206 | SM 44:2 | 0.0006 | 0.0006 |
| 68 | 11 | CE 15:0 | −0.0005 | 0.0005 |
| 69 | 268 | TG(56:6) [NL-20:4] | −0.0005 | 0.0005 |
| 70 | 295 | TG(O-54:4) [NL-18:2] | −0.0004 | 0.0004 |
| 71 | 19 | CE 18:3 | −0.0004 | 0.0004 |
| 72 | 63 | GM3(d18:1/22:0) | −0.0003 | 0.0003 |
| 73 | 70 | Hex1Cer(d18:2/24:0) | −0.0003 | 0.0003 |
| 74 | 3 | AcylCarnitine 14:0 | 0.0002 | 0.0002 |
| 75 | 66 | Hex1Cer(d18:1/18:0) | −0.0002 | 0.0002 |
| 76 | 209 | Sulfatide (d18:1:/16:0(OH)) | 0.0002 | 0.0002 |
| 77 | 62 | GM3(d18:1/18:0) | 0.0001 | 0.0001 |
| 78 | 93 | LPC 26:0 | −0.0001 | 0.0001 |
| 79 | 291 | TG(O-52:2) [NL-18:1] | 0.0001 | 0.0001 |
| 80 | 196 | SM 34:3 | −0.0001 | 0.0001 |
| 81 | 107 | LPI 18:0 | −0.0001 | 0.0001 |
| 82 | 6 | AcylCarnitine 16:0 | 0.0001 | 0.0001 |
| 83 | 85 | LPC 19:1 | −0.0001 | 0.0001 |
| 84 | 20 | CE 20:0 | 0.0001 | 0.0001 |
| 85 | 217 | TG(48:2) [NL-18:2] | 0.0001 | 0.0001 |
| 86 | 197 | SM 35:1 | 0.0000 | 0.0000 |
| 87 | 42 | Cer(d19:1/24:0) | 0.0000 | 0.0000 |
| 88 | 2 | AcylCarnitine 13:0 | 0.0000 | 0.0000 |
| 89 | 4 | AcylCarnitine 14:1 | 0.0000 | 0.0000 |
| 90 | 5 | AcylCarnitine 14:2 | 0.0000 | 0.0000 |
| 91 | 7 | AcylCarnitine 16:1 | 0.0000 | 0.0000 |
| 92 | 8 | AcylCarnitine 18:1 | 0.0000 | 0.0000 |
| 93 | 9 | AcylCarnitine 18:2 | 0.0000 | 0.0000 |
| 94 | 10 | CE 14:0 | 0.0000 | 0.0000 |
| 95 | 13 | CE 16:1 | 0.0000 | 0.0000 |
| 96 | 17 | CE 18:1 | 0.0000 | 0.0000 |
| 97 | 18 | CE 18:2 | 0.0000 | 0.0000 |
| 98 | 22 | CE 20:2 | 0.0000 | 0.0000 |
| 99 | 24 | CE 20:5 | 0.0000 | 0.0000 |
| 100 | 27 | Cer(d16:1/22:0) | 0.0000 | 0.0000 |
| 101 | 28 | Cer(d16:1/24:0) | 0.0000 | 0.0000 |
| 102 | 30 | Cer(d17:1/24:0) | 0.0000 | 0.0000 |
| 103 | 31 | Cer(d18:1/16:0) | 0.0000 | 0.0000 |
| 104 | 32 | Cer(d18:1/20:0) | 0.0000 | 0.0000 |
| 105 | 33 | Cer(d18:1/22:0) | 0.0000 | 0.0000 |
| 106 | 34 | Cer(d18:1/23:0) | 0.0000 | 0.0000 |
| 107 | 35 | Cer(d18:1/24:0) | 0.0000 | 0.0000 |
| 108 | 37 | Cer(d18:2/22:0) | 0.0000 | 0.0000 |
| 109 | 39 | Cer(d18:2/24:0) | 0.0000 | 0.0000 |
| 110 | 40 | Cer(d18:2/24:1) | 0.0000 | 0.0000 |
| 111 | 41 | Cer(d19:1/22:0) | 0.0000 | 0.0000 |
| 112 | 44 | Cer(m18:0/22:0) | 0.0000 | 0.0000 |
| 113 | 46 | Cer(m18:1/22:0) | 0.0000 | 0.0000 |
| 114 | 48 | Cer(m18:1/24:1) | 0.0000 | 0.0000 |
| 115 | 49 | Cer1P(d18:1/16:0) | 0.0000 | 0.0000 |
| 116 | 53 | DE(20:4) | 0.0000 | 0.0000 |
| 117 | 54 | DG 32:0 -(16:0) | 0.0000 | 0.0000 |
| 118 | 55 | DG 34:1 -(18:1) | 0.0000 | 0.0000 |
| 119 | 56 | DG 34:2 -(18:2) | 0.0000 | 0.0000 |
| 120 | 57 | DG 36:2 -(18:1) | 0.0000 | 0.0000 |
| 121 | 58 | DG 36:3 -(18:2) | 0.0000 | 0.0000 |
| 122 | 59 | DG 36:4 -(18:2) | 0.0000 | 0.0000 |
| 123 | 60 | DG 38:4 -(20:3) | 0.0000 | 0.0000 |
| 124 | 64 | GM3(d18:1/24:1) | 0.0000 | 0.0000 |
| 125 | 65 | Hex1Cer(d18:1/16:0) | 0.0000 | 0.0000 |
| 126 | 67 | Hex1Cer(d18:1/22:0) | 0.0000 | 0.0000 |
| 127 | 68 | Hex1Cer(d18:1/24:0) | 0.0000 | 0.0000 |
| 128 | 69 | Hex1Cer(d18:1/24:1) | 0.0000 | 0.0000 |
| 129 | 71 | Hex2Cer(d16:1/16:0) | 0.0000 | 0.0000 |
| 130 | 72 | Hex2Cer(d18:1/16:0) | 0.0000 | 0.0000 |
| 131 | 73 | Hex2Cer(d18:1/22:0) | 0.0000 | 0.0000 |
| 132 | 74 | Hex2Cer(d18:1/24:0) | 0.0000 | 0.0000 |
| 133 | 75 | Hex2Cer(d18:1/24:1) | 0.0000 | 0.0000 |
| 134 | 76 | Hex2Cer(d18:2/16:0) | 0.0000 | 0.0000 |
| 135 | 77 | Hex3Cer(d18:1/16:0) | 0.0000 | 0.0000 |
| 136 | 79 | LPC 17:0 | 0.0000 | 0.0000 |
| 137 | 80 | LPC 18:0 | 0.0000 | 0.0000 |
| 138 | 81 | LPC 18:1 | 0.0000 | 0.0000 |
| 139 | 82 | LPC 18:2 | 0.0000 | 0.0000 |
| 140 | 83 | LPC 18:3 | 0.0000 | 0.0000 |
| 141 | 84 | LPC 19:0 | 0.0000 | 0.0000 |
| 142 | 86 | LPC 20:0 | 0.0000 | 0.0000 |
| 143 | 87 | LPC 20:4 | 0.0000 | 0.0000 |
| 144 | 88 | LPC 20:5 | 0.0000 | 0.0000 |
| 145 | 89 | LPC 22:0 | 0.0000 | 0.0000 |
| 146 | 91 | LPC 22:6 | 0.0000 | 0.0000 |
| 147 | 92 | LPC 24:0 | 0.0000 | 0.0000 |
| 148 | 96 | LPC(O-22:1) | 0.0000 | 0.0000 |
| 149 | 99 | LPC(P-16:0) | 0.0000 | 0.0000 |
| 150 | 101 | LPE 16:0 | 0.0000 | 0.0000 |
| 151 | 102 | LPE 18:0 | 0.0000 | 0.0000 |
| 152 | 104 | LPE 18:2 | 0.0000 | 0.0000 |
| 153 | 106 | LPE 22:6 | 0.0000 | 0.0000 |
| 154 | 297 | LPE(P-16:0) | 0.0000 | 0.0000 |
| 155 | 298 | LPE(P-18:0) | 0.0000 | 0.0000 |
| 156 | 108 | PC 31:0 | 0.0000 | 0.0000 |
| 157 | 110 | PC 32:1 | 0.0000 | 0.0000 |
| 158 | 111 | PC 32:2 | 0.0000 | 0.0000 |
| 159 | 112 | PC 33:1 | 0.0000 | 0.0000 |
| 160 | 113 | PC 33:2 | 0.0000 | 0.0000 |
| 161 | 114 | PC 34:0 | 0.0000 | 0.0000 |
| 162 | 115 | PC 34:1 | 0.0000 | 0.0000 |
| 163 | 116 | PC 34:2 | 0.0000 | 0.0000 |
| 164 | 117 | PC 34:4 | 0.0000 | 0.0000 |
| 165 | 118 | PC 34:5 | 0.0000 | 0.0000 |
| 166 | 119 | PC 35:4 | 0.0000 | 0.0000 |
| 167 | 120 | PC 35:5 | 0.0000 | 0.0000 |
| 168 | 121 | PC 36:1 | 0.0000 | 0.0000 |
| 169 | 122 | PC 36:2 | 0.0000 | 0.0000 |
| 170 | 123 | PC 36:4 | 0.0000 | 0.0000 |
| 171 | 124 | PC 36:4(a) | 0.0000 | 0.0000 |
| 172 | 125 | PC 36:4(b) | 0.0000 | 0.0000 |
| 173 | 127 | PC 36:5(b) | 0.0000 | 0.0000 |
| 174 | 128 | PC 37:6 | 0.0000 | 0.0000 |
| 175 | 131 | PC 38:4(c) | 0.0000 | 0.0000 |
| 176 | 133 | PC 38:6(b) | 0.0000 | 0.0000 |
| 177 | 134 | PC 40:6 | 0.0000 | 0.0000 |
| 178 | 136 | PC(O-32:0) | 0.0000 | 0.0000 |
| 179 | 137 | PC(O-34:2) | 0.0000 | 0.0000 |
| 180 | 139 | PC(O-36:5) | 0.0000 | 0.0000 |
| 181 | 144 | PC(P-36:4) | 0.0000 | 0.0000 |
| 182 | 145 | PC(P-36:5) | 0.0000 | 0.0000 |
| 183 | 146 | PC(P-37:4) | 0.0000 | 0.0000 |
| 184 | 147 | PE 32:1 | 0.0000 | 0.0000 |
| 185 | 148 | PE 34:1 | 0.0000 | 0.0000 |
| 186 | 149 | PE 34:2 | 0.0000 | 0.0000 |
| 187 | 150 | PE 34:3 | 0.0000 | 0.0000 |
| 188 | 151 | PE 36:1 | 0.0000 | 0.0000 |
| 189 | 152 | PE 36:2 | 0.0000 | 0.0000 |
| 190 | 153 | PE 36:4 | 0.0000 | 0.0000 |
| 191 | 154 | PE 38:4 | 0.0000 | 0.0000 |
| 192 | 155 | PE 38:6 | 0.0000 | 0.0000 |
| 193 | 160 | PE(P-16:0/18:1) | 0.0000 | 0.0000 |
| 194 | 161 | PE(P-16:0/18:2) | 0.0000 | 0.0000 |
| 195 | 162 | PE(P-16:0/20:4) | 0.0000 | 0.0000 |
| 196 | 163 | PE(P-16:0/20:5) | 0.0000 | 0.0000 |
| 197 | 164 | PE(P-16:0/22:4) | 0.0000 | 0.0000 |
| 198 | 165 | PE(P-16:0/22:6) | 0.0000 | 0.0000 |
| 199 | 166 | PE(P-17:0/20:4) | 0.0000 | 0.0000 |
| 200 | 167 | PE(P-17:0/22:6) | 0.0000 | 0.0000 |
| 201 | 169 | PE(P-18:0/18:2) | 0.0000 | 0.0000 |
| 202 | 170 | PE(P-18:0/20:4) | 0.0000 | 0.0000 |
| 203 | 171 | PE(P-18:0/20:5) | 0.0000 | 0.0000 |
| 204 | 174 | PE(P-18:1/20:4) | 0.0000 | 0.0000 |
| 205 | 175 | PE(P-18:1/20:5) | 0.0000 | 0.0000 |
| 206 | 176 | PE(P-20:0/18:2) | 0.0000 | 0.0000 |
| 207 | 178 | PI 32:1 | 0.0000 | 0.0000 |
| 208 | 179 | PI 34:1 | 0.0000 | 0.0000 |
| 209 | 182 | PI 36:4 | 0.0000 | 0.0000 |
| 210 | 183 | PI 37:4 | 0.0000 | 0.0000 |
| 211 | 185 | PI 38:4 | 0.0000 | 0.0000 |
| 212 | 186 | PI 38:6 | 0.0000 | 0.0000 |
| 213 | 187 | PI 40:6 | 0.0000 | 0.0000 |
| 214 | 188 | PS 38:4 | 0.0000 | 0.0000 |
| 215 | 189 | S1P(d18:1) | 0.0000 | 0.0000 |
| 216 | 191 | SM 32:1 | 0.0000 | 0.0000 |
| 217 | 192 | SM 32:2 | 0.0000 | 0.0000 |
| 218 | 193 | SM 33:1 | 0.0000 | 0.0000 |
| 219 | 198 | SM 35:2 | 0.0000 | 0.0000 |
| 220 | 199 | SM 36:1 | 0.0000 | 0.0000 |
| 221 | 201 | SM 38:1 | 0.0000 | 0.0000 |
| 222 | 202 | SM 38:2 | 0.0000 | 0.0000 |
| 223 | 204 | SM 40:1 | 0.0000 | 0.0000 |
| 224 | 205 | SM 41:1 | 0.0000 | 0.0000 |
| 225 | 207 | SM 44:3 | 0.0000 | 0.0000 |
| 226 | 210 | Sulfatide (d18:1:/16:0) | 0.0000 | 0.0000 |
| 227 | 211 | TG(48:0) [NL-16:0] | 0.0000 | 0.0000 |
| 228 | 212 | TG(48:1) [NL-16:1] | 0.0000 | 0.0000 |
| 229 | 213 | TG(48:1) [NL-18:1] | 0.0000 | 0.0000 |
| 230 | 214 | TG(48:2) [NL-14:0] | 0.0000 | 0.0000 |
| 231 | 215 | TG(48:2) [NL-14:1] | 0.0000 | 0.0000 |
| 232 | 216 | TG(48:2) [NL-16:1] | 0.0000 | 0.0000 |
| 233 | 218 | TG(48:3) [NL-14:0] | 0.0000 | 0.0000 |
| 234 | 219 | TG(48:3) [NL-16:1] | 0.0000 | 0.0000 |
| 235 | 220 | TG(48:3) [NL-18:3] | 0.0000 | 0.0000 |
| 236 | 221 | TG(49:1) [NL-17:1] | 0.0000 | 0.0000 |
| 237 | 222 | TG(50:0) [NL-18:0] | 0.0000 | 0.0000 |
| 238 | 223 | TG(50:1) [NL-16:0] | 0.0000 | 0.0000 |
| 239 | 224 | TG(50:1) [NL-18:1] | 0.0000 | 0.0000 |
| 240 | 225 | TG(50:2) [NL-14:0] | 0.0000 | 0.0000 |
| 241 | 226 | TG(50:2) [NL-16:1] | 0.0000 | 0.0000 |
| 242 | 227 | TG(50:2) [NL-18:1] | 0.0000 | 0.0000 |
| 243 | 228 | TG(50:2) [NL-18:2] | 0.0000 | 0.0000 |
| 244 | 229 | TG(50:3) [NL-14:0] | 0.0000 | 0.0000 |
| 245 | 230 | TG(50:3) [NL-14:1] | 0.0000 | 0.0000 |
| 246 | 231 | TG(50:3) [NL-16:1] | 0.0000 | 0.0000 |
| 247 | 232 | TG(50:3) [NL-18:2] | 0.0000 | 0.0000 |
| 248 | 235 | TG(50:4) [NL-18:3] | 0.0000 | 0.0000 |
| 249 | 236 | TG(50:4) [NL-20:4] | 0.0000 | 0.0000 |
| 250 | 238 | TG(51:1) [NL-17:0] | 0.0000 | 0.0000 |
| 251 | 239 | TG(51:2) [NL-15:0] | 0.0000 | 0.0000 |
| 252 | 240 | TG(51:2) [NL-17:0] | 0.0000 | 0.0000 |
| 253 | 241 | TG(51:2) [NL-17:1] | 0.0000 | 0.0000 |
| 254 | 242 | TG(52:1) [NL-18:0] | 0.0000 | 0.0000 |
| 255 | 243 | TG(52:1) [NL-18:1] | 0.0000 | 0.0000 |
| 256 | 244 | TG(52:2) [NL-16:0] | 0.0000 | 0.0000 |
| 257 | 245 | TG(52:3) [NL-18:2] | 0.0000 | 0.0000 |
| 258 | 246 | TG(52:4) [NL-16:1] | 0.0000 | 0.0000 |
| 259 | 247 | TG(52:4) [NL-18:2] | 0.0000 | 0.0000 |
| 260 | 248 | TG(52:4) [NL-18:3] | 0.0000 | 0.0000 |
| 261 | 249 | TG(52:5) [NL-18:3] | 0.0000 | 0.0000 |
| 262 | 250 | TG(52:5) [NL-20:4] | 0.0000 | 0.0000 |
| 263 | 251 | TG(52:5) [NL-20:5] | 0.0000 | 0.0000 |
| 264 | 252 | TG(53:2) [NL-17:1] | 0.0000 | 0.0000 |
| 265 | 253 | TG(53:2) [NL-18:1] | 0.0000 | 0.0000 |
| 266 | 254 | TG(54:2) [NL-18:0] | 0.0000 | 0.0000 |
| 267 | 256 | TG(54:3) [NL-18:1] | 0.0000 | 0.0000 |
| 268 | 257 | TG(54:3) [NL-18:2] | 0.0000 | 0.0000 |
| 269 | 258 | TG(54:4) [NL-18:2] | 0.0000 | 0.0000 |
| 270 | 260 | TG(54:5) [NL-18:3] | 0.0000 | 0.0000 |
| 271 | 261 | TG(54:5) [NL-20:4] | 0.0000 | 0.0000 |
| 272 | 263 | TG(54:6) [NL-20:4] | 0.0000 | 0.0000 |
| 273 | 264 | TG(54:6) [NL-20:5] | 0.0000 | 0.0000 |
| 274 | 265 | TG(54:6) [NL-22:6] | 0.0000 | 0.0000 |
| 275 | 267 | TG(54:7) [NL-22:6] | 0.0000 | 0.0000 |
| 276 | 269 | TG(56:6) [NL-22:5] | 0.0000 | 0.0000 |
| 277 | 270 | TG(56:7) [NL-20:4] | 0.0000 | 0.0000 |
| 278 | 271 | TG(56:7) [NL-20:5] | 0.0000 | 0.0000 |
| 279 | 272 | TG(56:7) [NL-22:5] | 0.0000 | 0.0000 |
| 280 | 273 | TG(56:7) [NL-22:6] | 0.0000 | 0.0000 |
| 281 | 274 | TG(56:8) [NL-20:4] | 0.0000 | 0.0000 |
| 282 | 275 | TG(56:8) [NL-20:5] | 0.0000 | 0.0000 |
| 283 | 276 | TG(56:8) [NL-22:6] | 0.0000 | 0.0000 |
| 284 | 277 | TG(56:9) [NL-22:6] | 0.0000 | 0.0000 |
| 285 | 279 | TG(58:8) [NL-22:6] | 0.0000 | 0.0000 |
| 286 | 281 | TG(O-50:1) [NL-15:0] | 0.0000 | 0.0000 |
| 287 | 282 | TG(O-50:1) [NL-16:0] | 0.0000 | 0.0000 |
| 288 | 283 | TG(O-50:1) [NL-17:1] | 0.0000 | 0.0000 |
| 289 | 284 | TG(O-50:1) [NL-18:1] | 0.0000 | 0.0000 |
| 290 | 285 | TG(O-50:2) [NL-16:1] | 0.0000 | 0.0000 |
| 291 | 286 | TG(O-50:2) [NL-18:1] | 0.0000 | 0.0000 |
| 292 | 287 | TG(O-50:2) [NL-18:2] | 0.0000 | 0.0000 |
| 293 | 288 | TG(O-52:0) [NL-16:0] | 0.0000 | 0.0000 |
| 294 | 289 | TG(O-52:2) [NL-16:0] | 0.0000 | 0.0000 |
| 295 | 290 | TG(O-52:2) [NL-17:1] | 0.0000 | 0.0000 |
| 296 | 292 | TG(O-54:3) [NL-17:1] | 0.0000 | 0.0000 |
| 297 | 293 | TG(O-54:3) [NL-18:1] | 0.0000 | 0.0000 |
| 298 | 294 | TG(O-54:4) [NL-17:1] | 0.0000 | 0.0000 |

TABLE 21
Predictive models of cardiovascular disease events developed
using Ridge regression with the CLP2s lipids.

| | | | | Ridge |
|---|---|---|---|---|
| Rank# | Lipid # | Lipid/Covariate | Ridge | (ABS) |
| | | drage_00 | 0.0056 | 0.0056 |
| | | intercept | −0.1684 | 0.1684 |
| 1 | 190 | SM 31:1 | −0.0100 | 0.0100 |
| 2 | 135 | PC 40:8 | −0.0084 | 0.0084 |
| 3 | 156 | PE 40:6 | −0.0082 | 0.0082 |
| 4 | 45 | Cer(m18:1/20:0) | 0.0079 | 0.0079 |
| 5 | 100 | LPC(P-17:0) | 0.0078 | 0.0078 |
| 6 | 143 | PC(P-34:2) | −0.0076 | 0.0076 |
| 7 | 47 | Cer(m18:1/24:0) | 0.0075 | 0.0075 |
| 8 | 1 | AcylCarnitine 12:0 | 0.0075 | 0.0075 |
| 9 | 103 | LPE 18:1 | 0.0072 | 0.0072 |
| 10 | 15 | CE 17:0 | 0.0071 | 0.0071 |
| 11 | 109 | PC 32:0 | 0.0071 | 0.0071 |
| 12 | 12 | CE 16:0 | 0.0070 | 0.0070 |
| 13 | 134 | PC 40:6 | 0.0068 | 0.0068 |
| 14 | 52 | DE(18:2) | 0.0066 | 0.0066 |
| 15 | 169 | PE(P-18:0/18:2) | 0.0066 | 0.0066 |
| 16 | 217 | TG(48:2) [NL-18:2] | 0.0065 | 0.0065 |
| 17 | 97 | LPC(O-24:0) | −0.0064 | 0.0064 |
| 18 | 194 | SM 34:1 | 0.0063 | 0.0063 |
| 19 | 129 | PC 38:4(a) | −0.0061 | 0.0061 |
| 20 | 148 | PE 34:1 | 0.0061 | 0.0061 |
| 21 | 130 | PC 38:4(b) | 0.0059 | 0.0059 |
| 22 | 203 | SM 38:3 | −0.0058 | 0.0058 |
| 23 | 195 | SM 34:2 | −0.0056 | 0.0056 |
| 24 | 78 | LPC 14:0 | −0.0055 | 0.0055 |
| 25 | 147 | PE 32:1 | −0.0054 | 0.0054 |
| 26 | 23 | CE 20:4 | −0.0053 | 0.0053 |
| 27 | 200 | SM 36:3 | −0.0052 | 0.0052 |
| 28 | 55 | DG 34:1-(18:1) | −0.0049 | 0.0049 |
| 29 | 111 | PC 32:2 | −0.0048 | 0.0048 |
| 30 | 157 | PE(O-34:1) | −0.0048 | 0.0048 |
| 31 | 274 | TG(56:8) [NL-20:4] | 0.0047 | 0.0047 |
| 32 | 90 | LPC 22:5 | −0.0045 | 0.0045 |
| 33 | 35 | Cer(d18:1/24:0) | 0.0045 | 0.0045 |
| 34 | 140 | PC(P-30:0) | 0.0043 | 0.0043 |
| 35 | 255 | TG(54:2) [NL-20:1] | 0.0043 | 0.0043 |
| 36 | 178 | PI 32:1 | −0.0043 | 0.0043 |
| 37 | 250 | TG(52:5) [NL-20:4] | −0.0043 | 0.0043 |
| 38 | 41 | Cer(d19:1/22:0) | −0.0042 | 0.0042 |
| 39 | 3 | AcylCarnitine 14:0 | 0.0042 | 0.0042 |
| 40 | 192 | SM 32:2 | −0.0041 | 0.0041 |
| 41 | 77 | Hex3Cer(d18:1/16:0) | −0.0041 | 0.0041 |
| 42 | 7 | AcylCarnitine 16:1 | −0.0040 | 0.0040 |
| 43 | 181 | PI 36:2 | −0.0039 | 0.0039 |
| 44 | 43 | Cer(d19:1/24:1) | 0.0039 | 0.0039 |
| 45 | 262 | TG(54:6) [NL-18:3] | 0.0038 | 0.0038 |
| 46 | 81 | LPC 18:1 | 0.0038 | 0.0038 |
| 47 | 29 | Cer(d16:1/24:1) | 0.0037 | 0.0037 |
| 48 | 272 | TG(56:7) [NL-22:5] | 0.0037 | 0.0037 |
| 49 | 180 | PI 36:1 | 0.0037 | 0.0037 |
| 50 | 28 | Cer(d16:1/24:0) | −0.0036 | 0.0036 |
| 51 | 266 | TG(54:7) [NL-20:5] | 0.0036 | 0.0036 |
| 52 | 208 | Sph(d17:1) | −0.0036 | 0.0036 |
| 53 | 39 | Cer(d18:2/24:0) | −0.0036 | 0.0036 |
| 54 | 126 | PC 36:5(a) | −0.0036 | 0.0036 |
| 55 | 273 | TG(56:7) [NL-22:6] | −0.0036 | 0.0036 |
| 56 | 132 | PC 38:6(a) | −0.0035 | 0.0035 |
| 57 | 105 | LPE 20:4 | 0.0034 | 0.0034 |
| 58 | 57 | DG 36:2-(18:1) | −0.0034 | 0.0034 |
| 59 | 85 | LPC 19:1 | −0.0034 | 0.0034 |
| 60 | 36 | Cer(d18:1/24:1) | 0.0034 | 0.0034 |
| 61 | 201 | SM 38:1 | 0.0033 | 0.0033 |
| 62 | 173 | PE(P-18:0/22:6) | −0.0033 | 0.0033 |
| 63 | 153 | PE 36:4 | 0.0033 | 0.0033 |
| 64 | 245 | TG(52:3) [NL-18:2] | −0.0033 | 0.0033 |
| 65 | 264 | TG(54:6) [NL-20:5] | −0.0033 | 0.0033 |
| 66 | 75 | Hex2Cer(d18:1/24:1) | 0.0031 | 0.0031 |
| 67 | 83 | LPC 18:3 | 0.0029 | 0.0029 |
| 68 | 14 | CE 16:2 | −0.0029 | 0.0029 |
| 69 | 32 | Cer(d18:1/20:0) | 0.0029 | 0.0029 |
| 70 | 11 | CE 15:0 | −0.0029 | 0.0029 |
| 71 | 280 | TG(58:9) [NL-22:6] | 0.0028 | 0.0028 |
| 72 | 26 | CE 22:6 | −0.0028 | 0.0028 |
| 73 | 234 | TG(50:4) [NL-14:0] | 0.0027 | 0.0027 |
| 74 | 116 | PC 34:2 | 0.0027 | 0.0027 |
| 75 | 58 | DG 36:3-(18:2) | 0.0027 | 0.0027 |
| 76 | 202 | SM 38:2 | −0.0027 | 0.0027 |
| 77 | 38 | Cer(d18:2/23:0) | −0.0027 | 0.0027 |
| 78 | 128 | PC 37:6 | −0.0026 | 0.0026 |
| 79 | 171 | PE(P-18:0/20:5) | −0.0026 | 0.0026 |
| 80 | 155 | PE 38:6 | −0.0025 | 0.0025 |
| 81 | 186 | PI 38:6 | 0.0025 | 0.0025 |
| 82 | 168 | PE(P-18:0/18:1) | 0.0025 | 0.0025 |
| 83 | 96 | LPC(O-22:1) | −0.0025 | 0.0025 |
| 84 | 20 | CE 20:0 | 0.0025 | 0.0025 |
| 85 | 95 | LPC(O-22:0) | −0.0024 | 0.0024 |
| 86 | 42 | Cer(d19:1/24:0) | 0.0024 | 0.0024 |
| 87 | 185 | PI 38:4 | −0.0024 | 0.0024 |
| 88 | 124 | PC 36:4(a) | −0.0024 | 0.0024 |
| 89 | 123 | PC 36:4 | −0.0024 | 0.0024 |
| 90 | 82 | LPC 18:2 | −0.0024 | 0.0024 |
| 91 | 278 | TG(58:10) [NL-22:6] | 0.0024 | 0.0024 |
| 92 | 159 | PE(O-36:4) | −0.0024 | 0.0024 |
| 93 | 244 | TG(52:2) [NL-16:0] | −0.0023 | 0.0023 |
| 94 | 229 | TG(50:3) [NL-14:0] | 0.0023 | 0.0023 |
| 95 | 8 | AcylCarnitine 18:1 | 0.0023 | 0.0023 |
| 96 | 115 | PC 34:1 | −0.0023 | 0.0023 |
| 97 | 219 | TG(48:3) [NL-16:1] | −0.0023 | 0.0023 |
| 98 | 193 | SM 33:1 | 0.0022 | 0.0022 |
| 99 | 40 | Cer(d18:2/24:1) | 0.0022 | 0.0022 |
| 100 | 269 | TG(56:6) [NL-22:5] | −0.0022 | 0.0022 |
| 101 | 141 | PC(P-32:0) | 0.0021 | 0.0021 |
| 102 | 189 | S1P(d18:1) | −0.0021 | 0.0021 |
| 103 | 154 | PE 38:4 | −0.0021 | 0.0021 |
| 104 | 296 | Ubiquinone | 0.0021 | 0.0021 |
| 105 | 37 | Cer(d18:2/22:0) | 0.0021 | 0.0021 |
| 106 | 204 | SM 40:1 | 0.0020 | 0.0020 |
| 107 | 177 | PE(P-20:0/20:4) | −0.0019 | 0.0019 |
| 108 | 4 | AcylCarnitine 14:1 | −0.0019 | 0.0019 |
| 109 | 106 | LPE 22:6 | −0.0018 | 0.0018 |
| 110 | 108 | PC 31:0 | 0.0018 | 0.0018 |
| 111 | 196 | SM 34:3 | −0.0018 | 0.0018 |
| 112 | 27 | Cer(d16:1/22:0) | 0.0018 | 0.0018 |
| 113 | 268 | TG(56:6) [NL-20:4] | −0.0018 | 0.0018 |
| 114 | 276 | TG(56:8) [NL-22:6] | 0.0017 | 0.0017 |
| 115 | 127 | PC 36:5(b) | −0.0017 | 0.0017 |
| 116 | 166 | PE(P-17:0/20:4) | 0.0017 | 0.0017 |
| 117 | 249 | TG(52:5) [NL-18:3] | 0.0017 | 0.0017 |
| 118 | 63 | GM3(d18:1/22:0) | −0.0017 | 0.0017 |
| 119 | 34 | Cer(d18:1/23:0) | −0.0017 | 0.0017 |
| 120 | 182 | PI 36:4 | −0.0017 | 0.0017 |
| 121 | 247 | TG(52:4) [NL-18:2] | 0.0016 | 0.0016 |
| 122 | 263 | TG(54:6) [NL-20:4] | −0.0016 | 0.0016 |
| 123 | 64 | GM3(d18:1/24:1) | 0.0016 | 0.0016 |
| 124 | 175 | PE(P-18:1/20:5) | 0.0016 | 0.0016 |
| 125 | 298 | LPE(P-18:0) | 0.0016 | 0.0016 |
| 126 | 149 | PE 34:2 | 0.0015 | 0.0015 |
| 127 | 48 | Cer(m18:1/24:1) | −0.0015 | 0.0015 |
| 128 | 25 | CE 22:1 | 0.0015 | 0.0015 |
| 129 | 50 | COH (161) | 0.0014 | 0.0014 |
| 130 | 6 | AcylCarnitine 16:0 | 0.0014 | 0.0014 |
| 131 | 10 | CE 14:0 | −0.0014 | 0.0014 |
| 132 | 227 | TG(50:2) [NL-18:1] | −0.0014 | 0.0014 |
| 133 | 197 | SM 35:1 | 0.0014 | 0.0014 |
| 134 | 33 | Cer(d18:1/22:0) | −0.0014 | 0.0014 |
| 135 | 21 | CE 20:1 | 0.0014 | 0.0014 |
| 136 | 84 | LPC 19:0 | −0.0014 | 0.0014 |
| 137 | 215 | TG(48:2) [NL-14:1] | 0.0014 | 0.0014 |
| 138 | 112 | PC 33:1 | 0.0013 | 0.0013 |
| 139 | 232 | TG(50:3) [NL-18:2] | 0.0013 | 0.0013 |
| 140 | 179 | PI 34:1 | 0.0013 | 0.0013 |
| 141 | 261 | TG(54:5) [NL-20:4] | −0.0013 | 0.0013 |
| 142 | 92 | LPC 24:0 | −0.0013 | 0.0013 |
| 143 | 170 | PE(P-18:0/20:4) | −0.0013 | 0.0013 |
| 144 | 174 | PE(P-18:1/20:4) | −0.0013 | 0.0013 |
| 145 | 161 | PE(P-16:0/18:2) | −0.0012 | 0.0012 |
| 146 | 275 | TG(56:8) [NL-20:5] | 0.0012 | 0.0012 |
| 147 | 56 | DG 34:2-(18:2) | 0.0012 | 0.0012 |
| 148 | 206 | SM 44:2 | 0.0012 | 0.0012 |
| 149 | 167 | PE(P-17:0/22:6) | 0.0012 | 0.0012 |
| 150 | 270 | TG(56:7) [NL-20:4] | −0.0012 | 0.0012 |
| 151 | 51 | DE(18:1) | 0.0012 | 0.0012 |
| 152 | 66 | Hex1Cer(d18:1/18:0) | −0.0012 | 0.0012 |
| 153 | 205 | SM 41:1 | −0.0012 | 0.0012 |
| 154 | 49 | Cer1P(d18:1/16:0) | −0.0012 | 0.0012 |
| 155 | 91 | LPC 22:6 | 0.0012 | 0.0012 |
| 156 | 152 | PE 36:2 | 0.0012 | 0.0012 |
| 157 | 9 | AcylCarnitine 18:2 | −0.0012 | 0.0012 |
| 158 | 277 | TG(56:9) [NL-22:6] | 0.0012 | 0.0012 |
| 159 | 279 | TG(58:8) [NL-22:6] | 0.0011 | 0.0011 |
| 160 | 214 | TG(48:2) [NL-14:0] | 0.0011 | 0.0011 |
| 161 | 101 | LPE 16:0 | 0.0011 | 0.0011 |
| 162 | 73 | Hex2Cer(d18:1/22:0) | 0.0011 | 0.0011 |
| 163 | 2 | AcylCarnitine 13:0 | −0.0011 | 0.0011 |
| 164 | 235 | TG(50:4) [NL-18:3] | −0.0010 | 0.0010 |
| 165 | 226 | TG(50:2) [NL-16:1] | −0.0010 | 0.0010 |
| 166 | 210 | Sulfatide (d18:1:/16:0) | 0.0010 | 0.0010 |
| 167 | 136 | PC(O-32:0) | −0.0010 | 0.0010 |
| 168 | 287 | TG(O-50:2) [NL-18:2] | −0.0010 | 0.0010 |
| 169 | 131 | PC 38:4(c) | 0.0009 | 0.0009 |
| 170 | 53 | DE(20:4) | 0.0009 | 0.0009 |
| 171 | 110 | PC 32:1 | 0.0009 | 0.0009 |
| 172 | 187 | PI 40:6 | −0.0009 | 0.0009 |
| 173 | 256 | TG(54:3) [NL-18:1] | 0.0009 | 0.0009 |
| 174 | 93 | LPC 26:0 | −0.0008 | 0.0008 |
| 175 | 67 | Hex1Cer(d18:1/22:0) | 0.0008 | 0.0008 |
| 176 | 18 | CE 18:2 | −0.0008 | 0.0008 |
| 177 | 69 | Hex1Cer(d18:1/24:1) | −0.0008 | 0.0008 |
| 178 | 297 | LPE(P-16:0) | 0.0007 | 0.0007 |
| 179 | 19 | CE 18:3 | −0.0007 | 0.0007 |
| 180 | 125 | PC 36:4(b) | −0.0006 | 0.0006 |
| 181 | 107 | LPI 18:0 | −0.0006 | 0.0006 |
| 182 | 163 | PE(P-16:0/20:5) | 0.0006 | 0.0006 |
| 183 | 236 | TG(50:4) [NL-20:4] | −0.0006 | 0.0006 |
| 184 | 13 | CE 16:1 | 0.0006 | 0.0006 |
| 185 | 104 | LPE 18:2 | 0.0006 | 0.0006 |
| 186 | 230 | TG(50:3) [NL-14:1] | 0.0006 | 0.0006 |
| 187 | 198 | SM 35:2 | −0.0005 | 0.0005 |
| 188 | 113 | PC 33:2 | 0.0005 | 0.0005 |
| 189 | 65 | Hex1Cer(d18:1/16:0) | −0.0005 | 0.0005 |
| 190 | 102 | LPE 18:0 | −0.0005 | 0.0005 |
| 191 | 265 | TG(54:6) [NL-22:6] | 0.0005 | 0.0005 |
| 192 | 80 | LPC 18:0 | −0.0005 | 0.0005 |
| 193 | 188 | PS 38:4 | −0.0005 | 0.0005 |
| 194 | 183 | PI 37:4 | 0.0005 | 0.0005 |
| 195 | 89 | LPC 22:0 | −0.0005 | 0.0005 |
| 196 | 209 | Sulfatide (d18:1:/16:0(OH)) | 0.0005 | 0.0005 |
| 197 | 87 | LPC 20:4 | −0.0004 | 0.0004 |
| 198 | 88 | LPC 20:5 | 0.0004 | 0.0004 |
| 199 | 70 | Hex1Cer(d18:2/24:0) | 0.0004 | 0.0004 |
| 200 | 71 | Hex2Cer(d16:1/16:0) | −0.0004 | 0.0004 |
| 201 | 146 | PC(P-37:4) | 0.0004 | 0.0004 |
| 202 | 191 | SM 32:1 | 0.0004 | 0.0004 |
| 203 | 72 | Hex2Cer(d18:1/16:0) | −0.0003 | 0.0003 |
| 204 | 60 | DG 38:4-(20:3) | 0.0003 | 0.0003 |
| 205 | 31 | Cer(d18:1/16:0) | 0.0003 | 0.0003 |
| 206 | 99 | LPC(P-16:0) | −0.0003 | 0.0003 |
| 207 | 176 | PE(P-20:0/18:2) | −0.0003 | 0.0003 |
| 208 | 165 | PE(P-16:0/22:6) | −0.0003 | 0.0003 |
| 209 | 271 | TG(56:7) [NL-20:5] | −0.0003 | 0.0003 |
| 210 | 30 | Cer(d17:1/24:0) | 0.0002 | 0.0002 |
| 211 | 79 | LPC 17:0 | 0.0002 | 0.0002 |
| 212 | 122 | PC 36:2 | 0.0002 | 0.0002 |
| 213 | 241 | TG(51:2) [NL-17:1] | −0.0002 | 0.0002 |
| 214 | 216 | TG(48:2) [NL-16:1] | −0.0002 | 0.0002 |
| 215 | 144 | PC(P-36:4) | 0.0002 | 0.0002 |
| 216 | 231 | TG(50:3) [NL-16:1] | 0.0002 | 0.0002 |
| 217 | 286 | TG(O-50:2) [NL-18:1] | 0.0001 | 0.0001 |
| 218 | 267 | TG(54:7) [NL-22:6] | −0.0001 | 0.0001 |
| 219 | 44 | Cer(m18:0/22:0) | −0.0001 | 0.0001 |
| 220 | 199 | SM 36:1 | −0.0001 | 0.0001 |
| 221 | 248 | TG(52:4) [NL-18:3] | −0.0001 | 0.0001 |
| 222 | 260 | TG(54:5) [NL-18:3] | 0.0001 | 0.0001 |
| 223 | 218 | TG(48:3) [NL-14:0] | 0.0001 | 0.0001 |
| 224 | 213 | TG(48:1) [NL-18:1] | 0.0001 | 0.0001 |
| 225 | 294 | TG(O-54:4) [NL-17:1] | 0.0001 | 0.0001 |
| 226 | 5 | AcylCarnitine 14:2 | 0.0001 | 0.0001 |
| 227 | 240 | TG(51:2) [NL-17:0] | −0.0001 | 0.0001 |
| 228 | 285 | TG(O-50:2) [NL-16:1] | −0.0001 | 0.0001 |
| 229 | 46 | Cer(m18:1/22:0) | 0.0000 | 0.0000 |
| 230 | 162 | PE(P-16:0/20:4) | 0.0000 | 0.0000 |
| 231 | 133 | PC 38:6(b) | 0.0000 | 0.0000 |
| 232 | 246 | TG(52:4) [NL-16:1] | 0.0000 | 0.0000 |
| 233 | 24 | CE 20:5 | 0.0000 | 0.0000 |
| 234 | 145 | PC(P-36:5) | 0.0000 | 0.0000 |
| 235 | 54 | DG 32:0-(16:0) | 0.0000 | 0.0000 |

TABLE 22
Predictive models of cardiovascular disease events developed
using LASSO regression with the CLP2s lipids.

| | | | | LASSO |
|---|---|---|---|---|
| Rank# | Lipid # | Lipid/Covariate | LASSO | (ABS) |
| | | drage_00 | 0.0061 | 0.0061 |
| | | intercept | −0.1972 | 0.1972 |
| 1 | 190 | SM 31:1 | −0.0116 | 0.0116 |
| 2 | 135 | PC 40:8 | −0.0100 | 0.0100 |
| 3 | 15 | CE 17:0 | 0.0097 | 0.0097 |
| 4 | 195 | SM 34:2 | −0.0095 | 0.0095 |
| 5 | 126 | PC 36:5(a) | −0.0076 | 0.0076 |
| 6 | 97 | LPC(O-24:0) | −0.0075 | 0.0075 |
| 7 | 45 | Cer(m18:1/20:0) | 0.0068 | 0.0068 |
| 8 | 103 | LPE 18:1 | 0.0067 | 0.0067 |
| 9 | 12 | CE 16:0 | 0.0066 | 0.0066 |
| 10 | 156 | PE 40:6 | −0.0064 | 0.0064 |
| 11 | 47 | Cer(m18:1/24:0) | 0.0059 | 0.0059 |
| 12 | 109 | PC 32:0 | 0.0058 | 0.0058 |
| 13 | 1 | AcylCarnitine 12:0 | 0.0057 | 0.0057 |
| 14 | 194 | SM 34:1 | 0.0056 | 0.0056 |
| 15 | 100 | LPC(P-17:0) | 0.0056 | 0.0056 |
| 16 | 143 | PC(P-34:2) | −0.0051 | 0.0051 |
| 17 | 234 | TG(50:4) [NL-14:0] | 0.0048 | 0.0048 |
| 18 | 23 | CE 20:4 | −0.0047 | 0.0047 |
| 19 | 203 | SM 38:3 | −0.0046 | 0.0046 |
| 20 | 200 | SM 36:3 | −0.0045 | 0.0045 |
| 21 | 90 | LPC 22:5 | −0.0044 | 0.0044 |
| 22 | 129 | PC 38:4(a) | −0.0043 | 0.0043 |
| 23 | 157 | PE(O-34:1) | −0.0043 | 0.0043 |
| 24 | 52 | DE(18:2) | 0.0043 | 0.0043 |
| 25 | 180 | PI 36:1 | 0.0034 | 0.0034 |
| 26 | 36 | Cer(d18:1/24:1) | 0.0029 | 0.0029 |
| 27 | 14 | CE 16:2 | −0.0029 | 0.0029 |
| 28 | 262 | TG(54:6) [NL-18:3] | 0.0029 | 0.0029 |
| 29 | 130 | PC 38:4(b) | 0.0029 | 0.0029 |
| 30 | 208 | Sph(d17:1) | −0.0028 | 0.0028 |
| 31 | 29 | Cer(d16:1/24:1) | 0.0027 | 0.0027 |
| 32 | 43 | Cer(d19:1/24:1) | 0.0026 | 0.0026 |
| 33 | 217 | TG(48:2) [NL-18:2] | 0.0025 | 0.0025 |
| 34 | 181 | PI 36:2 | −0.0023 | 0.0023 |
| 35 | 141 | PC(P-32:0) | 0.0023 | 0.0023 |
| 36 | 38 | Cer(d18:2/23:0) | −0.0023 | 0.0023 |
| 37 | 204 | SM 40:1 | 0.0022 | 0.0022 |
| 38 | 159 | PE(O-36:4) | −0.0022 | 0.0022 |
| 39 | 78 | LPC 14:0 | −0.0022 | 0.0022 |
| 40 | 278 | TG(58:10) [NL-22:6] | 0.0021 | 0.0021 |
| 41 | 20 | CE 20:0 | 0.0020 | 0.0020 |
| 42 | 255 | TG(54:2) [NL-20:1] | 0.0020 | 0.0020 |
| 43 | 280 | TG(58:9) [NL-22:6] | 0.0019 | 0.0019 |
| 44 | 21 | CE 20:1 | 0.0019 | 0.0019 |
| 45 | 25 | CE 22:1 | 0.0019 | 0.0019 |
| 46 | 132 | PC 38:6(a) | −0.0016 | 0.0016 |
| 47 | 266 | TG(54:7) [NL-20:5] | 0.0016 | 0.0016 |
| 48 | 11 | CE 15:0 | −0.0015 | 0.0015 |
| 49 | 296 | Ubiquinone | 0.0014 | 0.0014 |
| 50 | 26 | CE 22:6 | −0.0014 | 0.0014 |
| 51 | 168 | PE(P-18:0/18:1) | 0.0013 | 0.0013 |
| 52 | 105 | LPE 20:4 | 0.0013 | 0.0013 |
| 53 | 140 | PC(P-30:0) | 0.0012 | 0.0012 |
| 54 | 50 | COH (161) | 0.0012 | 0.0012 |
| 55 | 96 | LPC(O-22:1) | −0.0012 | 0.0012 |
| 56 | 169 | PE(P-18:0/18:2) | 0.0011 | 0.0011 |
| 57 | 51 | DE(18:1) | 0.0008 | 0.0008 |
| 58 | 185 | PI 38:4 | −0.0008 | 0.0008 |
| 59 | 123 | PC 36:4 | −0.0008 | 0.0008 |
| 60 | 95 | LPC(O-22:0) | −0.0007 | 0.0007 |
| 61 | 148 | PE 34:1 | 0.0007 | 0.0007 |
| 62 | 93 | LPC 26:0 | −0.0007 | 0.0007 |
| 63 | 32 | Cer(d18:1/20:0) | 0.0006 | 0.0006 |
| 64 | 202 | SM 38:2 | −0.0005 | 0.0005 |
| 65 | 24 | CE 20:5 | −0.0005 | 0.0005 |
| 66 | 84 | LPC 19:0 | −0.0005 | 0.0005 |
| 67 | 111 | PC 32:2 | −0.0003 | 0.0003 |
| 68 | 3 | AcylCarnitine 14:0 | 0.0003 | 0.0003 |
| 69 | 177 | PE(P-20:0/20:4) | −0.0002 | 0.0002 |
| 70 | 19 | CE 18:3 | −0.0002 | 0.0002 |
| 71 | 66 | Hex1Cer(d18:1/18:0) | −0.0002 | 0.0002 |
| 72 | 206 | SM 44:2 | 0.0002 | 0.0002 |
| 73 | 31 | Cer(d18:1/16:0) | 0.0001 | 0.0001 |
| 74 | 70 | Hex1Cer(d18:2/24:0) | −0.0001 | 0.0001 |
| 75 | 107 | LPI 18:0 | −0.0001 | 0.0001 |
| 76 | 209 | Sulfatide (d18:1:/16:0(OH)) | 0.0001 | 0.0001 |
| 77 | 2 | AcylCarnitine 13:0 | 0.0000 | 0.0000 |
| 78 | 4 | AcylCarnitine 14:1 | 0.0000 | 0.0000 |
| 79 | 5 | AcylCarnitine 14:2 | 0.0000 | 0.0000 |
| 80 | 6 | AcylCarnitine 16:0 | 0.0000 | 0.0000 |
| 81 | 7 | AcylCarnitine 16:1 | 0.0000 | 0.0000 |
| 82 | 8 | AcylCarnitine 18:1 | 0.0000 | 0.0000 |
| 83 | 9 | AcylCarnitine 18:2 | 0.0000 | 0.0000 |
| 84 | 10 | CE 14:0 | 0.0000 | 0.0000 |
| 85 | 13 | CE 16:1 | 0.0000 | 0.0000 |
| 86 | 18 | CE 18:2 | 0.0000 | 0.0000 |
| 87 | 27 | Cer(d16:1/22:0) | 0.0000 | 0.0000 |
| 88 | 28 | Cer(d16:1/24:0) | 0.0000 | 0.0000 |
| 89 | 30 | Cer(d17:1/24:0) | 0.0000 | 0.0000 |
| 90 | 33 | Cer(d18:1/22:0) | 0.0000 | 0.0000 |
| 91 | 34 | Cer(d18:1/23:0) | 0.0000 | 0.0000 |
| 92 | 35 | Cer(d18:1/24:0) | 0.0000 | 0.0000 |
| 93 | 37 | Cer(d18:2/22:0) | 0.0000 | 0.0000 |
| 94 | 39 | Cer(d18:2/24:0) | 0.0000 | 0.0000 |
| 95 | 40 | Cer(d18:2/24:1) | 0.0000 | 0.0000 |
| 96 | 41 | Cer(d19:1/22:0) | 0.0000 | 0.0000 |
| 97 | 42 | Cer(d19:1/24:0) | 0.0000 | 0.0000 |
| 98 | 44 | Cer(m18:0/22:0) | 0.0000 | 0.0000 |
| 99 | 46 | Cer(m18:1/22:0) | 0.0000 | 0.0000 |
| 100 | 48 | Cer(m18:1/24:1) | 0.0000 | 0.0000 |
| 101 | 49 | Cer1P(d18:1/16:0) | 0.0000 | 0.0000 |
| 102 | 53 | DE(20:4) | 0.0000 | 0.0000 |
| 103 | 54 | DG 32:0-(16:0) | 0.0000 | 0.0000 |
| 104 | 55 | DG 34:1-(18:1) | 0.0000 | 0.0000 |
| 105 | 56 | DG 34:2-(18:2) | 0.0000 | 0.0000 |
| 106 | 57 | DG 36:2-(18:1) | 0.0000 | 0.0000 |
| 107 | 58 | DG 36:3-(18:2) | 0.0000 | 0.0000 |
| 108 | 60 | DG 38:4-(20:3) | 0.0000 | 0.0000 |
| 109 | 63 | GM3(d18:1/22:0) | 0.0000 | 0.0000 |
| 110 | 64 | GM3(d18:1/24:1) | 0.0000 | 0.0000 |
| 111 | 65 | Hex1Cer(d18:1/16:0) | 0.0000 | 0.0000 |
| 112 | 67 | Hex1Cer(d18:1/22:0) | 0.0000 | 0.0000 |
| 113 | 69 | Hex1Cer(d18:1/24:1) | 0.0000 | 0.0000 |
| 114 | 71 | Hex2Cer(d16:1/16:0) | 0.0000 | 0.0000 |
| 115 | 72 | Hex2Cer(d18:1/16:0) | 0.0000 | 0.0000 |
| 116 | 73 | Hex2Cer(d18:1/22:0) | 0.0000 | 0.0000 |
| 117 | 75 | Hex2Cer(d18:1/24:1) | 0.0000 | 0.0000 |
| 118 | 77 | Hex3Cer(d18:1/16:0) | 0.0000 | 0.0000 |
| 119 | 79 | LPC 17:0 | 0.0000 | 0.0000 |
| 120 | 80 | LPC 18:0 | 0.0000 | 0.0000 |
| 121 | 81 | LPC 18:1 | 0.0000 | 0.0000 |
| 122 | 82 | LPC 18:2 | 0.0000 | 0.0000 |
| 123 | 83 | LPC 18:3 | 0.0000 | 0.0000 |
| 124 | 85 | LPC 19:1 | 0.0000 | 0.0000 |
| 125 | 87 | LPC 20:4 | 0.0000 | 0.0000 |
| 126 | 88 | LPC 20:5 | 0.0000 | 0.0000 |
| 127 | 89 | LPC 22:0 | 0.0000 | 0.0000 |
| 128 | 91 | LPC 22:6 | 0.0000 | 0.0000 |
| 129 | 92 | LPC 24:0 | 0.0000 | 0.0000 |
| 130 | 99 | LPC(P-16:0) | 0.0000 | 0.0000 |
| 131 | 101 | LPE 16:0 | 0.0000 | 0.0000 |
| 132 | 102 | LPE 18:0 | 0.0000 | 0.0000 |
| 133 | 104 | LPE 18:2 | 0.0000 | 0.0000 |
| 134 | 106 | LPE 22:6 | 0.0000 | 0.0000 |
| 135 | 108 | PC 31:0 | 0.0000 | 0.0000 |
| 136 | 110 | PC 32:1 | 0.0000 | 0.0000 |
| 137 | 112 | PC 33:1 | 0.0000 | 0.0000 |
| 138 | 113 | PC 33:2 | 0.0000 | 0.0000 |
| 139 | 115 | PC 34:1 | 0.0000 | 0.0000 |
| 140 | 116 | PC 34:2 | 0.0000 | 0.0000 |
| 141 | 122 | PC 36:2 | 0.0000 | 0.0000 |
| 142 | 124 | PC 36:4(a) | 0.0000 | 0.0000 |
| 143 | 125 | PC 36:4(b) | 0.0000 | 0.0000 |
| 144 | 127 | PC 36:5(b) | 0.0000 | 0.0000 |
| 145 | 128 | PC 37:6 | 0.0000 | 0.0000 |
| 146 | 131 | PC 38:4(c) | 0.0000 | 0.0000 |
| 147 | 133 | PC 38:6(b) | 0.0000 | 0.0000 |
| 148 | 134 | PC 40:6 | 0.0000 | 0.0000 |
| 149 | 136 | PC(O-32:0) | 0.0000 | 0.0000 |
| 150 | 144 | PC(P-36:4) | 0.0000 | 0.0000 |
| 151 | 145 | PC(P-36:5) | 0.0000 | 0.0000 |
| 152 | 146 | PC(P-37:4) | 0.0000 | 0.0000 |
| 153 | 147 | PE 32:1 | 0.0000 | 0.0000 |
| 154 | 149 | PE 34:2 | 0.0000 | 0.0000 |
| 155 | 152 | PE 36:2 | 0.0000 | 0.0000 |
| 156 | 153 | PE 36:4 | 0.0000 | 0.0000 |
| 157 | 154 | PE 38:4 | 0.0000 | 0.0000 |
| 158 | 155 | PE 38:6 | 0.0000 | 0.0000 |
| 159 | 161 | PE(P-16:0/18:2) | 0.0000 | 0.0000 |
| 160 | 162 | PE(P-16:0/20:4) | 0.0000 | 0.0000 |
| 161 | 163 | PE(P-16:0/20:5) | 0.0000 | 0.0000 |
| 162 | 165 | PE(P-16:0/22:6) | 0.0000 | 0.0000 |
| 163 | 166 | PE(P-17:0/20:4) | 0.0000 | 0.0000 |
| 164 | 167 | PE(P-17:0/22:6) | 0.0000 | 0.0000 |
| 165 | 170 | PE(P-18:0/20:4) | 0.0000 | 0.0000 |
| 166 | 171 | PE(P-18:0/20:5) | 0.0000 | 0.0000 |
| 167 | 173 | PE(P-18:0/22:6) | 0.0000 | 0.0000 |
| 168 | 174 | PE(P-18:1/20:4) | 0.0000 | 0.0000 |
| 169 | 175 | PE(P-18:1/20:5) | 0.0000 | 0.0000 |
| 170 | 176 | PE(P-20:0/18:2) | 0.0000 | 0.0000 |
| 171 | 178 | PI 32:1 | 0.0000 | 0.0000 |
| 172 | 179 | PI 34:1 | 0.0000 | 0.0000 |
| 173 | 182 | PI 36:4 | 0.0000 | 0.0000 |
| 174 | 183 | PI 37:4 | 0.0000 | 0.0000 |
| 175 | 186 | PI 38:6 | 0.0000 | 0.0000 |
| 176 | 187 | PI 40:6 | 0.0000 | 0.0000 |
| 177 | 188 | PS 38:4 | 0.0000 | 0.0000 |
| 178 | 189 | S1P(d18:1) | 0.0000 | 0.0000 |
| 179 | 191 | SM 32:1 | 0.0000 | 0.0000 |
| 180 | 192 | SM 32:2 | 0.0000 | 0.0000 |
| 181 | 193 | SM 33:1 | 0.0000 | 0.0000 |
| 182 | 196 | SM 34:3 | 0.0000 | 0.0000 |
| 183 | 197 | SM 35:1 | 0.0000 | 0.0000 |
| 184 | 198 | SM 35:2 | 0.0000 | 0.0000 |
| 185 | 199 | SM 36:1 | 0.0000 | 0.0000 |
| 186 | 201 | SM 38:1 | 0.0000 | 0.0000 |
| 187 | 205 | SM 41:1 | 0.0000 | 0.0000 |
| 188 | 210 | Sulfatide (d18:1:/16:0) | 0.0000 | 0.0000 |
| 189 | 213 | TG(48:1) [NL-18:1] | 0.0000 | 0.0000 |
| 190 | 214 | TG(48:2) [NL-14:0] | 0.0000 | 0.0000 |
| 191 | 215 | TG(48:2) [NL-14:1] | 0.0000 | 0.0000 |
| 192 | 216 | TG(48:2) [NL-16:1] | 0.0000 | 0.0000 |
| 193 | 218 | TG(48:3) [NL-14:0] | 0.0000 | 0.0000 |
| 194 | 219 | TG(48:3) [NL-16:1] | 0.0000 | 0.0000 |
| 195 | 226 | TG(50:2) [NL-16:1] | 0.0000 | 0.0000 |
| 196 | 227 | TG(50:2) [NL-18:1] | 0.0000 | 0.0000 |
| 197 | 229 | TG(50:3) [NL-14:0] | 0.0000 | 0.0000 |
| 198 | 230 | TG(50:3) [NL-14:1] | 0.0000 | 0.0000 |
| 199 | 231 | TG(50:3) [NL-16:1] | 0.0000 | 0.0000 |
| 200 | 232 | TG(50:3) [NL-18:2] | 0.0000 | 0.0000 |
| 201 | 235 | TG(50:4) [NL-18:3] | 0.0000 | 0.0000 |
| 202 | 236 | TG(50:4) [NL-20:4] | 0.0000 | 0.0000 |
| 203 | 240 | TG(51:2) [NL-17:0] | 0.0000 | 0.0000 |
| 204 | 241 | TG(51:2) [NL-17:1] | 0.0000 | 0.0000 |
| 205 | 244 | TG(52:2) [NL-16:0] | 0.0000 | 0.0000 |
| 206 | 245 | TG(52:3) [NL-18:2] | 0.0000 | 0.0000 |
| 207 | 246 | TG(52:4) [NL-16:1] | 0.0000 | 0.0000 |
| 208 | 247 | TG(52:4) [NL-18:2] | 0.0000 | 0.0000 |
| 209 | 248 | TG(52:4) [NL-18:3] | 0.0000 | 0.0000 |
| 210 | 249 | TG(52:5) [NL-18:3] | 0.0000 | 0.0000 |
| 211 | 250 | TG(52:5) [NL-20:4] | 0.0000 | 0.0000 |
| 212 | 256 | TG(54:3) [NL-18:1] | 0.0000 | 0.0000 |
| 213 | 260 | TG(54:5) [NL-18:3] | 0.0000 | 0.0000 |
| 214 | 261 | TG(54:5) [NL-20:4] | 0.0000 | 0.0000 |
| 215 | 263 | TG(54:6) [NL-20:4] | 0.0000 | 0.0000 |
| 216 | 264 | TG(54:6) [NL-20:5] | 0.0000 | 0.0000 |
| 217 | 265 | TG(54:6) [NL-22:6] | 0.0000 | 0.0000 |
| 218 | 267 | TG(54:7) [NL-22:6] | 0.0000 | 0.0000 |
| 219 | 268 | TG(56:6) [NL-20:4] | 0.0000 | 0.0000 |
| 220 | 269 | TG(56:6) [NL-22:5] | 0.0000 | 0.0000 |
| 221 | 270 | TG(56:7) [NL-20:4] | 0.0000 | 0.0000 |
| 222 | 271 | TG(56:7) [NL-20:5] | 0.0000 | 0.0000 |
| 223 | 272 | TG(56:7) [NL-22:5] | 0.0000 | 0.0000 |
| 224 | 273 | TG(56:7) [NL-22:6] | 0.0000 | 0.0000 |
| 225 | 274 | TG(56:8) [NL-20:4] | 0.0000 | 0.0000 |
| 226 | 275 | TG(56:8) [NL-20:5] | 0.0000 | 0.0000 |
| 227 | 276 | TG(56:8) [NL-22:6] | 0.0000 | 0.0000 |
| 228 | 277 | TG(56:9) [NL-22:6] | 0.0000 | 0.0000 |
| 229 | 279 | TG(58:8) [NL-22:6] | 0.0000 | 0.0000 |
| 230 | 285 | TG(O-50:2) [NL-16:1] | 0.0000 | 0.0000 |
| 231 | 286 | TG(O-50:2) [NL-18:1] | 0.0000 | 0.0000 |
| 232 | 287 | TG(O-50:2) [NL-18:2] | 0.0000 | 0.0000 |
| 233 | 294 | TG(O-54:4) [NL-17:1] | 0.0000 | 0.0000 |
| 234 | 297 | LPE(P-16:0) | 0.0000 | 0.0000 |
| 235 | 298 | LPE(P-18:0) | 0.0000 | 0.0000 |

### Example 8: Prediabetes and Type 2 Diabetes (T2DM) Risk Score

Risk scores based on plasma lipidomic measures were generated. This score was derived from the fasting plasma lipidome using a comprehensive mass spectrometry approach to enable risk assessment for impaired glucose tolerance (IGT) without an oral glucose tolerance test (OGTT). The lipids in fasting plasma were useful to identify individuals who have isolated IGT, who are often overlooked in the current clinical pathway, and also useful for future T2D risk assessment.

### Development of a IGT Risk Model

AusDiab participants data was used. Preliminary lipidomic analysis had been done on small case-control subsets of this cohort previously [7, 8]. The AusDiab is a prospective study examining the prevalence and risk factors of T2DM and CVD in the Australian adult population [9]. T2DM was defined as fasting glucose measurement (FBG) of 7 mmol/L or greater or a 2-hour post load glucose measurement (2 h-PLG) of 11.1 mmol/L or greater. Prediabetes was defined as either impaired fasting glucose (FBG=6.1 to 6.9 mmol/L), impaired glucose tolerance (2 h-PLG=7.8 to 11 mmol/L), or a combination of both. As current definitions of IGT overlaps with impaired fasting glycaemia (IFG) (elevated 2 h-PLG but criteria for FBG is only <7 mmol/L), individuals with only IGT but otherwise healthy FBG levels (<6.1 mmol/L) were termed isolated IGT. Individuals with abnormally low 2 h-PLG measurements (2 h-PLG<3 mmol/L) were further excluded.

Predicted glycemic scores: A series of glycemic scores were generated by using all the Research Lipid List (Table 2) or the clinical platform subsets CLP1s (Table 4) and CLP2s (Table 6) to predict different glycemic measurements amenable to T2DM risk and diagnosis (FBG, 2 h-PLG and HbA1C) using either a ridge regression model or a LASSO model. The lipid data were scaled to the standard deviation and centred to the mean. This resulted in three additional scores for each individual (denoted as lipid-FBG, lipid-2 h-PLG and lipid-HbA1C respectively). The ridge and LASSO models for 2 h-PLG developed using CLP1s and CLP2s lipid lists are described as a list of the covariates and lipids used in each model together with their β-coefficients (Tables 23, 24, 25 and 26).

TABLE 23
Predictive models of 2h-PLG developed using
Ridge regression with the CLP1s lipids.

| Rank | Lipid | | | Ridge |
|---|---|---|---|---|
| # | # | Lipid/Covariate | Ridge | (ABS) |
| | | (Intercept) | 5.906 | 5.906 |
| 1 | 82 | LPC 18:2 | −0.379 | 0.379 |
| 2 | 86 | LPC 20:0 | 0.240 | 0.240 |
| 3 | 115 | PC(15:0_22:6) | −0.212 | 0.212 |
| 4 | 80 | LPC 18:0 | 0.202 | 0.202 |
| 5 | 5 | Acylcarnitine(14:2) | −0.167 | 0.167 |
| 6 | 4 | Acylcarnitine(14:1) | 0.160 | 0.160 |
| 7 | 137 | PC(40:8) | −0.158 | 0.158 |
| 8 | 7 | Acylcarnitine(16:1) | 0.149 | 0.149 |
| 9 | 201 | SM(d18:1/14:0)/SM(d16:1/16:0) | −0.148 | 0.148 |
| 10 | 200 | SM(d17:1/16:0) | 0.145 | 0.145 |
| 11 | 39 | Cer(d18:2/24:0) | −0.135 | 0.135 |
| 12 | 98 | LPC(O-24:2) | 0.132 | 0.132 |
| 13 | 91 | LPC 22:6 | 0.131 | 0.131 |
| 14 | 2 | Acylcarnitine(13:0) | −0.130 | 0.130 |
| 15 | 57 | DG(18:1_18:1) | −0.126 | 0.126 |
| 16 | 199 | SM(d17:1/14:0) | −0.122 | 0.122 |
| 17 | 175 | PE(P-18:0/22:6) | 0.116 | 0.116 |
| 18 | 101 | LPE 16:0 | −0.116 | 0.116 |
| 19 | 220 | TG(48:3) [NL-14:0] | −0.114 | 0.114 |
| 20 | 8 | Acylcarnitine(18:1) | 0.110 | 0.110 |
| 21 | 44 | Cer(m18:0/22:0) | 0.110 | 0.110 |
| 22 | 106 | LPE 22:6 | 0.108 | 0.108 |
| 23 | 133 | PC(34:5) | 0.105 | 0.105 |
| 24 | 122 | PC(16:0_22:6) | −0.104 | 0.104 |
| 25 | 111 | PC 36:2 | −0.101 | 0.101 |
| 26 | 60 | DG(18:2_18:2) | 0.098 | 0.098 |
| 27 | 54 | DG(16:0_16:0) | 0.097 | 0.097 |
| 28 | 164 | PE(P-16:0/20:4) | 0.095 | 0.095 |
| 29 | 76 | HexCer(d18:1/24:1) | −0.093 | 0.093 |
| 30 | 267 | TG(54:6) [NL-22:6] | 0.091 | 0.091 |
| 31 | 203 | SM(d18:1/18:0)/SM(d16:1/20:0) | 0.091 | 0.091 |
| 32 | 22 | CE(20:2) | 0.090 | 0.090 |
| 33 | 132 | PC(33:2) | 0.090 | 0.090 |
| 34 | 103 | LPE 18:1 | 0.090 | 0.090 |
| 35 | 285 | TG(O-50:1) [NL-17:1] | −0.089 | 0.089 |
| 36 | 191 | S1P(d18:1) | −0.089 | 0.089 |
| 37 | 11 | CE(15:0) | 0.087 | 0.087 |
| 38 | 221 | TG(48:3) [NL-16:1] | −0.085 | 0.085 |
| 39 | 141 | PC(O-36:5) | −0.083 | 0.083 |
| 40 | 116 | PC(16:0_16:0) | 0.083 | 0.083 |
| 41 | 3 | Acylcarnitine(14:0) | 0.082 | 0.082 |
| 42 | 246 | TG(52:2) [NL-16:0] | −0.078 | 0.078 |
| 43 | 171 | PE(P-18:0/18:2) | 0.077 | 0.077 |
| 44 | 99 | LPC(P-16:0) | −0.077 | 0.077 |
| 45 | 178 | PE(P-20:0/18:2) | −0.077 | 0.077 |
| 46 | 189 | PI(38:6) | −0.076 | 0.076 |
| 47 | 94 | LPC(O-20:0) | −0.076 | 0.076 |
| 48 | 291 | TG(O-52:2) [NL-16:0] | 0.076 | 0.076 |
| 49 | 131 | PC(33:1) | 0.076 | 0.076 |
| 50 | 65 | Hex2Cer(d16:1/16:0) | 0.075 | 0.075 |
| 51 | 15 | CE(17:0) | −0.075 | 0.075 |
| 52 | 211 | Sulfatide (d18:1:/16:0(OH)) | −0.075 | 0.075 |
| 53 | 18 | CE(18:2) | −0.073 | 0.073 |
| 54 | 92 | LPC 24:0 | −0.073 | 0.073 |
| 55 | 33 | Cer(d18:1/22:0) | 0.072 | 0.072 |
| 56 | 45 | Cer(m18:1/20:0) | 0.072 | 0.072 |
| 57 | 184 | PI(18:0_18:1) | 0.072 | 0.072 |
| 58 | 95 | LPC(O-22:0) | −0.071 | 0.071 |
| 59 | 250 | TG(52:4) [NL-18:3] | −0.071 | 0.071 |
| 60 | 105 | LPE 20:4 | 0.071 | 0.071 |
| 61 | 229 | TG(50:2) [NL-18:1] | −0.071 | 0.071 |
| 62 | 149 | PE 34:3 | 0.071 | 0.071 |
| 63 | 125 | PC(18:0_20:4) | 0.070 | 0.070 |
| 64 | 166 | PE(P-16:0/22:4) | −0.069 | 0.069 |
| 65 | 31 | Cer(d18:1/16:0) | 0.068 | 0.068 |
| 66 | 136 | PC(38:6) (a) | −0.067 | 0.067 |
| 67 | 228 | TG(50:2) [NL-16:1] | −0.066 | 0.066 |
| 68 | 278 | TG(56:8) [NL-22:6] | 0.065 | 0.065 |
| 69 | 126 | PC(18:0_22:6) | 0.065 | 0.065 |
| 70 | 202 | SM(d18:1/16:0) | −0.065 | 0.065 |
| 71 | 81 | LPC 18:1 | −0.064 | 0.064 |
| 72 | 233 | TG(50:3) [NL-16:1] | −0.064 | 0.064 |
| 73 | 198 | SM(44:3) | −0.063 | 0.063 |
| 74 | 135 | PC(38:4) (b) | 0.062 | 0.062 |
| 75 | 197 | SM(44:2) | 0.061 | 0.061 |
| 76 | 9 | Acylcarnitine(18:2) | −0.060 | 0.060 |
| 77 | 275 | TG(56:7) [NL-22:6] | 0.060 | 0.060 |
| 78 | 156 | PE(18:0_18:1) | −0.060 | 0.060 |
| 79 | 241 | TG(51:2) [NL-15:0] | −0.060 | 0.060 |
| 80 | 248 | TG(52:4) [NL-16:1] | 0.060 | 0.060 |
| 81 | 208 | SM(d18:2/18:1) | −0.059 | 0.059 |
| 82 | 162 | PE(P-16:0/18:1) | −0.058 | 0.058 |
| 83 | 268 | TG(54:7) [NL-20:5] | 0.058 | 0.058 |
| 84 | 213 | TG(48:0) [NL-16:0] | 0.057 | 0.057 |
| 85 | 6 | Acylcarnitine(16:0) | 0.057 | 0.057 |
| 86 | 30 | Cer(d17:1/24:0) | 0.057 | 0.057 |
| 87 | 130 | PC(32:2) | 0.056 | 0.056 |
| 88 | 40 | Cer(d18:2/24:1) | 0.056 | 0.056 |
| 89 | 225 | TG(50:1) [NL-16:0] | 0.056 | 0.056 |
| 90 | 143 | PC(P-16:0/16:0) | 0.056 | 0.056 |
| 91 | 114 | PC(15:0_20:4) | 0.055 | 0.055 |
| 92 | 260 | TG(54:4) [NL-18:2] | 0.055 | 0.055 |
| 93 | 146 | PC(P-16:0/20:4) | −0.054 | 0.054 |
| 94 | 53 | DE(20:4) | 0.053 | 0.053 |
| 95 | 42 | Cer(d19:1/24:0) | −0.053 | 0.053 |
| 96 | 287 | TG(O-50:2) [NL-16:1] | −0.052 | 0.052 |
| 97 | 205 | SM(d18:1/22:0)/SM(d16:1/24:0) | −0.052 | 0.052 |
| 98 | 10 | CE(14:0) | −0.052 | 0.052 |
| 99 | 259 | TG(54:3) [NL-18:2] | −0.051 | 0.051 |
| 100 | 286 | TG(O-50:1) [NL-18:1] | −0.051 | 0.051 |
| 101 | 240 | TG(51:1) [NL-17:0] | 0.050 | 0.050 |
| 102 | 117 | PC(16:0_18:0) | −0.050 | 0.050 |
| 103 | 256 | TG(54:2) [NL-18:0] | 0.049 | 0.049 |
| 104 | 281 | TG(58:8) [NL-22:6] | −0.048 | 0.048 |
| 105 | 180 | PI 38:3 | 0.048 | 0.048 |
| 106 | 157 | PE(18:0_20:4) | −0.047 | 0.047 |
| 107 | 279 | TG(56:9) [NL-22:6] | 0.047 | 0.047 |
| 108 | 14 | CE(16:2) | 0.047 | 0.047 |
| 109 | 102 | LPE 18:0 | 0.046 | 0.046 |
| 110 | 295 | TG(O-54:3) [NL-18:1] | 0.046 | 0.046 |
| 111 | 159 | PE(O-16:0/18:2) | 0.045 | 0.045 |
| 112 | 262 | TG(54:5) [NL-18:3] | −0.045 | 0.045 |
| 113 | 196 | SM(34:3) | 0.045 | 0.045 |
| 114 | 193 | SM 35:2 | 0.044 | 0.044 |
| 115 | 48 | Cer(m18:1/24:1) | −0.044 | 0.044 |
| 116 | 187 | PI(34:1) | 0.044 | 0.044 |
| 117 | 79 | LPC 17:0 | −0.044 | 0.044 |
| 118 | 88 | LPC 20:5 | −0.044 | 0.044 |
| 119 | 138 | PC(O-16:0/16:0) | 0.044 | 0.044 |
| 120 | 255 | TG(53:2) [NL-18:1] | 0.042 | 0.042 |
| 121 | 28 | Cer(d16:1/24:0) | −0.042 | 0.042 |
| 122 | 207 | SM(d18:2/16:0) | 0.041 | 0.041 |
| 123 | 249 | TG(52:4) [NL-18:2] | 0.041 | 0.041 |
| 124 | 167 | PE(P-16:0/22:6) | 0.041 | 0.041 |
| 125 | 288 | TG(O-50:2) [NL-18:1] | 0.041 | 0.041 |
| 126 | 274 | TG(56:7) [NL-22:5] | −0.041 | 0.041 |
| 127 | 74 | HexCer(d18:1/22:0) | 0.040 | 0.040 |
| 128 | 161 | PE(O-34:1) | −0.040 | 0.040 |
| 129 | 294 | TG(O-54:3) [NL-17:1] | 0.040 | 0.040 |
| 130 | 264 | TG(54:6) [NL-18:3] | 0.039 | 0.039 |
| 131 | 34 | Cer(d18:1/23:0) | 0.039 | 0.039 |
| 132 | 296 | TG(O-54:4) [NL-17:1] | 0.039 | 0.039 |
| 133 | 282 | TG(58:9) [NL-22:6] | 0.039 | 0.039 |
| 134 | 212 | Sulfatide (d18:1:/16:0) | −0.039 | 0.039 |
| 135 | 185 | PI(18:0_20:4) | −0.039 | 0.039 |
| 136 | 163 | PE(P-16:0/18:2) | −0.039 | 0.039 |
| 137 | 235 | TG(50:3) [NL-18:3] | −0.038 | 0.038 |
| 138 | 55 | DG(16:0_18:1) | −0.038 | 0.038 |
| 139 | 152 | PE(16:0_18:1) | −0.038 | 0.038 |
| 140 | 254 | TG(53:2) [NL-17:1] | 0.038 | 0.038 |
| 141 | 120 | PC(16:0_20:4) | −0.038 | 0.038 |
| 142 | 245 | TG(52:1) [NL-18:1] | −0.037 | 0.037 |
| 143 | 172 | PE(P-18:0/20:4) | 0.037 | 0.037 |
| 144 | 100 | LPC(P-17:0) | 0.037 | 0.037 |
| 145 | 284 | TG(O-50:1) [NL-16:0] | 0.036 | 0.036 |
| 146 | 69 | Hex2Cer(d18:1/24:1) | −0.036 | 0.036 |
| 147 | 247 | TG(52:3) [NL-18:2] | 0.035 | 0.035 |
| 148 | 21 | CE(20:1) | −0.035 | 0.035 |
| 149 | 160 | PE(O-16:0/20:4) | −0.035 | 0.035 |
| 150 | 96 | LPC(O-22:1) | −0.035 | 0.035 |
| 151 | 150 | PE 36:2 | −0.035 | 0.035 |
| 152 | 83 | LPC 18:3 | −0.034 | 0.034 |
| 153 | 113 | PC(14:0_20:4) | 0.034 | 0.034 |
| 154 | 168 | PE(P-17:0/20:4) | −0.033 | 0.033 |
| 155 | 63 | GM3(d18:1/22:0) | 0.033 | 0.033 |
| 156 | 121 | PC(16:0_20:5) | 0.033 | 0.033 |
| 157 | 142 | PC(P-16:0/14:0) | 0.033 | 0.033 |
| 158 | 148 | PC(P-37:4) | −0.033 | 0.033 |
| 159 | 24 | CE(20:5) | −0.032 | 0.032 |
| 160 | 173 | PE(P-18:0/20:5) | −0.032 | 0.032 |
| 161 | 97 | LPC(O-24:0) | −0.032 | 0.032 |
| 162 | 206 | SM(d18:2/14:0) | 0.031 | 0.031 |
| 163 | 183 | PI(16:0_20:4) | 0.031 | 0.031 |
| 164 | 108 | LPE(P-18:0) | −0.030 | 0.030 |
| 165 | 204 | SM(d18:1/20:0)/SM(d16:1/22:0) | −0.030 | 0.030 |
| 166 | 218 | TG(48:2) [NL-16:1] | −0.030 | 0.030 |
| 167 | 261 | TG(54:4) [NL-20:3] | 0.030 | 0.030 |
| 168 | 273 | TG(56:7) [NL-20:5] | 0.029 | 0.029 |
| 169 | 165 | PE(P-16:0/20:5) | 0.028 | 0.028 |
| 170 | 188 | PI(36:2) | −0.028 | 0.028 |
| 171 | 289 | TG(O-50:2) [NL-18:2] | −0.028 | 0.028 |
| 172 | 257 | TG(54:2) [NL-20:1] | 0.028 | 0.028 |
| 173 | 177 | PE(P-18:1/20:5) | −0.027 | 0.027 |
| 174 | 290 | TG(O-52:0) [NL-16:0] | −0.027 | 0.027 |
| 175 | 67 | Hex2Cer(d18:1/22:0) | 0.026 | 0.026 |
| 176 | 37 | Cer(d18:2/22:0) | 0.025 | 0.025 |
| 177 | 251 | TG(52:5) [NL-18:3] | 0.025 | 0.025 |
| 178 | 222 | TG(48:3) [NL-18:3] | 0.025 | 0.025 |
| 179 | 238 | TG(50:4) [NL-20:4] | −0.025 | 0.025 |
| 180 | 56 | DG(16:0_18:2) | 0.025 | 0.025 |
| 181 | 176 | PE(P-18:1/20:4) | 0.024 | 0.024 |
| 182 | 52 | DE(18:2) | 0.024 | 0.024 |
| 183 | 32 | Cer(d18:1/20:0) | −0.023 | 0.023 |
| 184 | 123 | PC(16:1_20:4) | 0.023 | 0.023 |
| 185 | 19 | CE(18:3) | 0.023 | 0.023 |
| 186 | 269 | TG(54:7) [NL-22:6] | 0.023 | 0.023 |
| 187 | 77 | HexCer(d18:2/24:0) | 0.023 | 0.023 |
| 188 | 224 | TG(50:0) [NL-18:0] | −0.023 | 0.023 |
| 189 | 217 | TG(48:2) [NL-14:1] | 0.023 | 0.023 |
| 190 | 58 | DG(18:1_18:2) | −0.022 | 0.022 |
| 191 | 90 | LPC 22:5 | 0.022 | 0.022 |
| 192 | 84 | LPC 19:0 | −0.022 | 0.022 |
| 193 | 20 | CE(20:0) | 0.021 | 0.021 |
| 194 | 272 | TG(56:7) [NL-20:4] | −0.021 | 0.021 |
| 195 | 107 | LPE(P-16:0) | −0.021 | 0.021 |
| 196 | 209 | SM(d18:2/20:0) | −0.021 | 0.021 |
| 197 | 118 | PC(16:0_18:1) | 0.020 | 0.020 |
| 198 | 297 | TG(O-54:4) [NL-18:2] | 0.020 | 0.020 |
| 199 | 239 | TG(51:0) [NL-16:0] | −0.020 | 0.020 |
| 200 | 231 | TG(50:3) [NL-14:0] | 0.020 | 0.020 |
| 201 | 78 | LPC 14:0 | 0.019 | 0.019 |
| 202 | 93 | LPC 26:0 | 0.019 | 0.019 |
| 203 | 158 | PE(18:0_22:6) | −0.018 | 0.018 |
| 204 | 230 | TG(50:2) [NL-18:2] | 0.018 | 0.018 |
| 205 | 70 | Hex2Cer(d18:2/16:0) | −0.018 | 0.018 |
| 206 | 13 | CE(16:1) | 0.017 | 0.017 |
| 207 | 186 | PI(18:0_22:6) | 0.017 | 0.017 |
| 208 | 271 | TG(56:6) [NL-22:5] | −0.017 | 0.017 |
| 209 | 87 | LPC 20:4 | −0.017 | 0.017 |
| 210 | 182 | PI(16:0_16:1) | −0.017 | 0.017 |
| 211 | 293 | TG(O-52:2) [NL-18:1] | 0.017 | 0.017 |
| 212 | 127 | PC(18:1_20:3) | −0.017 | 0.017 |
| 213 | 192 | SM 35:1 | −0.017 | 0.017 |
| 214 | 134 | PC(35:5) | 0.017 | 0.017 |
| 215 | 25 | CE(22:1) | −0.016 | 0.016 |
| 216 | 276 | TG(56:8) [NL-20:4] | 0.016 | 0.016 |
| 217 | 145 | PC(P-16:0/18:2) | −0.016 | 0.016 |
| 218 | 119 | PC(16:0_18:2) | 0.016 | 0.016 |
| 219 | 253 | TG(52:5) [NL-20:5] | 0.016 | 0.016 |
| 220 | 26 | CE(22:6) | −0.016 | 0.016 |
| 221 | 169 | PE(P-17:0/22:6) | −0.015 | 0.015 |
| 222 | 270 | TG(56:6) [NL-20:4] | −0.015 | 0.015 |
| 223 | 153 | PE(16:0_18:2) | 0.015 | 0.015 |
| 224 | 277 | TG(56:8) [NL-20:5] | −0.014 | 0.014 |
| 225 | 298 | Ubiquinone | 0.013 | 0.013 |
| 226 | 215 | TG(48:1) [NL-18:1] | 0.013 | 0.013 |
| 227 | 85 | LPC 19:1 | 0.013 | 0.013 |
| 228 | 292 | TG(O-52:2) [NL-17:1] | −0.013 | 0.013 |
| 229 | 38 | Cer(d18:2/23:0) | 0.013 | 0.013 |
| 230 | 237 | TG(50:4) [NL-18:3] | −0.013 | 0.013 |
| 231 | 266 | TG(54:6) [NL-20:5] | 0.012 | 0.012 |
| 232 | 242 | TG(51:2) [NL-17:0] | 0.012 | 0.012 |
| 233 | 29 | Cer(d16:1/24:1) | 0.012 | 0.012 |
| 234 | 243 | TG(51:2) [NL-17:1] | 0.012 | 0.012 |
| 235 | 73 | HexCer(d18:1/18:0) | −0.012 | 0.012 |
| 236 | 109 | LPI 18:0 | 0.011 | 0.011 |
| 237 | 179 | PE(P-20:0/20:4) | 0.011 | 0.011 |
| 238 | 112 | PC 36:4 | −0.011 | 0.011 |
| 239 | 41 | Cer(d19:1/22:0) | 0.011 | 0.011 |
| 240 | 36 | Cer(d18:1/24:1) | 0.011 | 0.011 |
| 241 | 216 | TG(48:2) [NL-14:0] | −0.011 | 0.011 |
| 242 | 223 | TG(49:1) [NL-17:1] | 0.011 | 0.011 |
| 243 | 64 | GM3(d18:1/24:1) | −0.010 | 0.010 |
| 244 | 71 | Hex3Cer(d18:1/16:0) | −0.010 | 0.010 |
| 245 | 128 | PC(18:2_18:2) | −0.010 | 0.010 |
| 246 | 263 | TG(54:5) [NL-20:4] | 0.010 | 0.010 |
| 247 | 226 | TG(50:1) [NL-18:1] | −0.010 | 0.010 |
| 248 | 140 | PC(O-36:0) | 0.010 | 0.010 |
| 249 | 154 | PE(16:0_20:4) | −0.010 | 0.010 |
| 250 | 68 | Hex2Cer(d18:1/24:0) | −0.009 | 0.009 |
| 251 | 16 | CE(18:0) | −0.009 | 0.009 |
| 252 | 234 | TG(50:3) [NL-18:2] | −0.009 | 0.009 |
| 253 | 252 | TG(52:5) [NL-20:4] | −0.008 | 0.008 |
| 254 | 227 | TG(50:2) [NL-14:0] | −0.008 | 0.008 |
| 255 | 174 | PE(P-18:0/22:4) | 0.008 | 0.008 |
| 256 | 66 | Hex2Cer(d18:1/16:0) | −0.008 | 0.008 |
| 257 | 17 | CE(18:1) | −0.007 | 0.007 |
| 258 | 62 | GM3(d18:1/18:0) | −0.007 | 0.007 |
| 259 | 23 | CE(20:4) | 0.007 | 0.007 |
| 260 | 47 | Cer(m18:1/24:0) | −0.006 | 0.006 |
| 261 | 46 | Cer(m18:1/22:0) | 0.006 | 0.006 |
| 262 | 35 | Cer(d18:1/24:0) | 0.006 | 0.006 |
| 263 | 49 | Cer1P(d18:1/16:0) | 0.006 | 0.006 |
| 264 | 244 | TG(52:1) [NL-18:0] | −0.006 | 0.006 |
| 265 | 129 | PC(32:1) | −0.006 | 0.006 |
| 266 | 50 | COH | 0.006 | 0.006 |
| 267 | 170 | PE(P-18:0/18:1) | −0.005 | 0.005 |
| 268 | 181 | PI(15-MHDA_20:4)/PI(17:0_20:4) | −0.005 | 0.005 |
| 269 | 110 | PC 31:0 | 0.005 | 0.005 |
| 270 | 258 | TG(54:3) [NL-18:1] | −0.005 | 0.005 |
| 271 | 61 | GM3(d18:1/16:0) | −0.004 | 0.004 |
| 272 | 194 | SM 38:3 | 0.004 | 0.004 |
| 273 | 12 | CE(16:0) | −0.004 | 0.004 |
| 274 | 283 | TG(O-50:1) [NL-15:0] | −0.004 | 0.004 |
| 275 | 151 | PE(16:0_16:1) | −0.004 | 0.004 |
| 276 | 190 | PS(38:4) | 0.004 | 0.004 |
| 277 | 139 | PC(O-34:2) | −0.004 | 0.004 |
| 278 | 104 | LPE 18:2 | 0.004 | 0.004 |
| 279 | 280 | TG(58:10) [NL-22:6] | 0.004 | 0.004 |
| 280 | 232 | TG(50:3) [NL-14:1] | −0.004 | 0.004 |
| 281 | 210 | Sph(d17:1) | 0.004 | 0.004 |
| 282 | 144 | PC(P-16:0/18:1) | 0.003 | 0.003 |
| 283 | 155 | PE(16:0_22:6) | 0.003 | 0.003 |
| 284 | 75 | HexCer(d18:1/24:0) | 0.003 | 0.003 |
| 285 | 147 | PC(P-16:0/20:5) | 0.002 | 0.002 |
| 286 | 72 | HexCer(d18:1/16:0) | −0.002 | 0.002 |
| 287 | 214 | TG(48:1) [NL-16:1] | 0.002 | 0.002 |
| 288 | 265 | TG(54:6) [NL-20:4] | 0.002 | 0.002 |
| 289 | 51 | DE(18:1) | −0.002 | 0.002 |
| 290 | 89 | LPC 22:0 | 0.002 | 0.002 |
| 291 | 219 | TG(48:2) [NL-18:2] | 0.002 | 0.002 |
| 292 | 59 | DG(18:1_20:3) | 0.002 | 0.002 |
| 293 | 124 | PC(18:0_18:1) | −0.001 | 0.001 |
| 294 | 43 | Cer(d19:1/24:1) | −0.001 | 0.001 |
| 295 | 1 | Acylcarnitine(12:0) | 0.001 | 0.001 |
| 296 | 236 | TG(50:4) [NL-14:0] | 0.001 | 0.001 |
| 297 | 27 | Cer(d16:1/22:0) | 0.000 | 0.000 |
| 298 | 195 | SM 41:1 | 0.000 | 0.000 |

TABLE 24
Predictive models of 2h-PLG developed using
LASSO regression with the CLP1s lipids.

| Rank | Lipid | | | LASSO |
|---|---|---|---|---|
| # | # | Lipid/Covariate | LASSO | (ABS) |
| | | (Intercept) | 5.906 | 5.906 |
| 1 | 82 | LPC 18:2 | −0.587 | 0.587 |
| 2 | 115 | PC(15:0_22:6) | −0.347 | 0.347 |
| 3 | 91 | LPC 22:6 | 0.264 | 0.264 |
| 4 | 80 | LPC 18:0 | 0.249 | 0.249 |
| 5 | 4 | Acylcarnitine(14:1) | 0.243 | 0.243 |
| 6 | 86 | LPC 20:0 | 0.236 | 0.236 |
| 7 | 5 | Acylcarnitine(14:2) | −0.212 | 0.212 |
| 8 | 132 | PC(33:2) | 0.192 | 0.192 |
| 9 | 200 | SM(d17:1/16:0) | 0.186 | 0.186 |
| 10 | 57 | DG(18:1_18:1) | −0.181 | 0.181 |
| 11 | 201 | SM(d18:1/14:0)/SM(d16:1/16:0) | −0.177 | 0.177 |
| 12 | 137 | PC(40:8) | −0.163 | 0.163 |
| 13 | 133 | PC(34:5) | 0.147 | 0.147 |
| 14 | 7 | Acylcarnitine(16:1) | 0.133 | 0.133 |
| 15 | 39 | Cer(d18:2/24:0) | −0.132 | 0.132 |
| 16 | 98 | LPC(O-24:2) | 0.131 | 0.131 |
| 17 | 175 | PE(P-18:0/22:6) | 0.130 | 0.130 |
| 18 | 2 | Acylcarnitine(13:0) | −0.130 | 0.130 |
| 19 | 60 | DG(18:2_18:2) | 0.120 | 0.120 |
| 20 | 267 | TG(54:6) [NL-22:6] | 0.119 | 0.119 |
| 21 | 199 | SM(d17:1/14:0) | −0.113 | 0.113 |
| 22 | 44 | Cer(m18:0/22:0) | 0.108 | 0.108 |
| 23 | 101 | LPE 16:0 | −0.107 | 0.107 |
| 24 | 103 | LPE 18:1 | 0.104 | 0.104 |
| 25 | 164 | PE(P-16:0/20:4) | 0.103 | 0.103 |
| 26 | 278 | TG(56:8) [NL-22:6] | 0.101 | 0.101 |
| 27 | 106 | LPE 22:6 | 0.101 | 0.101 |
| 28 | 220 | TG(48:3) [NL-14:0] | −0.099 | 0.099 |
| 29 | 191 | S1P(d18:1) | −0.096 | 0.096 |
| 30 | 111 | PC 36:2 | −0.095 | 0.095 |
| 31 | 122 | PC(16:0_22:6) | −0.095 | 0.095 |
| 32 | 141 | PC(O-36:5) | −0.095 | 0.095 |
| 33 | 99 | LPC(P-16:0) | −0.093 | 0.093 |
| 34 | 291 | TG(O-52:2) [NL-16:0] | 0.089 | 0.089 |
| 35 | 76 | HexCer(d18:1/24:1) | −0.088 | 0.088 |
| 36 | 131 | PC(33:1) | 0.087 | 0.087 |
| 37 | 11 | CE(15:0) | 0.087 | 0.087 |
| 38 | 285 | TG(O-50:1) [NL-17:1] | −0.086 | 0.086 |
| 39 | 33 | Cer(d18:1/22:0) | 0.082 | 0.082 |
| 40 | 22 | CE(20:2) | 0.081 | 0.081 |
| 41 | 246 | TG(52:2) [NL-16:0] | −0.076 | 0.076 |
| 42 | 211 | Sulfatide (d18:1:/16:0(OH)) | −0.076 | 0.076 |
| 43 | 116 | PC(16:0_16:0) | 0.076 | 0.076 |
| 44 | 6 | Acylcarnitine(16:0) | 0.076 | 0.076 |
| 45 | 18 | CE(18:2) | −0.074 | 0.074 |
| 46 | 54 | DG(16:0_16:0) | 0.072 | 0.072 |
| 47 | 8 | Acylcarnitine(18:1) | 0.070 | 0.070 |
| 48 | 94 | LPC(O-20:0) | −0.069 | 0.069 |
| 49 | 228 | TG(50:2) [NL-16:1] | −0.069 | 0.069 |
| 50 | 202 | SM(d18:1/16:0) | −0.067 | 0.067 |
| 51 | 65 | Hex2Cer(d16:1/16:0) | 0.067 | 0.067 |
| 52 | 156 | PE(18:0_18:1) | −0.066 | 0.066 |
| 53 | 178 | PE(P-20:0/18:2) | −0.065 | 0.065 |
| 54 | 203 | SM(d18:1/18:0)/SM(d16:1/20:0) | 0.064 | 0.064 |
| 55 | 130 | PC(32:2) | 0.063 | 0.063 |
| 56 | 31 | Cer(d18:1/16:0) | 0.062 | 0.062 |
| 57 | 229 | TG(50:2) [NL-18:1] | −0.062 | 0.062 |
| 58 | 15 | CE(17:0) | −0.061 | 0.061 |
| 59 | 162 | PE(P-16:0/18:1) | −0.061 | 0.061 |
| 60 | 114 | PC(15:0_20:4) | 0.060 | 0.060 |
| 61 | 184 | PI(18:0_18:1) | 0.060 | 0.060 |
| 62 | 198 | SM(44:3) | −0.060 | 0.060 |
| 63 | 105 | LPE 20:4 | 0.059 | 0.059 |
| 64 | 95 | LPC(O-22:0) | −0.059 | 0.059 |
| 65 | 143 | PC(P-16:0/16:0) | 0.058 | 0.058 |
| 66 | 221 | TG(48:3) [NL-16:1] | −0.058 | 0.058 |
| 67 | 40 | Cer(d18:2/24:1) | 0.055 | 0.055 |
| 68 | 268 | TG(54:7) [NL-20:5] | 0.054 | 0.054 |
| 69 | 92 | LPC 24:0 | −0.053 | 0.053 |
| 70 | 225 | TG(50:1) [NL-16:0] | 0.053 | 0.053 |
| 71 | 104 | LPE 18:2 | 0.051 | 0.051 |
| 72 | 146 | PC(P-16:0/20:4) | −0.051 | 0.051 |
| 73 | 250 | TG(52:4) [NL-18:3] | −0.050 | 0.050 |
| 74 | 260 | TG(54:4) [NL-18:2] | 0.049 | 0.049 |
| 75 | 79 | LPC 17:0 | −0.049 | 0.049 |
| 76 | 189 | PI(38:6) | −0.048 | 0.048 |
| 77 | 135 | PC(38:4) (b) | 0.047 | 0.047 |
| 78 | 96 | LPC(O-22:1) | −0.046 | 0.046 |
| 79 | 45 | Cer(m18:1/20:0) | 0.046 | 0.046 |
| 80 | 166 | PE(P-16:0/22:4) | −0.046 | 0.046 |
| 81 | 233 | TG(50:3) [NL-16:1] | −0.045 | 0.045 |
| 82 | 295 | TG(O-54:3) [NL-18:1] | 0.044 | 0.044 |
| 83 | 14 | CE(16:2) | 0.043 | 0.043 |
| 84 | 53 | DE(20:4) | 0.042 | 0.042 |
| 85 | 34 | Cer(d18:1/23:0) | 0.041 | 0.041 |
| 86 | 287 | TG(O-50:2) [NL-16:1] | −0.040 | 0.040 |
| 87 | 206 | SM(d18:2/14:0) | 0.040 | 0.040 |
| 88 | 138 | PC(O-16:0/16:0) | 0.038 | 0.038 |
| 89 | 288 | TG(O-50:2) [NL-18:1] | 0.037 | 0.037 |
| 90 | 10 | CE(14:0) | −0.036 | 0.036 |
| 91 | 136 | PC(38:6) (a) | −0.036 | 0.036 |
| 92 | 3 | Acylcarnitine(14:0) | 0.035 | 0.035 |
| 93 | 197 | SM(44:2) | 0.035 | 0.035 |
| 94 | 241 | TG(51:2) [NL-15:0] | −0.034 | 0.034 |
| 95 | 171 | PE(P-18:0/18:2) | 0.034 | 0.034 |
| 96 | 240 | TG(51:1) [NL-17:0] | 0.034 | 0.034 |
| 97 | 161 | PE(O-34:1) | −0.034 | 0.034 |
| 98 | 117 | PC(16:0_18:0) | −0.033 | 0.033 |
| 99 | 180 | PI 38:3 | 0.033 | 0.033 |
| 100 | 212 | Sulfatide (d18:1:/16:0) | −0.031 | 0.031 |
| 101 | 21 | CE(20:1) | −0.031 | 0.031 |
| 102 | 296 | TG(O-54:4) [NL-17:1] | 0.030 | 0.030 |
| 103 | 126 | PC(18:0_22:6) | 0.030 | 0.030 |
| 104 | 142 | PC(P-16:0/14:0) | 0.030 | 0.030 |
| 105 | 42 | Cer(d19:1/24:0) | −0.029 | 0.029 |
| 106 | 248 | TG(52:4) [NL-16:1] | 0.029 | 0.029 |
| 107 | 196 | SM(34:3) | 0.029 | 0.029 |
| 108 | 157 | PE(18:0_20:4) | −0.028 | 0.028 |
| 109 | 259 | TG(54:3) [NL-18:2] | −0.028 | 0.028 |
| 110 | 125 | PC(18:0_20:4) | 0.028 | 0.028 |
| 111 | 52 | DE(18:2) | 0.028 | 0.028 |
| 112 | 275 | TG(56:7) [NL-22:6] | 0.027 | 0.027 |
| 113 | 207 | SM(d18:2/16:0) | 0.027 | 0.027 |
| 114 | 69 | Hex2Cer(d18:1/24:1) | −0.026 | 0.026 |
| 115 | 108 | LPE(P-18:0) | −0.026 | 0.026 |
| 116 | 97 | LPC(O-24:0) | −0.026 | 0.026 |
| 117 | 148 | PC(P-37:4) | −0.026 | 0.026 |
| 118 | 77 | HexCer(d18:2/24:0) | 0.026 | 0.026 |
| 119 | 205 | SM(d18:1/22:0)/SM(d16:1/24:0) | −0.026 | 0.026 |
| 120 | 245 | TG(52:1) [NL-18:1] | −0.026 | 0.026 |
| 121 | 254 | TG(53:2) [NL-17:1] | 0.025 | 0.025 |
| 122 | 159 | PE(O-16:0/18:2) | 0.025 | 0.025 |
| 123 | 256 | TG(54:2) [NL-18:0] | 0.025 | 0.025 |
| 124 | 247 | TG(52:3) [NL-18:2] | 0.025 | 0.025 |
| 125 | 30 | Cer(d17:1/24:0) | 0.024 | 0.024 |
| 126 | 294 | TG(O-54:3) [NL-17:1] | 0.023 | 0.023 |
| 127 | 74 | HexCer(d18:1/22:0) | 0.022 | 0.022 |
| 128 | 279 | TG(56:9) [NL-22:6] | 0.020 | 0.020 |
| 129 | 204 | SM(d18:1/20:0)/SM(d16:1/22:0) | −0.019 | 0.019 |
| 130 | 208 | SM(d18:2/18:1) | −0.019 | 0.019 |
| 131 | 84 | LPC 19:0 | −0.018 | 0.018 |
| 132 | 261 | TG(54:4) [NL-20:3] | 0.018 | 0.018 |
| 133 | 24 | CE(20:5) | −0.017 | 0.017 |
| 134 | 286 | TG(O-50:1) [NL-18:1] | −0.016 | 0.016 |
| 135 | 81 | LPC 18:1 | −0.016 | 0.016 |
| 136 | 107 | LPE(P-16:0) | −0.015 | 0.015 |
| 137 | 63 | GM3(d18:1/22:0) | 0.014 | 0.014 |
| 138 | 120 | PC(16:0_20:4) | −0.014 | 0.014 |
| 139 | 66 | Hex2Cer(d18:1/16:0) | −0.013 | 0.013 |
| 140 | 213 | TG(48:0) [NL-16:0] | 0.012 | 0.012 |
| 141 | 149 | PE 34:3 | 0.012 | 0.012 |
| 142 | 37 | Cer(d18:2/22:0) | 0.012 | 0.012 |
| 143 | 297 | TG(O-54:4) [NL-18:2] | 0.011 | 0.011 |
| 144 | 67 | Hex2Cer(d18:1/22:0) | 0.010 | 0.010 |
| 145 | 188 | PI(36:2) | −0.010 | 0.010 |
| 146 | 160 | PE(O-16:0/20:4) | −0.010 | 0.010 |
| 147 | 119 | PC(16:0_18:2) | 0.009 | 0.009 |
| 148 | 185 | PI(18:0_20:4) | −0.008 | 0.008 |
| 149 | 255 | TG(53:2) [NL-18:1] | 0.007 | 0.007 |
| 150 | 25 | CE(22:1) | −0.006 | 0.006 |
| 151 | 71 | Hex3Cer(d18:1/16:0) | −0.006 | 0.006 |
| 152 | 177 | PE(P-18:1/20:5) | −0.006 | 0.006 |
| 153 | 109 | LPI 18:0 | 0.006 | 0.006 |
| 154 | 289 | TG(O-50:2) [NL-18:2] | −0.006 | 0.006 |
| 155 | 190 | PS(38:4) | 0.005 | 0.005 |
| 156 | 257 | TG(54:2) [NL-20:1] | 0.005 | 0.005 |
| 157 | 183 | PI(16:0_20:4) | 0.005 | 0.005 |
| 158 | 210 | Sph(d17:1) | 0.005 | 0.005 |
| 159 | 70 | Hex2Cer(d18:2/16:0) | −0.004 | 0.004 |
| 160 | 168 | PE(P-17:0/20:4) | −0.003 | 0.003 |
| 161 | 172 | PE(P-18:0/20:4) | 0.003 | 0.003 |
| 162 | 20 | CE(20:0) | 0.003 | 0.003 |
| 163 | 270 | TG(56:6) [NL-20:4] | −0.003 | 0.003 |
| 164 | 61 | GM3(d18:1/16:0) | −0.003 | 0.003 |
| 165 | 152 | PE(16:0_18:1) | −0.002 | 0.002 |
| 166 | 49 | Cer1P(d18:1/16:0) | 0.002 | 0.002 |
| 167 | 48 | Cer(m18:1/24:1) | −0.002 | 0.002 |
| 168 | 64 | GM3(d18:1/24:1) | 0.000 | 0.000 |
| 169 | 50 | COH | 0.000 | 0.000 |
| 170 | 93 | LPC 26:0 | 0.000 | 0.000 |
| 171 | 1 | Acylcarnitine(12:0) | 0.000 | 0.000 |
| 172 | 9 | Acylcarnitine(18:2) | 0.000 | 0.000 |
| 173 | 12 | CE(16:0) | 0.000 | 0.000 |
| 174 | 13 | CE(16:1) | 0.000 | 0.000 |
| 175 | 16 | CE(18:0) | 0.000 | 0.000 |
| 176 | 17 | CE(18:1) | 0.000 | 0.000 |
| 177 | 19 | CE(18:3) | 0.000 | 0.000 |
| 178 | 23 | CE(20:4) | 0.000 | 0.000 |
| 179 | 26 | CE(22:6) | 0.000 | 0.000 |
| 180 | 27 | Cer(d16:1/22:0) | 0.000 | 0.000 |
| 181 | 28 | Cer(d16:1/24:0) | 0.000 | 0.000 |
| 182 | 29 | Cer(d16:1/24:1) | 0.000 | 0.000 |
| 183 | 32 | Cer(d18:1/20:0) | 0.000 | 0.000 |
| 184 | 35 | Cer(d18:1/24:0) | 0.000 | 0.000 |
| 185 | 36 | Cer(d18:1/24:1) | 0.000 | 0.000 |
| 186 | 38 | Cer(d18:2/23:0) | 0.000 | 0.000 |
| 187 | 41 | Cer(d19:1/22:0) | 0.000 | 0.000 |
| 188 | 43 | Cer(d19:1/24:1) | 0.000 | 0.000 |
| 189 | 46 | Cer(m18:1/22:0) | 0.000 | 0.000 |
| 190 | 47 | Cer(m18:1/24:0) | 0.000 | 0.000 |
| 191 | 51 | DE(18:1) | 0.000 | 0.000 |
| 192 | 55 | DG(16:0_18:1) | 0.000 | 0.000 |
| 193 | 56 | DG(16:0_18:2) | 0.000 | 0.000 |
| 194 | 58 | DG(18:1_18:2) | 0.000 | 0.000 |
| 195 | 59 | DG(18:1_20:3) | 0.000 | 0.000 |
| 196 | 62 | GM3(d18:1/18:0) | 0.000 | 0.000 |
| 197 | 68 | Hex2Cer(d18:1/24:0) | 0.000 | 0.000 |
| 198 | 72 | HexCer(d18:1/16:0) | 0.000 | 0.000 |
| 199 | 73 | HexCer(d18:1/18:0) | 0.000 | 0.000 |
| 200 | 75 | HexCer(d18:1/24:0) | 0.000 | 0.000 |
| 201 | 78 | LPC 14:0 | 0.000 | 0.000 |
| 202 | 83 | LPC 18:3 | 0.000 | 0.000 |
| 203 | 85 | LPC 19:1 | 0.000 | 0.000 |
| 204 | 87 | LPC 20:4 | 0.000 | 0.000 |
| 205 | 88 | LPC 20:5 | 0.000 | 0.000 |
| 206 | 89 | LPC 22:0 | 0.000 | 0.000 |
| 207 | 90 | LPC 22:5 | 0.000 | 0.000 |
| 208 | 100 | LPC(P-17:0) | 0.000 | 0.000 |
| 209 | 102 | LPE 18:0 | 0.000 | 0.000 |
| 210 | 110 | PC 31:0 | 0.000 | 0.000 |
| 211 | 112 | PC 36:4 | 0.000 | 0.000 |
| 212 | 150 | PE 36:2 | 0.000 | 0.000 |
| 213 | 169 | PE(P-17:0/22:6) | 0.000 | 0.000 |
| 214 | 176 | PE(P-18:1/20:4) | 0.000 | 0.000 |
| 215 | 192 | SM 35:1 | 0.000 | 0.000 |
| 216 | 193 | SM 35:2 | 0.000 | 0.000 |
| 217 | 194 | SM 38:3 | 0.000 | 0.000 |
| 218 | 195 | SM 41:1 | 0.000 | 0.000 |
| 219 | 271 | TG(56:6) [NL-22:5] | 0.000 | 0.000 |
| 220 | 274 | TG(56:7) [NL-22:5] | 0.000 | 0.000 |
| 221 | 113 | PC(14:0_20:4) | 0.000 | 0.000 |
| 222 | 118 | PC(16:0_18:1) | 0.000 | 0.000 |
| 223 | 121 | PC(16:0_20:5) | 0.000 | 0.000 |
| 224 | 123 | PC(16:1_20:4) | 0.000 | 0.000 |
| 225 | 124 | PC(18:0_18:1) | 0.000 | 0.000 |
| 226 | 127 | PC(18:1_20:3) | 0.000 | 0.000 |
| 227 | 128 | PC(18:2_18:2) | 0.000 | 0.000 |
| 228 | 129 | PC(32:1) | 0.000 | 0.000 |
| 229 | 134 | PC(35:5) | 0.000 | 0.000 |
| 230 | 139 | PC(O-34:2) | 0.000 | 0.000 |
| 231 | 140 | PC(O-36:0) | 0.000 | 0.000 |
| 232 | 144 | PC(P-16:0/18:1) | 0.000 | 0.000 |
| 233 | 145 | PC(P-16:0/18:2) | 0.000 | 0.000 |
| 234 | 147 | PC(P-16:0/20:5) | 0.000 | 0.000 |
| 235 | 151 | PE(16:0_16:1) | 0.000 | 0.000 |
| 236 | 153 | PE(16:0_18:2) | 0.000 | 0.000 |
| 237 | 154 | PE(16:0_20:4) | 0.000 | 0.000 |
| 238 | 155 | PE(16:0_22:6) | 0.000 | 0.000 |
| 239 | 158 | PE(18:0_22:6) | 0.000 | 0.000 |
| 240 | 163 | PE(P-16:0/18:2) | 0.000 | 0.000 |
| 241 | 165 | PE(P-16:0/20:5) | 0.000 | 0.000 |
| 242 | 167 | PE(P-16:0/22:6) | 0.000 | 0.000 |
| 243 | 170 | PE(P-18:0/18:1) | 0.000 | 0.000 |
| 244 | 173 | PE(P-18:0/20:5) | 0.000 | 0.000 |
| 245 | 174 | PE(P-18:0/22:4) | 0.000 | 0.000 |
| 246 | 179 | PE(P-20:0/20:4) | 0.000 | 0.000 |
| 247 | 181 | PI(15-MHDA_20:4)/PI(17:0_20:4) | 0.000 | 0.000 |
| 248 | 182 | PI(16:0_16:1) | 0.000 | 0.000 |
| 249 | 186 | PI(18:0_22:6) | 0.000 | 0.000 |
| 250 | 187 | PI(34:1) | 0.000 | 0.000 |
| 251 | 209 | SM(d18:2/20:0) | 0.000 | 0.000 |
| 252 | 214 | TG(48:1) [NL-16:1] | 0.000 | 0.000 |
| 253 | 215 | TG(48:1) [NL-18:1] | 0.000 | 0.000 |
| 254 | 216 | TG(48:2) [NL-14:0] | 0.000 | 0.000 |
| 255 | 217 | TG(48:2) [NL-14:1] | 0.000 | 0.000 |
| 256 | 218 | TG(48:2) [NL-16:1] | 0.000 | 0.000 |
| 257 | 219 | TG(48:2) [NL-18:2] | 0.000 | 0.000 |
| 258 | 222 | TG(48:3) [NL-18:3] | 0.000 | 0.000 |
| 259 | 223 | TG(49:1) [NL-17:1] | 0.000 | 0.000 |
| 260 | 224 | TG(50:0) [NL-18:0] | 0.000 | 0.000 |
| 261 | 226 | TG(50:1) [NL-18:1] | 0.000 | 0.000 |
| 262 | 227 | TG(50:2) [NL-14:0] | 0.000 | 0.000 |
| 263 | 230 | TG(50:2) [NL-18:2] | 0.000 | 0.000 |
| 264 | 231 | TG(50:3) [NL-14:0] | 0.000 | 0.000 |
| 265 | 232 | TG(50:3) [NL-14:1] | 0.000 | 0.000 |
| 266 | 234 | TG(50:3) [NL-18:2] | 0.000 | 0.000 |
| 267 | 235 | TG(50:3) [NL-18:3] | 0.000 | 0.000 |
| 268 | 236 | TG(50:4) [NL-14:0] | 0.000 | 0.000 |
| 269 | 237 | TG(50:4) [NL-18:3] | 0.000 | 0.000 |
| 270 | 238 | TG(50:4) [NL-20:4] | 0.000 | 0.000 |
| 271 | 239 | TG(51:0) [NL-16:0] | 0.000 | 0.000 |
| 272 | 242 | TG(51:2) [NL-17:0] | 0.000 | 0.000 |
| 273 | 243 | TG(51:2) [NL-17:1] | 0.000 | 0.000 |
| 274 | 244 | TG(52:1) [NL-18:0] | 0.000 | 0.000 |
| 275 | 249 | TG(52:4) [NL-18:2] | 0.000 | 0.000 |
| 276 | 251 | TG(52:5) [NL-18:3] | 0.000 | 0.000 |
| 277 | 252 | TG(52:5) [NL-20:4] | 0.000 | 0.000 |
| 278 | 253 | TG(52:5) [NL-20:5] | 0.000 | 0.000 |
| 279 | 258 | TG(54:3) [NL-18:1] | 0.000 | 0.000 |
| 280 | 262 | TG(54:5) [NL-18:3] | 0.000 | 0.000 |
| 281 | 263 | TG(54:5) [NL-20:4] | 0.000 | 0.000 |
| 282 | 264 | TG(54:6) [NL-18:3] | 0.000 | 0.000 |
| 283 | 265 | TG(54:6) [NL-20:4] | 0.000 | 0.000 |
| 284 | 266 | TG(54:6) [NL-20:5] | 0.000 | 0.000 |
| 285 | 269 | TG(54:7) [NL-22:6] | 0.000 | 0.000 |
| 286 | 272 | TG(56:7) [NL-20:4] | 0.000 | 0.000 |
| 287 | 273 | TG(56:7) [NL-20:5] | 0.000 | 0.000 |
| 288 | 276 | TG(56:8) [NL-20:4] | 0.000 | 0.000 |
| 289 | 277 | TG(56:8) [NL-20:5] | 0.000 | 0.000 |
| 290 | 280 | TG(58:10) [NL-22:6] | 0.000 | 0.000 |
| 291 | 281 | TG(58:8) [NL-22:6] | 0.000 | 0.000 |
| 292 | 282 | TG(58:9) [NL-22:6] | 0.000 | 0.000 |
| 293 | 283 | TG(O-50:1) [NL-15:0] | 0.000 | 0.000 |
| 294 | 284 | TG(O-50:1) [NL-16:0] | 0.000 | 0.000 |
| 295 | 290 | TG(O-52:0) [NL-16:0] | 0.000 | 0.000 |
| 296 | 292 | TG(O-52:2) [NL-17:1] | 0.000 | 0.000 |
| 297 | 293 | TG(O-52:2) [NL-18:1] | 0.000 | 0.000 |
| 298 | 298 | Ubiquinone | 0.000 | 0.000 |

TABLE 25
Predictive models of 2h-PLG developed using
Ridge regression with the CLP2s lipids.

| | | | | Ridge |
|---|---|---|---|---|
| Rank # | Lipid # | Lipid/Covariate | Ridge | (ABS) |
| | | (Intercept) | 5.906 | 5.906 |
| 1 | 73 | LPC 18:2 | −0.452 | 0.452 |
| 2 | 71 | LPC 18:0 | 0.263 | 0.263 |
| 3 | 101 | PC(15:0_22:6) | −0.231 | 0.231 |
| 4 | 81 | LPC 22:6 | 0.198 | 0.198 |
| 5 | 4 | Acylcarnitine(14:1) | 0.198 | 0.198 |
| 6 | 171 | SM(d18:1/14:0)/SM(d16:1/16:0) | −0.174 | 0.174 |
| 7 | 119 | PC(40:8) | −0.173 | 0.173 |
| 8 | 170 | SM(d17:1/16:0) | 0.170 | 0.170 |
| 9 | 5 | Acylcarnitine(14:2) | −0.165 | 0.165 |
| 10 | 36 | Cer(d18:2/24:0) | −0.163 | 0.163 |
| 11 | 7 | Acylcarnitine(16:1) | 0.160 | 0.160 |
| 12 | 54 | DG(18:1_18:1) | −0.145 | 0.145 |
| 13 | 2 | Acylcarnitine(13:0) | −0.145 | 0.145 |
| 14 | 116 | PC(33:2) | 0.141 | 0.141 |
| 15 | 8 | Acylcarnitine(18:1) | 0.137 | 0.137 |
| 16 | 188 | TG(48:3) [NL-14:0] | −0.134 | 0.134 |
| 17 | 89 | LPE 16:0 | −0.134 | 0.134 |
| 18 | 99 | PC 36:2 | −0.132 | 0.132 |
| 19 | 169 | SM(d17:1/14:0) | −0.130 | 0.130 |
| 20 | 94 | LPE 22:6 | 0.125 | 0.125 |
| 21 | 114 | PC(32:2) | 0.125 | 0.125 |
| 22 | 41 | Cer(m18:0/22:0) | 0.121 | 0.121 |
| 23 | 147 | PE(P-18:0/22:6) | 0.116 | 0.116 |
| 24 | 107 | PC(16:0_22:6) | −0.114 | 0.114 |
| 25 | 51 | DG(16:0_16:0) | 0.113 | 0.113 |
| 26 | 215 | TG(54:6) [NL-22:6] | 0.106 | 0.106 |
| 27 | 91 | LPE 18:1 | 0.102 | 0.102 |
| 28 | 84 | LPC(O-22:0) | −0.100 | 0.100 |
| 29 | 67 | HexCer(d18:1/24:1) | −0.099 | 0.099 |
| 30 | 160 | PI(38:6) | −0.097 | 0.097 |
| 31 | 15 | CE(17:0) | −0.097 | 0.097 |
| 32 | 205 | TG(52:4) [NL-18:3] | −0.095 | 0.095 |
| 33 | 189 | TG(48:3) [NL-16:1] | −0.094 | 0.094 |
| 34 | 144 | PE(P-18:0/18:2) | 0.093 | 0.093 |
| 35 | 201 | TG(52:2) [NL-16:0] | −0.092 | 0.092 |
| 36 | 216 | TG(54:7) [NL-20:5] | 0.092 | 0.092 |
| 37 | 117 | PC(38:4) (b) | 0.091 | 0.091 |
| 38 | 87 | LPC(P-16:0) | −0.090 | 0.090 |
| 39 | 72 | LPC 18:1 | −0.089 | 0.089 |
| 40 | 11 | CE(15:0) | 0.089 | 0.089 |
| 41 | 42 | Cer(m18:1/20:0) | 0.088 | 0.088 |
| 42 | 162 | S1P(d18:1) | −0.087 | 0.087 |
| 43 | 155 | PI(18:0_18:1) | 0.087 | 0.087 |
| 44 | 178 | SM(d18:2/18:1) | −0.087 | 0.087 |
| 45 | 102 | PC(16:0_16:0) | 0.086 | 0.086 |
| 46 | 212 | TG(54:6) [NL-18:3] | 0.085 | 0.085 |
| 47 | 30 | Cer(d18:1/22:0) | 0.083 | 0.083 |
| 48 | 124 | PC(P-16:0/20:4) | −0.083 | 0.083 |
| 49 | 79 | LPC 22:0 | 0.082 | 0.082 |
| 50 | 138 | PE(P-16:0/20:4) | 0.081 | 0.081 |
| 51 | 194 | TG(50:3) [NL-16:1] | −0.081 | 0.081 |
| 52 | 226 | TG(56:8) [NL-22:6] | 0.079 | 0.079 |
| 53 | 90 | LPE 18:0 | 0.078 | 0.078 |
| 54 | 59 | Hex2Cer(d16:1/16:0) | 0.078 | 0.078 |
| 55 | 181 | Sulfatide (d18:1:/16:0(OH)) | −0.076 | 0.076 |
| 56 | 208 | TG(54:2) [NL-20:1] | 0.075 | 0.075 |
| 57 | 172 | SM(d18:1/16:0) | −0.074 | 0.074 |
| 58 | 115 | PC(33:1) | 0.073 | 0.073 |
| 59 | 133 | PE(18:0_20:4) | −0.071 | 0.071 |
| 60 | 191 | TG(50:2) [NL-18:1] | −0.071 | 0.071 |
| 61 | 129 | PE(16:0_18:1) | −0.071 | 0.071 |
| 62 | 122 | PC(P-16:0/16:0) | 0.071 | 0.071 |
| 63 | 227 | TG(56:9) [NL-22:6] | 0.070 | 0.070 |
| 64 | 106 | PC(16:0_20:5) | 0.070 | 0.070 |
| 65 | 203 | TG(52:4) [NL-16:1] | 0.069 | 0.069 |
| 66 | 28 | Cer(d18:1/16:0) | 0.068 | 0.068 |
| 67 | 93 | LPE 20:4 | 0.067 | 0.067 |
| 68 | 109 | PC(18:0_20:4) | 0.066 | 0.066 |
| 69 | 190 | TG(50:2) [NL-16:1] | −0.064 | 0.064 |
| 70 | 223 | TG(56:7) [NL-22:6] | 0.063 | 0.063 |
| 71 | 150 | PE(P-20:0/18:2) | −0.063 | 0.063 |
| 72 | 229 | TG(58:8) [NL-22:6] | −0.063 | 0.063 |
| 73 | 175 | SM(d18:1/22:0)/SM(d16:1/24:0) | −0.063 | 0.063 |
| 74 | 53 | DG(16:0_18:2) | 0.062 | 0.062 |
| 75 | 39 | Cer(d19:1/24:0) | −0.062 | 0.062 |
| 76 | 173 | SM(d18:1/18:0)/SM(d16:1/20:0) | 0.062 | 0.062 |
| 77 | 110 | PC(18:0_22:6) | 0.061 | 0.061 |
| 78 | 143 | PE(P-18:0/18:1) | −0.061 | 0.061 |
| 79 | 146 | PE(P-18:0/20:5) | −0.059 | 0.059 |
| 80 | 3 | Acylcarnitine(14:0) | 0.059 | 0.059 |
| 81 | 118 | PC(38:6) (a) | −0.058 | 0.058 |
| 82 | 69 | LPC 14:0 | 0.058 | 0.058 |
| 83 | 70 | LPC 17:0 | −0.058 | 0.058 |
| 84 | 10 | CE(14:0) | −0.057 | 0.057 |
| 85 | 27 | Cer(d17:1/24:0) | 0.057 | 0.057 |
| 86 | 204 | TG(52:4) [NL-18:2] | 0.057 | 0.057 |
| 87 | 50 | DE(20:4) | 0.056 | 0.056 |
| 88 | 234 | TG(O-54:4) [NL-17:1] | 0.056 | 0.056 |
| 89 | 14 | CE(16:2) | 0.051 | 0.051 |
| 90 | 45 | Cer(m18:1/24:1) | −0.051 | 0.051 |
| 91 | 82 | LPC 24:0 | −0.050 | 0.050 |
| 92 | 16 | CE(18:2) | −0.050 | 0.050 |
| 93 | 31 | Cer(d18:1/23:0) | 0.050 | 0.050 |
| 94 | 222 | TG(56:7) [NL-22:5] | −0.049 | 0.049 |
| 95 | 127 | PE 36:2 | −0.049 | 0.049 |
| 96 | 108 | PC(16:1_20:4) | 0.049 | 0.049 |
| 97 | 167 | SM(34:3) | 0.049 | 0.049 |
| 98 | 210 | TG(54:5) [NL-18:3] | −0.049 | 0.049 |
| 99 | 168 | SM(44:2) | 0.049 | 0.049 |
| 100 | 183 | TG(48:1) [NL-18:1] | 0.049 | 0.049 |
| 101 | 176 | SM(d18:2/14:0) | 0.048 | 0.048 |
| 102 | 37 | Cer(d18:2/24:1) | 0.048 | 0.048 |
| 103 | 219 | TG(56:6) [NL-22:5] | −0.048 | 0.048 |
| 104 | 88 | LPC(P-17:0) | 0.046 | 0.046 |
| 105 | 145 | PE(P-18:0/20:4) | 0.046 | 0.046 |
| 106 | 9 | Acylcarnitine(18:2) | −0.046 | 0.046 |
| 107 | 230 | TG(58:9) [NL-22:6] | 0.045 | 0.045 |
| 108 | 78 | LPC 20:5 | −0.045 | 0.045 |
| 109 | 232 | TG(O-50:2) [NL-18:1] | 0.045 | 0.045 |
| 110 | 66 | HexCer(d18:1/22:0) | 0.045 | 0.045 |
| 111 | 52 | DG(16:0_18:1) | −0.044 | 0.044 |
| 112 | 137 | PE(P-16:0/18:2) | −0.044 | 0.044 |
| 113 | 185 | TG(48:2) [NL-14:1] | 0.043 | 0.043 |
| 114 | 62 | Hex2Cer(d18:1/24:1) | −0.042 | 0.042 |
| 115 | 21 | CE(20:5) | −0.042 | 0.042 |
| 116 | 164 | SM 35:2 | 0.041 | 0.041 |
| 117 | 163 | SM 35:1 | −0.040 | 0.040 |
| 118 | 141 | PE(P-17:0/20:4) | −0.040 | 0.040 |
| 119 | 130 | PE(16:0_18:2) | 0.039 | 0.039 |
| 120 | 154 | PI(16:0_20:4) | 0.039 | 0.039 |
| 121 | 126 | PC(P-37:4) | −0.038 | 0.038 |
| 122 | 197 | TG(50:4) [NL-18:3] | −0.037 | 0.037 |
| 123 | 231 | TG(O-50:2) [NL-16:1] | −0.036 | 0.036 |
| 124 | 221 | TG(56:7) [NL-20:5] | 0.036 | 0.036 |
| 125 | 156 | PI(18:0_20:4) | −0.036 | 0.036 |
| 126 | 49 | DE(18:2) | 0.034 | 0.034 |
| 127 | 135 | PE(O-16:0/20:4) | −0.034 | 0.034 |
| 128 | 18 | CE(20:0) | 0.034 | 0.034 |
| 129 | 6 | Acylcarnitine(16:0) | 0.033 | 0.033 |
| 130 | 182 | Sulfatide (d18:1:/16:0) | −0.033 | 0.033 |
| 131 | 55 | DG(18:1_18:2) | 0.032 | 0.032 |
| 132 | 202 | TG(52:3) [NL-18:2] | 0.032 | 0.032 |
| 133 | 75 | LPC 19:0 | 0.032 | 0.032 |
| 134 | 214 | TG(54:6) [NL-20:5] | 0.032 | 0.032 |
| 135 | 95 | LPE(P-16:0) | −0.030 | 0.030 |
| 136 | 17 | CE(18:3) | 0.030 | 0.030 |
| 137 | 224 | TG(56:8) [NL-20:4] | 0.029 | 0.029 |
| 138 | 96 | LPE(P-18:0) | −0.029 | 0.029 |
| 139 | 29 | Cer(d18:1/20:0) | −0.029 | 0.029 |
| 140 | 57 | GM3(d18:1/22:0) | 0.028 | 0.028 |
| 141 | 187 | TG(48:2) [NL-18:2] | 0.027 | 0.027 |
| 142 | 177 | SM(d18:2/16:0) | 0.027 | 0.027 |
| 143 | 206 | TG(52:5) [NL-18:3] | 0.027 | 0.027 |
| 144 | 105 | PC(16:0_20:4) | −0.026 | 0.026 |
| 145 | 198 | TG(50:4) [NL-20:4] | −0.026 | 0.026 |
| 146 | 149 | PE(P-18:1/20:5) | −0.026 | 0.026 |
| 147 | 142 | PE(P-17:0/22:6) | −0.025 | 0.025 |
| 148 | 34 | Cer(d18:2/22:0) | 0.025 | 0.025 |
| 149 | 132 | PE(16:0_22:6) | 0.025 | 0.025 |
| 150 | 207 | TG(52:5) [NL-20:4] | −0.024 | 0.024 |
| 151 | 98 | PC 31:0 | −0.024 | 0.024 |
| 152 | 148 | PE(P-18:1/20:4) | 0.023 | 0.023 |
| 153 | 152 | PI(15-MHDA_20:4)/ | 0.023 | 0.023 |
| | | PI(17:0_20:4) | | |
| 154 | 25 | Cer(d16:1/24:0) | −0.023 | 0.023 |
| 155 | 134 | PE(18:0_22:6) | −0.023 | 0.023 |
| 156 | 77 | LPC 20:4 | −0.022 | 0.022 |
| 157 | 22 | CE(22:1) | −0.022 | 0.022 |
| 158 | 111 | PC(18:1_20:3) | 0.022 | 0.022 |
| 159 | 92 | LPE 18:2 | 0.022 | 0.022 |
| 160 | 35 | Cer(d18:2/23:0) | 0.020 | 0.020 |
| 161 | 151 | PE(P-20:0/20:4) | 0.019 | 0.019 |
| 162 | 121 | PC(P-16:0/14:0) | 0.019 | 0.019 |
| 163 | 140 | PE(P-16:0/22:6) | 0.019 | 0.019 |
| 164 | 157 | PI(18:0_22:6) | 0.019 | 0.019 |
| 165 | 1 | Acylcarnitine(12:0) | −0.019 | 0.019 |
| 166 | 83 | LPC 26:0 | 0.018 | 0.018 |
| 167 | 104 | PC(16:0_18:2) | 0.018 | 0.018 |
| 168 | 85 | LPC(O-22:1) | 0.018 | 0.018 |
| 169 | 225 | TG(56:8) [NL-20:5] | −0.018 | 0.018 |
| 170 | 233 | TG(O-50:2) [NL-18:2] | −0.018 | 0.018 |
| 171 | 195 | TG(50:3) [NL-18:2] | −0.017 | 0.017 |
| 172 | 200 | TG(51:2) [NL-17:1] | 0.017 | 0.017 |
| 173 | 199 | TG(51:2) [NL-17:0] | 0.017 | 0.017 |
| 174 | 192 | TG(50:3) [NL-14:0] | 0.017 | 0.017 |
| 175 | 13 | CE(16:1) | −0.017 | 0.017 |
| 176 | 179 | SM(d18:2/20:0) | −0.017 | 0.017 |
| 177 | 32 | Cer(d18:1/24:0) | 0.017 | 0.017 |
| 178 | 33 | Cer(d18:1/24:1) | 0.017 | 0.017 |
| 179 | 131 | PE(16:0_20:4) | 0.015 | 0.015 |
| 180 | 61 | Hex2Cer(d18:1/22:0) | 0.014 | 0.014 |
| 181 | 68 | HexCer(d18:2/24:0) | 0.014 | 0.014 |
| 182 | 235 | Ubiquinone | 0.014 | 0.014 |
| 183 | 60 | Hex2Cer(d18:1/16:0) | −0.014 | 0.014 |
| 184 | 76 | LPC 19:1 | 0.014 | 0.014 |
| 185 | 120 | PC(O-16:0/16:0) | 0.014 | 0.014 |
| 186 | 136 | PE(O-34:1) | −0.013 | 0.013 |
| 187 | 209 | TG(54:3) [NL-18:1] | 0.013 | 0.013 |
| 188 | 63 | Hex3Cer(d18:1/16:0) | −0.013 | 0.013 |
| 189 | 44 | Cer(m18:1/24:0) | −0.013 | 0.013 |
| 190 | 186 | TG(48:2) [NL-16:1] | −0.012 | 0.012 |
| 191 | 193 | TG(50:3) [NL-14:1] | −0.012 | 0.012 |
| 192 | 97 | LPI 18:0 | 0.012 | 0.012 |
| 193 | 165 | SM 38:3 | 0.012 | 0.012 |
| 194 | 56 | DG(18:1_20:3) | −0.011 | 0.011 |
| 195 | 38 | Cer(d19:1/22:0) | 0.011 | 0.011 |
| 196 | 174 | SM(d18:1/20:0)/SM(d16:1/22:0) | −0.011 | 0.011 |
| 197 | 19 | CE(20:1) | −0.011 | 0.011 |
| 198 | 20 | CE(20:4) | 0.010 | 0.010 |
| 199 | 159 | PI(36:2) | −0.010 | 0.010 |
| 200 | 40 | Cer(d19:1/24:1) | −0.010 | 0.010 |
| 201 | 153 | PI(16:0_16:1) | −0.010 | 0.010 |
| 202 | 48 | DE(18:1) | −0.010 | 0.010 |
| 203 | 218 | TG(56:6) [NL-20:4] | −0.009 | 0.009 |
| 204 | 166 | SM 41:1 | 0.009 | 0.009 |
| 205 | 125 | PC(P-16:0/20:5) | 0.009 | 0.009 |
| 206 | 184 | TG(48:2) [NL-14:0] | 0.009 | 0.009 |
| 207 | 139 | PE(P-16:0/20:5) | 0.009 | 0.009 |
| 208 | 180 | Sph(d17:1) | 0.008 | 0.008 |
| 209 | 43 | Cer(m18:1/22:0) | 0.008 | 0.008 |
| 210 | 47 | COH | 0.007 | 0.007 |
| 211 | 46 | Cer1P(d18:1/16:0) | 0.007 | 0.007 |
| 212 | 217 | TG(54:7) [NL-22:6] | 0.007 | 0.007 |
| 213 | 211 | TG(54:5) [NL-20:4] | 0.007 | 0.007 |
| 214 | 158 | PI(34:1) | 0.006 | 0.006 |
| 215 | 123 | PC(P-16:0/18:2) | −0.006 | 0.006 |
| 216 | 65 | HexCer(d18:1/18:0) | −0.006 | 0.006 |
| 217 | 213 | TG(54:6) [NL-20:4] | −0.006 | 0.006 |
| 218 | 23 | CE(22:6) | −0.005 | 0.005 |
| 219 | 86 | LPC(O-24:0) | −0.005 | 0.005 |
| 220 | 161 | PS(38:4) | −0.005 | 0.005 |
| 221 | 80 | LPC 22:5 | 0.004 | 0.004 |
| 222 | 58 | GM3(d18:1/24:1) | −0.004 | 0.004 |
| 223 | 113 | PC(32:1) | 0.004 | 0.004 |
| 224 | 74 | LPC 18:3 | −0.003 | 0.003 |
| 225 | 100 | PC 36:4 | 0.003 | 0.003 |
| 226 | 112 | PC(18:2_18:2) | −0.003 | 0.003 |
| 227 | 196 | TG(50:4) [NL-14:0] | −0.002 | 0.002 |
| 228 | 24 | Cer(d16:1/22:0) | 0.002 | 0.002 |
| 229 | 128 | PE(16:0_16:1) | −0.002 | 0.002 |
| 230 | 103 | PC(16:0_18:1) | −0.001 | 0.001 |
| 231 | 26 | Cer(d16:1/24:1) | 0.000 | 0.000 |
| 232 | 64 | HexCer(d18:1/16:0) | 0.000 | 0.000 |
| 233 | 12 | CE(16:0) | 0.000 | 0.000 |
| 234 | 220 | TG(56:7) [NL-20:4] | 0.000 | 0.000 |
| 235 | 228 | TG(58:10) [NL-22:6] | 0.000 | 0.000 |

TABLE 26
Predictive models of 2h-PLG developed using
LASSO regression with the CLP2s lipids.

| | | | | LASSO |
|---|---|---|---|---|
| Rank # | Lipid # | Lipid/Covariate | LASSO | (ABS) |
| | | (Intercept) | 5.906 | 5.906 |
| 1 | 73 | LPC 18:2 | −0.643 | 0.643 |
| 2 | 71 | LPC 18:0 | 0.353 | 0.353 |
| 3 | 81 | LPC 22:6 | 0.329 | 0.329 |
| 4 | 101 | PC(15:0_22:6) | −0.328 | 0.328 |
| 5 | 116 | PC(33:2) | 0.229 | 0.229 |
| 6 | 4 | Acylcarnitine(14:1) | 0.222 | 0.222 |
| 7 | 171 | SM(d18:1/14:0)/SM(d16:1/16:0) | −0.196 | 0.196 |
| 8 | 170 | SM(d17:1/16:0) | 0.191 | 0.191 |
| 9 | 5 | Acylcarnitine(14:2) | −0.170 | 0.170 |
| 10 | 114 | PC(32:2) | 0.167 | 0.167 |
| 11 | 119 | PC(40:8) | −0.162 | 0.162 |
| 12 | 54 | DG(18:1_18:1) | −0.144 | 0.144 |
| 13 | 36 | Cer(d18:2/24:0) | −0.143 | 0.143 |
| 14 | 2 | Acylcarnitine(13:0) | −0.143 | 0.143 |
| 15 | 7 | Acylcarnitine(16:1) | 0.133 | 0.133 |
| 16 | 215 | TG(54:6) [NL-22:6] | 0.128 | 0.128 |
| 17 | 99 | PC 36:2 | −0.126 | 0.126 |
| 18 | 8 | Acylcarnitine(18:1) | 0.125 | 0.125 |
| 19 | 41 | Cer(m18:0/22:0) | 0.115 | 0.115 |
| 20 | 94 | LPE 22:6 | 0.114 | 0.114 |
| 21 | 169 | SM(d17:1/14:0) | −0.113 | 0.113 |
| 22 | 91 | LPE 18:1 | 0.110 | 0.110 |
| 23 | 147 | PE(P-18:0/22:6) | 0.108 | 0.108 |
| 24 | 107 | PC(16:0_22:6) | −0.106 | 0.106 |
| 25 | 216 | TG(54:7) [NL-20:5] | 0.106 | 0.106 |
| 26 | 189 | TG(48:3) [NL-16:1] | −0.099 | 0.099 |
| 27 | 51 | DG(16:0_16:0) | 0.098 | 0.098 |
| 28 | 89 | LPE 16:0 | −0.095 | 0.095 |
| 29 | 188 | TG(48:3) [NL-14:0] | −0.091 | 0.091 |
| 30 | 102 | PC(16:0_16:0) | 0.091 | 0.091 |
| 31 | 201 | TG(52:2) [NL-16:0] | −0.091 | 0.091 |
| 32 | 162 | S1P(d18:1) | −0.091 | 0.091 |
| 33 | 15 | CE(17:0) | −0.088 | 0.088 |
| 34 | 84 | LPC(O-22:0) | −0.086 | 0.086 |
| 35 | 30 | Cer(d18:1/22:0) | 0.086 | 0.086 |
| 36 | 117 | PC(38:4) (b) | 0.084 | 0.084 |
| 37 | 67 | HexCer(d18:1/24:1) | −0.084 | 0.084 |
| 38 | 11 | CE(15:0) | 0.083 | 0.083 |
| 39 | 87 | LPC(P-16:0) | −0.082 | 0.082 |
| 40 | 226 | TG(56:8) [NL-22:6] | 0.080 | 0.080 |
| 41 | 205 | TG(52:4) [NL-18:3] | −0.078 | 0.078 |
| 42 | 181 | Sulfatide (d18:1:/16:0(OH)) | −0.077 | 0.077 |
| 43 | 92 | LPE 18:2 | 0.076 | 0.076 |
| 44 | 172 | SM(d18:1/16:0) | −0.076 | 0.076 |
| 45 | 124 | PC(P-16:0/20:4) | −0.075 | 0.075 |
| 46 | 160 | PI(38:6) | −0.075 | 0.075 |
| 47 | 59 | Hex2Cer(d16:1/16:0) | 0.074 | 0.074 |
| 48 | 194 | TG(50:3) [NL-16:1] | −0.071 | 0.071 |
| 49 | 122 | PC(P-16:0/16:0) | 0.070 | 0.070 |
| 50 | 212 | TG(54:6) [NL-18:3] | 0.069 | 0.069 |
| 51 | 155 | PI(18:0_18:1) | 0.066 | 0.066 |
| 52 | 138 | PE(P-16:0/20:4) | 0.066 | 0.066 |
| 53 | 79 | LPC 22:0 | 0.065 | 0.065 |
| 54 | 129 | PE(16:0_18:1) | −0.064 | 0.064 |
| 55 | 191 | TG(50:2) [NL-18:1] | −0.064 | 0.064 |
| 56 | 28 | Cer(d18:1/16:0) | 0.064 | 0.064 |
| 57 | 208 | TG(54:2) [NL-20:1] | 0.062 | 0.062 |
| 58 | 190 | TG(50:2) [NL-16:1] | −0.061 | 0.061 |
| 59 | 42 | Cer(m18:1/20:0) | 0.060 | 0.060 |
| 60 | 176 | SM(d18:2/14:0) | 0.060 | 0.060 |
| 61 | 72 | LPC 18:1 | −0.059 | 0.059 |
| 62 | 31 | Cer(d18:1/23:0) | 0.058 | 0.058 |
| 63 | 143 | PE(P-18:0/18:1) | −0.054 | 0.054 |
| 64 | 39 | Cer(d19:1/24:0) | −0.053 | 0.053 |
| 65 | 93 | LPE 20:4 | 0.051 | 0.051 |
| 66 | 204 | TG(52:4) [NL-18:2] | 0.051 | 0.051 |
| 67 | 178 | SM(d18:2/18:1) | −0.050 | 0.050 |
| 68 | 234 | TG(O-54:4) [NL-17:1] | 0.049 | 0.049 |
| 69 | 115 | PC(33:1) | 0.049 | 0.049 |
| 70 | 150 | PE(P-20:0/18:2) | −0.049 | 0.049 |
| 71 | 227 | TG(56:9) [NL-22:6] | 0.047 | 0.047 |
| 72 | 203 | TG(52:4) [NL-16:1] | 0.047 | 0.047 |
| 73 | 37 | Cer(d18:2/24:1) | 0.047 | 0.047 |
| 74 | 50 | DE(20:4) | 0.046 | 0.046 |
| 75 | 146 | PE(P-18:0/20:5) | −0.045 | 0.045 |
| 76 | 14 | CE(16:2) | 0.045 | 0.045 |
| 77 | 10 | CE(14:0) | −0.045 | 0.045 |
| 78 | 145 | PE(P-18:0/20:4) | 0.044 | 0.044 |
| 79 | 16 | CE(18:2) | −0.044 | 0.044 |
| 80 | 144 | PE(P-18:0/18:2) | 0.039 | 0.039 |
| 81 | 173 | SM(d18:1/18:0)/SM(d16:1/20:0) | 0.039 | 0.039 |
| 82 | 126 | PC(P-37:4) | −0.039 | 0.039 |
| 83 | 70 | LPC 17:0 | −0.037 | 0.037 |
| 84 | 49 | DE(18:2) | 0.037 | 0.037 |
| 85 | 232 | TG(O-50:2) [NL-18:1] | 0.036 | 0.036 |
| 86 | 109 | PC(18:0_20:4) | 0.034 | 0.034 |
| 87 | 167 | SM(34:3) | 0.034 | 0.034 |
| 88 | 6 | Acylcarnitine(16:0) | 0.033 | 0.033 |
| 89 | 202 | TG(52:3) [NL-18:2] | 0.033 | 0.033 |
| 90 | 175 | SM(d18:1/22:0)/SM(d16:1/24:0) | −0.031 | 0.031 |
| 91 | 66 | HexCer(d18:1/22:0) | 0.031 | 0.031 |
| 92 | 219 | TG(56:6) [NL-22:5] | −0.031 | 0.031 |
| 93 | 62 | Hex2Cer(d18:1/24:1) | −0.030 | 0.030 |
| 94 | 95 | LPE(P-16:0) | −0.029 | 0.029 |
| 95 | 231 | TG(O-50:2) [NL-16:1] | −0.026 | 0.026 |
| 96 | 182 | Sulfatide (d18:1:/16:0) | −0.025 | 0.025 |
| 97 | 27 | Cer(d17:1/24:0) | 0.025 | 0.025 |
| 98 | 135 | PE(O-16:0/20:4) | −0.025 | 0.025 |
| 99 | 133 | PE(18:0_20:4) | −0.025 | 0.025 |
| 100 | 168 | SM(44:2) | 0.024 | 0.024 |
| 101 | 18 | CE(20:0) | 0.024 | 0.024 |
| 102 | 152 | PI(15-MHDA_20:4)/ | 0.023 | 0.023 |
| | | PI(17:0_20:4) | | |
| 103 | 183 | TG(48:1) [NL-18:1] | 0.023 | 0.023 |
| 104 | 21 | CE(20:5) | −0.022 | 0.022 |
| 105 | 3 | Acylcarnitine(14:0) | 0.021 | 0.021 |
| 106 | 22 | CE(22:1) | −0.019 | 0.019 |
| 107 | 96 | LPE(P-18:0) | −0.019 | 0.019 |
| 108 | 154 | PI(16:0_20:4) | 0.019 | 0.019 |
| 109 | 177 | SM(d18:2/16:0) | 0.018 | 0.018 |
| 110 | 230 | TG(58:9) [NL-22:6] | 0.017 | 0.017 |
| 111 | 156 | PI(18:0_20:4) | −0.017 | 0.017 |
| 112 | 57 | GM3(d18:1/22:0) | 0.017 | 0.017 |
| 113 | 45 | Cer(m18:1/24:1) | −0.017 | 0.017 |
| 114 | 210 | TG(54:5) [NL-18:3] | −0.015 | 0.015 |
| 115 | 110 | PC(18:0_22:6) | 0.013 | 0.013 |
| 116 | 187 | TG(48:2) [NL-18:2] | 0.013 | 0.013 |
| 117 | 68 | HexCer(d18:2/24:0) | 0.012 | 0.012 |
| 118 | 104 | PC(16:0_18:2) | 0.012 | 0.012 |
| 119 | 163 | SM 35:1 | −0.011 | 0.011 |
| 120 | 221 | TG(56:7) [NL-20:5] | 0.011 | 0.011 |
| 121 | 136 | PE(O-34:1) | −0.011 | 0.011 |
| 122 | 118 | PC(38:6) (a) | −0.010 | 0.010 |
| 123 | 223 | TG(56:7) [NL-22:6] | 0.010 | 0.010 |
| 124 | 34 | Cer(d18:2/22:0) | 0.009 | 0.009 |
| 125 | 60 | Hex2Cer(d18:1/16:0) | −0.009 | 0.009 |
| 126 | 97 | LPI 18:0 | 0.009 | 0.009 |
| 127 | 35 | Cer(d18:2/23:0) | 0.009 | 0.009 |
| 128 | 82 | LPC 24:0 | −0.009 | 0.009 |
| 129 | 180 | Sph(d17:1) | 0.008 | 0.008 |
| 130 | 108 | PC(16:1_20:4) | 0.008 | 0.008 |
| 131 | 121 | PC(P-16:0/14:0) | 0.007 | 0.007 |
| 132 | 63 | Hex3Cer(d18:1/16:0) | −0.007 | 0.007 |
| 133 | 33 | Cer(d18:1/24:1) | 0.006 | 0.006 |
| 134 | 111 | PC(18:1_20:3) | 0.006 | 0.006 |
| 135 | 90 | LPE 18:0 | 0.005 | 0.005 |
| 136 | 29 | Cer(d18:1/20:0) | −0.005 | 0.005 |
| 137 | 120 | PC(O-16:0/16:0) | 0.004 | 0.004 |
| 138 | 17 | CE(18:3) | 0.004 | 0.004 |
| 139 | 46 | Cer1P(d18:1/16:0) | 0.004 | 0.004 |
| 140 | 48 | DE(18:1) | −0.004 | 0.004 |
| 141 | 149 | PE(P-18:1/20:5) | −0.003 | 0.003 |
| 142 | 233 | TG(O-50:2) [NL-18:2] | −0.003 | 0.003 |
| 143 | 83 | LPC 26:0 | 0.002 | 0.002 |
| 144 | 75 | LPC 19:0 | 0.001 | 0.001 |
| 145 | 235 | Ubiquinone | 0.001 | 0.001 |
| 146 | 53 | DG(16:0_18:2) | 0.001 | 0.001 |
| 147 | 61 | Hex2Cer(d18:1/22:0) | 0.001 | 0.001 |
| 148 | 47 | COH | 0.000 | 0.000 |
| 149 | 151 | PE(P-20:0/20:4) | 0.000 | 0.000 |
| 150 | 1 | Acylcarnitine(12:0) | 0.000 | 0.000 |
| 151 | 9 | Acylcarnitine(18:2) | 0.000 | 0.000 |
| 152 | 12 | CE(16:0) | 0.000 | 0.000 |
| 153 | 13 | CE(16:1) | 0.000 | 0.000 |
| 154 | 19 | CE(20:1) | 0.000 | 0.000 |
| 155 | 20 | CE(20:4) | 0.000 | 0.000 |
| 156 | 23 | CE(22:6) | 0.000 | 0.000 |
| 157 | 24 | Cer(d16:1/22:0) | 0.000 | 0.000 |
| 158 | 25 | Cer(d16:1/24:0) | 0.000 | 0.000 |
| 159 | 26 | Cer(d16:1/24:1) | 0.000 | 0.000 |
| 160 | 32 | Cer(d18:1/24:0) | 0.000 | 0.000 |
| 161 | 38 | Cer(d19:1/22:0) | 0.000 | 0.000 |
| 162 | 40 | Cer(d19:1/24:1) | 0.000 | 0.000 |
| 163 | 43 | Cer(m18:1/22:0) | 0.000 | 0.000 |
| 164 | 44 | Cer(m18:1/24:0) | 0.000 | 0.000 |
| 165 | 52 | DG(16:0_18:1) | 0.000 | 0.000 |
| 166 | 55 | DG(18:1_18:2) | 0.000 | 0.000 |
| 167 | 56 | DG(18:1_20:3) | 0.000 | 0.000 |
| 168 | 58 | GM3(d18:1/24:1) | 0.000 | 0.000 |
| 169 | 64 | HexCer(d18:1/16:0) | 0.000 | 0.000 |
| 170 | 65 | HexCer(d18:1/18:0) | 0.000 | 0.000 |
| 171 | 85 | LPC(O-22:1) | 0.000 | 0.000 |
| 172 | 86 | LPC(O-24:0) | 0.000 | 0.000 |
| 173 | 69 | LPC 14:0 | 0.000 | 0.000 |
| 174 | 74 | LPC 18:3 | 0.000 | 0.000 |
| 175 | 76 | LPC 19:1 | 0.000 | 0.000 |
| 176 | 77 | LPC 20:4 | 0.000 | 0.000 |
| 177 | 78 | LPC 20:5 | 0.000 | 0.000 |
| 178 | 80 | LPC 22:5 | 0.000 | 0.000 |
| 179 | 88 | LPC(P-17:0) | 0.000 | 0.000 |
| 180 | 98 | PC 31:0 | 0.000 | 0.000 |
| 181 | 100 | PC 36:4 | 0.000 | 0.000 |
| 182 | 127 | PE 36:2 | 0.000 | 0.000 |
| 183 | 141 | PE(P-17:0/20:4) | 0.000 | 0.000 |
| 184 | 142 | PE(P-17:0/22:6) | 0.000 | 0.000 |
| 185 | 148 | PE(P-18:1/20:4) | 0.000 | 0.000 |
| 186 | 164 | SM 35:2 | 0.000 | 0.000 |
| 187 | 165 | SM 38:3 | 0.000 | 0.000 |
| 188 | 166 | SM 41:1 | 0.000 | 0.000 |
| 189 | 222 | TG(56:7) [NL-22:5] | 0.000 | 0.000 |
| 190 | 103 | PC(16:0_18:1) | 0.000 | 0.000 |
| 191 | 105 | PC(16:0_20:4) | 0.000 | 0.000 |
| 192 | 106 | PC(16:0_20:5) | 0.000 | 0.000 |
| 193 | 112 | PC(18:2_18:2) | 0.000 | 0.000 |
| 194 | 113 | PC(32:1) | 0.000 | 0.000 |
| 195 | 123 | PC(P-16:0/18:2) | 0.000 | 0.000 |
| 196 | 125 | PC(P-16:0/20:5) | 0.000 | 0.000 |
| 197 | 128 | PE(16:0_16:1) | 0.000 | 0.000 |
| 198 | 130 | PE(16:0_18:2) | 0.000 | 0.000 |
| 199 | 131 | PE(16:0_20:4) | 0.000 | 0.000 |
| 200 | 132 | PE(16:0_22:6) | 0.000 | 0.000 |
| 201 | 134 | PE(18:0_22:6) | 0.000 | 0.000 |
| 202 | 137 | PE(P-16:0/18:2) | 0.000 | 0.000 |
| 203 | 139 | PE(P-16:0/20:5) | 0.000 | 0.000 |
| 204 | 140 | PE(P-16:0/22:6) | 0.000 | 0.000 |
| 205 | 153 | PI(16:0_16:1) | 0.000 | 0.000 |
| 206 | 157 | PI(18:0_22:6) | 0.000 | 0.000 |
| 207 | 158 | PI(34:1) | 0.000 | 0.000 |
| 208 | 159 | PI(36:2) | 0.000 | 0.000 |
| 209 | 161 | PS(38:4) | 0.000 | 0.000 |
| 210 | 174 | SM(d18:1/20:0)/SM(d16:1/22:0) | 0.000 | 0.000 |
| 211 | 179 | SM(d18:2/20:0) | 0.000 | 0.000 |
| 212 | 184 | TG(48:2) [NL-14:0] | 0.000 | 0.000 |
| 213 | 185 | TG(48:2) [NL-14:1] | 0.000 | 0.000 |
| 214 | 186 | TG(48:2) [NL-16:1] | 0.000 | 0.000 |
| 215 | 192 | TG(50:3) [NL-14:0] | 0.000 | 0.000 |
| 216 | 193 | TG(50:3) [NL-14:1] | 0.000 | 0.000 |
| 217 | 195 | TG(50:3) [NL-18:2] | 0.000 | 0.000 |
| 218 | 196 | TG(50:4) [NL-14:0] | 0.000 | 0.000 |
| 219 | 197 | TG(50:4) [NL-18:3] | 0.000 | 0.000 |
| 220 | 198 | TG(50:4) [NL-20:4] | 0.000 | 0.000 |
| 221 | 199 | TG(51:2) [NL-17:0] | 0.000 | 0.000 |
| 222 | 200 | TG(51:2) [NL-17:1] | 0.000 | 0.000 |
| 223 | 206 | TG(52:5) [NL-18:3] | 0.000 | 0.000 |
| 224 | 207 | TG(52:5) [NL-20:4] | 0.000 | 0.000 |
| 225 | 209 | TG(54:3) [NL-18:1] | 0.000 | 0.000 |
| 226 | 211 | TG(54:5) [NL-20:4] | 0.000 | 0.000 |
| 227 | 213 | TG(54:6) [NL-20:4] | 0.000 | 0.000 |
| 228 | 214 | TG(54:6) [NL-20:5] | 0.000 | 0.000 |
| 229 | 217 | TG(54:7) [NL-22:6] | 0.000 | 0.000 |
| 230 | 218 | TG(56:6) [NL-20:4] | 0.000 | 0.000 |
| 231 | 220 | TG(56:7) [NL-20:4] | 0.000 | 0.000 |
| 232 | 224 | TG(56:8) [NL-20:4] | 0.000 | 0.000 |
| 233 | 225 | TG(56:8) [NL-20:5] | 0.000 | 0.000 |
| 234 | 228 | TG(58:10) [NL-22:6] | 0.000 | 0.000 |
| 235 | 229 | TG(58:8) [NL-22:6] | 0.000 | 0.000 |

IGT risk assessment: Two models in classification of individuals with isolated IGT were examined in the context of a pre-screened cohort (HbA1c<6%, FBG<6.1 mmol/L). Model 1 consisted of the AUSDRISK score, FBG and HbA1C. Model 2 comprised of the same variable as Model 1 with the inclusion of lipid derived 2 h-PLG. Models were optimised to classify isolated IGT.

The modelling was done in a 5-fold cross validation framework. The training set comprised of ⅘ths of the cohort to develop the coefficients for the predicted score and subsequently train the IGT risk assessment model. The model was then applied to the hold-out ⅕^{th} test set. This was repeated until all samples had a risk score. The optimal threshold for reporting classification performance was selected using Youden's index.

The Plasma Lipidome Improved Classification of Impaired Glucose Tolerance from a Subclinical Population.

The performance of multivariate models in classifying individuals likely to have isolated IGT (2 h-PLG>7.7 mmol/L) was examined using a base model (comprising of AUSDRISK, fasting blood glucose and HbA1c) and the base model with lipidomic data (lipid-2 h-PLG). The analysis involved individuals with FBG<6.1 mmol/L and Hba1c<6% which would be classified as low-risk under the current Australian framework (FIG. 21). In total 939 out of 8,134 individuals were classified as isolated IGT. The addition of lipid species into risk assessment models significantly improved classification of impaired glucose tolerance (0.831 vs 0.695) from a population where fasting and point of care glucose measures are within healthy ranges (HbA1c<6%, FBG <6.1 mmol/L, Table 16). Improvements to classification were maintained when using a smaller subset of lipids (CLP1 and CLP2) to predict 2 h-PLG for assessment (AUC of 0.830 and 0.828 respectively). Using specific optimal cut-offs for the clinical subset, as determined by Youden's Index, large reclassifications of true negative individuals (4,534 to 5,324) were observed, and improvements to classification of true positive (630 to 726). In contrast to the base model, false positives and negatives were decreased and improvements to continuous net reclassification index (NRI) was observed in both lipid models. When the smaller lipid sets CLP1 and CLP2 were used, the gains in model performance were very similar.

TABLE 27
IGT classification performance of base and lipid models

| | | | Base + | | | | | |
|---|---|---|---|---|---|---|---|---|
| | | | Research | | | | | |
| | Base | | Lipid List | | Base + CLP 1 | | Base + CLP 2 | |
| AUC | 0.695 | | 0.831 | | 0.830 | | 0.828 | |
| Sensitivity | 0.67 | | 0.77 | | 0.78 | | 0.73 | |
| Specificity | 0.63 | | 0.74 | | 0.73 | | 0.78 | |
| PPV | 0.19 | | 0.28 | | 0.27 | | 0.30 | |
| NPV | 0.94 | | 0.96 | | 0.96 | | 0.96 | |
| TN | 4534 | 63.02% | 5324 | 74.00% | 5252 | 73.00% | 5633 | 78.29% |
| TP | 630 | 67.09% | 726 | 77.32% | 730 | 77.74% | 683 | 72.74% |
| FP | 2661 | 36.98% | 1871 | 26.00% | 1943 | 27.00% | 1562 | 21.71% |
| FN | 309 | 32.91% | 213 | 22.68% | 209 | 22.26% | 256 | 27.26% |

| | | | | |
|---|---|---|---|---|
| Total NGT | 7195 | 7195 | 7195 | 7195 |
| Total IGT | 939 | 939 | 939 | 939 |
| NRI (total) | — | 0.901 | 0.889 | 0.862 |
| NRI (event) | — | 0.429 | 0.423 | 0.397 |
| NRI (non- | — | 0.472 | 0.466 | 0.464 |
| event) | | | | |

Base model comprises of AUSDRISK, fasting blood glucose and HbA1c. Base + lipid models includes the lipid predicted 2h-PLG with the base model. PPV, positive predictive value, NPV, negative predictive value, TN, true negative, TP, true positive, FP, false positive, FN, false negative, NGT, normal glucose tolerance, IGT, impaired glucose tolerance, NRI, net reclassification index (continuous).

### The Inclusion of CLP Improved Assessment of Future Impaired Glucose Tolerance.

The model performance in classifying individuals that develop impairment to glucose tolerance at 5 years (2 h-PLG at 5-year follow up exceeding 7.7 mmol/L) was then examined. The same approach was applied but using a subset of individuals that were normoglycemic at baseline (FBG<6.1 mmol/L, 2 h-PLG<7.8 mmol/L) and also had 5-year follow up measurements for 2 h-PLG. In total, 302 individuals presented with 2h-PLG>7.7 mmol/L at 5-year follow up despite being normoglycemic at baseline.

Contrasting to the base model of AUSDRISK, fasting blood glucose and HbA1c, addition of the CLP1 or CLP2 lipids (as a predicted 2 h-PLG score) significantly improved classification (AUC 0.678 increased to 0.745 and 0.749 respectively, Table 28). Inclusion of CLP lipids improved performance over the base model even with baseline measured 2 h-PLG (AUC 0.749 vs 0.774 and 0.768, Table 17). In both instances, improvements to continuous net reclassification indexes were observed relative to the non-lipid models, with smaller improvements over the base model with 2 h-PLG included.

TABLE 28
5-year-IGT classification performance of base and lipid models

| | | | | | | | Base + 2h- | | Base + 2h- | | Base + 2h- | |
|---|---|---|---|---|---|---|---|---|---|---|---|---|
| | Base | | Base + CLP 1 | | Base + CLP 2 | | PLG | | PLG + CLP 1 | | PLG + CLP 2 | |
| AUC | 0.678 | | 0.745 | | 0.749 | | 0.749 | | 0.774 | | 0.768 | |
| Sensitivity | 0.65 | | 0.67 | | 0.66 | | 0.67 | | 0.71 | | 0.71 | |
| Specificity | 0.63 | | 0.71 | | 0.74 | | 0.71 | | 0.73 | | 0.72 | |
| PPV | 0.12 | | 0.15 | | 0.16 | | 0.15 | | 0.17 | | 0.16 | |
| NPV | 0.96 | | 0.97 | | 0.97 | | 0.97 | | 0.97 | | 0.97 | |
| TN | 2483 | 63.85% | 2762 | 71.02% | 2865 | 73.67% | 2770 | 71.23% | 2839 | 73.00% | 2782 | 71.54% |
| TP | 197 | 65.23% | 202 | 66.89% | 200 | 66.23% | 202 | 66.89% | 213 | 70.53% | 215 | 71.19% |
| FP | 1406 | 36.15% | 1127 | 28.98% | 1024 | 26.33% | 1119 | 28.77% | 1050 | 27.00% | 1107 | 28.46% |
| FN | 105 | 34.77% | 100 | 33.11% | 102 | 33.77% | 100 | 33.11% | 89 | 29.47% | 87 | 28.81% |

| | | | | | | |
|---|---|---|---|---|---|---|
| Total NGT | 3889 | 3889 | 3889 | 3889 | 3889 | 3889 |
| Total IGT | 302 | 302 | 302 | 302 | 302 | 302 |
| NRI (total) | — | 0.550 | 0.543 | — | 0.403 | 0.328 |
| NRI (event) | — | 0.252 | 0.238 | — | 0.219 | 0.152 |
| NRI (non- | — | 0.298 | 0.304 | — | 0.185 | 0.176 |
| event) | | | | | | |

Base model comprises of AUSDRISK, fasting blood glucose and HbA1c. Base + Lipid model includes the lipid predicted 2h-PLG with the base model. Base + 2h-PLG includes 2-hour post load glucose measured at baseline. PPV, positive predictive value, NPV, negative predictive value, TN, true negative, TP, true positive, FP, false positive, FN, false negative, NGT, normal glucose tolerance, IGT, impaired glucose tolerance, NRI, net reclassification index (continuous)..

### The Plasma Lipidome Improved Classification of Future Impaired Glucose Tolerance in a Subclinical Population.

Whether incorporation of the CLP measurements would improve existing models in identifying future risk of T2DM (5-year) was then examined. We included all individuals with FBG<6.1 mmol/L, HbA1c<6%, 2 h-PLG<11.1 mmol/L and had 5-year follow up data. In total, 103 out of 4,712 individuals were classified as T2DM at 5-year follow-up. We observed similar improvements in classification models with the inclusion of CLP1 or CLP2 measurements.

TABLE 29
5-year-T2DM classification performance of base and lipid models

| | Base | | Base + CLP 1 | | Base + CLP 2 | |
|---|---|---|---|---|---|---|
| AUC | 0.751 | | 0.783 | | 0.789 | |
| Sensitivity | 0.68 | | 0.72 | | 0.74 | |
| Specificity | 0.72 | | 0.76 | | 0.73 | |
| PPV | 0.05 | | 0.06 | | 0.06 | |
| NPV | 0.99 | | 0.99 | | 0.99 | |
| TN | 3338 | 72.42% | 3485 | 75.61% | 3382 | 73.38% |
| TP | 70 | 67.96% | 74 | 71.84% | 76 | 73.79% |
| FP | 1271 | 27.58% | 1124 | 24.39% | 1227 | 26.62% |
| FN | 33 | 32.04% | 29 | 28.16% | 27 | 26.21% |

| | | | |
|---|---|---|---|
| Total NGT | 4609 | 4609 | 4609 |
| Total IGT | 103 | 103 | 103 |
| NRI (total) | — | 0.326 | 0.407 |
| NRI (event) | — | 0.087 | 0.184 |
| NRI (non- | — | 0.238 | 0.223 |
| event) | | | |

Base model comprises of AUSDRISK, fasting blood glucose and HbA1c. Base + Lipid model includes the lipid predicted 2h-PLG with the base model. PPV, positive predictive value, NPV, negative predictive value, TN, true negative, TP, true positive, FP, false positive, FN, false negative, NGT, normal glucose tolerance, IGT, impaired glucose tolerance, NRI, net reclassification index (continuous).

Impairment to glucose tolerance remains challenging for early detection owing to the requirement of an oral glucose tolerance test. This results in under detection of symptomless individuals that would receive the most benefit from T2DM preventative interventions. In the AusdDiab cohort, 11.5% (939/8134) of the ‘low risk’ individuals would be clinically diagnosed as prediabetic if an OGTT was performed. The lipidomics approach described herein was applied in tandem with traditional risk factors obtainable without an OGTT (AUSDRISK, fasting glucose and Hba1c). Significant improvements to all metrics using both the research lipid platform and the clinical subset lists were observed. The lipid classification models performed better than baseline for classifying both prevalent IGT and 5-year risk of IGT and T2DM.

As an OGTT is currently required for IGT diagnosis, application of this model would benefit the risk assessment stage. Sensitivity thresholds can be adjusted to accommodate different clinical application paths, including a high-sensitivity test as an assessment tool for to augment traditional risk factors, dictating whether an individual would require an OGTT before clinical symptoms.

The targeted CLP lipid lists utilised small amounts of blood that can be taken in tandem with a fasting glucose test. This offers significant logistical advantages over OGTT and can be used in tandem as a pre-screen assessment together with fasting glucose and Hba1c thus reducing the unnecessary need of an early OGTT, and improves conventional screening approaches. As IGT can exist independently of the other traditional risk factor, the CLP would significantly assist in identifying high-risk individuals that would have otherwise been missed under the current framework.

TABLE 30
List of lipid species and their transitions monitored via MRM
in the CLP2 lipid list and in CLP2s, CLP3, CLP4 and CLP5.

| Lipid | | | Precursor | Product | | | | |
|---|---|---|---|---|---|---|---|---|
| Number | Lipid Class | Compound Name | (m/z) | (m/z) | CLP2s | CLP3 | CLP4 | CLP5 |
| 1 | AC | AC(10:0) | 316.3 | 85.1 | | | | |
| 2 | AC | AC(12:0) | 344.3 | 85.1 | y | y | y | y |
| 3 | AC | AC(12:1) | 342.3 | 85.1 | | | | |
| 4 | AC | AC(13:0) | 358.3 | 85.1 | y | y | y | y |
| 5 | AC | AC(14:0) | 372.3 | 85.1 | y | y | y | y |
| 6 | AC-OH | AC(14:0)-OH | 388.3 | 85.1 | | | | |
| 7 | AC | AC(14:1) | 370.3 | 85.1 | y | y | y | y |
| 8 | AC-OH | AC(14:1)-OH | 386.3 | 85.1 | | | | |
| 9 | AC | AC(14:2) | 368.3 | 85.1 | y | y | y | y |
| 10 | AC | AC(16:0) | 400.4 | 85.1 | y | y | y | y |
| 11 | AC | AC(16:1) | 398.3 | 85.1 | y | y | y | y |
| 12 | AC | AC(18:1) | 426.4 | 85.1 | y | y | y | y |
| 13 | AC | AC(18:2) | 424.3 | 85.1 | y | y | y | y |
| 14 | AC | AC(24:0) | 512.3 | 85.1 | | | | |
| 15 | AC | AC(26:0) | 540.3 | 85.1 | | | | |
| 16 | AC | AC(26:1) | 538.3 | 85.1 | | | | |
| 17 | CE | CE(14:0) | 614.6 | 369.3 | y | | y | |
| 18 | CE | CE(15:0) | 628.6 | 369.3 | y | y | y | y |
| 19 | CE | CE(16:0) | 642.6 | 369.3 | y | y | y | y |
| 20 | CE | CE(16:1) | 640.6 | 369.3 | y | y | y | y |
| 21 | CE | CE(16:2) | 638.6 | 369.3 | y | | y | |
| 22 | CE | CE(17:0) | 656.6 | 369.3 | y | y | | |
| 23 | CE | CE(18:2) | 666.6 | 369.3 | y | y | y | y |
| 24 | CE | CE(18:3) | 664.6 | 369.3 | y | | | |
| 25 | CE | CE(20:0) | 698.7 | 369.3 | y | y | y | y |
| 26 | CE | CE(20:1) | 696.7 | 369.3 | y | y | | |
| 27 | CE | CE(20:4) | 690.6 | 369.3 | y | y | y | y |
| 28 | CE | CE(20:5) | 688.6 | 369.3 | y | y | y | y |
| 29 | CE | CE(22:1) | 724.7 | 369.3 | y | y | | |
| 30 | CE | CE(22:6) | 714.6 | 369.3 | y | y | y | y |
| 31 | Cer(d) | Cer(d16:1/22:0) | 594.6 | 236.3 | y | y | y | y |
| 32 | Cer(d) | Cer(d16:1/24:0) | 622.6 | 236.3 | y | y | y | y |
| 33 | Cer(d) | Cer(d16:1/24:1) | 620.6 | 236.3 | y | y | y | y |
| 34 | Cer(d) | Cer(d17:1/24:0) | 636.6 | 250.3 | y | y | y | y |
| 35 | Cer(d) | Cer(d18:1/16:0) | 538.5 | 264.3 | y | y | y | |
| 36 | Cer(d) | Cer(d18:1/20:0) | 594.6 | 264.3 | y | y | | |
| 37 | Cer(d) | Cer(d18:1/22:0) | 622.6 | 264.3 | y | y | y | y |
| 38 | Cer(d) | Cer(d18:1/23:0) | 636.6 | 264.3 | y | y | y | y |
| 39 | Cer(d) | Cer(d18:1/24:0) | 650.6 | 264.3 | y | y | | |
| 40 | Cer(d) | Cer(d18:1/24:1) | 648.6 | 264.3 | y | y | | |
| 41 | Cer(d) | Cer(d18:2/22:0) | 620.6 | 262.3 | y | y | y | y |
| 42 | Cer(d) | Cer(d18:2/23:0) | 634.6 | 262.3 | y | y | y | y |
| 43 | Cer(d) | Cer(d18:2/24:0) | 648.6 | 262.3 | y | y | y | y |
| 44 | Cer(d) | Cer(d18:2/24:1) | 646.6 | 262.3 | y | y | y | y |
| 45 | Cer(d) | Cer(d19:1/22:0) | 636.6 | 278.3 | y | y | y | y |
| 46 | Cer(d) | Cer(d19:1/24:0) | 664.6 | 278.3 | y | y | y | y |
| 47 | Cer(d) | Cer(d19:1/24:1) | 662.6 | 278.3 | y | y | y | y |
| 48 | Cer(m) | Cer(m18:0/22:0) | 608.6 | 268.4 | y | y | y | y |
| 49 | Cer(m) | Cer(m18:1/20:0) | 578.6 | 266.4 | y | y | y | y |
| 50 | Cer(m) | Cer(m18:1/22:0) | 606.6 | 266.4 | y | y | y | y |
| 51 | Cer(m) | Cer(m18:1/24:0) | 634.6 | 266.4 | y | y | y | y |
| 52 | Cer(m) | Cer(m18:1/24:1) | 632.6 | 266.4 | y | y | y | y |
| 53 | C1P | Cer1P(d18:1/16:0) | 618.4 | 264.3 | y | y | y | y |
| 54 | COH | COH | 369.4 | 161.2 | y | y | y | y |
| 55 | DE | DE(18:1) | 666.6 | 367.4 | y | y | y | y |
| 56 | DE | DE(18:2) | 664.6 | 367.4 | y | y | y | y |
| 57 | DE | DE(20:4) | 688.6 | 367.4 | y | y | y | y |
| 58 | DG | DG(16:0_16:0) | 586.5 | 313.2 | y | y | | |
| 59 | DG | DG(16:0_18:1) | 612.6 | 313.3 | y | y | | |
| 60 | DG | DG(16:0_18:2) | 610.5 | 313.2 | y | y | | |
| 61 | DG | DG(18:1_18:1) | 638.6 | 339.3 | y | y | | |
| 62 | DG | DG(18:1_18:2) | 636.6 | 339.3 | y | y | | |
| 63 | DG | DG(18:1_20:3) | 662.6 | 339.3 | y | y | | |
| 64 | dimethyl-CE | dimethyl-CE(18:2) | 694.6 | 397.3 | | | | |
| 65 | dimethyl-CE | dimethyl-CE(20:4) | 718.6 | 397.3 | | | | |
| 66 | FFA | FA(14:0) | 227.2 | 227.2 | | | | |
| 67 | FFA | FA(16:0) | 255.2 | 255.2 | | | | |
| 68 | FFA | FA(16:1) | 253.2 | 253.2 | | | | |
| 69 | FFA | FA(17:1) | 267.2 | 267.2 | | | | |
| 70 | FFA | FA(18:1) | 281.3 | 281.2 | | | | |
| 71 | FFA | FA(18:2) | 279.2 | 279.2 | | | | |
| 72 | FFA | FA(18:3) | 277.2 | 277.2 | | | | |
| 73 | FFA | FA(20:4) | 303.2 | 303.2 | | | | |
| 74 | FFA | FA(22:4) | 331.3 | 331.3 | | | | |
| 75 | FFA | FA(22:5) | 329.3 | 329.2 | | | | |
| 76 | FFA | FA(22:6) | 327.2 | 327.2 | | | | |
| 77 | GM3 | GM3(d18:1/22:0) | 1237.8 | 264.3 | y | y | y | y |
| 78 | GM3 | GM3(d18:1/24:1) | 1263.8 | 264.3 | y | y | y | y |
| 79 | Hex2Cer | Hex2Cer(d16:1/16:0) | 834.6 | 236.3 | y | y | y | y |
| 80 | Hex2Cer | Hex2Cer(d18:1/16:0) | 862.6 | 264.3 | y | y | | |
| 81 | Hex2Cer | Hex2Cer(d18:1/22:0) | 946.7 | 264.3 | y | y | | |
| 82 | Hex2Cer | Hex2Cer(d18:1/24:1) | 972.7 | 264.3 | y | y | | |
| 83 | Hex3Cer | Hex3Cer(d18:1/16:0) | 1024.7 | 264.3 | y | y | | |
| 84 | HexCer | HexCer(d18:1/16:0) | 700.6 | 264.3 | y | y | | |
| 85 | HexCer | HexCer(d18:1/18:0) | 728.6 | 264.3 | y | y | | |
| 86 | HexCer | HexCer(d18:1/22:0) | 784.7 | 264.3 | y | y | | |
| 87 | HexCer | HexCer(d18:1/24:1) | 810.7 | 264.3 | y | y | | |
| 88 | HexCer | HexCer(d18:2/24:0) | 810.7 | 262.3 | y | y | y | y |
| 89 | LPC | LPC(14:0) | 468.3 | 184.1 | y | | | |
| 90 | LPC | LPC(17:0) | 510.4 | 184.1 | y | y | y | y |
| 91 | LPC | LPC(18:0) | 524.4 | 184.1 | y | y | y | y |
| 92 | LPC | LPC(18:1) | 522.4 | 184.1 | y | | | |
| 93 | LPC | LPC(18:2) | 520.3 | 184.1 | y | | | |
| 94 | LPC | LPC(18:3) | 518.3 | 184.1 | y | y | y | y |
| 95 | LPC | LPC(19:0) | 538.4 | 184.1 | y | y | y | y |
| 96 | LPC | LPC(19:1) | 536.4 | 184.1 | y | y | y | y |
| 97 | LPC | LPC(20:4) | 544.3 | 184.1 | y | y | y | y |
| 98 | LPC | LPC(20:5) | 542.3 | 184.1 | y | | | |
| 99 | LPC | LPC(22:0) | 580.4 | 184.1 | y | y | y | y |
| 100 | LPC | LPC(22:5) | 570.4 | 184.1 | y | y | y | y |
| 101 | LPC | LPC(22:6) | 568.3 | 184.1 | y | y | y | y |
| 102 | LPC | LPC(24:0) | 608.5 | 184.1 | y | y | y | y |
| 103 | LPC | LPC(26:0) | 636.5 | 184.1 | y | y | y | y |
| 104 | LPC(O) | LPC(O-22:0) | 566.5 | 104.1 | y | y | y | y |
| 105 | LPC(O) | LPC(O-22:1) | 564.4 | 104.1 | y | y | y | y |
| 106 | LPC(O) | LPC(O-24:0) | 594.5 | 104.1 | y | y | y | y |
| 107 | LPC(P) | LPC(P-16:0) | 480.3 | 104.1 | y | y | y | y |
| 108 | LPC(P) | LPC(P-17:0) | 494.3 | 104.1 | y | y | y | y |
| 109 | LPE | LPE(16:0) | 454.3 | 313.3 | y | y | y | y |
| 110 | LPE | LPE(18:0) | 482.3 | 341.3 | y | y | y | y |
| 111 | LPE | LPE(18:1) | 480.3 | 339.3 | y | y | y | y |
| 112 | LPE | LPE(18:2) | 478.3 | 337.3 | y | y | y | y |
| 113 | LPE | LPE(20:4) | 502.3 | 361.3 | y | y | y | y |
| 114 | LPE | LPE(22:6) | 526.3 | 385.3 | y | y | y | y |
| 115 | LPE(P) | LPE(P-16:0) | 438.3 | 266.4 | y | y | y | y |
| 116 | LPE(P) | LPE(P-18:0) | 466.3 | 294.4 | y | y | y | y |
| 117 | LPI | LPI(18:0) | 618.3 | 341.3 | y | y | y | y |
| 118 | methyl-CE | methyl-CE(18:0) | 684.6 | 383.3 | | | | |
| 119 | methyl-CE | methyl-CE(18:2) | 680.6 | 383.3 | | | | |
| 120 | methyl-CE | methyl-CE(20:4) | 704.6 | 383.3 | | | | |
| 121 | methyl-DE | methyl-DE(18:1) | 680.6 | 381.4 | | | | |
| 122 | methyl-DE | methyl-DE(18:2) | 678.6 | 381.4 | | | | |
| 123 | PC | PC(15:0_22:6) | 792.6 | 184.1 | y | y | y | y |
| 124 | PC | PC(16:0_16:0) | 734.6 | 184.1 | y | y | y | |
| 125 | PC | PC(16:0_18:1) | 760.6 | 184.1 | y | y | y | y |
| 126 | PC | PC(16:0_18:2) | 758.6 | 184.1 | y | y | y | y |
| 127 | PC | PC(16:0_20:4) | 782.6 | 184.1 | y | y | y | y |
| 128 | PC | PC(16:0_20:5) | 780.5 | 184.1 | y | | | |
| 129 | PC | PC(16:0_22:6) | 806.6 | 184.1 | y | y | y | y |
| 130 | PC | PC(16:1_20:4) | 780.5 | 184.1 | y | | | |
| 131 | PC | PC(18:0_20:4) | 810.6 | 184.1 | y | y | y | y |
| 132 | PC | PC(18:0_22:6) | 834.6 | 184.1 | y | y | y | y |
| 133 | PC | PC(18:1_20:3) | 810.6 | 184.1 | y | y | y | y |
| 134 | PC | PC(18:2_18:2) | 782.6 | 184.1 | y | y | y | y |
| 135 | PC | PC(31:0) | 720.6 | 184.1 | y | | | |
| 136 | PC | PC(31:1) | 718.5 | 184.1 | | | | |
| 137 | PC | PC(32:1) | 732.6 | 184.1 | y | y | y | y |
| 138 | PC | PC(32:2) | 730.5 | 184.1 | y | y | y | y |
| 139 | PC | PC(33:1) | 746.6 | 184.1 | y | y | y | y |
| 140 | PC | PC(33:2) | 744.6 | 184.1 | y | y | y | y |
| 141 | PC | PC(36:2) | 786.6 | 184.1 | y | | | |
| 142 | PC | PC(36:4) | 782.6 | 184.1 | y | y | y | y |
| 143 | PC | PC(38:4) (b) | 810.6 | 184.1 | y | y | y | y |
| 144 | PC | PC(38:6) (a) | 806.6 | 184.1 | y | y | y | y |
| 145 | PC | PC(40:8) | 830.6 | 184.1 | y | y | y | y |
| 146 | PC | PC(42:10) | 854.5 | 184.1 | | | | |
| 147 | PC | PC(42:2) | 870.5 | 184.1 | | | | |
| 148 | PC | PC(42:3) | 868.5 | 184.1 | | | | |
| 149 | PC | PC(42:4) | 866.5 | 184.1 | | | | |
| 150 | PC | PC(42:7) | 860.5 | 184.1 | | | | |
| 151 | PC | PC(42:8) | 858.5 | 184.1 | | | | |
| 152 | PC(O) | PC(O-16:0/16:0) | 720.6 | 184.1 | y | y | | |
| 153 | PC(O) | PC(O-42:8) | 844.5 | 184.1 | | | | |
| 154 | PC(O) | PC(O-46:7) | 902.5 | 184.1 | | | | |
| 155 | PC(P) | PC(P-16:0/14:0) | 690.4 | 184.1 | y | y | y | y |
| 156 | PC(P) | PC(P-16:0/16:0) | 718.5 | 184.1 | y | y | | |
| 157 | PC(P) | PC(P-16:0/18:2) | 742.5 | 184.1 | y | y | y | y |
| 158 | PC(P) | PC(P-16:0/20:4) | 766.5 | 184.1 | y | y | | |
| 159 | PC(P) | PC(P-16:0/20:5) | 764.6 | 184.1 | y | y | y | y |
| 160 | PC(P) | PC(P-17:0/20:4) | 780.5 | 184.1 | y | y | y | y |
| 161 | PE | PE(16:0_16:1) | 690.5 | 549.5 | y | y | y | y |
| 162 | PE | PE(16:0_18:1) | 718.5 | 577.5 | y | y | y | y |
| 163 | PE | PE(16:0_18:2) | 716.5 | 575.5 | y | y | y | y |
| 164 | PE | PE(16:0_20:4) | 740.5 | 599.5 | y | y | y | y |
| 165 | PE | PE(16:0_22:6) | 764.5 | 623.5 | y | y | y | y |
| 166 | PE | PE(18:0_20:4) | 768.6 | 627.5 | y | y | y | y |
| 167 | PE | PE(18:0_22:6) | 792.6 | 651.5 | y | y | y | y |
| 168 | PE | PE(36:2) | 744.6 | 603.5 | y | y | | |
| 169 | PE(O) | PE(O-16:0/20:4) | 726.5 | 585.5 | y | y | y | y |
| 170 | PE(O) | PE(O-34:1) | 704.6 | 563.5 | y | y | y | y |
| 171 | PE(P) | PE(P-16:0/18:2) | 700.5 | 337.3 | y | y | y | y |
| 172 | PE(P) | PE(P-16:0/20:4) | 724.5 | 361.3 | y | y | y | y |
| 173 | PE(P) | PE(P-16:0/20:5) | 722.5 | 359.3 | y | y | y | y |
| 174 | PE(P) | PE(P-16:0/22:6) | 748.5 | 385.3 | y | y | y | y |
| 175 | PE(P) | PE(P-17:0/20:4) | 738.6 | 361.3 | y | y | y | y |
| 176 | PE(P) | PE(P-17:0/22:6) | 762.6 | 385.3 | y | y | y | y |
| 177 | PE(P) | PE(P-18:0/18:1) | 730.6 | 339.3 | y | y | y | y |
| 178 | PE(P) | PE(P-18:0/18:2) | 728.6 | 337.3 | y | y | y | y |
| 179 | PE(P) | PE(P-18:0/20:4) | 752.6 | 361.3 | y | y | y | y |
| 180 | PE(P) | PE(P-18:0/20:5) | 750.5 | 359.3 | y | y | y | y |
| 181 | PE(P) | PE(P-18:0/22:6) | 776.6 | 385.3 | y | y | y | y |
| 182 | PE(P) | PE(P-18:1/20:4) | 750.5 | 361.3 | y | y | y | y |
| 183 | PE(P) | PE(P-18:1/20:5) | 748.5 | 359.3 | y | y | y | y |
| 184 | PE(P) | PE(P-20:0/18:2) | 756.6 | 337.3 | y | y | y | y |
| 185 | PE(P) | PE(P-20:0/20:4) | 780.6 | 361.3 | y | y | y | y |
| 186 | PI | PI(15-MHDA_20:4) & | 890.6 | 613.6 | y | y | y | y |
| | | PI(17:0_20:4) | | | | | | |
| 187 | PI | PI(16:0_16:1) | 826.5 | 549.5 | y | | y | |
| 188 | PI | PI(16:0_20:4) | 876.6 | 599.6 | y | | | |
| 189 | PI | PI(18:0_18:1) | 882.6 | 605.6 | y | | | |
| 190 | PI | PI(18:0_20:4) | 904.6 | 627.6 | y | y | | |
| 191 | PI | PI(18:0_22:6) | 928.6 | 651.6 | y | y | | |
| 192 | PI | PI(34:1) | 854.6 | 577.6 | y | | | |
| 193 | PI | PI(36:2) | 880.6 | 603.6 | y | y | | |
| 194 | PI | PI(38:6) | 900.6 | 623.6 | y | | | |
| 195 | PIP1 | PIP1(38:4) | 984.7 | 627.7 | | | | |
| 196 | PS | PS(38:4) | 812.5 | 627.5 | y | y | | |
| 197 | S1P | S1P(d18:1) | 380.2 | 264.3 | y | y | y | y |
| 198 | SHexCer | SHexCer(d18:1/16:0(OH)) | 796.8 | 264.3 | y | y | y | y |
| 199 | SHexCer | SHexCer(d18:1/16:0) | 780.8 | 264.3 | y | y | y | y |
| 200 | SM | SM(34:3) | 699.5 | 184.1 | y | y | y | y |
| 201 | SM | SM(35:1) | 717.6 | 184.1 | y | y | y | y |
| 202 | SM | SM(35:2) | 715.6 | 184.1 | y | y | y | y |
| 203 | SM | SM(38:3) | 755.6 | 184.1 | y | y | y | y |
| 204 | SM | SM(41:1) | 801.7 | 184.1 | y | y | y | y |
| 205 | SM | SM(44:2) | 841.6 | 184.1 | y | y | y | y |
| 206 | SM | SM(d17:1/14:0) | 661.5 | 184.1 | y | y | y | y |
| 207 | SM | SM(d17:1/16:0) | 689.6 | 184.1 | y | y | y | y |
| 208 | SM | SM(d18:1/14:0) & | 675.5 | 184.1 | y | y | y | y |
| | | SM(d16:1/16:0) | | | | | | |
| 209 | SM | SM(d18:1/16:0) | 703.6 | 184.1 | y | y | y | y |
| 210 | SM | SM(d18:1/18:0) & | 731.6 | 184.1 | y | | y | |
| | | SM(d16:1/20:0) | | | | | | |
| 211 | SM | SM(d18:1/20:0) & | 759.6 | 184.1 | y | y | y | y |
| | | SM(d16:1/22:0) | | | | | | |
| 212 | SM | SM(d18:1/22:0) & | 787.7 | 184.1 | y | y | y | y |
| | | SM(d16:1/24:0) | | | | | | |
| 213 | SM | SM(d18:2/14:0) | 673.5 | 184.1 | y | y | y | y |
| 214 | SM | SM(d18:2/16:0) | 701.6 | 184.1 | y | y | y | y |
| 215 | SM | SM(d18:2/18:1) | 727.6 | 184.1 | y | y | y | y |
| 216 | SM | SM(d18:2/20:0) | 757.6 | 184.1 | y | y | y | y |
| 217 | TG [NL] | TG(48:1) [NL-18:1] | 822.8 | 523.5 | y | | | |
| 218 | TG [NL] | TG(48:2) [NL-14:0] | 820.8 | 575.5 | y | | | |
| 219 | TG [NL] | TG(48:2) [NL-14:1] | 820.8 | 577.5 | y | | y | |
| 220 | TG [NL] | TG(48:2) [NL-16:1] | 820.8 | 549.5 | y | | | |
| 221 | TG [NL] | TG(48:2) [NL-18:2] | 820.8 | 523.5 | y | | | |
| 222 | TG [NL] | TG(48:3) [NL-14:0] | 818.8 | 573.5 | y | | | |
| 223 | TG [NL] | TG(48:3) [NL-16:1] | 818.8 | 547.5 | y | | | |
| 224 | TG [NL] | TG(50:2) [NL-16:1] | 848.8 | 577.5 | y | | | |
| 225 | TG [NL] | TG(50:2) [NL-18:1] | 848.8 | 549.5 | y | | y | |
| 226 | TG [NL] | TG(50:3) [NL-14:0] | 846.8 | 601.5 | y | y | y | y |
| 227 | TG [NL] | TG(50:3) [NL-14:1] | 846.8 | 603.5 | y | | | |
| 228 | TG [NL] | TG(50:3) [NL-16:1] | 846.8 | 575.5 | y | | y | |
| 229 | TG [NL] | TG(50:3) [NL-18:2] | 846.8 | 549.5 | y | | | |
| 230 | TG [NL] | TG(50:4) [NL-14:0] | 844.8 | 599.5 | y | | | |
| 231 | TG [NL] | TG(50:4) [NL-18:3] | 844.8 | 549.5 | y | y | y | y |
| 232 | TG [NL] | TG(50:4) [NL-20:4] | 844.8 | 523.5 | y | y | y | y |
| 233 | TG [NL] | TG(51:2) [NL-17:0] | 862.8 | 575.5 | y | y | y | y |
| 234 | TG [NL] | TG(51:2) [NL-17:1] | 862.8 | 577.5 | y | y | y | y |
| 235 | TG [NL] | TG(52:2) [NL-16:0] | 876.8 | 603.5 | y | y | | |
| 236 | TG [NL] | TG(52:3) [NL-18:2] | 874.8 | 577.5 | y | y | y | y |
| 237 | TG [NL] | TG(52:4) [NL-16:1] | 872.8 | 601.5 | y | y | y | y |
| 238 | TG [NL] | TG(52:4) [NL-18:2] | 872.8 | 575.5 | y | y | y | y |
| 239 | TG [NL] | TG(52:4) [NL-18:3] | 872.8 | 577.5 | y | y | y | y |
| 240 | TG [NL] | TG(52:5) [NL-18:3] | 870.8 | 575.5 | y | y | y | y |
| 241 | TG [NL] | TG(52:5) [NL-20:4] | 870.8 | 549.5 | y | y | y | y |
| 242 | TG [NL] | TG(54:2) [NL-20:1] | 904.8 | 577.5 | y | y | y | y |
| 243 | TG [NL] | TG(54:3) [NL-18:1] | 902.8 | 603.5 | y | y | y | y |
| 244 | TG [NL] | TG(54:5) [NL-18:3] | 898.8 | 603.5 | y | y | y | y |
| 245 | TG [NL] | TG(54:5) [NL-20:4] | 898.8 | 577.5 | y | y | y | y |
| 246 | TG [NL] | TG(54:6) [NL-18:3] | 896.8 | 601.5 | y | y | y | y |
| 247 | TG [NL] | TG(54:6) [NL-20:4] | 896.8 | 575.5 | y | y | y | y |
| 248 | TG [NL] | TG(54:6) [NL-20:5] | 896.8 | 577.5 | y | y | y | y |
| 249 | TG [NL] | TG(54:6) [NL-22:6] | 896.8 | 551.5 | y | y | y | y |
| 250 | TG [NL] | TG(54:7) [NL-20:5] | 894.8 | 575.5 | y | y | y | y |
| 251 | TG [NL] | TG(54:7) [NL-22:6] | 894.8 | 549.5 | y | y | y | y |
| 252 | TG [NL] | TG(56:6) [NL-20:4] | 924.8 | 603.5 | y | y | y | y |
| 253 | TG [NL] | TG(56:6) [NL-22:5] | 924.8 | 577.5 | y | y | y | y |
| 254 | TG [NL] | TG(56:7) [NL-20:4] | 922.8 | 601.5 | y | y | y | y |
| 255 | TG [NL] | TG(56:7) [NL-20:5] | 922.8 | 603.5 | y | y | y | y |
| 256 | TG [NL] | TG(56:7) [NL-22:5] | 922.8 | 575.5 | y | y | y | y |
| 257 | TG [NL] | TG(56:7) [NL-22:6] | 922.8 | 577.5 | y | y | y | y |
| 258 | TG [NL] | TG(56:8) [NL-20:4] | 920.8 | 599.5 | y | y | | |
| 259 | TG [NL] | TG(56:8) [NL-20:5] | 920.8 | 601.5 | y | y | y | y |
| 260 | TG [NL] | TG(56:8) [NL-22:6] | 920.8 | 575.5 | y | y | y | y |
| 261 | TG [NL] | TG(56:9) [NL-22:6] | 918.8 | 573.5 | y | y | y | y |
| 262 | TG [NL] | TG(58:10) [NL-22:6] | 944.9 | 599.5 | y | y | y | y |
| 263 | TG [NL] | TG(58:8) [NL-22:6] | 948.8 | 603.5 | y | y | y | y |
| 264 | TG [NL] | TG(58:9) [NL-22:6] | 946.9 | 601.5 | y | y | y | y |
| 265 | TG(O) [NL] | TG(O-50:2) [NL-16:1] | 834.8 | 563.5 | y | y | y | y |
| 266 | TG(O) [NL] | TG(O-50:2) [NL-18:1] | 834.8 | 535.5 | y | y | y | y |
| 267 | TG(O) [NL] | TG(O-50:2) [NL-18:2] | 834.8 | 537.5 | y | y | y | y |
| 268 | TG(O) [NL] | TG(O-54:4) [NL-17:1] | 886.8 | 601.5 | y | y | y | y |
| 269 | Ubiquinone | Ubiquinone | 880.7 | 197 | y | y | y | y |

### Example 9: Improvements in the Design and Measurement of CLP2

A small number of changes have been made to increase the accuracy of quantitation of lipids previously listed in CLP lipid lists.

The following MRM transitions were added. They do not measure additional lipid species, but rather they consist of the [M+2+H]/[M+2+H]transitions of lipid species that are already monitored and detailed in the CLP1 and CLP2 lists. These additions have been made for species that are typically high in abundance, thus there is a possibility that in some samples, detector saturation may occur, and measurement accuracy may be compromised. By measuring the [M+2+H]/[M+2+H]transition for these species, which is naturally much lower in abundance, this potential issue can be avoided. Accordingly, these transitions are monitored in addition to the M+H ions.

TABLE 31
[M + 2 + H] MRM transitions used as an alternative means to monitor
three high abundance PC species and an internal standard.

| | | Precursor | Product |
|---|---|---|---|
| Lipid Class | Compound Name | (m/z) | (m/z) |
| PC | PC 34:1 [+2/+2] | 762.6 | 186.1 |
| PC | PC 34:2 [+2/+2] | 760.6 | 186.1 |
| PC | PC 36:2 [+2/+2] | 788.6 | 186.1 |
| PC | PC(15:0/18:1) d7 [+2/+2] | 755.6 | 186.1 |

### Example 10: A mBMI Score Using about 50 Lipid Species

In order to demonstrate the use of a smaller subset of lipid species in the calculation of mBMI, the following analysis on the AusDiab cohort was performed.

The analysis encompassed two distinct lipidome models: a Ridge regression model using the full CLP2 lipidomic panel comprising ~250 lipid species, and LASSO model where we imposed a constraint on the number of lipid species included in the model, limiting it to ~50 lipid species. To achieve this, lambda parameters were selected within a restricted range spanning from 0.16 to 6.3.

A single LASSO model containing 50 lipid species was first generated, and used to establish the list of 50 lipid species (Table 32).

To assess the performance of the Ridge and LASSO models, a 10-fold cross-validation framework was then implemented to randomly partition the AusDiab dataset, repeatedly designating one-fold as the validation set and merging the remaining folds as the training set. Within each iteration of the training set, Ridge or LASSO regression was applied to establish the relationships between BMI and the entire lipidome, while adjusting for age and sex. Continuous predictors underwent standardisation to achieve zero-mean and unit-variance.

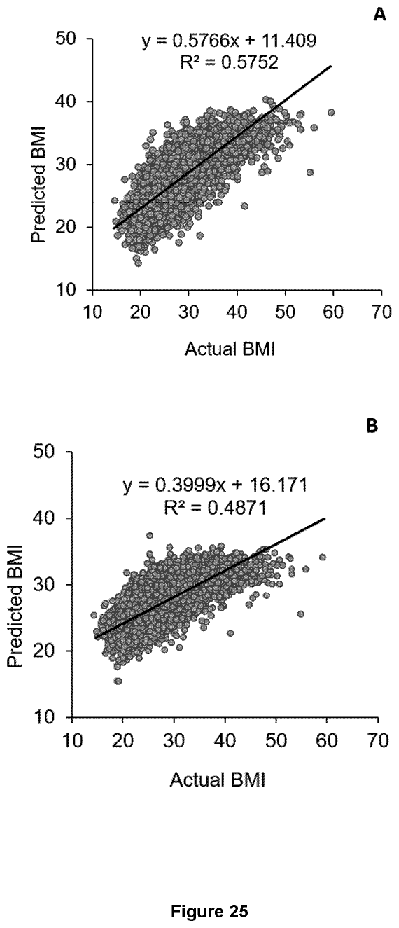

Prediction performance was evaluated using R2, computed as the square of the Pearson correlation between predicted BMI and actual BMI. The analysis revealed that the predicted BMI derived from the CLP2, ~250 lipids could account for up to 57.5% of the variance in BMI. When restricting the clinical lipidome panel to 50 species in the LASSO model, R2 of 48.7% was achieved (FIG. 25).

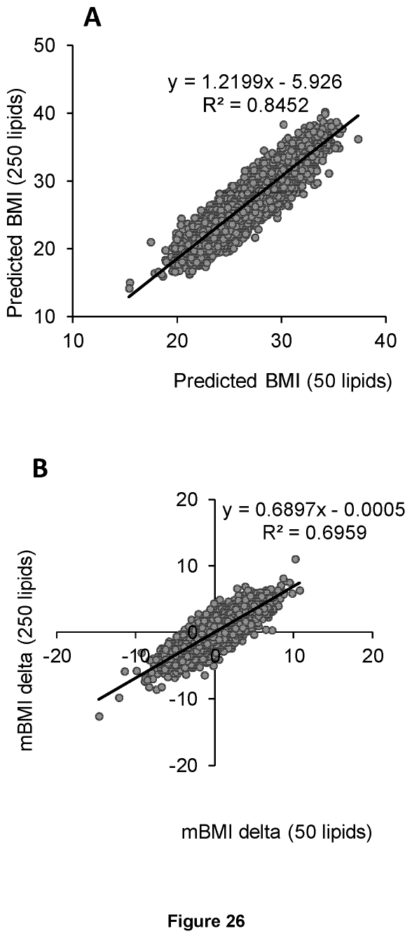

The correlation between predicted BMI using the 50 lipid subset of the CLP2 lipids, and predicted BMI using ~250 CLP2 lipids in the AusDiab cohort was also compared (R2=84.5%) (FIG. 26).

The metabolic BMI delta computed from a model with just 50 lipids was strongly correlated with the metabolic BMI delta obtained using the ~250 lipid species (CLP2) (R2=69.6%) (FIG. 26). These strong correlations demonstrate the ability of a subset of about 50 lipids to perform similarly to the full ~250 lipid species in the CLP2 lipid set.

This lipid list serves as an illustrative example for how a smaller subset of the CLP2 lipids, approximately 50 in total, could be used to create a model of comparable performance. A person skilled in the art would appreciate that these approximately 50 lipids could be easily substituted for an alternate, highly correlated subset of approximately 50 lipids (selected from the CLP2 lipid list) to yield a model of comparable performance. Thus, there are many combinations of 50 lipid species (selected from the CLP2 list and based on the principles demonstrated herein) that can predict BMI with similar efficiency.

TABLE 32
Predictive models of BMI developed using LASSO regression
with an approximately 50 lipid subset the CLP2s lipids

| | | | LASSO | LASSO (ABS) |
|---|---|---|---|---|
| Rank # | Lipid # | Lipid/Covariate | coefficient | coefficient |
| 1 | 214 | SM(d18:2/16:0) | 1.39553 | 1.39553 |
| 2 | 200 | SM(34:3) | 0.725004 | 0.725004 |
| 3 | 145 | PC(40:8) | −0.65731 | 0.65731 |
| 4 | 123 | PC(15:0_22:6) | −0.61525 | 0.61525 |
| 5 | 105 | LPC(O-22:1) | −0.56991 | 0.56991 |
| 6 | 78 | GM3(d18:1/24:1) | −0.56879 | 0.56879 |
| 7 | 241 | TG(52:5) [NL-20:4] | 0.493576 | 0.493576 |
| 8 | 48 | Cer(m18:0/22:0) | 0.460099 | 0.460099 |
| 9 | 103 | LPC(26:0) | −0.45991 | 0.45991 |
| 10 | 249 | TG(54:6) [NL-22:6] | 0.34461 | 0.34461 |
| 11 | 209 | SM(d18:1/16:0) | −0.3275 | 0.3275 |
| 12 | 184 | PE(P-20:0/18:2) | −0.32311 | 0.32311 |
| 13 | 187 | PI(16:0_16:1) | 0.283687 | 0.283687 |
| 14 | 157 | PC(P-16:0/18:2) | −0.22577 | 0.22577 |
| 15 | 102 | LPC(24:0) | −0.22238 | 0.22238 |
| 16 | 132 | PC(18:0_22:6) | 0.219229 | 0.219229 |
| 17 | 45 | Cer(d19:1/22:0) | 0.21241 | 0.21241 |
| 18 | 49 | Cer(m18:1/20:0) | 0.211391 | 0.211391 |
| 19 | 140 | PC(33:2) | −0.17862 | 0.17862 |
| 20 | 91 | LPC(18:0) | 0.171972 | 0.171972 |
| 21 | 2 | AC(12:0) | −0.16855 | 0.16855 |
| 22 | 164 | PE(16:0_20:4) | −0.15884 | 0.15884 |
| 23 | 204 | SM(41:1) | 0.124063 | 0.124063 |
| 24 | 255 | TG(56:7) [NL-20:5] | 0.118254 | 0.118254 |
| 25 | 88 | HexCer(d18:2/24:0) | −0.09589 | 0.09589 |
| 26 | 43 | Cer(d18:2/24:0) | −0.09133 | 0.09133 |
| 27 | 173 | PE(P-16:0/20:5) | 0.089682 | 0.089682 |
| 28 | 198 | SHexCer(d18:1/ | −0.08855 | 0.08855 |
| | | 16:0(OH)) | | |
| 29 | 83 | Hex3Cer(d18:1/16:0) | −0.08717 | 0.08717 |
| 30 | 90 | LPC(17:0) | −0.08551 | 0.08551 |
| 31 | 96 | LPC(19:1) | −0.08293 | 0.08293 |
| 32 | 53 | Cer1P(d18:1/16:0) | −0.08158 | 0.08158 |
| 33 | 55 | DE(18:1) | 0.080642 | 0.080642 |
| 34 | 58 | DG(16:0_16:0) | 0.071854 | 0.071854 |
| 35 | 115 | LPE(P-16:0) | 0.071069 | 0.071069 |
| 36 | 104 | LPC(O-22:0) | −0.0544 | 0.0544 |
| 37 | 82 | Hex2Cer(d18:1/24:1) | 0.048737 | 0.048737 |
| 38 | 142 | PC(36:4) | −0.04525 | 0.04525 |
| 39 | 199 | SHexCer(d18:1/16:0) | −0.03792 | 0.03792 |
| 40 | 35 | Cer(d18:1/16:0) | −0.0254 | 0.0254 |
| 41 | 59 | DG(16:0_18:1) | 0.019411 | 0.019411 |
| 42 | 172 | PE(P-16:0/20:4) | 0.019269 | 0.019269 |
| 43 | 165 | PE(16:0_22:6) | −0.00938 | 0.00938 |
| 44 | 213 | SM(d18:2/14:0) | 0.006055 | 0.006055 |
| 45 | 50 | Cer(m18:1/22:0) | 0.005197 | 0.005197 |
| 46 | 20 | CE(16:1) | 0.003573 | 0.003573 |
| 47 | 18 | CE(15:0) | −0.00223 | 0.00223 |
| 48 | 34 | Cer(d17:1/24:0) | −0.001 | 0.001 |
| 49 | 234 | TG(51:2) [NL-17:1] | 0.00059 | 0.00059 |
| 50 | 12 | AC(18:1) | 0.000581 | 0.000581 |

### Example 11: a mAge Score Using about 50 Lipid Species

In order to demonstrate the use of a smaller subset of lipid species in the calculation of mAge, two sets of analyses were performed using the clinical lipid panel consisting of ~250 lipid species measured on the entire AusDiab cohort:

The analyses encompassed two distinct lipidome models: a Ridge regression model using the full CLP2 lipidomic panel comprising ~250 lipid species and LASSO model where a constraint on the number of lipid species included in the model was imposed, limiting it to ~50 lipid species. To achieve this, lambda parameters were selected within a restricted range spanning from 0.32 to 3.16.

A single LASSO model containing 50 lipid species was first generated, and used to establish the list of 50 lipid species (Table 33).

To assess the performance of the Ridge and LASSO models, a 10-fold cross-validation framework was implemented to randomly partition the AusDiab dataset, repeatedly designating one-fold as the validation set and merging the remaining folds as the training set. Within each iteration of the training set, Ridge or LASSO regression was applied to establish the relationships between Age and the entire lipidome, while adjusting for BMI and sex. Continuous predictors underwent standardisation to achieve zero-mean and unit-variance.

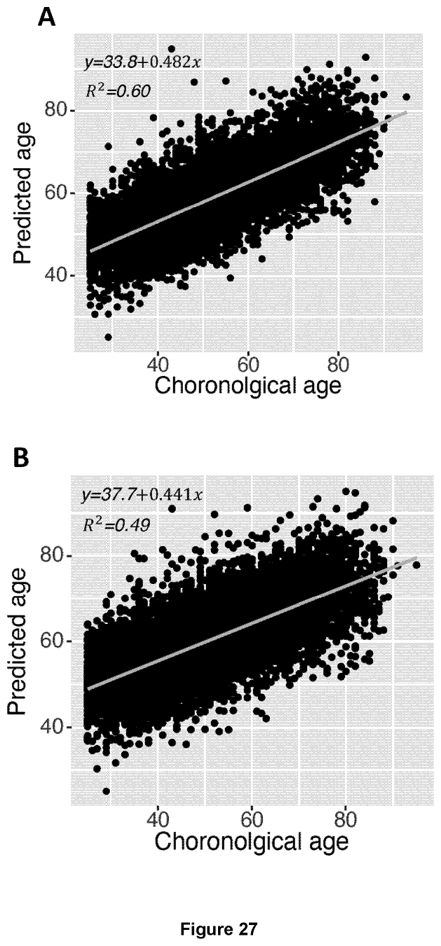

Prediction performance was evaluated using R2, computed as the square of the Pearson correlation between predicted Age and chronological Age. The analysis revealed that the predicted Age derived from the CLP2, ~250 lipids could account for up to 60% of the variance in BMI. When restricting the clinical lipidome panel to 50 species in the LASSO model, R2 of 49% was achieved (FIG. 27).

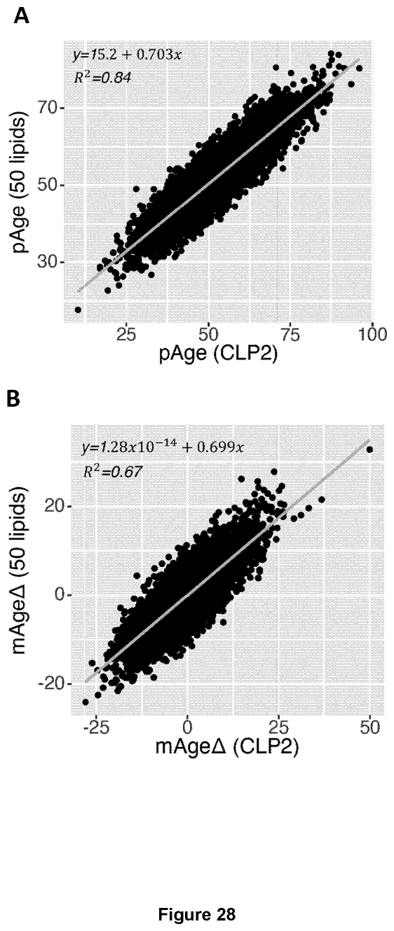

The correlation between predicted Age using 50 lipid subsets of the CLP2 lipids, and predicted Age using ~250 CLP2 lipids in the AusDiab cohort was also compared (R2=84%) (FIG. 28).

The metabolic Age delta computed from a model with just 50 lipids was strongly correlated with the metabolic Age delta obtained using the ~250 lipid species (CLP2) (R2=67%). These strong correlations demonstrate the ability of a subset of about 50 lipids to perform similarly to the full ~250 lipid species in the CLP2 lipid set (FIG. 28).

This lipid list serves as an illustrative example for how a smaller subset of the CLP2 lipids, approximately 50 in total, could be used to create a model of comparable performance. A person skilled in the art would appreciate that these approximately 50 lipids could be easily substituted for an alternate, highly correlated subset of approximately 50 lipids (selected from the CLP2 lipid list) to yield a model of comparable performance. Thus, there are many combinations of 50 lipid species (selected from the CLP2 list and based on the principles demonstrated herein) that can predict age with similar efficiency.

TABLE 33
Predictive model of Age developed using LASSO regression
with an approximately 50 lipid subset the CLP2 lipids

| Rank # | Lipid # | Lipid/Covariate | LASSO | LASSO (ABS) |
|---|---|---|---|---|
| 1 | 51 | Cer(m18:1/24:0) | 2.681683 | 2.681683 |
| 2 | 207 | SM(d17:1/16:0) | 2.461363 | 2.461363 |
| 3 | 157 | PC(P-16:0/18:2) | −2.43134 | 2.43134 |
| 4 | 212 | SM(d18:1/22:0) & | −2.31413 | 2.31413 |
| | | SM(d16:1/24:0) | | |
| 5 | 214 | SM(d18:2/16:0) | 2.168726 | 2.168726 |
| 6 | 80 | Hex2Cer(d18:1/16:0) | −1.78226 | 1.78226 |
| 7 | 178 | PE(P-18:0/18:2) | 1.763973 | 1.763973 |
| 8 | 132 | PC(18:0_22:6) | 1.677574 | 1.677574 |
| 9 | 22 | CE(17:0) | −1.65542 | 1.65542 |
| 10 | 192 | PI(34:1) | −1.54075 | 1.54075 |
| 11 | 11 | AC(16:1) | 1.443498 | 1.443498 |
| 12 | 124 | PC(16:0_16:0) | 1.330664 | 1.330664 |
| 13 | 169 | PE(O-16:0/20:4) | −1.20551 | 1.20551 |
| 14 | 81 | Hex2Cer(d18:1/22:0) | 1.047627 | 1.047627 |
| 15 | 59 | DG(16:0_18:1) | −0.99253 | 0.99253 |
| 16 | 197 | S1P(d18:1) | −0.94492 | 0.94492 |
| 17 | 205 | SM(44:2) | 0.932161 | 0.932161 |
| 18 | 10 | AC(16:0) | −0.85666 | 0.85666 |
| 19 | 9 | AC(14:2) | 0.836427 | 0.836427 |
| 20 | 4 | AC(13:0) | 0.758412 | 0.758412 |
| 21 | 35 | Cer(d18:1/16:0) | 0.689649 | 0.689649 |
| 22 | 152 | PC(O-16:0/16:0) | −0.68585 | 0.68585 |
| 23 | 145 | PC(40:8) | −0.68518 | 0.68518 |
| 24 | 50 | Cer(m18:1/22:0) | 0.680683 | 0.680683 |
| 25 | 210 | SM(d18:1/18:0) & | −0.63191 | 0.63191 |
| | | SM(d16:1/20:0) | | |
| 26 | 163 | PE(16:0_18:2) | 0.587537 | 0.587537 |
| 27 | 252 | TG(56:6) [NL-20:4] | 0.587144 | 0.587144 |
| 28 | 36 | Cer(d18:1/20:0) | 0.581617 | 0.581617 |
| 29 | 13 | AC(18:2) | 0.544193 | 0.544193 |
| 30 | 18 | CE(15:0) | 0.534583 | 0.534583 |
| 31 | 159 | PC(P-16:0/20:5) | 0.470061 | 0.470061 |
| 32 | 12 | AC(18:1) | 0.43879 | 0.43879 |
| 33 | 78 | GM3(d18:1/24:1) | 0.4241 | 0.4241 |
| 34 | 105 | LPC(O-22:1) | −0.3779 | 0.3779 |
| 35 | 174 | PE(P-16:0/22:6) | −0.36965 | 0.36965 |
| 36 | 25 | CE(20:0) | −0.33699 | 0.33699 |
| 37 | 209 | SM(d18:1/16:0) | 0.327396 | 0.327396 |
| 38 | 40 | Cer(d18:1/24:1) | 0.321663 | 0.321663 |
| 39 | 186 | PI(15-MHDA_20:4) & | 0.28568 | 0.28568 |
| | | PI(17:0_20:4) | | |
| 40 | 24 | CE(18:3) | 0.259685 | 0.259685 |
| 41 | 198 | SHexCer(d18:1/ | −0.25125 | 0.25125 |
| | | 16:0(OH)) | | |
| 42 | 5 | AC(14:0) | 0.235766 | 0.235766 |
| 43 | 88 | HexCer(d18:2/24:0) | 0.215976 | 0.215976 |
| 44 | 26 | CE(20:1) | −0.14152 | 0.14152 |
| 45 | 265 | TG(O-50:2) [NL-16:1] | −0.09936 | 0.09936 |
| 46 | 58 | DG(16:0_16:0) | −0.09908 | 0.09908 |
| 47 | 199 | SHexCer(d18:1/16:0) | 0.081087 | 0.081087 |
| 48 | 226 | TG(50:3) [NL-14:0] | 0.04638 | 0.04638 |
| 49 | 184 | PE(P-20:0/18:2) | 0.010248 | 0.010248 |

### Example 12: A Cardiovascular Disease (CVD) Risk Score Using about 50 Lipid Species

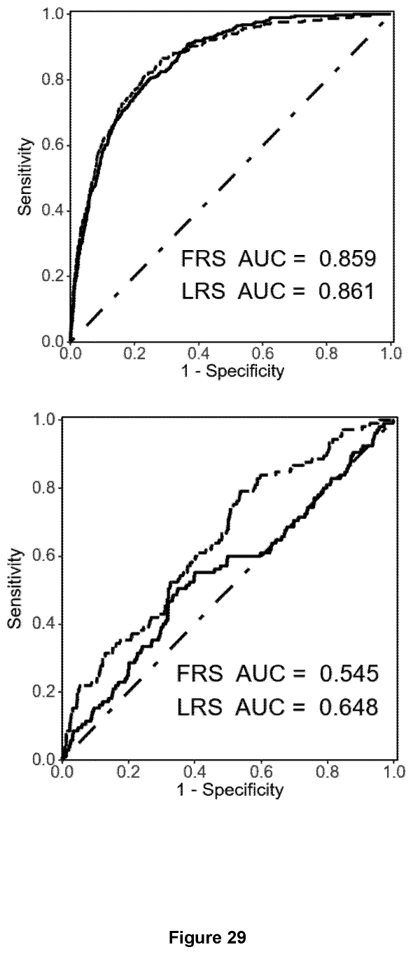

To assess whether a lipid model (LRS) containing ~50 lipid species would perform comparably to a model containing a larger number of lipid species, a LRS50 model was developed using LASSO feature selection, which selected 50 lipids as predictors from the list of ~250 lipids species in the CLP2. The LRS50 performance was assessed by two metrics: the receiver operating characteristic-area under the curve (ROC-AUC) and the net reclassification improvement (NRI). FIG. 29 shows the AUC plots of LRS50 for the whole population and the intermediate-risk group, respectively, in the Ausdiab. The AUC for the LRS50 (0.648) was improved over the AUC for the FRS (0.545) and similar to the AUC for the CLP2 model (0.653, see Example 7).

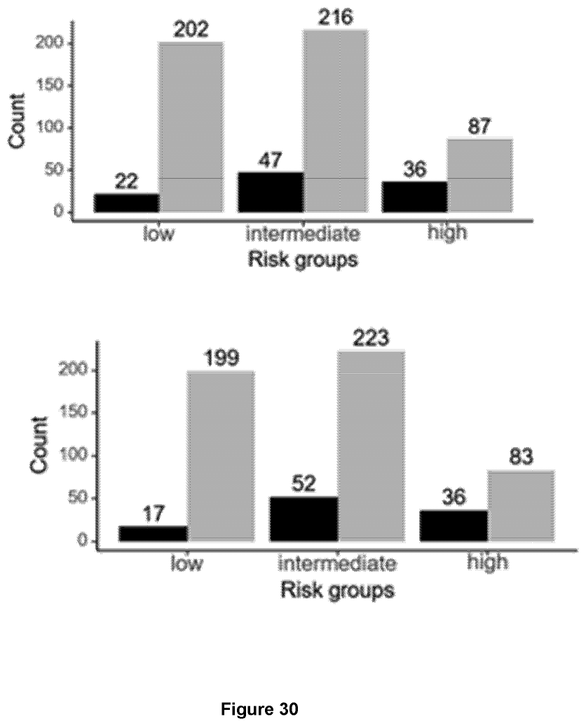

FIG. 30 shows the reclassification of the intermediate-risk group in Ausdiab cohort using either the LRS based on the full lipid panel (CLP0 in Example 6) or the LRS50. In the LRS50 model, the reclassification from the FRS intermediate risk group to the high risk group is comparable (36 events reclassified) while the reclassification from the FRS intermediate risk group to the low risk group is improved with the LRS50 (17 events vs. 22 events). These data were subsequently used for the NRI calculation. Table 34 lists the raw counts and NRI statistics comparing LRS50 to the LRS full lipidome in the intermediate-risk group in Ausdiab.

In summary, for the LRS50, the metrics of AUC and NRI were significantly improved as compared to the FRS in the intermediate-risk subgroup of Ausdiab, and were similar to what was observed with the LRS derived from full lipid panel (see Example 7). The 50 lipid species and their coefficients in the LRS50 model are shown in Table 35.

TABLE 34
Model performance and net reclassification of the LRS models
Comparison of raw counts and NRI statistics for LRS models
derived from both the full lipid panel and a selected subset
of 50 lipids (LRS50), in the FRS classified intermediate-risk
group of the AusDiab cohort. The LRS50 model demonstrated
comparable levels of NRI events and non-events to the LRS
model derived from the full lipid panel, with both showing
the same proportions (0.343) of cases moving up from
intermediate-risk to high-risk. Events: incident CVD events;
Nonevents: controls; Upward reclassification: moving to a
higher risk level as per LRS; Downward reclassification: moving
to a lower risk level as per LRS; Proportion upward/downward
events: the proportions of events (cases) moving up/down in
risk level; Proportion upward/downward nonevents: the proportions
of nonevents (controls) moving up/down in risk level; NRI:
net reclassification improvement, indicating changes in
risk stratification by LRS as compared to FRS.

| | | | |
|---|---|---|---|
| Counts and NRI stats | Events | Counts and NRI stats | Non events |

LRS based on the full lipid panel

| | | | |
|---|---|---|---|
| n | 105 | n | 505 |
| Downward | 22 | Downward | 202 |
| reclassification n | | reclassification n | |
| Unchanged | 47 | Unchanged | 216 |
| classification n | | classification n | |
| Upward | 36 | Upward | 87 |
| reclassification n | | reclassification n | |
| Proportion upward | 0.343 | Proportion | 0.400 |
| | | downward | |
| Proportion | 0.210 | Proportion upward | 0.172 |
| downward | | | |
| NRI events | 0.13 | NRI nonevents | 0.23 |

LRS based on 50 lipids (LRS50)

| | | | |
|---|---|---|---|
| n | 105 | n | 505 |
| Downward | 22 | Downward | 199 |
| reclassification n | | reclassification n | |
| Unchanged | 47 | Unchanged | 223 |
| classification n | | classification n | |
| Upward | 36 | Upward | 83 |
| reclassification n | | reclassification n | |
| Proportion upward | 0.343 | Proportion | 0.394 |
| | | downward | |
| Proportion | 0.210 | Proportion upward | 0.164 |
| downward | | | |
| NRI events | 0.18 | NRI nonevents | 0.23 |

TABLE 35
Predictive models of LRS developed using LASSO regression
with an approximately 50 lipid subset the CLP2 lipids.

| Rank # | Lipid # | Lipid/Covariate | LASSO | LASSO (ABS) |
|---|---|---|---|---|
| 1 | 213 | SM(d18:2/14:0) | −0.02271272 | 0.02271272 |
| 2 | 145 | PC(40:8) | −0.012829835 | 0.012829835 |
| 3 | 22 | CE(17:0) | 0.00743583 | 0.00743583 |
| 4 | 51 | Cer(m18:1/24:0) | 0.006178197 | 0.006178197 |
| 5 | 49 | Cer(m18:1/20:0) | 0.005899114 | 0.005899114 |
| 6 | 19 | CE(16:0) | 0.005530479 | 0.005530479 |
| 7 | 165 | PE(16:0_22:6) | −0.005484566 | 0.005484566 |
| 8 | 221 | TG(48:2) [NL-18:2] | 0.00523232 | 0.00523232 |
| 9 | 230 | TG(50:4) [NL-14:0] | 0.004905973 | 0.004905973 |
| 10 | 2 | AC(12:0) | 0.004647223 | 0.004647223 |
| 11 | 157 | PC(P-16:0/18:2) | −0.00461755 | 0.00461755 |
| 12 | 27 | CE(20:4) | −0.004544153 | 0.004544153 |
| 13 | 123 | PC(15:0_22:6) | −0.003465211 | 0.003465211 |
| 14 | 155 | PC(P-16:0/14:0) | 0.003382628 | 0.003382628 |
| 15 | 124 | PC(16:0_16:0) | 0.003323943 | 0.003323943 |
| 16 | 26 | CE(20:1) | 0.003186843 | 0.003186843 |
| 17 | 21 | CE(16:2) | −0.002957416 | 0.002957416 |
| 18 | 264 | TG(58:9) [NL-22:6] | 0.002940777 | 0.002940777 |
| 19 | 170 | PE(O-34:1) | −0.002899718 | 0.002899718 |
| 20 | 189 | PI(18:0_18:1) | 0.002821471 | 0.002821471 |
| 21 | 40 | Cer(d18:1/24:1) | 0.002714779 | 0.002714779 |
| 22 | 169 | PE(O-16:0/20:4) | −0.002603198 | 0.002603198 |
| 23 | 209 | SM(d18:1/16:0) | 0.002499028 | 0.002499028 |
| 24 | 56 | DE(18:2) | 0.00240927 | 0.00240927 |
| 25 | 206 | SM(d17:1/14:0) | −0.002225878 | 0.002225878 |
| 26 | 215 | SM(d18:2/18:1) | −0.002195909 | 0.002195909 |
| 27 | 29 | CE(22:1) | 0.001788525 | 0.001788525 |
| 28 | 105 | LPC(O-22:1) | −0.001724128 | 0.001724128 |
| 29 | 107 | LPC(P-16:0) | −0.001709006 | 0.001709006 |
| 30 | 193 | PI(36:2) | −0.001584123 | 0.001584123 |
| 31 | 47 | Cer(d19:1/24:1) | 0.00152513 | 0.00152513 |
| 32 | 269 | Ubiquinone | 0.001511886 | 0.001511886 |
| 33 | 25 | CE(20:0) | 0.001488116 | 0.001488116 |
| 34 | 249 | TG(54:6) [NL-22:6] | 0.001440333 | 0.001440333 |
| 35 | 177 | PE(P-18:0/18:1) | 0.001324517 | 0.001324517 |
| 36 | 167 | PE(18:0_22:6) | −0.001120117 | 0.001120117 |
| 37 | 54 | COH | 0.001079799 | 0.001079799 |
| 38 | 242 | TG(54:2) [NL-20:1] | 0.000934959 | 0.000934959 |
| 39 | 55 | DE(18:1) | 0.000830678 | 0.000830678 |
| 40 | 12 | AC(18:1) | 0.000302306 | 0.000302306 |
| 41 | 35 | Cer(d18:1/16:0) | 0.00024927 | 0.00024927 |
| 42 | 36 | Cer(d18:1/20:0) | 0.000235805 | 0.000235805 |
| 43 | 28 | CE(20:5) | −0.000173186 | 0.000173186 |
| 44 | 24 | CE(18:3) | −0.000154855 | 0.000154855 |
| 45 | 138 | PC(32:2) | −0.000129948 | 0.000129948 |
| 46 | 178 | PE(P-18:0/18:2) | 0.000124759 | 0.000124759 |
| 47 | 20 | CE(16:1) | −0.000102464 | 0.000102464 |
| 48 | 156 | PC(P-16:0/16:0) | 6.24868E−05 | 6.24868E−05 |
| 49 | 196 | PS(38:4) | 2.61261E−05 | 2.61261E−05 |
| 50 | 246 | TG(54:6) [NL-18:3] | 1.72696E−05 | 1.72696E−05 |

Applications in Integrating with Other Omics-Derived Scores

The LRS models described here have the potential for enhanced performance through integration with additional omics-derived risk scores. For example, the LRS can be seamlessly combined with a polygenic risk score for CVD prediction, which has the potential to further improve the AUC and NRI in risk stratification efforts.

### CONCLUSION

In summary, the LRS model derived from the CLP lists and the LRS50 with 50 lipids predict incident CVD as accurately as the LRS derived from the whole lipidome. In particular, for the intermediate risk group, the LRS derived from the CLP and the LRS50 with 50 lipids achieved the same level of improved performance and reclassification as the LRS derived from whole lipidome when compared to the original FRS. With the CLP demonstrating its potential in clinical translation of lipidomic data, both LRS and LRS50 models implemented with a simple, fast, and clinically viable blood test may satisfy the unmet needs of the early detection of CVD.

### Example 13: a Prediabetes and Type 2 Diabetes (T2DM) Risk Score Using about 50 Lipid Species

A Prediabetes and Type 2 Diabetes (T2DM) Risk Score generated using lipidomic data from a patient, and utilising approximately 50 lipid species is described. This risk score's performance is comparable to one generated using a larger number of lipids, such as the CLP2 list.

The AusDiab dataset was filtered to only include individuals with normal glucose tolerance and impaired glucose tolerance (fasting glucose <6.1 and 2 h-PLG<11.1). Two models to predict 2 h hour post load glucose (2 h-PLG) in the AusDiab study were then developed.

The analysis encompassed two distinct lipidome sets. 1) the full ~250 CLP model using a ridge regression, and 2) the ~50-subset CLP model using a LASSO model with a lambda limit to maintain ~50 lipids. Models were optimised to minimise the mean square error in predicting 2 h-PLG.

We first generated a single LASSO model containing ~50 lipid species and used this to establish the list of lipid species (Table 36).

To assess the performance of the Ridge and LASSO models, a 10-fold cross-validation framework was implemented to randomly partition the AusDiab dataset, repeatedly designating one-fold as the validation set and merging the remaining folds as the training set. Within each iteration of the training set, Ridge or LASSO regression was applied to establish the relationships between 2 h-PLG and the lipid sets. Continuous predictors underwent standardisation to achieve zero-mean and unit-variance.

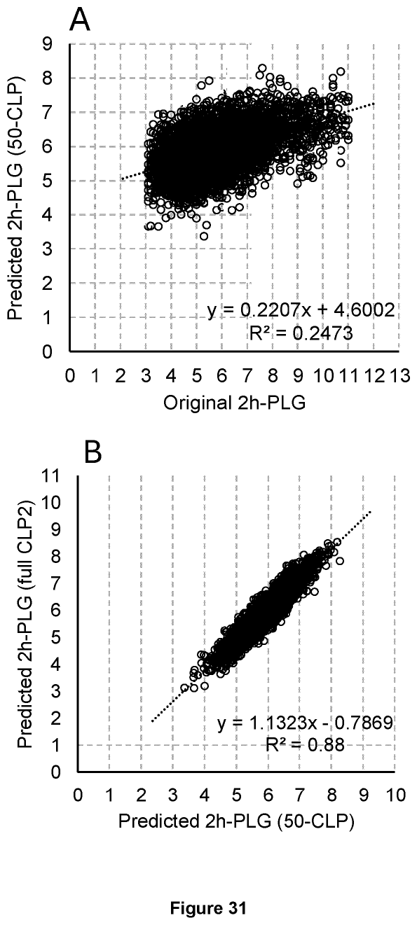

The correlation between the ~50-CLP predicted 2 h-PLG and the original (measured) 2 h-PLG had an R2=0.2473 (FIG. 31A). The correlation between the 50-CLP predicted 2 h-PLG and the ~250 CLP model predicted 2 h-PLG had a R2=0.88 (FIG. 31B).

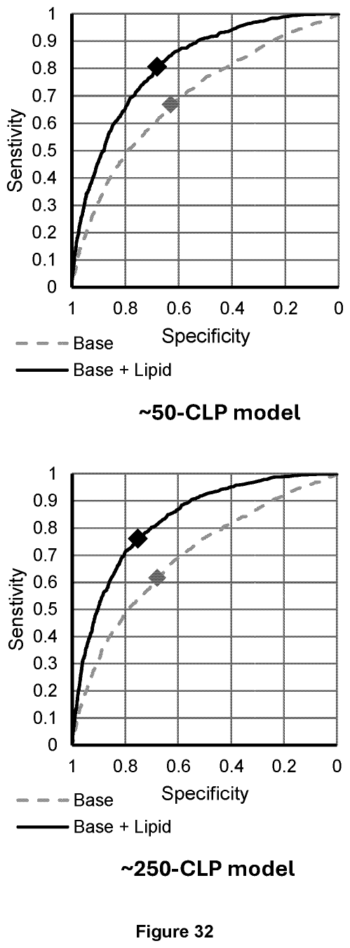

The ability for ~50-CLP 2 h-PLG to identify individuals at baseline that are likely to have impaired glucose tolerance was then assessed. Under the same 10-fold cross validation framework, the model to predict risk of impaired glucose tolerance was applied. Contrasting against the base model of traditional clinical factors (AUSDRISK, HbA1c and fasting glucose), a substantial improvement in predictive power is obtained (AUC of 0.695 vs 0.816, FIG. 32) corresponding to a NRI of 0.827, event NRI of 0.4185, and non-event NRI of 0.408 (Table 37). The cut-off derived with Youden's index highlights a positive predictive value improvement of 0.25, up from 0.19. Similarly, the negative predictive value increases from 0.94 to 0.96. Further contrasted against the original ~250 CLP lipid model (FIG. 32; Table 38), ~50-CLP demonstrated very similar performance to the ~250-CLP models.

TABLE 36
Predictive models of 2h-PLG developed using LASSO regression
with an approximately 50 lipid subset the CLP2 lipids

| Rank # | Lipid/Covariate | LASSO | LASSO (ABS) |
|---|---|---|---|
| 1 | LPC(18:2) | −2.35599 | 2.355992 |
| 2 | PC(40:8) | −1.21053 | 1.210533 |
| 3 | LPC(18:0) | 1.021479 | 1.021479 |
| 4 | AC(18:1) | 0.883297 | 0.883297 |
| 5 | AC(16:1) | 0.840505 | 0.840505 |
| 6 | LPE(22:6) | 0.813213 | 0.813213 |
| 7 | Cer(m18:0/22:0) | 0.701608 | 0.701608 |
| 8 | SHexCer(d18:1/16:0(OH)) | −0.68797 | 0.687968 |
| 9 | LPC(O-22:0) | −0.59574 | 0.595743 |
| 10 | TG(50:2) [NL-18:1] | −0.5084 | 0.508401 |
| 11 | TG(O-54:4) [NL-17:1] | 0.472333 | 0.472333 |
| 12 | TG(54:6) [NL-22:6] | 0.398753 | 0.398753 |
| 13 | TG(56:8) [NL-20:4] | 0.320235 | 0.320235 |
| 14 | Cer(d18:1/16:0) | 0.312991 | 0.312991 |
| 15 | Cer(d18:1/22:0) | 0.299865 | 0.299865 |
| 16 | PC(32:2) | 0.296901 | 0.296901 |
| 17 | Cer(d18:1/20:0) | 0.267326 | 0.267326 |
| 18 | TG(52:2) [NL-16:0] | −0.26399 | 0.263994 |
| 19 | PC(P-17:0/20:4) | −0.2369 | 0.236901 |
| 20 | S1P(d18:1) | −0.23643 | 0.236427 |
| 21 | DE(18:2) | 0.220666 | 0.220666 |
| 22 | CE(17:0) | −0.21946 | 0.219457 |
| 23 | AC(13:0) | −0.20177 | 0.20177 |
| 24 | PC(38:4) (b) | 0.201466 | 0.201466 |
| 25 | SHexCer(d18:1/16:0) | −0.19605 | 0.19605 |
| 26 | PI(16:0_20:4) | 0.194532 | 0.194532 |
| 27 | DE(20:4) | 0.189751 | 0.189751 |
| 28 | PC(16:0_16:0) | 0.141012 | 0.141012 |
| 29 | AC(14:1) | 0.132572 | 0.132572 |
| 30 | TG(54:3) [NL-18:1] | −0.13058 | 0.130579 |
| 31 | LPC(O-22:1) | −0.12162 | 0.121617 |
| 32 | PE(P-20:0/18:2) | −0.08141 | 0.081408 |
| 33 | LPC(26:0) | −0.07012 | 0.070122 |
| 34 | DG(16:0_16:0) | 0.06629 | 0.06629 |
| 35 | PC(P-16:0/20:5) | −0.06575 | 0.065755 |
| 36 | SM(d18:1/18:0) & | 0.054742 | 0.054742 |
| | SM(d16:1/20:0) | | |
| 37 | TG(50:3) [NL-14:1] | −0.04805 | 0.048054 |
| 38 | Hex2Cer(d16:1/16:0) | 0.036311 | 0.036311 |
| 39 | TG(54:6) [NL-20:4] | 0.030513 | 0.030513 |
| 40 | PE(16:0_22:6) | −0.02793 | 0.02793 |
| 41 | Cer(d18:2/24:0) | −0.00459 | 0.004591 |
| 42 | Cer(m18:1/20:0) | 0.003637 | 0.003637 |

TABLE 37
Sensitivity and Specificity in classifying individuals with
impaired glucose tolerance using the 50-CLP predicted 2h-PLG
Base model - AUSDRISK, HbA1c and fasting blood glucose,
Base + lipid model - AUSDRISK, HbA1c, fasting blood
glucose and the 50-CLP predicted 2h-PLG. PPV, positive
predictive value, NPV, negative predictive value, NGT,
normal tolerance, IGT, impaired glucose tolerance

| | Base | Base + Lipid |
|---|---|---|
| Sensitivity | 0.67 | 0.81 |
| Specificity | 0.63 | 0.68 |
| 1-Specificity | 0.37 | 0.32 |
| PPV | 0.19 | 0.25 |
| NPV | 0.94 | 0.96 |

| | | | | |
|---|---|---|---|---|
| True Negative | 4531 | 62.97% | 4898 | 68.08% |
| True Positive | 629 | 66.99% | 758 | 80.72% |
| False Positive | 2664 | 37.03% | 2297 | 31.92% |
| False Negative | 310 | 33.01% | 181 | 19.28% |

| | | |
|---|---|---|
| Total NGT | 7195 | 7195 |
| Total IGT | 939 | 939 |
| AUC | 0.695 | 0.816 |

TABLE 38
Sensitivity and Specificity in classifying individuals with impaired
glucose tolerance using the full CLP predicted 2h-PLG
Base model - AUSDRISK, HbA1c and fasting blood glucose, Base + lipid
model - AUSDRISK, HbA1c, fasting blood glucose and the 50-CLP
predicted 2h-PLG. PPV, positive predictive value, NPV, negative
predictive value, NGT, normal tolerance, IGT,
impaired glucose tolerance.

| | Base | Base + Lipid |
|---|---|---|
| Sensitivity | 0.62 | 0.76 |
| Specificity | 0.68 | 0.75 |
| 1-Specificity | 0.32 | 0.25 |
| PPV | 0.20 | 0.29 |
| NPV | 0.93 | 0.96 |

| | | | | |
|---|---|---|---|---|
| True Negative | 4896 | 68.05% | 5416 | 75.27% |
| True Positive | 580 | 61.77% | 715 | 76.14% |
| False Positive | 2299 | 31.95% | 1779 | 24.72% |
| False Negative | 359 | 38.23% | 224 | 23.85% |

| | | |
|---|---|---|
| Total NGT | 7195 | 7195 |
| Total IGT | 939 | 939 |
| AUC | 0.695 | 0.830 |

### REFERENCES

1. World Health Organization (WHO), Reducing the burden of noncommunicable diseases through strengthening prevention and control of diabetes. 2021.
2. Department of Health, Australian National Diabetes Strategy (2021-2030). 2021, Commonwealth of Australia, Canberra.
3. Meyer, C., et al., Different mechanisms for impaired fasting glucose and impaired postprandial glucose tolerance in humans. Diabetes Care, 2006. 29(8): p. 1909-14.
4. Campbell, M. D., et al., Benefit of lifestyle-based T2DM prevention is influenced by prediabetes phenotype. Nature Reviews Endocrinology, 2020. 16(7): p. 395-400.
5. Barry, E., et al., Efficacy and effectiveness of screen and treat policies in prevention of type 2 diabetes: systematic review and meta-analysis of screening tests and interventions. bmj, 2017. 356.
6. Bell, K., et al., A position statement on screening and management of prediabetes in adults in primary care in Australia. Diabetes Research and Clinical Practice, 2020. 164: p. 108188.
7. Meikle, P. J., et al., Plasma lipid profiling shows similar associations with prediabetes and type 2 diabetes. PLoS One, 2013. 8(9): p. e74341.
8. Wong, G., et al., Inclusion of plasma lipid species improves classification of individuals at risk of type 2 diabetes. PLoS One, 2013. 8(10): p. e76577.
9. Dunstan, D. W., et al., The Australian diabetes, obesity and lifestyle study (AusDiab)—methods and response rates. Diabetes research and clinical practice, 2002. 57(2): p. 119-129.

1. A method of determining metabolic health of a subject, the method comprising:
(i) detecting in a biological sample from the subject a level of at least about 50 lipid species selected from Table 4 and/or 6 and/or 30; and
(ii) comparing the levels of the lipid species detected in the biological sample to reference lipid species levels,
wherein the comparison is determinative of the metabolic health of the subject.

2. The method of claim 1, further comprising a step of assessing metabolic health in the subject based on the comparison of the lipid species in the biological sample to reference lipid species levels.

3. The method of claim 1 or 2, wherein metabolic health of the subject is at least one of:
(i) metabolic age;
(ii) metabolic body mass index (mBMI);
(iii) 2 hr post load glucose level;
(iv) plasmalogen level or plasmalogen relative to the phospholipid level;
(v) risk of developing cardiovascular disease; and
(vi) risk of diabetes.

4. The method of any one of claims 1 to 3, wherein the metabolic health of the subject is the metabolic body mass index (mBMI) of the subject, And wherein the comparison is determinative of the mBMI of the subject.

5. The method of claim 4, wherein at least 40 of the about 50 lipid species are selected from Table 32.

6. The method of claim 4 or claim 5, wherein the about 50 lipid species comprise the lipids in Table 32.

7. The method of any one of claims 4 to 6, further comprising a step of assessing mBMI in the subject based on the comparison of the lipid species in the biological sample to reference lipid species levels.

8. The method of any one of claims 1 to 3, wherein the metabolic health of the subject is the risk of developing cardiovascular disease in the subject, and wherein the comparison is determinative of the risk of developing cardiovascular disease in the subject, optionally further comprising calculating a cardiovascular disease (CVD) risk score of the subject,

9. The method of claim 8, wherein at least 40 of the about 50 lipid species are selected from Table 35.

10. The method of claim 8 or claim 9, wherein the about 50 lipid species comprise the lipids in Table 35.

11. The method of any one of claims 8 to 10 further comprising:
(i) standardising each continuous variable;
(ii) log transforming each lipid concentration; and
(iii) further standardising the variables prior to their use as the predictors of a CVD event.

12. The method according to any one of claims 8 to 11, further comprising a step of confirming that the subject has, or is likely to develop cardiovascular disease based on the comparison of the lipid species in the biological sample to reference lipid species levels.

13. The method of any one of claims 1 to 3 or 8 to 12, further comprising treating the subject if the subject has or is likely to develop cardiovascular disease.

14. The method according to claim 13, wherein the treatment comprises one or more of:
(i) a further diagnostic step, including any one or more of a blood test, electrocardiogram (ECG), exercise stress test, echocardiogram (ultrasound), nuclear cardiac stress test, coronary angiogram, magnetic resonance imaging (MRI), coronary computed tomography angiogram (CCTA) and a CT scan;
(ii) medication, including any one or more of blood-thinners, statins, beta blockers, nitrates, angiotensin-converting enzyme (ACE) inhibitors, angiotensin-2 receptor blockers, calcium channel blockers and diuretics; and
(iii) lifestyle changes, including any one or more of changes to diet, changes to exercise, increased aerobic activity, quitting or reducing smoking and weight loss.

15. The method of any one of claims 1 to 3, wherein the metabolic health of the subject is the risk of developing diabetes or impaired glucose tolerance in the subject, wherein the comparison is determinative of the risk of developing diabetes or impaired glucose tolerance in the subject.

16. The method of claim 15, wherein at least 35 of the about 50 lipid species are selected from Table 36.

17. The method of claim 15 or claim 16, wherein the about 50 lipid species comprise the lipids in Table 36.

18. The method according to any one of claims 15 to 17, further comprising a step of confirming that the subject is likely to develop impaired glucose tolerance and/or diabetes based on the comparison of the lipid species in the biological sample to lipid species reference levels, optionally further comprising calculating a diabetes or impaired glucose tolerance risk score.

19. The method of any one of claims 15 to 18, further comprising treating the subject if the subject is likely to develop diabetes or impaired glucose tolerance.

20. The method according to claim 19, wherein the treatment comprises any one or more of insulin pumps, islet cell transplant, supplements, medication, weight loss, weight loss surgery, dietary changes, exercise, insulin treatment, emotional support and mental health support.

21. The method according to any one of claims 15 to 20, wherein the diabetes is type II diabetes.

22. The method of any one of claims 1 to 3, wherein the metabolic health of the subject is the metabolic age of the subject, wherein the comparison is determinative of the metabolic age in the subject.

23. The method of claim 22, wherein at least 40 of the about 50 lipid species are selected from Table 33.

24. The method of claim 22 or claim 23, wherein the about 50 lipid species comprise the lipids in Table 33.

25. The method according to any one of claims 22 to 24, further comprising a step of confirming metabolic age in the subject based on the comparison of the lipid species in the biological sample to reference lipid species levels.

26. The method of any one of claims 1 to 25, wherein the method comprises detecting in a biological sample from the subject a level of at least 50 lipid species or at least 100 lipid species selected from Table 4 and/or 6.

27. The method of any one of claims 1 to 26, wherein the method further comprises detecting in the biological sample from the subject a level of at least one additional lipid species not defined in Table 4 and/or 6 and/or 30.

28. A method of calculating a metabolic health score of a subject comprising:
(i) obtaining lipid profile data from a biological sample taken from the subject, wherein the lipid profile data comprises at least about 50 lipid species selected from Table 4 and/or 6 and/or 30;
(ii) normalising the numeric values of the lipid profile data against a reference sample; and
(iii) refining the discriminatory power of one or more lipid species by statistically weighting one or several of the numeric values associated therewith according to a predetermined equation; and
(iv) normalising the resulting metabolic health score of the subject to a reference population.

29. A method of calculating a metabolic health score of a subject comprising:
(i) obtaining lipid profile data from a biological sample taken from the subject, wherein the lipid profile data comprises at least about 50 lipid species selected from Table 2, and/or 4 and/or 6 and/or 30;
(ii) normalising the numeric values of the lipid profile data against a reference sample; and
(iii) refining the discriminatory power of one or more lipid species by statistically weighting one or several of the numeric values associated therewith according to a predetermined equation;
(iv) optionally adding one or more additional, statistically weighted, risk factors into the model, taken from the list of, age, sex, BMI, waste circumference, diabetes status, cholesterol, glucose tolerance, HDL-cholesterol, triglycerides, blood pressure, blood pressure medication, fasting glucose, HbA1C, ethnicity, family history of disease, smoking status, exercise levels, diet, place of birth; and
(iv) normalising the resulting metabolic health score of the subject to a reference population.

30. The method of claim 28 or claim 29, wherein the lipid profile data comprises two or more lipid species selected from the group set forth in Table 3 and/or 5.

31. The method according to any one of claims 28 to 30, further comprising calculating the sum of the weighted lipids.

32. The method according to any one of claims 28 to 31, wherein the metabolic health score is at least one of:
(i) a metabolic age score;
(ii) a metabolic body mass index (mBMI) score;
(iii) a phospholipid level;
(iv) 2-hr post load glucose score;
(v) a plasmalogen score or plasmalogen relative to the phospholipid level;
(vi) a cardiovascular disease score; and
(vii) a diabetes risk score.

33. The method of any one of claims 1 to 32, wherein the biological sample is selected from the group consisting of blood, plasma, serum, dried blood spots and dried plasma spots.

34. A panel or kit for use in a method according to any one of claims 1 to 33, wherein the kit comprises one or both of:
(i) a set of stable labelled isotopes or non-physiological lipid standards for quantification of the lipid species;
(ii) reference plasma samples for standardisation of the resulting lipid measures.

35. A method of generating a lipid profile in a sample from a subject, the method comprising:
(i) obtaining lipid profile data from a biological sample taken from the subject, wherein the lipid profile data comprises at least about 50 lipid species selected from Table 4 and/or 6 and/or 30; and
(ii) normalising the numeric values of the lipid profile data against a reference sample,
wherein the discriminatory power of one or more lipid species can be refined by statistically weighting one or several of the numeric values associated therewith according to a predetermined equation; and
wherein the resulting metabolic health score of the subject can be normalised to a reference population.

36. A method of calculating a metabolic health score of a subject, the method comprising:
(i) refining a discriminatory power of one or more lipid species by statistically weighting one or several of the numeric values associated therewith according to a predetermined equation; and
(ii) normalising the resulting metabolic health score of the subject to a reference population,
wherein lipid profile data can be obtained from a biological sample taken from the subject, wherein the lipid profile data comprises at least about 50 lipid species selected from Table 4 and/or 6 and/or 30; and
wherein the numeric values of the lipid profile data can be normalised against a reference sample.

37. A lipidomics assay for determining metabolic health of a subject according to any one of claims 1 to 36, the method comprising:
(i) separating in a sample obtained from a subject the sample analytes by liquid chromatographic separation; and
(ii) analysing the analytes using a mass spectrometer;
wherein the total run time is less than 15 minutes; and
wherein the resulting data can accurately assign individual analytes to a single lipid species.

38. The lipidomics assay of claim 37, wherein the total run time is not more than 5 minutes.

39. The lipidomics assay of claim 37 or claim 38, wherein the biological sample is at least one of plasma, serum, dried blood spots, and dried plasma spots.
